(19) United States
(12) Patent Application Publication
(10) Pub. No.: US 2024/0101645 A1
(43) Pub. Date: Mar. 28, 2024

(54) MONOCLONAL ANTIBODIES AGAINST CORONAVIRUSES AND USES THEREOF

(71) Applicants: THE HENRY M. JACKSON FOUNDATION FOR THE ADVANCEMENT OF MILITARY MEDICINE, INC., Bethesda, MD (US); THE GOVERNMENT OF THE UNITED STATES, AS REPRESENTED BY THE SECRETARY OF THE ARMY, Fort Detrick, MD (US)

(72) Inventors: Shelly KREBS, Silver Spring, MD (US); Kayvon MODJARRAD, Silver Spring, MD (US); Nelson MICHAEL, Silver Spring, MD (US); Vincent DUSSUPT, Silver Spring, MD (US); Gina C. DONOFRIO, Silver Spring, MD (US); Samantha TOWNSLEY, Silver Spring, MD (US)

(21) Appl. No.: 18/273,309

(22) PCT Filed: Jan. 24, 2022
(86) PCT No.: PCT/US2022/013565

§ 371 Date: Jul. 20, 2023

Related U.S. Application Data
(60) Provisional application No. 63/140,763, filed on Jan. 22, 2021.
(60) Provisional application No. 63/194,095, filed on May 27, 2021.

(51) Int. Cl. C07K 16/10; A61P 37/04

(52) U.S. Cl. CPC C07K 16/1003; A61P 37/04; A61K 2039/505

(57) ABSTRACT

The present disclosure is directed to monoclonal antibodies, or antigen-binding fragments thereof, that bind to coronavirus spike proteins, including the spike protein of SARS-CoV-2. These antibodies demonstrate high affinity binding to epitopes on the coronavirus spike protein and/or broad cross-reactivity to the spike protein of various coronaviruses. Compositions comprising the anti-coronavirus antibodies, nucleic acids encoding for the antibodies, recombinant expression vectors, and host cells are also disclosed. The present disclosure is also directed to methods of diagnosing, preventing or treating coronavirus infections, including infections or disease caused by SARS-CoV-2.

### CROSS-REFERENCE TO RELATED APPLICATIONS

This application claims the benefit of, and relies on the filing date of, U.S. provisional patent application No. 63/194,095, filed 27 May 2021 and U.S. provisional patent application No. 63/140,763 filed 22 Jan. 2021, the entire contents of each of which are incorporated herein by reference.

### GOVERNMENT INTEREST

This invention was made with government support under W81XWH-18-2-0040 awarded by United States Army Medical Research and Materiel Command. The government has certain rights in the invention.

### SEQUENCE LISTING

This application contains a Sequence Listing which has been submitted electronically in ASCII format and is hereby incorporated by reference in its entirety. Said ASCII copy, created on 24 Jan. 2022, is named HMJ-174-PCT_SL.txt and is 608 kilobytes in size.

### FIELD

This application relates generally to antibodies against coronaviruses and methods of using the same to detect, prevent, or treat coronavirus infections.

### BACKGROUND

Coronaviruses (CoVs) refer to a family of enveloped, positive-sense, single-stranded, and highly diverse RNA viruses with four genera (alpha, beta, gamma, and delta). Among these genera, the α-coronavirus and β-coronavirus are capable of crossing animal-human barriers. The coronaviruses infecting humans (hCoVs) include the beta-genera CoVs, namely Severe Acute Respiratory Syndrome (SARS)-CoV-1, SARS-CoV-2, Middle East Respiratory Syndrome (MERS)-CoV (MERS-CoV), hCoV-HKU1, and hCoV-OC43 and the α-genera CoVs, hCoV-NL63 and hCoV-229E.

SARS-CoV-1 first emerged in Foshan, China in November 2002 and was subsequently transported to Hong Kong in February 2003, from where it spread globally. The epidemic was contained in July 2003 as the transmission chain of SARS-CoV-1 in Taiwan was interrupted. While there were four instances of SARS-CoV-1 reemergence that occurred chronologically in Singapore, Taipei, Guangdong and Beijing, SARS-CoV-1 infected human cases have not been reported since May 2004. However, in April 2012, MERS-CoV subsequently emerged in Jordan. Since it first emerged, MERS-CoV has been causing persistent endemics in countries within and outside the Middle East.

While the population continues to be threatened by MERS-CoV, SARS-CoV-2 has recently emerged and led to a global health crisis. On 30 Jan. 2020, the World Health Organization (WHO) declared SARS-CoV-2 to be a public health emergency of international concern and a pandemic on 11 Mar. 2020. SARS-CoV-2 first occurred in Wuhan, China in December 2019, after which it swiftly spread across China and as of January 2021 continues to aggressively infect people globally. New variants of SARS-CoV-2 have also emerged due to mutations in the viral genome, including new variants recently reported in Great Britain, South Africa, Brazil, and the United States.

While hCoV-HKU1, hCoV-OC43, hCoV-NL63 and hCoV-229E mainly cause asymptomatic or mild respiratory and gastrointestinal infections and account for approximately 5-30% of common colds, the effects of other hCoVs including SARS-CoV-1, MERS-CoV and SARS-CoV-2 can be more severe. For example, while symptoms of COVID-19 caused by SARS-CoV-2 are mostly mild, such as fever, coughing, and breathlessness, older adults and those with chronic diseases may experience severe symptoms, including severe pneumonia and organ dysfunction. As of 20 Jan. 2021, SARS-CoV-2 has infected more than 97,230,986 people worldwide, contributing to more than 2,080,350 deaths, with a mortality rate of 2.14%.

While vaccines are presently available against SARS-CoV-2, no therapeutics or vaccines have been approved for treating or preventing infections caused by other coronaviruses. Further, the approved SARS-CoV-2 vaccines may not be effective against all SARS-CoV-2 variants. Accordingly, there remains a need in the art for therapeutics, diagnostics, and vaccines that may be used to treat, diagnose, or prevent current and future coronavirus infections, including infections caused by SARS-CoV-2.

### SUMMARY

The present disclosure is directed to newly discovered human monoclonal antibodies, or antigen-binding fragments thereof, that bind to the spike protein of various coronaviruses, including one or more of SARS-CoV-1, SARS-CoV-2, Middle East Respiratory Syndrome (MERS)-CoV (MERS-CoV), hCoV-HKU1, hCoV-OC43, hCoV-NL63 and/or hCoV-229E.

The human antibodies are described, in part, by the amino acid sequence of their heavy and light chain variable regions, as well as the amino acid sequences of their complementarity determining regions (CDRs) and are referred to by the following designations: COV_1007, COV_1037, COV_1045, COV_1046, COV_1201, COV_2004, COV_2008, COV_2014, COV_2018, COV_2024, COV_2025, COV_2027, COV_2028, COV_2035, COV_2037, COV_2038, COV_2039, COV_2054, COV_2056, COV_2057, COV_2063, COV_2091, COV_2100, COV_2103, COV_2108, COV_2123, COV_2125, COV_2134, COV_2151, COV_2165, COV_2172, COV_2173, COV_2193, COV_2196, COV_3000, COV_3005, COV_3013, COV_3019, COV_3028, COV_3031, COV_3033, COV_3037, COV_3040, COV_3043, COV_3053, COV_3088, COV_1012, COV_1025, COV_1032, COV_1050, COV_1056, COV_1060, COV_1063, COV_1071, COV_1076, COV_1082, COV_1085, COV_1086, COV_1087, COV_1097, COV_1116, COV_1118, COV_1122, COV_1131, COV_1136, COV_1144, COV_1145, COV_1149, COV_1151, COV_1154, COV_1165, COV_1166, COV_1170, COV_1172, COV_1177, COV_1184, COV_1198, COV_2032, COV_2048, COV_2055, COV_2056, COV_2064, COV_2066, COV_2077, COV_2093, COV_2137, COV_2143, COV_2169, COV_2172, COV_2174, COV_2205, COV_2215, COV_3049, COV_3069, COV_3077, COV_3079, COV_3100, COV_3103, COV_3129, or COV_3137 antibody. These antibodies are also described by their functional properties, including one or more of:
(a) high affinity binding to the coronavirus spike protein, as measured, for example, by dissociation constant (K_{D}), such as a K_{D} less than 25 μM and optionally a K_{D} less than 15 pM, 10 pM, 5 pM, 2 pM, or 1 μM;
(b) cross-reactivity among different coronaviruses, including one or more of SARS-CoV-1, SARS-CoV-2, Middle East Respiratory Syndrome (MERS)-CoV (MERS-CoV), hCoV-HKU1, hCoV-OC43, hCoV-NL63 and/or hCoV-229E;
(c) ability to bind one or more epitopes in the coronavirus spike protein, including the receptor-binding domain of the S1 subunit, the N-terminal domain of the S1 subunit or the S2 subunit;
(d) ability to block coronavirus, including SARS-CoV-2, interaction with cellular receptors allowing these antibodies, individually or in combination, to be useful in methods for preventing infection or decreasing disease severity as described herein; and
(e) ability to neutralize coronavirus variants, including one or more of the circulating SARS-CoV-2 variants: B.1.1.7, B.1427/429, B 1.351, P.1, B.1.526a, and B.1,526b.

The broad cross-reactivity of some of the human antibodies disclosed herein, suggests that they recognize a conserved region of the coronavirus spike protein and, therefore, can be effectively used against coronavirus variants, such as existing SARS-CoV-2 variants, that have emerged due to natural mutations in the viral genome, as well as new variants that may emerge in the future.

Compositions comprising the anti-coronavirus antibodies and antigen-binding fragments thereof of the disclosure, nucleic acids encoding for the antibodies, recombinant expression vectors, host cells, and immunogenic compositions comprising an amino acid sequence to which the instant anti-coronavirus antibodies bind are also disclosed. The present disclosure is also directed to methods of diagnosing, preventing or treating coronavirus infections, including infections and disease caused by SARS-CoV-2, such as COVID-19.

### BRIEF DESCRIPTION OF THE DRAWINGS

The accompanying drawings, which are incorporated in and constitute a part of this specification, illustrate certain embodiments, and together with the written description, serve to explain certain principles of the compositions and methods disclosed herein.

FIGS. 1 (and FIG. 16) depict the organization of different structural motifs of a coronaviral spike protein as described in the detailed description.

FIGS. 2A-2J (and FIGS. 17a-17j as described in legend) depict the isolation of SARS-CoV-2 neutralizing antibodies from a convalescent donor as described in the Examples. 2A, Plasma neutralization against SARS-CoV-2 and from convalescent (C) and healthy (H) donors. Convalescent donor #3, darkened circle, was selected for B cell sorting based on high plasma neutralization against IL1/2020 and high magnitude binding antibodies to NTD, RBD, and S trimer measured in a multiplex bead-based assay. Bars indicate median value. 2B, The percentage of isolated monoclonal antibodies binding to subdomains of Spike (S). 2C, Neutralization potency of isolated WRAIR mAbs segregated by subdomain binding specificity. IC50=50% inhibitory concentration (μg ml⁻¹) from the SARS-CoV-2 (IL1/2020) pseudotyped assay. Shown are the mean IC50 values calculated from 3 independent experiments. 2D, Correlation between neutralization potency (IC50) for NTD-(left, n=14 XY pairs) and RBD-(right, n=18 XY pairs) directed mAbs and their respective binding magnitude to the SARS-CoV-2 stabilized S trimer, obtained from a single experiment. A significant (inverse) correlation was only observed for the WRAIR RBD-directed mAbs. Spearman r values are indicated above each graph with p values (two-tailed). 2E, 2F, Neutralization curves of the most potent NTD- and RBD-directed neutralizing antibodies as measured in the (2E) pseudotyped and (2F) authentic SARS-CoV-2 assays, using strains IL1/2020 and INMI1/2020, respectively, which share identical S sequence. Plotted are mean±s.e.m from 3 (2e) or 2 (2f) independent experiments. The IC50 (pg ml⁻¹) for each mAb is indicated in parenthesis and calculated using a 5-parameter regression analysis. 2G, Correlation between the pseudotyped (pSV) and authentic virus assays, n=24 XY pairs. Spearman r value is indicated above the graph with p value (two-tailed). 2H, NTD and RBD WRAIR mAb binding to cell-surface expressed S using 293F cells as measured by flow cytometry. Black line indicates mean value and asterisks represent significance by two-tailed Mann-Whitney t-test, p=0.0009. Dotted line indicates positivity threshold. 2I, 2J, Assessment of NTD and RBD mAbs recruitment of (2I) Fc-mediated complement (ADCD) and (2J) phagocytic activities (ADCP and ADNP). ADCD was measured using a S-expressing 293F cell line whereas phagocytic activities were determined using the stabilized S trimer. Black horizontal line indicates mean value and asterisks represent significance by two-tailed Mann-Whitney t-test, p<0.0001. Dotted line indicates positivity threshold. 2H-2J, shown are representative data (n=2) from a single experiment. In panels 2C, 2D, 2H-2J, neutralizing and non-neutralizing mAbs are in closed and open circles, respectively, while the control RBD mAb CR3022 is shown as a gray closed circle in panels 2H-2J.

FIGS. 3A-3C (and FIGS. 18a-18c as described in legend) depict epitope mapping and structural characterization of WRAIR NTD mAbs as described in the Examples. 3A, epitope binning of NTD-directed mAbs via a Bio-Layer Interferometry (BLI)-based competition assay. Values represent the % residual binding of the indicated second antibody after saturation of the antigen (NTD domain) with the indicated first antibody. Shading from dark to light indicates competition strength ranging from strong (0-25%), to intermediate (25--50%), to none (>50%). Competition groups are indicated by boxes. 3A (CONT.), binding responses of NTD-directed mAbs, segregated by competition group, to the stabilized S trimer measured by BLI. 3B, epitope mapping of NTD A mAbs using a shotgun mutagenesis platform. Heat map shows % binding to NTD mutants, harboring a single change to Alanine at the indicated position, relative to wild-type. 3B (CONT.), The NTD (residues 14-303) is shown in the context of SARS-CoV-2 trimer (PDB 6ZGE) with loops N1, N3 and N5 colored in shades of grey. 3B, CONT. 2 and 3B (CONT. 3), Key binding residues are shown on the NTD structure with side chains shown in stick representation. 3C, Residues identified in the viral escape assay in the presence of NTD antibodies at the indicated concentrations. 3C (CONT.), The same residues are shown in stick representation on the NTD structure.

FIGS. 4A-4G (and FIGS. 19a-19g as described in legend) depict the structure and epitope determination of SARS-CoV-2 RBD targeting mAbs as described in the Examples. 4A, Epitope binning of RBD-directed mAbs via a BLI-based competition assay. Values represent the % residual binding of the indicated second antibody after saturation of the antigen (RBD molecule) with the indicated first antibody. Shading from dark to light indicates competition strength ranging from strong (0-25%), to intermediate (25-50%), to lack thereof (>50%). Competition groups are indicated by black boxes. Control antibodies RBD-A, RBD-B and RBD-C were CC12.1, CC12.16 and CR3022, respectively. 4B-4D, Top panel: Representative crystal structures of RBD targeting antibodies for WRAIR RBD group A, B and C are shown. RBD A mAbs, WRAIR-2125 and WRAIR-2173 target the ACE2 binding site. RBD B mAb, WRAIR-2057 recognizes a novel epitope on the “side” of the RBD distal from the ACE2 binding site centered on residue E465. RBD C mAb, WRAIR-2151 targets a CR3022-like site on the RBD. Bottom panel: Epitope footprints of respective antibodies are shown on the surface of the RBD and shaded based on the antibody heavy and light chain colors. RBD contacting residues are shown as sticks, with residues seen in VOCs highlighted in bold. 4E, Structures of WRAIR RBD A. B and C antibodies are shown on a single RBD molecule to highlight the different recognition modes. 4F, RBD A, B and C epitopes are shown on the RBD surface with the ACE2 binding interface highlighted in black/white line. 4G, Epitope mapping of WRAIR-2125 and-2173 contact residues identified in the shotgun mutagenesis and viral escape experiments, or both are shown in stick representation.

FIGS. 5A-5C (and FIGS. 20a-20c as described in legend) depict WRAIR mAbs showing low-dose prophylactic protection in the K-18 hACE-2 mouse model as described in the Examples. 5A-5C, Antibodies were infused intravenously at a single high dose of 400 pg (20 mg kg⁻¹) (5A, 5A (CONT., CONT. 2)) or low doses of 20 μg (1 mg kg⁻¹) and lower (5B, 5C) into groups of mice (n=15/group). Mice were challenged intranasally 24 hours later with 1.25×10⁴ viral particles (1.25×10⁴ plaque-forming units) of SARS-CoV-2 (WA1/2020). SARS-CoV-2 viral loads in lung tissue were measured 2 days post-challenge in a subset of animals (n==5/group) by plaque assay. Bars indicate the mean group value with standard deviation. The remaining mice (n=10/group) were assessed daily for weight and clinical symptoms. 5C, Assessment of Fc effector functions on animal protection for NTD and RBD antibodies. Wild-type and LALA-PG versions of mAb WRAIR-2039 (NTD) and WRAIR-2123 (RBD) were compared at 20 μg (1 mg kg-r). For weight loss and viral load in lungs, asterisks indicate significance compared to the ZIKV_MZ4 monoclonal antibody isotype control group, by one-way ANOVA with Dunnett's multiple comparisons test. Survival curves were compared individually to the isotype control using a Mantel-Cox log-rank test. For all tests, ****P<0.0001, ***P<0.001, **P<0.01, *P<0.5 and ns: not significant (P>0.5).

FIGS. 6A-6F (and FIGS. 21a-21f as described in legend) depict NTD/RBD mAb combinations showing low dose in vivo protection and a higher genetic barrier for viral escape as described in the Examples. 6A, Binding competition to the stabilized trimer as described in FIG. 2a. 6B, Negative-stain 3D reconstruction of SARS-CoV-2 spike in complex with WRAIR-2025 (NTD) and WRAIR-2173 (RBD) Fabs. 6C, Prophylactic treatment in the K-18 hACE-2 SARS-CoV-2 mouse model. Antibodies were infused intravenously at a dose of 20 μg (1 mg kg⁻¹) as single mAbs or combinations (1:1 ratio) into groups of mice (n=15/group). Mice were challenged intranasally 24 hours later with 1.25×10⁴ viral particles (1.25×10⁴ plaque-forming units) of SARS-CoV-2 (WA1/2020). SARS-CoV-2 viral loads in lung tissue were measured 2 days post-challenge in a subset of animals (n=5/group) by plaque assay. Bars indicate the mean group value with standard deviation. The remaining mice (n=10/group) were assessed daily for weight and clinical symptoms. 6E, Therapeutic treatment in the K-18 hACE-2 SARS-COV-2 mouse model. Antibodies were infused intravenously at the indicated dose 24 hours after challenge, performed as indicated above. Mice (n=15/group) were assessed daily for weight and clinical symptoms. 6D-6E, For weight loss and viral load in lungs, asterisks indicate significance compared to the ZIKV_MZ4 isotype control group by one-way ANOVA with Dunnett's multiple comparisons test. Survival curves were compared individually to the isotype control using a Mantel-Cox log-rank test. For all analysis, ****P<0.0001, ***P<0.001, **P<0.01, *P<0.5 and ns: not significant (P>0.5). 6F, Viral titers of a replicative rVSV/SARS-CoV2/GFP virus obtained after two passages in the presence of single mAbs or combinations. Plotted are means from 2 independent experiments.

FIGS. 7A-7D (and FIGS. 22a-22d as described in legend) depict WRAIR mAb binding and neutralization against current circulating variants of concern (VOC) as described in the Examples. 7A, Binding of NTD-(7A) and RBD-(7a (CONT.) left) mAbs to stabilized S trimer (S-2P) or RBD mutants (7A (CONT.), right) harboring mutations present in VOC and VOI assessed by BLI. Heat-map shows the log 2 fold change in binding relative to a WA1/2020 D614G S-2P spike or WA1/2020 RBD proteins with loss and gain in binding. 7B, Neutralization activity of NTD and RBD mAbs, either singly or in combinations, against a panel of pseudotyped viruses representing the current circulating VOCs. Heat-map indicates IC50 values (μg ml⁻¹) ranging from very potent (hatched), to intermediate (light dots), to poorly neutralizing (black), with non-neutralizing mAbs in white. 7C, Same data as in (7B) but represented as fold change in IC50 relative to the IL1/2020 virus. Neutralization escape is defined as a fold increase in IC50 >100. 7D, Comparison of epitopes (outlined) between WRAIR-2125, S2E12, REGN10933 and LY-CoV555 with RBD neutralization escape residues shown in stick representation. FIG. 7D (CONT.), left, WRAIR-2125 and S2E12 heavy and light chain CDR loop contact residues are shown in ribbon representation, with the RBD shown in surface representation, residue F486 is indicated. FIG. 7D (CONT.) right, Antibody buried surface areas for the four RBD residues that differ in VOC/VOI.

FIGS. 8A-8D (and FIGS. 23a-23d as described in legend) depict the serology of convalescent donors and the sorting strategy used to isolate SARS-CoV-2 reactive B cells as described in the Examples. 8A, Correlation of plasma binding magnitude and neutralization potency of convalescent COVID-19 donors. Spearman r and p (two-tailed) values are indicated above each graph. n=41 XY pairs for each of the test. 8B, Antigens used to sort SARS-CoV-2 positive B cells. Two sorting strategies were performed using either a stabilized S trimer (HexaPro) or a multivalent Spike ferritin nanoparticle (SpFN) displaying 8 Spike trimers, to isolate antibodies targeting conformational or quaternary epitopes, in addition to S1, RBD and S2 SARS-CoV-2 subdomain antigens (FIG. 8B, CONT.). 8C, Gating strategy used to sort antigen⁺ B cells with the percentage of SARS-CoV-2 antigen positive B cells from a pre-pandemic donor and SARS-CoV-2 convalescent Donor #3 obtained with the two complementary sorting approaches, using the stabilized S trimer (8C (CONT.)) or SpFN (FIG. 8C (CONT2.). 8D, Individual CD19+ SARS-CoV-2 reactive B cells encoding SARS-CoV-2 neutralizing mAbs indicated in the flow cytometry plots for the stabilized S trimer (FIG. 8D (CONT.), left) and SpFN nanoparticle (FIG. 8D (CONT.)) sorts.

FIGS. 9A-9D (and FIGS. 24a-24d as described in legend) depict the genetics and cross-reactivity of characterized WRAIR NTD and RBD mAbs as described in the Examples. 9A, Gene assignment was performed with IgBlast and CDRs were annotated using IMGT. SHM, somatic hypermutation; CDR, complementarity determining region. Sequence identifiers corresponding to the CDRs depicted in the table are shown. Sequence identifiers corresponding to the CDRs depicted in the table are shown. SEQ ID NOS: 1109-1138 correspond to CDRH1, SEQ ID NOS: 1139-1168 correspond to CDRH2; SEQ ID NOS: 1169-1198 correspond to CDRH3; SEQ ID NOS: 1199-1228 correspond to CDRL1; SEQ ID NOS: 1229-1258 correspond to CDRL3. IC50 values from the pseudovirus neutralization assay, as well as competition groups are indicated. Only two clonally related mAbs were identified, WRAIR-2008 and-2037. 9B, 9C, Binding of WRAIR NTD (9B) and RBD (9C) mAbs to a panel of 26 human CoV and HIV control (gp140 and gp41) antigens, assessed in a multiplex bead-based assay. Binding magnitude is expressed as signal/noise (S/N) ratio, with noise calculated from an HIV mAb antibody control (MZ4). S/N≥10 (dotted line) were considered positive based on negative control binding. 9D, SARS-CoV-1 (Sinol-11) neutralization activity in a pseudotyped assay. Data are mean from two independent experiments. WRAIR-2063 is indicated with the IC50 (μg ml⁻¹) indicated in parenthesis. The CR3022 positive control is shown. Other WRAIR RBD and NTD nAbs that did not neutralize SARS-CoV-1 are also depicted.

FIGS. 10A-10C (and FIGS. 25a-25c as described in legend) depict the binding affinity and functional characteristics of WRAIR mAbs against SARS-CoV-2 IL1/2020 as described in the Examples. 10A, Binding affinity constants of WRAIR RBD and NTD mAbs measured against their respective domains using BLI. At least 4 curves from a dilution series were used to calculate the equilibrium dissociation constant (KD) using a 1:1 binding model. KD values are shown. 10B, Neutralization curves of WRAIR RBD (10B) and NTD (10B (CONT.)) mAbs obtained in the pSV assay. Shown are means from at least 2 independent experiments. Error bars were omitted for clarity. 10C, Comparison of neutralization activities between IgG1 and Fabs. Potent NTD- or RBD-neutralizing antibodies were assessed for neutralization in the PSV assay either as IgG1 or Fabs. Shown is the fold increase in IC50 observed with the Fab versions of each mAb compared to its IgG1 counterpart. IC50 (pg ml⁻¹) values obtained with Fabs are indicated in parentheses.

FIGS. 11A-11C (and FIGS. 26a-26c as described in legend) depict the Fc effector functions of WRAIR mAbs against SARS-CoV-2 IL1/2020 as described in the Examples. 11A, Titrations of WRAIR NTD (11A and 11A (CONT.)) and RBD mAbs (11A (CONT. 2) and 11A (CONT. 3)) in the Fc effector function assays. Antibodies are shaded according to their competition groups. Fc effector functions were measured twice and shown are data from a single representative experiment. A Fc mutant control (LALA-PG) is shown for reference. 11B, Correlation between neutralization activity and phagocytic activities for all mAbs. Spearman r and p (two-tailed) values are indicated above the graph, n=31 XY pairs. 11C, Difference in ADNP score between NTD neutralizing and non-neutralizing mAbs. Black horizontal line indicates mean value and asterisks represent significance by two-tailed Mann-Whitney t-test, p=0.0006, n=7. Dotted line indicates positivity threshold.

FIGS. 12A-12G (and FIGS. 27a-27g as described in legend) depict epitope mapping and structural characterization WRAIR RBD mAbs as described in the Examples. 12A, ACE2 blocking activity of WRAIR RBD mAbs. WRAIR RBD A (12A) and B and C (12A (CONT.)). mAbs were assessed for their ability to block hACE2 binding to SARS-CoV-2 RBD in a BLI-based assay. The half maximal effective concentration (EC50) in μg ml⁻¹ is indicated in parentheses. 12B-12D, Details of RBD A (WRAIR-2125 and-2173) (12B), RBD B (WRAIR-2057) (12C) and RBD C (WRAIR-2151) (12D) epitopes. Antibody residues are shown in stick representation and RBD residues are shown in line representation. Contributing heavy and light chain CDRs are shown and labelled. CDR loops are designated using the Kabat numbering system. 12E, Epitope mapping of RBD A mAbs using a shotgun mutagenesis platform. Heat map shows % binding to RBD mutants, harboring a single change to Alanine at the indicated position, relative to wild-type. 12F, Residues identified in the viral escape assay in presence of RBD antibodies at the indicated concentrations. Asterisks indicate mutations found only in half of the sequences obtained. 12G, Structures of WRAIR RBD A, B and C antibodies are overlaid on previously reported antibodies (representing frequently observed SARS-CoV-2 epitopes) (Rapalog et al., Science, 2021, 371: 823-829). 12G and 12G (CONT.), left panel: WRAIR-2057 antibody and epitope is shown on the RBD surface in the context of previously reported antibody classes. 12 G (CONT.), right panel: WRAIR-2125, WRAIR-2173 and WRAIR-2151 are shown with representative Class 1, 2, 3 and 4 mAbs.

FIGS. 13a-13b (and FIGS. 28a-28c as described in legend) depict structures as described in the Examples. 13A, Left panel: C002 structure is overlaid onto the WRAIR-2125 structure. 13A, right panel: Frequently occurring SARS-CoV-2 VOC residues are shown as sticks on the RBD surface with WRAIR-2125 and C002 epitopes indicated. 13A (CONT.): Buried surface area (BSA) for VOC residues, related to the mAbs WRAIR-2125 and C002 are shown as dot plots. T symbol is used to designate the “tip” of the RBD molecule. 13C, MAb CV38-142 structure is overlaid onto the WRAIR-2057-RBD complex structure.

FIGS. 14A-14D (and FIGS. 29a-29d as described in legend) depict the characterization of LALA-PG mutant mAbs and negative stain electron microscopy of Spike-Fab complexes. 14A, Characterization of the LALA-PG mutant forms of the NTD mAbs WRAIR-2039, 2025 and 2004 (circles) and RBD mAbs WRAIR-2173 and 2123 (squares) in cell surface S binding (left), ADCD (middle) and ADNP (right) assays. Non-significant differences in binding to cell surface S was observed between WT and mutants in a Wilcoxon matched-pairs signed rank test, P==0.3125. LALA-PG mutants did not show any activity in the ADCD and ADNP assays. 14B, Neutralization curves of WT and LALA-PG versions of WRAIR-2039 (NTD) and-2123 (RBD) mAbs obtained in the pSV assay. Shown are mean SD from at least 2 independent experiments. IC50 (μg ml⁻¹) are indicated in parentheses in the legend. 14C, EM analysis of WRAIR-2173 and WRAIR-2025 Fabs in complex with SARS-CoV-2 spike (S-2P) trimer. Left panel: Raw image (top) and two-dimensional class particle averages (bottom, 5 averages shown). The black bar represents 500 Å. Right panel: Gold-standard FSC curves for the EM 3D reconstruction. 14C (CONT.): Negative-stain 3D reconstruction of SARS-CoV-2 spike and Fab complex. The structural model of the SARS-CoV-2 trimer (PDB 6X2B) in complex with WRAIR-2173 and WRAIR-2025 Fabs is shown in ribbon representation while negative-stain electron density map is shown as a white transparent surface. A feature less and unbiased (lowpass filter: 100 Å) 3D model of SARS-CoV-2 spike trimer (PDB: 6VXX) and 12,574 particles were used to perform the 3D reconstruction from a single experiment. 14D, EM analysis of WRAIR-2025 Fab in complex with SARS-CoV-2 spike (S-2P) trimer. Left panel: Raw image (top) and two-dimensional class averages (bottom, 5 averages shown) of Fab-Spike particles. The black scale bar represents 500 Å. Right panel: Gold-standard FSC curves for the EM 3D reconstruction. 14D (CONT).: Negative-stain 3D reconstruction of SARS-CoV-2 Spike and Fab complex. The structural model of the SARS-CoV-2 trimer (PDB 6VXX) and WRAIR-2025 Fab is shown in ribbon representation while negative-stain electron density map is shown as a white transparent surface. A feature less and unbiased (lowpass filter: 100 Å) 3D model of SARS-CoV-2 spike trimer (PDB: 6VXX) and 3,364 particles were used to perform the 3D reconstruction from a single experiment.

FIGS. 15A-B (and FIGS. 30a-30b as described in the legend) depict the functional activities of WRAIR mAb combinations against SARS-CoV-2 IL1/2020 and characterization of variant binding as described in the Examples. 15A, Functional activities of NTD/RBD (FIG. 15A) and RBD/RBD (FIG. 15A (CONT.) mAb combinations. WRAIR-2039, -2025 (NTD) and WRAIR-2123, -2125 and -2173 (RBD) were mixed in a 1:1 ratio as indicated and the cocktail was assessed for neutralization (pSV assay) and Fc effector functions. Single mAbs or cocktail were tested at the same final antibody concentration. IC50 values (μg ml⁻¹) obtained in the pSV assay from 2 independent experiments are indicated in parentheses. ADCD and ADNP activities were measured twice and shown are data from a single representative experiment. The WRAIR-2039 LALA-PG negative control is shown as open circles and dotted line. 15B, Effect of mutations present in VOC and VOI on the binding on-rate and off-rate of NTD-directed mAbs. Binding on-rates (left) and off-rates (right) to stabilized S trimer (S-2P) harboring mutations present in the indicated variant was assessed by BLI. On- and off-rates were obtained by fitting the binding curves of mAbs at a single concentration of 200 nM using a 1:1 binding model. Heat-map shows the log 2 fold change relative to a WA1/2020 D614G S-2P spike protein with negatively impacted mAbs (by either a decrease in on-rate or increase in off-rate) as shown. mAb with improved binding kinetics, compared to the WT are shown. NB indicates absence of value due to lack of binding.

### DETAILED DESCRIPTION

Reference will now be made in detail to various exemplary embodiments, examples of which are illustrated in the accompanying drawings. It is to be understood that the following detailed description is provided to give the reader a fuller understanding of certain embodiments, features, and details of aspects of the disclosure, and should not be interpreted as a limitation of the scope of the disclosure.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

The term “antibody” or “antibodies” as used in this disclosure refers to an immunoglobulin or an antigen-binding fragment thereof As will be understood by those in the art, the immunological binding reagents encompassed by the term “antibody” or “antibodies” extend to all antibodies from all species, and antigen binding fragments thereof and include, unless otherwise specified, polyclonal, monoclonal (mAb or mAbs), monospecific, bispecific, polyspecific, humanized, human, camelised, mouse, non-human primates, single-chain, chimeric, synthetic, recombinant, hybrid, mutated, CDR-grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda.

The terms “antigen-binding domain” and “antigen-binding fragment” refer to a part of an antibody molecule that comprises amino acids responsible for the specific binding between antibody and antigen. For certain antigens, the antigen-binding domain or antigen-binding fragment of an antibody molecule may only bind to a part of the antigen. The part of the antigen that is specifically recognized and bound by the antibody is referred to as the “epitope” or “antigenic determinant.” Antigen-binding domains and antigen-binding fragments include Fab (Fragment antigen-binding); a F(ab′)₂ fragment, a bivalent fragment having two Fab fragments linked by a disulfide bridge at the hinge region; Fv fragment; a single chain Fv fragment (scFv) see e.g., Bird et al. (1988) Science 242:423-426; and Huston et al. (1988) Proc. Natl. Acad. Sci. USA 85:5879-5883); a Fd fragment having the two V_{H} and CHI domains; dAb (Ward et al., (1989) Nature 341:544-546), and other antibody fragments that retain antigen-binding function. The Fab fragment has V_{H}-C_{H}1 and V_{L}-C_{L} domains covalently linked by a disulfide bond between the constant regions. The Fv fragment is smaller and has V_{H} and V_{L} domains non-covalently linked. To overcome the tendency of non-covalently linked domains to dissociate, a scFv can be constructed. The scFv contains a flexible polypeptide that links (1) the C-terminus of V_{H} to the N-terminus of V_{L}, or (2) the C-terminus of V_{L} to the N-terminus of V_{H}. These antibody fragments are obtained using conventional techniques known to those with skill in the art, and the fragments are evaluated for function in the same manner as are intact antibodies.

The terms “(cross)-binding” and “(cross)-reactivity” are used interchangeably herein to mean the ability of an antibody to bind to similar target on multiple Coronaviruses. The extent to which one antibody is able to bind with another coronavirus target, and therefore whether it can be said to cross-bind, as used herein, can be determined using binding assays to multiple antigens from multiple coronaviruses. One particularly suitable quantitative cross-binding assay is described in the Examples. Briefly, binding of monoclonal antibodies (mAbs) to SARS-CoV-2, SARS-CoV, MERS-CoV, hCoV-HKU1, hCoV-OC43, hCoV-NL63 and hCoV-229E epitopes was evaluated using a multiplex Luminex assay. Antigens from were covalently coupled to uniquely coded carboxylated magnetic microspheres (Luminex Corp., Austin TX), and microspheres were activated by incubation in buffer containing 1-Ethyl-3[3-dimethylaminopropyl]carbodiimide hydrochloride and N-hydroxysulfosuccinimide for 20 min. Following activation, beads were incubated with antigen or streptavidin for 2 hr to allow coupling via the primary amine. Biotinylated antigens were then bound to streptavidin-coated microspheres for 2 hr followed by addition of free biotin to quench the reaction. Following coupling, coated microspheres were washed and stored at −80° C. in PBS containing 0.1% BSA, 0.05% sodium azide and 0.02% Tween-20. Purified mAbs were incubated with a cocktail of coronavirus antigen-coupled beads for 2 hours at room temperature. Following 3 washes with PBS containing 0.1% BSA, 0.05% sodium azide and 0.02% Tween-20, bound antibodies were detected by incubation R-phycoerthrin (PE)-conjugated mouse anti-human IgG (0.5 μg/mL, Southern Biotech, Birmingham AL) for 1 hour at room temp followed by a final wash and resuspension in Luminex® sheath fluid (Luminex Corp). Samples were assayed on a FlexMap3D® with xPONENT v4.2 software (Luminex Corp). CR3022, P2B-2F6, and VRC01 mAbs were included on each assay plate in addition to no-sample control wells. For each antigen assayed, the median fluorescent intensity (MFI) from samples (signal) was normalized by dividing by the MFI of no-sample wells (noise). If the signal:noise ratio was greater than 5 or 10, mAbs were reported as positive for cross-antigen binding.

The term “Epitope binning” is a term used to describe segmentation of a panel of monoclonal antibodies into bins based upon the antigen region, or epitope, bound by each antibody. This grouping is performed using cross competition assays. One particularly suitable quantitative cross-competition assay is described in the Examples. Briefly, epitopes of monoclonal antibodies are mapped by binding competition against e.g., a set of characterized control antibodies (RBD) using Bio-Layer Interferometry (BLI). Streptavidin (SA) sensors (ForteBio) loaded with either biotinylated antigen proteins are immersed into wells containing a first competing antibody to saturate all binding sites. Next, biosensors are dipped into wells containing the second antibody, in the presence of the first competing antibody (all at 100 nM), and then binding is measured after time for association. Residual binding signal of ae second antibody is expressed as a percentage of the maximum binding signal obtained in absence of the first competing antibody, ran in parallel. Antibodies are defined as competing when a binding signal of the second antibody is reduced to less than 30% of its maximum binding capacity and non-competing when binding was greater than 70%.

As used herein, a “therapeutically effective amount” of an antibody refers to an amount of an antibody that is effective, upon single or multiple dose administration to a subject (such as a human patient) at treating and/or preventing coronavirus infection.

The terms “treatment of a coronavirus infection” or “treating a corona virus infection” and the like refer to any treatment of any disease (e.g., COVID-19) or condition in a subject caused by a coronavirus infection and includes inhibiting a disease, condition, or symptom of a coronavirus infection, e.g., arresting its development and/or delaying or preventing its onset or manifestation in the subject; relieving a disease, condition, or symptom of a coronavirus, e.g., causing regression of the condition or disease and/or one or more of its symptoms (e.g., fever, shortness of breath); or preventing or reducing the recurrence or relapse of a disease, condition, or symptom of a coronavirus infection.

The term “coronavirus infection” refers to an infection due to a coronavirus including an infection due to SARS-CoV-1, SARS-CoV-2, Middle East Respiratory Syndrome (MERS)-CoV (MERS-CoV), hCoV-HKU1, and hCoV-OC43 and the α-genera CoVs, hCoV-NL63 and hCoV-229E.

The term “coronavirus” refers to any coronavirus, e.g. a human coronavirus, such as SARS-CoV-1, SARS-CoV-2, Middle East Respiratory Syndrome (MERS)-CoV (MERS-CoV), hCoV-HKU1, and hCoV-OC43 and the α-genera CoVs, hCoV-NL63 and hCoV-229E.

The term “COVID-19” refers to coronavirus disease 2019, the disease caused by SARS-CoV-2.

The terms “subject,” “host,” “patient,” and “individual” are used interchangeably herein to refer to any mammalian subject for whom diagnosis or therapy is desired, particularly humans.

The term “pharmaceutically acceptable excipient” means solvents, diluents, dispersion media, coatings, antibacterial agents and antifungal agents, isotonic agents, solid and liquid fillers, and absorption delaying agents, and the like, that are suitable for administration into a human. The use of such media and agents for pharmaceutically active substances is well known in the art.

The term “human antibody” refers to an antibody having variable and constant regions corresponding substantially to human germline immunoglobulin sequences. A human antibody may also include amino acid residues not encoded by human germline immunoglobulin sequences (e.g., mutations introduced by random or site-specific mutagenesis in vitro or by somatic mutation in vivo), for example in the CDRs, and in particular, CDR3. As disclosed herein, a human antibody may be produced using recombinant methods.

The term “recombinant antibody” refers to an antibody produced or expressed using a recombinant expression vector, where the expression vector comprises a nucleic acid encoding the recombinant antibody, such that introduction of the expression vector into an appropriate host cell or transgenic animal results in the production or expression of the recombinant antibody.

As is known in the art, recombinant antibodies, are not merely proteins isolated from a human donor, but are proteins that are produced in a host cell or transgenic animal. Appropriate host cells and suitable transgenic animals for production of the antibodies of the disclosure are described in Gene Expression Systems, Academic Press, eds. Fernandez et al., 1999. Suitable production hosts include yeast, mammalian, bacterial or insect cells or transgenic animals such as transgenic Drosophila or mice. The recombinant antibodies of the disclosure are glycosylated. The amount of glycosylation by weight for the IgG, IgM, IgA, IgD and IgE is typically about 3% a 12%, 10%, 13% and 12%, respectively. The glycosylation pattern of a recombinant human protein varies from the glycosylation pattern of its natural human protein counterpart since glycosylation is dependent upon the type of host cell or organism used to express the recombinant protein.

As is also known in the art, the glycosylation patterns of recombinant antibodies are not the same as those of any existing natural counterparts, even when the antibodies are expressed in human cells. See Nallet et al., New Biotechnology, 2012, 29: 471-476 who report that IgG expressed in a human embryonic kidney cell line results in similar, but not identical, glycosylation patterns in comparison to those expressed in humans. Further, Luac et al., Biochimica et Biophysica Acta, 2015, 1860: 1574-1582 reports that variation in glycosylation patterns for IgG differ between and within humans. Accordingly, the recombinant monoclonal antibodies of the instant disclosure are structurally distinguishable from antibodies obtained from human donors.

The term “neutralizing antibody” refers to an antibody whose binding an antigen results in inhibition of the biological activity of that antigen, respectively. For example, a “coronavirus neutralizing antibody “or “a SARS-CoV-2 neutralizing antibody” refers to an antibody whose binding to a coronavirus, such as SARS-CoV-2, results in the inhibition of the biological activity of the coronavirus. This inhibition of the biological activity of coronavirus, such as SARS-CoV-2 can be assessed by measuring one or more indicators of coronavirus activity biological activity, such as an ability to enter host cells using, e.g. a plaque assay as known in the art.

The term “isolated antibody,” refers to an antibody that is substantially free of its natural environment, including other antibodies having different antigenic specificities (e.g., an isolated antibody that specifically binds a coronavirus, such as SARS-CoV-2, is substantially free of antibodies that specifically bind other epitopes or other antigens than a coronavirus, unless the isolated antibody is combined with one or more isolated antibodies of interest, such as an antibody that specifically binds a second coronavirus).

The term “isolated nucleic acid,” as used in the context of a nucleic acid encoding an antibody, or antigen-binding fragment thereof, refers to a nucleic acid molecule in which the nucleotide sequences encoding the antibody, or antigen-binding fragment thereof, are free of other nucleotide sequences encoding antibodies or portions thereof that bind antigens other than a coronavirus, which other sequences may naturally flank the nucleic acid in human genomic DNA. Thus, for example, an isolated nucleic acid encoding a V_{H} region of a coronavirus antibody contains no other sequences encoding other V_{H} regions that bind antigens other than the coronavirus.

The term “identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988).

Typical methods to determine identity are designed to give the largest match between the sequences tested. Methods to determine identity and similarity are codified in publicly available computer programs. Typical computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity. IgBlast may also be used to determine germline V, D and J gene matches to a query sequence, which is available on the world wide web at ncbi.nlm.nih.gov/igblast/.

The term “preventing” or “prevention” refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset, such as exposure to a coronavirus, e.g. SARS-CoV-2.

The term “prophylaxis” is related to “prevention” and refers to a measure or procedure the purpose of which is to prevent, rather than to treat or cure a disease.

Antibodies

Antibodies, also known as immunoglobulins, are typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG₁, IgG₂, IgG₃, IgG₄, IgA₁, and IgA₂. Each light chain includes an N-terminal variable (V) domain (V_{L}) and a constant (C) domain (C_{L}). Each heavy chain includes an N-terminal V domain (V_{H}), three or four C domains (CHs), and a hinge region. The C_{H} domain most proximal to V_{H} is designated as CHI. The V_{H} and V_{L} domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3. Identification and numbering of framework and CDR residues is as described by Chothia et al., Structural determinants in the sequences of immunoglobulin variable domain, J Mol Biol 1998, 278:457-79, which is hereby incorporated by reference in its entirety.

CDR3 is typically the greatest source of molecular diversity within the antibody-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988. One of skill in the art will recognize that each subunit structure, e.g., a CH, VH, CL, VL, CDR, FR structure, comprises active fragments, e.g., the portion of the VH, VL, or CDR subunit the binds to the antigen, i.e., the antigen-binding fragment, or, e.g., the portion of the CH subunit that binds to and/or activates, e.g., an Fc receptor and/or complement. The CDRs typically refer to the Kabat CDRs, as described in Sequences of Proteins of Immunological Interest, US Department of Health and Human Services (1991), eds. Kabat et al. Another standard for characterizing the antigen binding site is to refer to the hypervariable loops as described by Chothia. See, e.g., Chothia, D. et al. (1992) J Mol. Biol. 227:799-817; and Tomlinson et al. (1995) EMBO J 14:4628-4638. Still another standard is the AbM definition used by Oxford Molecular's AbM antibody modeling software. See, generally, e.g., Protein Sequence and Structure Analysis ofAntibody Variable Domains. In: Antibody Engineering Lab Manual (Ed.: Duebel, S. and Kontermann, R., Springer-Verlag, Heidelberg). Embodiments described with respect to Kabat CDRs can alternatively be implemented using similar described relationships with respect to Chothia hypervariable loops or to the AbM-defined loops. Another standard for residue numbering that can be used is IMGT (Lefranc et al., Dev & Comp Immunol, 27(1):55-77 (2003).

The Fab fragment (Fragment antigen-binding) consists of V_{H}-C_{H}1 and V_{L}-C_{L} domains covalently linked by a disulfide bond between the constant regions. The Fᵥ fragment is smaller and consists of V_{H} and V_{L} domains non-covalently linked. To overcome the tendency of non-covalently linked domains to dissociate, a single chain Fᵥ fragment (scFᵥ) can be constructed. The scFᵥ contains a flexible connector, usually a polypeptide, that links (1) the C-terminus of V_{H} to the N-terminus of V_{L}, or (2) the C-terminus of V_{L} to the N-terminus of V_{H}.

In addition, protein engineering can recombinantly generate variable regions or graft or conjugate variable region sequences on a multi-domain and multi-function protein. Such proteins can have specific antigen binding properties, but are not typically referred to as monoclonal antibodies per se. Protein engineering can also be used to produce recombinant, polyclonal, bispecific, bivalent, multivalent and heteroconjugate antibodies. For example, it is possible to generate a bispecific antibody comprising antigen-binding fragments from two different mAbs that are described in this application, including a bispecific antibody that comprises 1) a first antigen binding fragment of a mAb, as disclosed herein, that binds to a receptor-binding domain of the S1 subunit and a second antigen-binding fragment of a mAb, as disclosed herein, that binds to the N-terminal domain of the S1 subunit; 2) a first antigen binding fragment of a mAb, as disclosed herein, that binds to a receptor-binding domain of the S1 subunit and a second antigen-binding fragment of a mAb, as disclosed herein, that binds to the S2 subunit; 3) a first antigen binding fragment of a mAb, as disclosed herein, that binds to the N-terminal domain of the S1 subunit and a second antigen-binding fragment of a mAb, as disclosed herein, that binds to the S2 subunit; 4) a first antigen binding fragment of a mAb, as disclosed herein, that binds to a receptor-binding domain of the S1 subunit and a second antigen-binding fragment of a mAb, as disclosed herein, that binds to a receptor-binding domain of the S1 subunit, wherein the first and second antigen-binding fragments are from different mAbs; 5) a first antigen binding fragment of a mAb, as disclosed herein, that binds to the N-terminal domain of the S1 subunit and a second antigen-binding fragment of a mAb, as disclosed herein, that binds to the N-terminal domain of the S1 subunit, wherein the first and second antigen-binding fragments are from different mAbs; 6) a first antigen binding fragment of a mAb, as disclosed herein, that binds to the S2 subunit and a second antigen-binding fragment of a mAb, as disclosed herein, that binds to the S2 subunit, wherein the first and second antigen-binding fragments are from different mAbs; 7) a first antigen binding fragment of a mAb, as disclosed herein, that binds to the S1 subunit and a second antigen binding fragment of a mAb, as disclosed herein, that binds to the S1 subunit, wherein the first and second antigen-binding fragments are from different mAbs; or 8) a first antigen binding fragment of a mAb, as disclosed herein, that binds to the S1 subunit and a second antigen binding fragment of a mAb, as disclosed herein, that binds to the S2 subunit.

. By way of further example, the first antigen-binding fragment binds to a receptor-binding domain of the S1 subunit of SARS-CoV2 and is from one of the following mAbs: COV_2123, COV_2125, or COV_2173, while the second antigen-binding fragment binds to the N-terminal domain of the S1 subunit of SARS-CoV2 and is from one of the following mAbs: COV_2004, COV_2025, or COV_2039.

It is also possible to modify an antibody to increase productivity and functionality and/or when relevant, to decrease possible immunogenicity. In addition, monoclonal antibodies may be modified at either the DNA sequence level to improve expression by removing hairpins or other secondary structure, by optimizing codon utilization, or at the amino acid level to improve expression or stability. For example, it is possible to remove residues such as unpaired cysteines to reduce aggregation, to alter glycosylation sites, or to substitute residues prone to deamidation or oxidization.

In some embodiments, an Fc portion of an antibody or antigen-binding fragment described herein is modified to increase its antibody serum-half life in vivo. In some embodiments, an Fc modified antibody or antigen-binding fragment thereof extends its therapeutic and/or protective activity. Such modifications to the Fc region can circumvent the need for frequent administration and/or allow for lower dosing, resulting in improved patient compliance and/or lower costs in comparison to an antibody or antigen-binding fragment thereof with an unmodified Fc region.

In some embodiments, the Fc modification confers a longer circulation half-life. Typically, the modification relies on improving the interaction between the IgG Fc domain and the neonatal Fc receptor (FcRn), a ubiquitously expressed cellular receptor which binds to internalized IgG at endosomal pH (5.5-6.0), prevents lysosomal degradation and promotes recycling to the extracellular fluid (Roopenian and Akilesh, Nat. Rev. Immunol. 2007 September; 7(9):715-25). Fc engineering for higher FcRn binding affinity at endosomal pH has yielded several Fc mutations capable of improving IgG half-life, as assessed in non-human primates and in human FcRn transgenic mice models.

For example, the Fc modification may comprise an “LS” or so-called “XTEND™” mutation (M428L/N434S) developed by Xencor Corp. XTEND™ may provide an 11-fold increase in binding at pH 6.0 relative to wild-type IgG1, which is a 4.2-fold improvement in serum half-life in transgenic mice and 3.2-fold in non-human primates. As described in Zalevsky et al., 2010, Nat. Biotechnol., 2010 February; 28(2): 157-159, XTEND™ Fc was tested in xenograft mouse models that express human FcRn as either an anti-VEGF or anti-EGFR IgG₁ antibody, which resulted in extended serum half-life as well as reduced tumor burden relative to those of wild-type IgG₁. As described in Roth et al., 2018, XTEND™ has been adapted to ravulizumab (ALXN1210), resulting in a serum half-life of ˜49.7 days. Ravulizumab was approved by United States Food and Drug Administration on December 2018 for the treatment of paroxysmal nocturnal hemoglobinuria/hemolytic-uremic syndrome (Roth et al., BloodAdv., 2018 Sep. 11; 2(17):2176-2185). XTEND™ has also been adapted to VRC01-LS, which is under clinical evaluation for the prevention of human immunodeficiency virus (Gaudinski et al., PLoS Med. 2018 Jan. 24; 15(1):e1002493).

It may also be desirable to modify an antibody to improve effector function, e.g., so as to enhance antigen-dependent cell-mediated cytotoxicity (ADCC) and/or complement dependent cytotoxicity (CDC) of the antagonist. One or more amino acid substitutions or the introduction of cysteine in the Fc region may be made, thereby improving internalization capability and/or increased complement-mediated cell killing and ADCC. See Caron et al., J. Ex. Med. 176:1191-1195 (1991) and Shopes, B.J. Immunol. 148:2918-2022 (1992), incorporated herein by reference in their entirety. An antibody fusion protein may be prepared that has dual Fc regions with both enhanced complement lysis and ADCC capabilities. Typical Fc receptors that bind to an Fc region of an antibody (e.g., an IgG antibody) include, but are not limited to, receptors of the FcγRI, FcγRII, and FcγRIII and FcRn subclasses, including allelic variants and alternatively spliced forms of these receptors. Fc receptors are reviewed in Ravetch and Kinet, Annu. Rev. Immunol 9:457-92, 1991; Capel et al., Immunomethods 4:25-34, 1994; and de Haas et al., J Lab. Clin. Med. 126:330-41, 1995). It is also possible to couple or join an antibody to another agent, such as a cytotoxic agent, drug, or therapeutic. In order to avoid possible effects due to antibody dependent enhancement (ADE), the Fc-binding domain of monoclonal antibodies may be mutated to prevent uptake into immune cells. Such mutations include those that abrogate the binding of antibodies to Fcγ receptors, such as LALA (L234A L235A), LALA-PG (L234A L235A P329G), and elimination of the glycosylation site at N297.

Anti-coronavirus antibodies described in this application may optionally comprise antibody constant regions, such as human constant regions, or parts thereof. For example, a V_{L} domain may be attached at its C-terminal end to a light chain constant domain like C_{κ} or Cλ. Similarly, a V_{H} domain or portion thereof may be attached to all or part of a heavy chain like IgA, IgD, IgE, IgG, and IgM, and any isotype subclass. Constant regions are known in the art (see, for example, Kabat et al., Sequences of Proteins of Immunological Interest, No. 91-3242, National Institutes of Health Publications, Bethesda, MD (1991)).

In some embodiments, the constant region is a human constant region. Typically, the source of the heavy chain variable domain and the light chain variable domain is different from the source of the human constant region. For example, using recombinant technology, the antibodies of the disclosure can include any human constant region of interest. In this way, the antibodies disclosed herein can be designed to include a human constant region that is different from the human constant region of the antibody obtained from the participants described in the Examples, e.g. participant RV229H.211 or R229H.213.

The antibodies of this disclosure may be tagged with a detectable or functional label. These labels include radiolabels (e.g., ¹³¹1 or ⁹⁹Tc), enzymatic labels (e.g., horseradish peroxidase or alkaline phosphatase), fluorescent labels, chemiluminescent labels, bioluminescent labels, and other chemical moieties (e.g., streptavidin/biotin, avidin/biotin).

Anti-Coronavirus Antibodies

This disclosure provides antibodies, including human, recombinant monoclonal antibodies, that bind to a coronavirus. Some of the antibodies have been shown to bind to SARS—Co-V2 with high affinity, e.g., a dissociation constant (K_{D}) less than 25 pM, with some having even lower K_{D}, including less than 10 pM, less 2 pM or even less than 1 pM. Some of the antibodies have been shown to possess broad cross-reactivity against different coronaviruses. In some embodiments, the present antibodies are capable of binding to one or more of SARS-CoV-1, SARS-CoV-2, Middle East Respiratory Syndrome (MERS)-CoV (MERS-CoV), hCoV-HKU1, hCoV-OC43, hCoV-NL63 and/or hCoV-229E. The variable heavy and light chain regions of the antibodies disclosed herein were sequenced from antibodies that had been isolated from individuals following seasonal coronavirus infection, or infection with SARS-CoV-2. The present monoclonal antibodies may be used to neutralize SARS-CoV-2, bind SARS-CoV-2 with high affinity, or have bind to epitopes on several different coronavirus spike proteins as herein described. The targets of the present antibodies reveal an area of vulnerability on the coronavirus to target in a vaccine. As also herein described, the antibodies of the disclosure may be used therapeutically for prevention or treatment of a disease, such as COVID-19, that is caused by a caused by a coronavirus, such as SARS-CoV-2 or SARS-CoV-1. In some embodiments, the antibodies of the disclosure have capability of being used commercially in antigen-capture SARS-CoV-2 diagnostic assays.

In some embodiments, the present antibodies bind to the spike (S) protein of a coronavirus, thereby inhibiting viral entry into host cells.

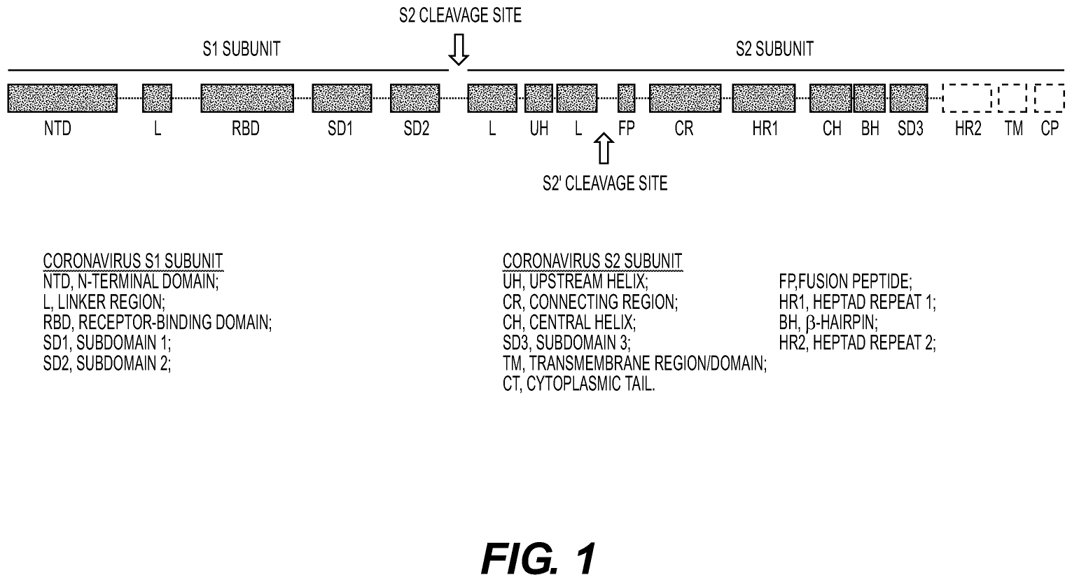

As depicted in FIG. 1, the S protein of a coronavirus, such as SARS-CoV-1, SARS—Co-V2 and MERS-CoV consists of S1 and S2 subunits. Typically, the receptor-binding domain (RBD) in the S1 subunit first binds the angiotensin-converting enzyme 2 (ACE2) receptor on cells to mediate viral entry via the formation of the RBD-ACE2 complex. The S protein then undergoes a conformational change, leading to membrane fusion mediated by the S2 subunit. The S protein forms a homotrimer and can undergo spontaneous conformational changes with one or more RBDs, switching from a ‘lying down’ position to a ‘standing up’ position to enable receptor binding.

In some embodiments, the instant antibodies bind to RBD in the S1 subunit. In some embodiments, the antibodies binding to S1 RBD inhibit viral entry into host cells. In some embodiments, the antibodies of the disclosure do not bind to the S1 RBD subunit. In some embodiments, the antibodies of the disclosure bind to the N-terminal domain (NTD) on the S1 subunit or other epitopes on the S1 subunit. In some embodiments, the antibodies of the disclosure bind to the S2 subunit. In some embodiments, the antibodies binding to the S i NTD or the S2 subunit prevent conformational changes of S or inhibit membrane fusion and viral entry.

In some embodiments, the human, recombinant, monoclonal antibody binds to a coronavirus, such as to the S1 RBD, S1 NTD, S1 subunit, or S2 subunit with a dissociation constant (K_{D}) equal to or less than 500 pM, 250 pM, 200 pM, 150 pM, 100 μM (10⁻¹⁰M), 25 μM, 15 pM, 10 μM (10⁻¹¹M), 2 pM, 1 μM (10⁻¹²M), 0.1 μM (10⁻¹³M), 0.01 μM (10⁻¹⁴M), or 0.001 μM (10⁻¹⁵M). The dissociation constant may be measured using techniques known in the art, such as described in the Examples.

In some embodiments, the human, monoclonal, recombinant antibody is a neutralizing antibody. Neutralization may be assessed using techniques known in the art, such as described in the Examples.

In one embodiment, the antibody is an isolated COV_1007 antibody. As used herein, the prefix “COV” “CoV”, followed by a number, is used interchangeably with the prefix “WRAIR.” Accordingly, the term “COV_1007,” “Cov_1007,” “COV-1007,” or WRAIR-1007, for example, refer to the same antibody. All of the antibodies described herein may be interchangeably describes as “COV” or “WRAIR” antibodies.

“COV_1007” refers to a monoclonal antibody, or antigen-binding fragment thereof, that binds to the S1 NTD of a coronavirus, such as SARS-CoV-2, wherein the antibody comprises 1) a heavy chain variable domain comprising the amino acid sequence of SEQ ID NO:2 and a light chain variable domain comprising the amino acid sequence of SEQ ID NO:7; or 2) a heavy chain variable domain comprising a CDR1 comprising the amino acid sequence of SEQ ID NO:3, a CDR2 comprising the amino acid sequence of SEQ ID NO:4, and a CDR3 comprising the amino acid sequence of SEQ ID NO:5 and a light chain variable domain comprising a CDR1 comprising the amino acid sequence of SEQ ID NO:8, a CDR2 comprising the amino acid sequence of SEQ ID NO:9, and a CDR3 comprising the amino acid sequence of SEQ ID NO:10.

In one embodiment, the antibody is an isolated COV_1037 antibody. As used herein, the term “COV_1037” refers to a monoclonal antibody, or antigen-binding fragment thereof, that binds to the S1 NTD or S1 RBD domain of a coronavirus, such as SARS-CoV-2, wherein the antibody comprises 1) a heavy chain variable domain comprising the amino acid sequence of SEQ ID NO:12 and a light chain variable domain comprising the amino acid sequence of SEQ ID NO:17; or 2) a heavy chain variable domain comprising a CDR1 comprising the amino acid sequence of SEQ ID NO:13, a CDR2 comprising the amino acid sequence of SEQ ID NO: 14, and a CDR3 comprising the amino acid sequence of SEQ ID NO:15 and a light chain variable domain comprising a CDR1 comprising the amino acid sequence of SEQ ID NO:18, a CDR2 comprising the amino acid sequence of SEQ ID NO:19, and a CDR3 comprising the amino acid sequence of SEQ ID NO:20.

In one embodiment, the antibody is an isolated COV_1045 antibody. As used herein, the term “COV_1045” refers to a monoclonal antibody, or antigen-binding fragment thereof, that binds to the S2 subunit of a coronavirus, such as SARS-CoV-2, wherein the antibody comprises 1) a heavy chain variable domain comprising the amino acid sequence of SEQ ID NO:22 and a light chain variable domain comprising the amino acid sequence of SEQ ID NO:27; or 2) a heavy chain variable domain comprising a CDR1 comprising the amino acid sequence of SEQ ID NO:23, a CDR2 comprising the amino acid sequence of SEQ ID NO:24, and a CDR3 comprising the amino acid sequence of SEQ ID NO:25 and a light chain variable domain comprising a CDR1 comprising the amino acid sequence of SEQ ID NO:28, a CDR2 comprising the amino acid sequence of SEQ ID NO:29, and a CDR3 comprising the amino acid sequence of SEQ ID NO:30.

In one embodiment, the antibody is an isolated COV_1046 antibody. As used herein, the term “COV_1046” refers to a monoclonal antibody, or antigen-binding fragment thereof, that binds to the S2 subunit of a coronavirus, such as SARS-CoV-2, wherein the antibody comprises 1) a heavy chain variable domain comprising the amino acid sequence of SEQ ID NO:32 and a light chain variable domain comprising the amino acid sequence of SEQ ID NO: 37; or 2) a heavy chain variable domain comprising a CDR1 comprising the amino acid sequence of SEQ ID NO:33, a CDR2 comprising the amino acid sequence of SEQ ID NO:34, and a CDR3 comprising the amino acid sequence of SEQ ID NO:35 and a light chain variable domain comprising a CDR1 comprising the amino acid sequence of SEQ ID NO:38, a CDR2 comprising the amino acid sequence of SEQ ID NO:39, and a CDR3 comprising the amino acid sequence of SEQ ID NO:40.

In one embodiment, the antibody is an isolated COV_1201 antibody. As used herein, the term “COV_1201” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:42 and a light chain variable domain comprising the amino acid sequence of SEQ ID NO:47; or 2) a heavy chain variable domain comprising a CDR1 comprising the amino acid sequence of SEQ ID NO:43, a CDR2 comprising the amino acid sequence of SEQ ID NO:44, and a CDR3 comprising the amino acid sequence of SEQ ID NO:45 and a light chain variable domain comprising a CDR1 comprising the amino acid sequence of SEQ ID NO:48, a CDR2 comprising the amino acid sequence of SEQ ID NO:49, and a CDR3 comprising the amino acid sequence of SEQ ID NO:50.

In one embodiment, the antibody is an isolated COV_2004 antibody. As used herein, the term “COV_2004” refers to a monoclonal antibody, or antigen-binding fragment thereof, that binds to the S1 NTD domain of a coronavirus, such as SARS-CoV-2, wherein the antibody comprises 1) a heavy chain variable domain comprising the amino acid sequence of SEQ ID NO:52 and a light chain variable domain comprising the amino acid sequence of SEQ ID NO:57; 2) a heavy chain variable domain comprising a CDR1 comprising the amino acid sequence of SEQ ID NO:53, a CDR2 comprising the amino acid sequence of SEQ ID NO:54, and a CDR3 comprising the amino acid sequence of SEQ ID NO:55 and a light chain variable domain comprising a CDR1 comprising the amino acid sequence of SEQ ID NO:58, a CDR2 comprising the amino acid sequence of SEQ ID NO:59, and a CDR3 comprising the amino acid sequence of SEQ ID NO:60; or 3) a heavy chain variable domain comprising a CDR1 comprising the amino acid sequence of SEQ ID NO:1109, a CDR2 comprising the amino acid sequence of SEQ ID NO:1139, and a CDR3 comprising the amino acid sequence of SEQ ID NO:1169 and a light chain variable domain comprising a CDR1 comprising the amino acid sequence of SEQ ID NO:1199, a CDR2 comprising the amino acid sequence AAS, and a CDR3 comprising the amino acid sequence of SEQ ID NO:1229.

In one embodiment, the antibody is an isolated COV_2008 antibody. As used herein, the term “COV_2008” refers to a monoclonal antibody, or antigen-binding fragment thereof, that binds to the S1 NTD domain of a coronavirus, such as SARS-CoV-2, wherein the antibody comprises 1) a heavy chain variable domain comprising the amino acid sequence of SEQ ID NO:62 and a light chain variable domain comprising the amino acid sequence of SEQ ID NO:67; 2) a heavy chain variable domain comprising a CDR1 comprising the amino acid sequence of SEQ ID NO:63, a CDR2 comprising the amino acid sequence of SEQ ID NO:64, and a CDR3 comprising the amino acid sequence of SEQ ID NO:65 and a light chain variable domain comprising a CDR1 comprising the amino acid sequence of SEQ ID NO:68, a CDR2 comprising the amino acid sequence of SEQ ID NO:69, and a CDR3 comprising the amino acid sequence of SEQ ID NO:70; or 3) a heavy chain variable domain comprising a CDR1 comprising the amino acid sequence of SEQ ID NO:1110, a CDR2 comprising the amino acid sequence of SEQ ID NO: 1140, and a CDR3 comprising the amino acid sequence of SEQ ID NO: 1170 and a light chain variable domain comprising a CDR1 comprising the amino acid sequence of SEQ ID NO: 1200, a CDR2 comprising the amino acid sequence KIS, and a CDR3 comprising the amino acid sequence of SEQ ID NO: 1230.

In one embodiment, the antibody is an isolated COV_2014 antibody. As used herein, the term “COV_2014” refers to a monoclonal antibody, or antigen-binding fragment thereof, that binds to the S1 NTD of a coronavirus, such as SARS-CoV-2, wherein the antibody comprises 1) a heavy chain variable domain comprising the amino acid sequence of SEQ ID NO:72 and a light chain variable domain comprising the amino acid sequence of SEQ ID NO:77; 2) a heavy chain variable domain comprising a CDR1 comprising the amino acid sequence of SEQ ID NO:73, a CDR2 comprising the amino acid sequence of SEQ ID NO:74, and a CDR3 comprising the amino acid sequence of SEQ ID NO:75 and a light chain variable domain comprising a CDR1 comprising the amino acid sequence of SEQ ID NO:78, a CDR2 comprising the amino acid sequence of SEQ ID NO:79, and a CDR3 comprising the amino acid sequence of SEQ ID NO:80; or 3) a heavy chain variable domain comprising a CDR1 comprising the amino acid sequence of SEQ ID NO:1118, a CDR2 comprising the amino acid sequence of SEQ ID NO: 1148, and a CDR3 comprising the amino acid sequence of SEQ ID NO: 1178 and a light chain variable domain comprising a CDR1 comprising the amino acid sequence of SEQ ID NO: 1208, a CDR2 comprising the amino acid sequence AAS, and a CDR3 comprising the amino acid sequence of SEQ ID NO: 1238.

In one embodiment, the antibody is an isolated COV_2018 antibody. As used herein, the term “COV_2018” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:82 and a light chain variable domain comprising the amino acid sequence of SEQ ID NO:87; 2) a heavy chain variable domain comprising a CDR1 comprising the amino acid sequence of SEQ ID NO:83, a CDR2 comprising the amino acid sequence of SEQ ID NO:84, and a CDR3 comprising the amino acid sequence of SEQ ID NO:85 and a light chain variable domain comprising a CDR1 comprising the amino acid sequence of SEQ ID NO:88, a CDR2 comprising the amino acid sequence of SEQ ID NO:89, and a CDR3 comprising the amino acid sequence of SEQ ID NO:90; or 3) a heavy chain variable domain comprising a CDR1 comprising the amino acid sequence of SEQ ID NO:1130, a CDR2 comprising the amino acid sequence of SEQ ID NO: 1160, and a CDR3 comprising the amino acid sequence of SEQ ID NO: 1190 and a light chain variable domain comprising a CDR1 comprising the amino acid sequence of SEQ ID NO: 1220, a CDR2 comprising the amino acid sequence NAS, and a CDR3 comprising the amino acid sequence of SEQ ID NO: 1250.

In one embodiment, the antibody is an isolated COV_2024 antibody. As used herein, the term “COV_2024” refers to a monoclonal antibody, or antigen-binding fragment thereof, that binds to the S2 subunit of a coronavirus, such as SARS-CoV-2, wherein the antibody comprises 1) a heavy chain variable domain comprising the amino acid sequence of SEQ ID NO:92 and a light chain variable domain comprising the amino acid sequence of SEQ ID NO:97; or 2) a heavy chain variable domain comprising a CDR1 comprising the amino acid sequence of SEQ ID NO:93, a CDR2 comprising the amino acid sequence of SEQ ID NO:94, and a CDR3 comprising the amino acid sequence of SEQ ID NO:95 and a light chain variable domain comprising a CDR1 comprising the amino acid sequence of SEQ ID NO:98, a CDR2 comprising the amino acid sequence of SEQ ID NO:99, and a CDR3 comprising the amino acid sequence of SEQ ID NO:100.

In one embodiment, the antibody is an isolated COV_2025 antibody. As used herein, the term “COV_2025” refers to a monoclonal antibody, or antigen-binding fragment thereof, that binds to the S1 NTD domain of a coronavirus, such as SARS-CoV-2, wherein the antibody comprises 1) a heavy chain variable domain comprising the amino acid sequence of SEQ ID NO:102 and a light chain variable domain comprising the amino acid sequence of SEQ ID NO: 107; 2) a heavy chain variable domain comprising a CDR1 comprising the amino acid sequence of SEQ ID NO:103, a CDR2 comprising the amino acid sequence of SEQ ID NO:104, and a CDR3 comprising the amino acid sequence of SEQ ID NO:105 and a light chain variable domain comprising a CDR1 comprising the amino acid sequence of SEQ ID NO:108, a CDR2 comprising the amino acid sequence of SEQ ID NO:109, and a CDR3 comprising the amino acid sequence of SEQ ID NO:110; or 3) a heavy chain variable domain comprising a CDR1 comprising the amino acid sequence of SEQ ID NO:1111, a CDR2 comprising the amino acid sequence of SEQ ID NO: 1141, and a CDR3 comprising the amino acid sequence of SEQ ID NO: 1171 and a light chain variable domain comprising a CDR1 comprising the amino acid sequence of SEQ ID NO: 1201, a CDR2 comprising the amino acid sequence EVT, and a CDR3 comprising the amino acid sequence of SEQ ID NO: 1231.

In one embodiment, the antibody is an isolated COV_2027 antibody. As used herein, the term “COV_2027” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:112 and a light chain variable domain comprising the amino acid sequence of SEQ ID NO: 117; 2) a heavy chain variable domain comprising a CDR1 comprising the amino acid sequence of SEQ ID NO:113, a CDR2 comprising the amino acid sequence of SEQ ID NO:114, and a CDR3 comprising the amino acid sequence of SEQ ID NO:115 and a light chain variable domain comprising a CDR1 comprising the amino acid sequence of SEQ ID NO:118, a CDR2 comprising the amino acid sequence of SEQ ID NO:119, and a CDR3 comprising the amino acid sequence of SEQ ID NO:120 or 3) a heavy chain variable domain comprising a CDR1 comprising the amino acid sequence of SEQ ID NO:1123, a CDR2 comprising the amino acid sequence of SEQ ID NO: 1153, and a CDR3 comprising the amino acid sequence of SEQ ID NO: 1183 and a light chain variable domain comprising a CDR1 comprising the amino acid sequence of SEQ ID NO: 1213, a CDR2 comprising the amino acid sequence GAS, and a CDR3 comprising the amino acid sequence of SEQ ID NO: 1243.

In one embodiment, the antibody is an isolated COV_2028 antibody. As used herein, the term “COV_2028” refers to a monoclonal antibody, or antigen-binding fragment thereof, that binds to the S1 NTD domain of a coronavirus, such as SARS-CoV-2, wherein the antibody comprises 1) a heavy chain variable domain comprising the amino acid sequence of SEQ ID NO:122 and a light chain variable domain comprising the amino acid sequence of SEQ ID NO: 127; 2) a heavy chain variable domain comprising a CDR1 comprising the amino acid sequence of SEQ ID NO:123, a CDR2 comprising the amino acid sequence of SEQ ID NO:124, and a CDR3 comprising the amino acid sequence of SEQ ID NO:125 and a light chain variable domain comprising a CDR1 comprising the amino acid sequence of SEQ ID NO:128, a CDR2 comprising the amino acid sequence of SEQ ID NO:129, and a CDR3 comprising the amino acid sequence of SEQ ID NO:130; or 3) a heavy chain variable domain comprising a CDR1 comprising the amino acid sequence of SEQ ID NO:1116, a CDR2 comprising the amino acid sequence of SEQ ID NO: 1146, and a CDR3 comprising the amino acid sequence of SEQ ID NO: 1176 and a light chain variable domain comprising a CDR1 comprising the amino acid sequence of SEQ ID NO: 1206, a CDR2 comprising the amino acid sequence EVS, and a CDR3 comprising the amino acid sequence of SEQ ID NO: 1236.

In one embodiment, the antibody is an isolated COV_2035 antibody. As used herein, the term “COV_2035” refers to a monoclonal antibody, or antigen-binding fragment thereof, that binds to the S1 NTD domain of a coronavirus, such as SARS-CoV-2, wherein the antibody comprises 1) a heavy chain variable domain comprising the amino acid sequence of SEQ ID NO:132 and a light chain variable domain comprising the amino acid sequence of SEQ ID NO: 137; 2) a heavy chain variable domain comprising a CDR1 comprising the amino acid sequence of SEQ ID NO:133, a CDR2 comprising the amino acid sequence of SEQ ID NO:134, and a CDR3 comprising the amino acid sequence of SEQ ID NO:135 and a light chain variable domain comprising a CDR1 comprising the amino acid sequence of SEQ ID NO:138, a CDR2 comprising the amino acid sequence of SEQ ID NO:139, and a CDR3 comprising the amino acid sequence of SEQ ID NO: 140; or 3) a heavy chain variable domain comprising a CDR1 comprising the amino acid sequence of SEQ ID NO:1112, a CDR2 comprising the amino acid sequence of SEQ ID NO: 1142, and a CDR3 comprising the amino acid sequence of SEQ ID NO: 1172 and a light chain variable domain comprising a CDR1 comprising the amino acid sequence of SEQ ID NO: 1202, a CDR2 comprising the amino acid sequence EVS, and a CDR3 comprising the amino acid sequence of SEQ ID NO: 1232.

In one embodiment, the antibody is an isolated COV_2037 antibody. As used herein, the term “COV_2037” refers to a monoclonal antibody, or antigen-binding fragment thereof, that binds to the S1 NTD domain of a coronavirus, such as SARS-CoV-2, wherein the antibody comprises 1) a heavy chain variable domain comprising the amino acid sequence of SEQ ID NO: 142 and a light chain variable domain comprising the amino acid sequence of SEQ ID NO: 147; 2) a heavy chain variable domain comprising a CDR1 comprising the amino acid sequence of SEQ ID NO:143, a CDR2 comprising the amino acid sequence of SEQ ID NO:144, and a CDR3 comprising the amino acid sequence of SEQ ID NO:145 and a light chain variable domain comprising a CDR1 comprising the amino acid sequence of SEQ ID NO:148, a CDR2 comprising the amino acid sequence of SEQ ID NO:149, and a CDR3 comprising the amino acid sequence of SEQ ID NO:150; or 3) a heavy chain variable domain comprising a CDR1 comprising the amino acid sequence of SEQ ID NO:1113, a CDR2 comprising the amino acid sequence of SEQ ID NO: 1143, and a CDR3 comprising the amino acid sequence of SEQ ID NO: 1173 and a light chain variable domain comprising a CDR1 comprising the amino acid sequence of SEQ ID NO: 1203, a CDR2 comprising the amino acid sequence KIS, and a CDR3 comprising the amino acid sequence of SEQ ID NO: 1233.

In one embodiment, the antibody is an isolated COV_2039 antibody. As used herein, the term “COV_2039” refers to a monoclonal antibody, or antigen-binding fragment thereof, that binds to the S1 NTD domain of a coronavirus, such as SARS-CoV-2, wherein the antibody comprises 1) a heavy chain variable domain comprising the amino acid sequence of SEQ ID NO:152 and a light chain variable domain comprising the amino acid sequence of SEQ ID NO: 157; 2) a heavy chain variable domain comprising a CDR1 comprising the amino acid sequence of SEQ ID NO:153, a CDR2 comprising the amino acid sequence of SEQ ID NO:154, and a CDR3 comprising the amino acid sequence of SEQ ID NO:155 and a light chain variable domain comprising a CDR1 comprising the amino acid sequence of SEQ ID NO:158, a CDR2 comprising the amino acid sequence of SEQ ID NO:159, and a CDR3 comprising the amino acid sequence of SEQ ID NO:160; or 3) wherein the antibody comprises a heavy chain variable domain comprising a CDR1 comprising the amino acid sequence of SEQ ID NO:1114, a CDR2 comprising the amino acid sequence of SEQ ID NO: 1144, and a CDR3 comprising the amino acid sequence of SEQ ID NO: 1174 and a light chain variable domain comprising a CDR1 comprising the amino acid sequence of SEQ ID NO: 1204, a CDR2 comprising the amino acid sequence ANS, and a CDR3 comprising the amino acid sequence of SEQ ID NO: 1234.

In one embodiment, the antibody is an isolated COV_2054 antibody. As used herein, the term “COV_2054” refers to a monoclonal antibody, or antigen-binding fragment thereof, that binds to the S1 NTD domain of a coronavirus, such as SARS-CoV-2, wherein the antibody comprises 1) a heavy chain variable domain comprising the amino acid sequence of SEQ ID NO:162 and a light chain variable domain comprising the amino acid sequence of SEQ ID NO: 167; 2) a heavy chain variable domain comprising a CDR1 comprising the amino acid sequence of SEQ ID NO:163, a CDR2 comprising the amino acid sequence of SEQ ID NO:164, and a CDR3 comprising the amino acid sequence of SEQ ID NO:165 and a light chain variable domain comprising a CDR1 comprising the amino acid sequence of SEQ ID NO:168, a CDR2 comprising the amino acid sequence of SEQ ID NO:169, and a CDR3 comprising the amino acid sequence of SEQ ID NO:170; 3) a heavy chain variable domain comprising a CDR1 comprising the amino acid sequence of SEQ ID NO:1120, a CDR2 comprising the amino acid sequence of SEQ ID NO: 1150, and a CDR3 comprising the amino acid sequence of SEQ ID NO: 1180 and a light chain variable domain comprising a CDR1 comprising the amino acid sequence of SEQ ID NO: 1210, a CDR2 comprising the amino acid sequence EAS, and a CDR3 comprising the amino acid sequence of SEQ ID NO: 1240.

In one embodiment, the antibody is an isolated COV_2056 antibody. As used herein, the term “COV_2056” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:432 and a light chain variable domain comprising the amino acid sequence of SEQ ID NO:437; 2) a heavy chain variable domain comprising a CDR1 comprising the amino acid sequence of SEQ ID NO:433, a CDR2 comprising the amino acid sequence of SEQ ID NO:434, and a CDR3 comprising the amino acid sequence of SEQ ID NO:435 and a light chain variable domain comprising a CDR1 comprising the amino acid sequence of SEQ ID NO:438, a CDR2 comprising the amino acid sequence of SEQ ID NO:439, and a CDR3 comprising the amino acid sequence of SEQ ID NO:440; or 3) a heavy chain variable domain comprising a CDR1 comprising the amino acid sequence of SEQ ID NO:1131, a CDR2 comprising the amino acid sequence of SEQ ID NO: 1161, and a CDR3 comprising the amino acid sequence of SEQ ID NO: 1191 and a light chain variable domain comprising a CDR1 comprising the amino acid sequence of SEQ ID NO: 1221, a CDR2 comprising the amino acid sequence AAS, and a CDR3 comprising the amino acid sequence of SEQ ID NO: 1251.

In one embodiment, the antibody is an isolated COV_2057 antibody. As used herein, the term “COV_2057” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO: 172 and a light chain variable domain comprising the amino acid sequence of SEQ ID NO: 177; 2) a heavy chain variable domain comprising a CDR1 comprising the amino acid sequence of SEQ ID NO:173, a CDR2 comprising the amino acid sequence of SEQ ID NO:174, and a CDR3 comprising the amino acid sequence of SEQ ID NO:175 and a light chain variable domain comprising a CDR1 comprising the amino acid sequence of SEQ ID NO:178, a CDR2 comprising the amino acid sequence of SEQ ID NO:179, and a CDR3 comprising the amino acid sequence of SEQ ID NO: 180; or 3) a heavy chain variable domain comprising a CDR1 comprising the amino acid sequence of SEQ ID NO:1132, a CDR2 comprising the amino acid sequence of SEQ ID NO: 1162, and a CDR3 comprising the amino acid sequence of SEQ ID NO: 1192 and a light chain variable domain comprising a CDR1 comprising the amino acid sequence of SEQ ID NO: 1222, a CDR2 comprising the amino acid sequence AAS, and a CDR3 comprising the amino acid sequence of SEQ ID NO: 1252.

In one embodiment, the antibody is an isolated COV_2063 antibody. As used herein, the term “COV_2063” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:182 and a light chain variable domain comprising the amino acid sequence of SEQ ID NO: 187; 2) a heavy chain variable domain comprising a CDR1 comprising the amino acid sequence of SEQ ID NO:183, a CDR2 comprising the amino acid sequence of SEQ ID NO:184, and a CDR3 comprising the amino acid sequence of SEQ ID NO:185 and a light chain variable domain comprising a CDR1 comprising the amino acid sequence of SEQ ID NO:188, a CDR2 comprising the amino acid sequence of SEQ ID NO:189, and a CDR3 comprising the amino acid sequence of SEQ ID NO: 190; or 3) a heavy chain variable domain comprising a CDR1 comprising the amino acid sequence of SEQ ID NO:1133, a CDR2 comprising the amino acid sequence of SEQ ID NO: 1163, and a CDR3 comprising the amino acid sequence of SEQ ID NO: 1193 and a light chain variable domain comprising a CDR1 comprising the amino acid sequence of SEQ ID NO: 1223, a CDR2 comprising the amino acid sequence KVS, and a CDR3 comprising the amino acid sequence of SEQ ID NO: 1253.

In one embodiment, the antibody is an isolated COV_2091 antibody. As used herein, the term “COV_2091” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:192 and a light chain variable domain comprising the amino acid sequence of SEQ ID NO: 197; 2) a heavy chain variable domain comprising a CDR1 comprising the amino acid sequence of SEQ ID NO:193, a CDR2 comprising the amino acid sequence of SEQ ID NO:194, and a CDR3 comprising the amino acid sequence of SEQ ID NO:195 and a light chain variable domain comprising a CDR1 comprising the amino acid sequence of SEQ ID NO:198, a CDR2 comprising the amino acid sequence of SEQ ID NO:199, and a CDR3 comprising the amino acid sequence of SEQ ID NO:200; or 3) a heavy chain variable domain comprising a CDR1 comprising the amino acid sequence of SEQ ID NO:1134, a CDR2 comprising the amino acid sequence of SEQ ID NO: 1164, and a CDR3 comprising the amino acid sequence of SEQ ID NO: 1194 and a light chain variable domain comprising a CDR1 comprising the amino acid sequence of SEQ ID NO: 1224, a CDR2 comprising the amino acid sequence AAS, and a CDR3 comprising the amino acid sequence of SEQ ID NO: 1254.

In one embodiment, the antibody is an isolated COV_2100 antibody. As used herein, the term “COV_2100” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:202 and a light chain variable domain comprising the amino acid sequence of SEQ ID NO:207; 2) a heavy chain variable domain comprising a CDR1 comprising the amino acid sequence of SEQ ID NO:203, a CDR2 comprising the amino acid sequence of SEQ ID NO:204, and a CDR3 comprising the amino acid sequence of SEQ ID NO:205 and a light chain variable domain comprising a CDR1 comprising the amino acid sequence of SEQ ID NO:208, a CDR2 comprising the amino acid sequence of SEQ ID NO:209, and a CDR3 comprising the amino acid sequence of SEQ ID NO:210; or 3) a heavy chain variable domain comprising a CDR1 comprising the amino acid sequence of SEQ ID NO:1136, a CDR2 comprising the amino acid sequence of SEQ ID NO: 1166, and a CDR3 comprising the amino acid sequence of SEQ ID NO: 1196 and a light chain variable domain comprising a CDR1 comprising the amino acid sequence of SEQ ID NO: 1226, a CDR2 comprising the amino acid sequence AAS, and a CDR3 comprising the amino acid sequence of SEQ ID NO: 1256.

In one embodiment, the antibody is an isolated COV_2103 antibody. As used herein, the term “COV_2103” refers to a monoclonal antibody, or antigen-binding fragment thereof, that binds to the S1 NTD domain of a coronavirus, such as SARS-CoV-2, wherein the antibody comprises 1) a heavy chain variable domain comprising the amino acid sequence of SEQ ID NO:212 and a light chain variable domain comprising the amino acid sequence of SEQ ID NO:217; 2) a heavy chain variable domain comprising a CDR1 comprising the amino acid sequence of SEQ ID NO:213, a CDR2 comprising the amino acid sequence of SEQ ID NO:214, and a CDR3 comprising the amino acid sequence of SEQ ID NO:215 and a light chain variable domain comprising a CDR1 comprising the amino acid sequence of SEQ ID NO:218, a CDR2 comprising the amino acid sequence of SEQ ID NO:219, and a CDR3 comprising the amino acid sequence of SEQ ID NO:220; or 3) a heavy chain variable domain comprising a CDR1 comprising the amino acid sequence of SEQ ID NO:1121, a CDR2 comprising the amino acid sequence of SEQ ID NO: 1151, and a CDR3 comprising the amino acid sequence of SEQ ID NO: 1181 and a light chain variable domain comprising a CDR1 comprising the amino acid sequence of SEQ ID NO: 1211, a CDR2 comprising the amino acid sequence KAS, and a CDR3 comprising the amino acid sequence of SEQ ID NO: 1241.

In one embodiment, the antibody is an isolated COV_2108 antibody. As used herein, the term “COV_2108” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:222 and a light chain variable domain comprising the amino acid sequence of SEQ ID NO:227; 2) a heavy chain variable domain comprising a CDR1 comprising the amino acid sequence of SEQ ID NO:223, a CDR2 comprising the amino acid sequence of SEQ ID NO:224, and a CDR3 comprising the amino acid sequence of SEQ ID NO:225 and a light chain variable domain comprising a CDR1 comprising the amino acid sequence of SEQ ID NO:228, a CDR2 comprising the amino acid sequence of SEQ ID NO:229, and a CDR3 comprising the amino acid sequence of SEQ ID NO:230; or 3) a heavy chain variable domain comprising a CDR1 comprising the amino acid sequence of SEQ ID NO:1137, a CDR2 comprising the amino acid sequence of SEQ ID NO: 1167, and a CDR3 comprising the amino acid sequence of SEQ ID NO: 1197 and a light chain variable domain comprising a CDR1 comprising the amino acid sequence of SEQ ID NO: 1227, a CDR2 comprising the amino acid sequence TTS, and a CDR3 comprising the amino acid sequence of SEQ ID NO: 1257.

In one embodiment, the antibody is an isolated COV_2123 antibody. As used herein, the term “COV_2123” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:232 and a light chain variable domain comprising the amino acid sequence of SEQ ID NO:237; 2) a heavy chain variable domain comprising a CDR1 comprising the amino acid sequence of SEQ ID NO:233, a CDR2 comprising the amino acid sequence of SEQ ID NO:234, and a CDR3 comprising the amino acid sequence of SEQ ID NO:235 and a light chain variable domain comprising a CDR1 comprising the amino acid sequence of SEQ ID NO:238, a CDR2 comprising the amino acid sequence of SEQ ID NO:239, and a CDR3 comprising the amino acid sequence of SEQ ID NO:240 or 3) a heavy chain variable domain comprising a CDR1 comprising the amino acid sequence of SEQ ID NO:1124, a CDR2 comprising the amino acid sequence of SEQ ID NO: 1154, and a CDR3 comprising the amino acid sequence of SEQ ID NO: 1184 and a light chain variable domain comprising a CDR1 comprising the amino acid sequence of SEQ ID NO: 1214, a CDR2 comprising the amino acid sequence DAS, and a CDR3 comprising the amino acid sequence of SEQ ID NO: 1244.

In one embodiment, the antibody is an isolated COV_2125 antibody. As used herein, the term “COV_2125” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:242 and a light chain variable domain comprising the amino acid sequence of SEQ ID NO:247; 2) a heavy chain variable domain comprising a CDR1 comprising the amino acid sequence of SEQ ID NO:243, a CDR2 comprising the amino acid sequence of SEQ ID NO:244, and a CDR3 comprising the amino acid sequence of SEQ ID NO:245 and a light chain variable domain comprising a CDR1 comprising the amino acid sequence of SEQ ID NO:248, a CDR2 comprising the amino acid sequence of SEQ ID NO:249, and a CDR3 comprising the amino acid sequence of SEQ ID NO:250; or 3) a heavy chain variable domain comprising a CDR1 comprising the amino acid sequence of SEQ ID NO:1125, a CDR2 comprising the amino acid sequence of SEQ ID NO: 1155, and a CDR3 comprising the amino acid sequence of SEQ ID NO: 1185 and a light chain variable domain comprising a CDR1 comprising the amino acid sequence of SEQ ID NO: 1215, a CDR2 comprising the amino acid sequence AAS, and a CDR3 comprising the amino acid sequence of SEQ ID NO: 1245.

In one embodiment, the antibody is an isolated COV_2134 antibody. As used herein, the term “COV_2134” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:252 and a light chain variable domain comprising the amino acid sequence of SEQ ID NO:257; 2) a heavy chain variable domain comprising a CDR1 comprising the amino acid sequence of SEQ ID NO:253, a CDR2 comprising the amino acid sequence of SEQ ID NO:254, and a CDR3 comprising the amino acid sequence of SEQ ID NO:255 and a light chain variable domain comprising a CDR1 comprising the amino acid sequence of SEQ ID NO:258, a CDR2 comprising the amino acid sequence of SEQ ID NO:259, and a CDR3 comprising the amino acid sequence of SEQ ID NO:260; or a heavy chain variable domain comprising a CDR1 comprising the amino acid sequence of SEQ ID NO:1135, a CDR2 comprising the amino acid sequence of SEQ ID NO: 1165, and a CDR3 comprising the amino acid sequence of SEQ ID NO: 1195 and a light chain variable domain comprising a CDR1 comprising the amino acid sequence of SEQ ID NO: 1225, a CDR2 comprising the amino acid sequence QDS, and a CDR3 comprising the amino acid sequence of SEQ ID NO: 1255.

In one embodiment, the antibody is an isolated COV_2151 antibody. As used herein, the term “COV_2151” refers to a monoclonal antibody, or antigen-binding fragment thereof, that binds to the S1 RBD domain of a coronavirus, such as the RBD domain, such as the RBD domain of SARS-CoV-2, wherein the antibody comprises 1) a heavy chain variable domain comprising the amino acid sequence of SEQ ID NO:262 and a light chain variable domain comprising the amino acid sequence of SEQ ID NO:267; 2) a heavy chain variable domain comprising a CDR1 comprising the amino acid sequence of SEQ ID NO:263, a CDR2 comprising the amino acid sequence of SEQ ID NO:264, and a CDR3 comprising the amino acid sequence of SEQ ID NO:265 and a light chain variable domain comprising a CDR1 comprising the amino acid sequence of SEQ ID NO:268, a CDR2 comprising the amino acid sequence of SEQ ID NO:269, and a CDR3 comprising the amino acid sequence of SEQ ID NO:270; or 3) a heavy chain variable domain comprising a CDR1 comprising the amino acid sequence of SEQ ID NO:1138, a CDR2 comprising the amino acid sequence of SEQ ID NO: 1168, and a CDR3 comprising the amino acid sequence of SEQ ID NO: 1198 and a light chain variable domain comprising a CDR1 comprising the amino acid sequence of SEQ ID NO: 1228, a CDR2 comprising the amino acid sequence EDN, and a CDR3 comprising the amino acid sequence of SEQ ID NO: 1258.

In one embodiment, the antibody is an isolated COV_2165 antibody. As used herein, the term “COV_2165” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:272 and a light chain variable domain comprising the amino acid sequence of SEQ ID NO:277; 2) a heavy chain variable domain comprising a CDR1 comprising the amino acid sequence of SEQ ID NO:273, a CDR2 comprising the amino acid sequence of SEQ ID NO:274, and a CDR3 comprising the amino acid sequence of SEQ ID NO:275 and a light chain variable domain comprising a CDR1 comprising the amino acid sequence of SEQ ID NO:278, a CDR2 comprising the amino acid sequence of SEQ ID NO:279, and a CDR3 comprising the amino acid sequence of SEQ ID NO:280; or 3) a heavy chain variable domain comprising a CDR1 comprising the amino acid sequence of SEQ ID NO:1126, a CDR2 comprising the amino acid sequence of SEQ ID NO: 1156, and a CDR3 comprising the amino acid sequence of SEQ ID NO: 1186 and a light chain variable domain comprising a CDR1 comprising the amino acid sequence of SEQ ID NO: 1216, a CDR2 comprising the amino acid sequence DAS, and a CDR3 comprising the amino acid sequence of SEQ ID NO: 1246.

In one embodiment, the antibody is an isolated COV_2172 antibody. As used herein, the term “COV_2172” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:442 and a light chain variable domain comprising the amino acid sequence of SEQ ID NO:447; 2) a heavy chain variable domain comprising a CDR1 comprising the amino acid sequence of SEQ ID NO:443, a CDR2 comprising the amino acid sequence of SEQ ID NO:444, and a CDR3 comprising the amino acid sequence of SEQ ID NO:445 and a light chain variable domain comprising a CDR1 comprising the amino acid sequence of SEQ ID NO:448, a CDR2 comprising the amino acid sequence of SEQ ID NO:449, and a CDR3 comprising the amino acid sequence of SEQ ID NO:450; or 3) a heavy chain variable domain comprising a CDR1 comprising the amino acid sequence of SEQ ID NO:1127, a CDR2 comprising the amino acid sequence of SEQ ID NO: 1157, and a CDR3 comprising the amino acid sequence of SEQ ID NO: 1187 and a light chain variable domain comprising a CDR1 comprising the amino acid sequence of SEQ ID NO: 1217, a CDR2 comprising the amino acid sequence DVS, and a CDR3 comprising the amino acid sequence of SEQ ID NO: 1247.

In one embodiment, the antibody is an isolated COV_2173 antibody. As used herein, the term “COV_2173” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:282 and a light chain variable domain comprising the amino acid sequence of SEQ ID NO:287; 2) a heavy chain variable domain comprising a CDR1 comprising the amino acid sequence of SEQ ID NO:283, a CDR2 comprising the amino acid sequence of SEQ ID NO:284, and a CDR3 comprising the amino acid sequence of SEQ ID NO:285 and a light chain variable domain comprising a CDR1 comprising the amino acid sequence of SEQ ID NO:288, a CDR2 comprising the amino acid sequence of SEQ ID NO:289, and a CDR3 comprising the amino acid sequence of SEQ ID NO:290; or 3) a heavy chain variable domain comprising a CDR1 comprising the amino acid sequence of SEQ ID NO:1128, a CDR2 comprising the amino acid sequence of SEQ ID NO: 1158, and a CDR3 comprising the amino acid sequence of SEQ ID NO: 1188 and a light chain variable domain comprising a CDR1 comprising the amino acid sequence of SEQ ID NO: 1218, a CDR2 comprising the amino acid sequence GNN, and a CDR3 comprising the amino acid sequence of SEQ ID NO: 1248.

In one embodiment, the antibody is an isolated COV_2193 antibody. As used herein, the term “COV_2193” refers to a monoclonal antibody, or antigen-binding fragment thereof, that binds to the S1 NTD domain of a coronavirus, such as SARS-CoV-2, wherein the antibody comprises 1) a heavy chain variable domain comprising the amino acid sequence of SEQ ID NO: 292 and a light chain variable domain comprising the amino acid sequence of SEQ ID NO:297; 2) a heavy chain variable domain comprising a CDR1 comprising the amino acid sequence of SEQ ID NO:293, a CDR2 comprising the amino acid sequence of SEQ ID NO:294, and a CDR3 comprising the amino acid sequence of SEQ ID NO:295 and a light chain variable domain comprising a CDR1 comprising the amino acid sequence of SEQ ID NO:298, a CDR2 comprising the amino acid sequence of SEQ ID NO:299, and a CDR3 comprising the amino acid sequence of SEQ ID NO:300; o 3) a heavy chain variable domain comprising a CDR1 comprising the amino acid sequence of SEQ ID NO:1122, a CDR2 comprising the amino acid sequence of SEQ ID NO: 1152, and a CDR3 comprising the amino acid sequence of SEQ ID NO: 1182 and a light chain variable domain comprising a CDR1 comprising the amino acid sequence of SEQ ID NO: 1212, a CDR2 comprising the amino acid sequence LKN, and a CDR3 comprising the amino acid sequence of SEQ ID NO: 1242.

In one embodiment, the antibody is an isolated COV_2196 antibody. As used herein, the term “COV_2196” refers to a monoclonal antibody, or antigen-binding fragment thereof, that binds to S1 NTD domain of a coronavirus, such as SARS-CoV-2, wherein the antibody comprises 1) a heavy chain variable domain comprising the amino acid sequence of SEQ ID NO:302 and a light chain variable domain comprising the amino acid sequence of SEQ ID NO:307; 2) a heavy chain variable domain comprising a CDR1 comprising the amino acid sequence of SEQ ID NO:303, a CDR2 comprising the amino acid sequence of SEQ ID NO:304, and a CDR3 comprising the amino acid sequence of SEQ ID NO:305 and a light chain variable domain comprising a CDR1 comprising the amino acid sequence of SEQ ID NO:308, a CDR2 comprising the amino acid sequence of SEQ ID NO:309, and a CDR3 comprising the amino acid sequence of SEQ ID NO:310; or 3) a heavy chain variable domain comprising a CDR1 comprising the amino acid sequence of SEQ ID NO:1115, a CDR2 comprising the amino acid sequence of SEQ ID NO: 1145, and a CDR3 comprising the amino acid sequence of SEQ ID NO: 1175 and a light chain variable domain comprising a CDR1 comprising the amino acid sequence of SEQ ID NO: 1205, a CDR2 comprising the amino acid sequence KDT, and a CDR3 comprising the amino acid sequence of SEQ ID NO: 1235.

In one embodiment, the antibody is an isolated COV_3000 antibody. As used herein, the term “COV_3000” refers to a monoclonal antibody, or antigen-binding fragment thereof, that binds to the S2 subunit of a coronavirus, such as SARS-CoV-2, wherein the antibody comprises 1) a heavy chain variable domain comprising the amino acid sequence of SEQ ID NO:312 and a light chain variable domain comprising the amino acid sequence of SEQ ID NO:317; or 2) a heavy chain variable domain comprising a CDR1 comprising the amino acid sequence of SEQ ID NO:313, a CDR2 comprising the amino acid sequence of SEQ ID NO:314, and a CDR3 comprising the amino acid sequence of SEQ ID NO:315 and alight chain variable domain comprising a CDR1 comprising the amino acid sequence of SEQ ID NO:318, a CDR2 comprising the amino acid sequence of SEQ ID NO:319, and a CDR3 comprising the amino acid sequence of SEQ ID NO:320.

In one embodiment, the antibody is an isolated COV_3005 antibody. As used herein, the term “COV_3005” refers to a monoclonal antibody, or antigen-binding fragment thereof, that binds to the S2 subunit of a coronavirus, such as SARS-CoV-2, wherein the antibody comprises 1) a heavy chain variable domain comprising the amino acid sequence of SEQ ID NO:322 and a light chain variable domain comprising the amino acid sequence of SEQ ID NO:327; or 2) a heavy chain variable domain comprising a CDR1 comprising the amino acid sequence of SEQ ID NO:323, a CDR2 comprising the amino acid sequence of SEQ ID NO:324, and a CDR3 comprising the amino acid sequence of SEQ ID NO:325 and alight chain variable domain comprising a CDR1 comprising the amino acid sequence of SEQ ID NO:328, a CDR2 comprising the amino acid sequence of SEQ ID NO:329, and a CDR3 comprising the amino acid sequence of SEQ ID NO:330.

In one embodiment, the antibody is an isolated COV_3013 antibody. As used herein, the term “COV_3013” refers to a monoclonal antibody, or antigen-binding fragment thereof, that binds to the S2 subunit of a coronavirus, such as SARS-CoV-2, wherein the antibody comprises 1) a heavy chain variable domain comprising the amino acid sequence of SEQ ID NO:332 and a light chain variable domain comprising the amino acid sequence of SEQ ID NO:337; or 2) a heavy chain variable domain comprising a CDR1 comprising the amino acid sequence of SEQ ID NO:333, a CDR2 comprising the amino acid sequence of SEQ ID NO:334, and a CDR3 comprising the amino acid sequence of SEQ ID NO:335 and alight chain variable domain comprising a CDR1 comprising the amino acid sequence of SEQ ID NO:338, a CDR2 comprising the amino acid sequence of SEQ ID NO:339, and a CDR3 comprising the amino acid sequence of SEQ ID NO:340.

In one embodiment, the antibody is an isolated COV_3019 antibody. As used herein, the term “COV_3019” refers to a monoclonal antibody, or antigen-binding fragment thereof, that binds to the S2 subunit of a coronavirus, such as SARS-CoV-2, wherein the antibody comprises 1) a heavy chain variable domain comprising the amino acid sequence of SEQ ID NO:342 and a light chain variable domain comprising the amino acid sequence of SEQ ID NO:347; or 2) a heavy chain variable domain comprising a CDR1 comprising the amino acid sequence of SEQ ID NO:343, a CDR2 comprising the amino acid sequence of SEQ ID NO:344, and a CDR3 comprising the amino acid sequence of SEQ ID NO:345 and alight chain variable domain comprising a CDR1 comprising the amino acid sequence of SEQ ID NO:348, a CDR2 comprising the amino acid sequence of SEQ ID NO:349, and a CDR3 comprising the amino acid sequence of SEQ ID NO:350.

In one embodiment, the antibody is an isolated COV_3028 antibody. As used herein, the term “COV_3028” refers to a monoclonal antibody, or antigen-binding fragment thereof, that binds to the S2 subunit of a coronavirus, such as SARS-CoV-2, wherein the antibody comprises 1) a heavy chain variable domain comprising the amino acid sequence of SEQ ID NO:352 and a light chain variable domain comprising the amino acid sequence of SEQ ID NO:357; or 2) a heavy chain variable domain comprising a CDR1 comprising the amino acid sequence of SEQ ID NO:353, a CDR2 comprising the amino acid sequence of SEQ ID NO:354, and a CDR3 comprising the amino acid sequence of SEQ ID NO:355 and alight chain variable domain comprising a CDR1 comprising the amino acid sequence of SEQ ID NO:358, a CDR2 comprising the amino acid sequence of SEQ ID NO:359, and a CDR3 comprising the amino acid sequence of SEQ ID NO:360.

In one embodiment, the antibody is an isolated COV_3031 antibody. As used herein, the term “COV_3031” refers to a monoclonal antibody, or antigen-binding fragment thereof, that binds to the S2 subunit of a coronavirus, such as SARS-CoV-2, wherein the antibody comprises 1) a heavy chain variable domain comprising the amino acid sequence of SEQ ID NO:362 and a light chain variable domain comprising the amino acid sequence of SEQ ID NO:367; or 2) a heavy chain variable domain comprising a CDR1 comprising the amino acid sequence of SEQ ID NO:363, a CDR2 comprising the amino acid sequence of SEQ ID NO:364, and a CDR3 comprising the amino acid sequence of SEQ ID NO:365 and a light chain variable domain comprising a CDR1 comprising the amino acid sequence of SEQ ID NO:368, a CDR2 comprising the amino acid sequence of SEQ ID NO:369, and a CDR3 comprising the amino acid sequence of SEQ ID NO:370.

In one embodiment, the antibody is an isolated COV_3033 antibody. As used herein, the term “COV_3033” refers to a monoclonal antibody, or antigen-binding fragment thereof, that binds to the S2 subunit of a coronavirus, such as SARS-CoV-2, wherein the antibody comprises 1) a heavy chain variable domain comprising the amino acid sequence of SEQ ID NO:372 and a light chain variable domain comprising the amino acid sequence of SEQ ID NO:377; or 2) a heavy chain variable domain comprising a CDR1 comprising the amino acid sequence of SEQ ID NO:373, a CDR2 comprising the amino acid sequence of SEQ ID NO:374, and a CDR3 comprising the amino acid sequence of SEQ ID NO:375 and a light chain variable domain comprising a CDR1 comprising the amino acid sequence of SEQ ID NO:378, a CDR2 comprising the amino acid sequence of SEQ ID NO:379, and a CDR3 comprising the amino acid sequence of SEQ ID NO:380.

In one embodiment, the antibody is an isolated COV_3037 antibody. As used herein, the term “COV_3037” refers to a monoclonal antibody, or antigen-binding fragment thereof, that binds to the S2 subunit of a coronavirus, such as SARS-CoV-2, wherein the antibody comprises 1) a heavy chain variable domain comprising the amino acid sequence of SEQ ID NO:382 and a light chain variable domain comprising the amino acid sequence of SEQ ID NO:387; or 2) a heavy chain variable domain comprising a CDR1 comprising the amino acid sequence of SEQ ID NO:383, a CDR2 comprising the amino acid sequence of SEQ ID NO:384, and a CDR3 comprising the amino acid sequence of SEQ ID NO:385 and alight chain variable domain comprising a CDR1 comprising the amino acid sequence of SEQ ID NO:388, a CDR2 comprising the amino acid sequence of SEQ ID NO:389, and a CDR3 comprising the amino acid sequence of SEQ ID NO:390.

In one embodiment, the antibody is an isolated COV_3040 antibody. As used herein, the term “COV_3040” refers to a monoclonal antibody, or antigen-binding fragment thereof, that binds to the S2 subunit of a coronavirus, such as SARS-CoV-2, wherein the antibody comprises 1) a heavy chain variable domain comprising the amino acid sequence of SEQ ID NO:392 and a light chain variable domain comprising the amino acid sequence of SEQ ID NO:397; or 2) a heavy chain variable domain comprising a CDR1 comprising the amino acid sequence of SEQ ID NO:393, a CDR2 comprising the amino acid sequence of SEQ ID NO:394, and a CDR3 comprising the amino acid sequence of SEQ ID NO:395 and alight chain variable domain comprising a CDR1 comprising the amino acid sequence of SEQ ID NO:398, a CDR2 comprising the amino acid sequence of SEQ ID NO:399, and a CDR3 comprising the amino acid sequence of SEQ ID NO:400.

In one embodiment, the antibody is an isolated COV_3043 antibody. As used herein, the term “COV_3043” refers to a monoclonal antibody, or antigen-binding fragment thereof, that binds to the S2 subunit of a coronavirus, such as SARS-CoV-2, wherein the antibody comprises 1) a heavy chain variable domain comprising the amino acid sequence of SEQ ID NO:402 and a light chain variable domain comprising the amino acid sequence of SEQ ID NO:407; or 2) a heavy chain variable domain comprising a CDR1 comprising the amino acid sequence of SEQ ID NO:403, a CDR2 comprising the amino acid sequence of SEQ ID NO:404, and a CDR3 comprising the amino acid sequence of SEQ ID NO:405 and a light chain variable domain comprising a CDR1 comprising the amino acid sequence of SEQ ID NO:408, a CDR2 comprising the amino acid sequence of SEQ ID NO:409, and a CDR3 comprising the amino acid sequence of SEQ ID NO:410.

In one embodiment, the antibody is an isolated COV_3053 antibody. As used herein, the term “COV_3053” refers to a monoclonal antibody, or antigen-binding fragment thereof, that binds to the S1 NTD or S1 RBD domain of a coronavirus, such as SARS-CoV-2, wherein the antibody comprises 1) a heavy chain variable domain comprising the amino acid sequence of SEQ ID NO:412 and a light chain variable domain comprising the amino acid sequence of SEQ ID NO:417; or 2) a heavy chain variable domain comprising a CDR1 comprising the amino acid sequence of SEQ ID NO:413, a CDR2 comprising the amino acid sequence of SEQ ID NO:414, and a CDR3 comprising the amino acid sequence of SEQ ID NO:415 and a light chain variable domain comprising a CDR1 comprising the amino acid sequence of SEQ ID NO:418, a CDR2 comprising the amino acid sequence of SEQ ID NO:419, and a CDR3 comprising the amino acid sequence of SEQ ID NO:420.

In one embodiment, the antibody is an isolated COV_3088 antibody. As used herein, the term “COV_3088” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:422 and a light chain variable domain comprising the amino acid sequence of SEQ ID NO:427; or 2) a heavy chain variable domain comprising a CDR1 comprising the amino acid sequence of SEQ ID NO:423, a CDR2 comprising the amino acid sequence of SEQ ID NO:424, and a CDR3 comprising the amino acid sequence of SEQ ID NO:425 and a light chain variable domain comprising a CDR1 comprising the amino acid sequence of SEQ ID NO:428, a CDR2 comprising the amino acid sequence of SEQ ID NO:429, and a CDR3 comprising the amino acid sequence of SEQ ID NO:430.

In one embodiment, the antibody is an isolated COV_1012 antibody. As used herein, the term “COV_1012” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:452 and a light chain variable domain comprising the amino acid sequence of SEQ ID NO:457; or 2) a heavy chain variable domain comprising a CDR1 comprising the amino acid sequence of SEQ ID NO:453, a CDR2 comprising the amino acid sequence of SEQ ID NO:454, and a CDR3 comprising the amino acid sequence of SEQ ID NO:455 and a light chain variable domain comprising a CDR1 comprising the amino acid sequence of SEQ ID NO:458, a CDR2 comprising the amino acid sequence of SEQ ID NO:459, and a CDR3 comprising the amino acid sequence of SEQ ID NO:460.

In one embodiment, the antibody is an isolated COV_1025 antibody. As used herein, the term “COV_1025” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:462 and a light chain variable domain comprising the amino acid sequence of SEQ ID NO:467; or 2) a heavy chain variable domain comprising a CDR1 comprising the amino acid sequence of SEQ ID NO:463, a CDR2 comprising the amino acid sequence of SEQ ID NO:464, and a CDR3 comprising the amino acid sequence of SEQ ID NO:465 and alight chain variable domain comprising a CDR1 comprising the amino acid sequence of SEQ ID NO:468, a CDR2 comprising the amino acid sequence of SEQ ID NO:469, and a CDR3 comprising the amino acid sequence of SEQ ID NO:470.

In one embodiment, the antibody is an isolated COV_1032 antibody. As used herein, the term “COV_1032” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:472 and a light chain variable domain comprising the amino acid sequence of SEQ ID NO:477; or 2) a heavy chain variable domain comprising a CDR1 comprising the amino acid sequence of SEQ ID NO:473, a CDR2 comprising the amino acid sequence of SEQ ID NO:474, and a CDR3 comprising the amino acid sequence of SEQ ID NO:475 and a light chain variable domain comprising a CDR1 comprising the amino acid sequence of SEQ ID NO:478, a CDR2 comprising the amino acid sequence of SEQ ID NO:479, and a CDR3 comprising the amino acid sequence of SEQ ID NO:480.

In one embodiment, the antibody is an isolated COV_1050 antibody. As used herein, the term “COV_1050” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:482 and a light chain variable domain comprising the amino acid sequence of SEQ ID NO:487; or 2) a heavy chain variable domain comprising a CDR1 comprising the amino acid sequence of SEQ ID NO:483, a CDR2 comprising the amino acid sequence of SEQ ID NO:484, and a CDR3 comprising the amino acid sequence of SEQ ID NO:485 and a light chain variable domain comprising a CDR1 comprising the amino acid sequence of SEQ ID NO:488, a CDR2 comprising the amino acid sequence of SEQ ID NO:489, and a CDR3 comprising the amino acid sequence of SEQ ID NO:490.

In one embodiment, the antibody is an isolated COV_1056 antibody. As used herein, the term “COV_1056” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO: 492 and a light chain variable domain comprising the amino acid sequence of SEQ ID NO:497; or 2) a heavy chain variable domain comprising a CDR1 comprising the amino acid sequence of SEQ ID NO:493, a CDR2 comprising the amino acid sequence of SEQ ID NO:494, and a CDR3 comprising the amino acid sequence of SEQ ID NO:495 and a light chain variable domain comprising a CDR1 comprising the amino acid sequence of SEQ ID NO:498, a CDR2 comprising the amino acid sequence of SEQ ID NO:499, and a CDR3 comprising the amino acid sequence of SEQ ID NO:500.

In one embodiment, the antibody is an isolated COV_1060 antibody. As used herein, the term “COV_1060” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:502 and a light chain variable domain comprising the amino acid sequence of SEQ ID NO:507; or 2) a heavy chain variable domain comprising a CDR1 comprising the amino acid sequence of SEQ ID NO:503, a CDR2 comprising the amino acid sequence of SEQ ID NO:504, and a CDR3 comprising the amino acid sequence of SEQ ID NO:505 and alight chain variable domain comprising a CDR1 comprising the amino acid sequence of SEQ ID NO:508, a CDR2 comprising the amino acid sequence of SEQ ID NO:509, and a CDR3 comprising the amino acid sequence of SEQ ID NO:510.

In one embodiment, the antibody is an isolated COV_1063 antibody. As used herein, the term “COV_1063” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:512 and a light chain variable domain comprising the amino acid sequence of SEQ ID NO:517; or 2) a heavy chain variable domain comprising a CDR1 comprising the amino acid sequence of SEQ ID NO:513, a CDR2 comprising the amino acid sequence of SEQ ID NO:514, and a CDR3 comprising the amino acid sequence of SEQ ID NO:515 and alight chain variable domain comprising a CDR1 comprising the amino acid sequence of SEQ ID NO:518, a CDR2 comprising the amino acid sequence of SEQ ID NO:519, and a CDR3 comprising the amino acid sequence of SEQ ID NO:520.

In one embodiment, the antibody is an isolated COV_1071 antibody. As used herein, the term “COV_1071” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:522 and a light chain variable domain comprising the amino acid sequence of SEQ ID NO:527; or 2) a heavy chain variable domain comprising a CDR1 comprising the amino acid sequence of SEQ ID NO:523, a CDR2 comprising the amino acid sequence of SEQ ID NO:524, and a CDR3 comprising the amino acid sequence of SEQ ID NO:525 and alight chain variable domain comprising a CDR1 comprising the amino acid sequence of SEQ ID NO:528, a CDR2 comprising the amino acid sequence of SEQ ID NO:529, and a CDR3 comprising the amino acid sequence of SEQ ID NO:530.

In one embodiment, the antibody is an isolated COV_1076 antibody. As used herein, the term “COV_1076” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:532 and a light chain variable domain comprising the amino acid sequence of SEQ ID NO:537; or 2) a heavy chain variable domain comprising a CDR1 comprising the amino acid sequence of SEQ ID NO:533, a CDR2 comprising the amino acid sequence of SEQ ID NO:534, and a CDR3 comprising the amino acid sequence of SEQ ID NO:535 and a light chain variable domain comprising a CDR1 comprising the amino acid sequence of SEQ ID NO:538, a CDR2 comprising the amino acid sequence of SEQ ID NO:539, and a CDR3 comprising the amino acid sequence of SEQ ID NO:540.

In one embodiment, the antibody is an isolated COV_1082 antibody. As used herein, the term “COV_1082” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:542 and a light chain variable domain comprising the amino acid sequence of SEQ ID NO:547; or 2) a heavy chain variable domain comprising a CDR1 comprising the amino acid sequence of SEQ ID NO:543, a CDR2 comprising the amino acid sequence of SEQ ID NO:544, and a CDR3 comprising the amino acid sequence of SEQ ID NO:545 and alight chain variable domain comprising a CDR1 comprising the amino acid sequence of SEQ ID NO:548, a CDR2 comprising the amino acid sequence of SEQ ID NO:549, and a CDR3 comprising the amino acid sequence of SEQ ID NO:550.

In one embodiment, the antibody is an isolated COV_1085 antibody. As used herein, the term “COV_1085” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:552 and a light chain variable domain comprising the amino acid sequence of SEQ ID NO:557; or 2) a heavy chain variable domain comprising a CDR1 comprising the amino acid sequence of SEQ ID NO:553, a CDR2 comprising the amino acid sequence of SEQ ID NO:554, and a CDR3 comprising the amino acid sequence of SEQ ID NO:555 and alight chain variable domain comprising a CDR1 comprising the amino acid sequence of SEQ ID NO:558, a CDR2 comprising the amino acid sequence of SEQ ID NO:559, and a CDR3 comprising the amino acid sequence of SEQ ID NO:560.

In one embodiment, the antibody is an isolated COV_1086 antibody. As used herein, the term “COV_1086” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:562 and a light chain variable domain comprising the amino acid sequence of SEQ ID NO:567; or 2) a heavy chain variable domain comprising a CDR1 comprising the amino acid sequence of SEQ ID NO:563, a CDR2 comprising the amino acid sequence of SEQ ID NO:564, and a CDR3 comprising the amino acid sequence of SEQ ID NO:565 and alight chain variable domain comprising a CDR1 comprising the amino acid sequence of SEQ ID NO:568, a CDR2 comprising the amino acid sequence of SEQ ID NO:569, and a CDR3 comprising the amino acid sequence of SEQ ID NO:570.

In one embodiment, the antibody is an isolated COV_1087 antibody. As used herein, the term “COV_1087” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:572 and a light chain variable domain comprising the amino acid sequence of SEQ ID NO:577; or 2) a heavy chain variable domain comprising a CDR1 comprising the amino acid sequence of SEQ ID NO:573, a CDR2 comprising the amino acid sequence of SEQ ID NO:574, and a CDR3 comprising the amino acid sequence of SEQ ID NO:575 and a light chain variable domain comprising a CDR1 comprising the amino acid sequence of SEQ ID NO:578, a CDR2 comprising the amino acid sequence of SEQ ID NO:579, and a CDR3 comprising the amino acid sequence of SEQ ID NO:580.

In one embodiment, the antibody is an isolated COV_1097 antibody. As used herein, the term “COV_1097” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:582 and a light chain variable domain comprising the amino acid sequence of SEQ ID NO:587; or 2) a heavy chain variable domain comprising a CDR1 comprising the amino acid sequence of SEQ ID NO:583, a CDR2 comprising the amino acid sequence of SEQ ID NO:584, and a CDR3 comprising the amino acid sequence of SEQ ID NO:585 and alight chain variable domain comprising a CDR1 comprising the amino acid sequence of SEQ ID NO:588, a CDR2 comprising the amino acid sequence of SEQ ID NO:589, and a CDR3 comprising the amino acid sequence of SEQ ID NO:590.

In one embodiment, the antibody is an isolated COV_1116 antibody. As used herein, the term “COV_1116” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:592 and a light chain variable domain comprising the amino acid sequence of SEQ ID NO:597; or 2) a heavy chain variable domain comprising a CDR1 comprising the amino acid sequence of SEQ ID NO:593, a CDR2 comprising the amino acid sequence of SEQ ID NO:594, and a CDR3 comprising the amino acid sequence of SEQ ID NO:595 and a light chain variable domain comprising a CDR1 comprising the amino acid sequence of SEQ ID NO:598, a CDR2 comprising the amino acid sequence of SEQ ID NO:599, and a CDR3 comprising the amino acid sequence of SEQ ID NO:600.

In one embodiment, the antibody is an isolated COV_1118 antibody. As used herein, the term “COV_1118” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:602 and a light chain variable domain comprising the amino acid sequence of SEQ ID NO:607; or 2) a heavy chain variable domain comprising a CDR1 comprising the amino acid sequence of SEQ ID NO:603, a CDR2 comprising the amino acid sequence of SEQ ID NO:604, and a CDR3 comprising the amino acid sequence of SEQ ID NO:605 and a light chain variable domain comprising a CDR1 comprising the amino acid sequence of SEQ ID NO:608, a CDR2 comprising the amino acid sequence of SEQ ID NO:609, and a CDR3 comprising the amino acid sequence of SEQ ID NO:610.

In one embodiment, the antibody is an isolated COV_1122 antibody. As used herein, the term “COV_1122” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:612 and a light chain variable domain comprising the amino acid sequence of SEQ ID NO:617; or 2) a heavy chain variable domain comprising a CDR1 comprising the amino acid sequence of SEQ ID NO:613, a CDR2 comprising the amino acid sequence of SEQ ID NO:614, and a CDR3 comprising the amino acid sequence of SEQ ID NO:615 and alight chain variable domain comprising a CDR1 comprising the amino acid sequence of SEQ ID NO:618, a CDR2 comprising the amino acid sequence of SEQ ID NO:619, and a CDR3 comprising the amino acid sequence of SEQ ID NO:620.

In one embodiment, the antibody is an isolated COV_1131 antibody. As used herein, the term “COV_1131” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:622 and a light chain variable domain comprising the amino acid sequence of SEQ ID NO:627; or 2) a heavy chain variable domain comprising a CDR1 comprising the amino acid sequence of SEQ ID NO:623, a CDR2 comprising the amino acid sequence of SEQ ID NO:624, and a CDR3 comprising the amino acid sequence of SEQ ID NO:625 and a light chain variable domain comprising a CDR1 comprising the amino acid sequence of SEQ ID NO:628, a CDR2 comprising the amino acid sequence of SEQ ID NO:629, and a CDR3 comprising the amino acid sequence of SEQ ID NO:630.

In one embodiment, the antibody is an isolated COV_1136 antibody. As used herein, the term “COV_1136” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:632 and a light chain variable domain comprising the amino acid sequence of SEQ ID NO:637; or 2) a heavy chain variable domain comprising a CDR1 comprising the amino acid sequence of SEQ ID NO:633, a CDR2 comprising the amino acid sequence of SEQ ID NO:634, and a CDR3 comprising the amino acid sequence of SEQ ID NO:635 and alight chain variable domain comprising a CDR1 comprising the amino acid sequence of SEQ ID NO:638, a CDR2 comprising the amino acid sequence of SEQ ID NO:639, and a CDR3 comprising the amino acid sequence of SEQ ID NO:640.

In one embodiment, the antibody is an isolated COV_1144 antibody. As used herein, the term “COV_1144” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:642 and a light chain variable domain comprising the amino acid sequence of SEQ ID NO:647; or 2) a heavy chain variable domain comprising a CDR1 comprising the amino acid sequence of SEQ ID NO:643, a CDR2 comprising the amino acid sequence of SEQ ID NO:644, and a CDR3 comprising the amino acid sequence of SEQ ID NO:645 and a light chain variable domain comprising a CDR1 comprising the amino acid sequence of SEQ ID NO:648, a CDR2 comprising the amino acid sequence of SEQ ID NO:649, and a CDR3 comprising the amino acid sequence of SEQ ID NO:650.

In one embodiment, the antibody is an isolated COV_1145 antibody. As used herein, the term “COV_1145” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:652 and a light chain variable domain comprising the amino acid sequence of SEQ ID NO:657; or 2) a heavy chain variable domain comprising a CDR1 comprising the amino acid sequence of SEQ ID NO:653, a CDR2 comprising the amino acid sequence of SEQ ID NO:654, and a CDR3 comprising the amino acid sequence of SEQ ID NO:655 and alight chain variable domain comprising a CDR1 comprising the amino acid sequence of SEQ ID NO:658, a CDR2 comprising the amino acid sequence of SEQ ID NO:659, and a CDR3 comprising the amino acid sequence of SEQ ID NO:660.

In one embodiment, the antibody is an isolated COV_1149 antibody. As used herein, the term “COV_1149” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:662 and a light chain variable domain comprising the amino acid sequence of SEQ ID NO:667; or 2) a heavy chain variable domain comprising a CDR1 comprising the amino acid sequence of SEQ ID NO:663, a CDR2 comprising the amino acid sequence of SEQ ID NO:664, and a CDR3 comprising the amino acid sequence of SEQ ID NO:665 and a light chain variable domain comprising a CDR1 comprising the amino acid sequence of SEQ ID NO:668, a CDR2 comprising the amino acid sequence of SEQ ID NO:669, and a CDR3 comprising the amino acid sequence of SEQ ID NO:670.

In one embodiment, the antibody is an isolated COV_1151 antibody. As used herein, the term “COV_1151” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:672 and a light chain variable domain comprising the amino acid sequence of SEQ ID NO:677; or 2) a heavy chain variable domain comprising a CDR1 comprising the amino acid sequence of SEQ ID NO:673, a CDR2 comprising the amino acid sequence of SEQ ID NO:674, and a CDR3 comprising the amino acid sequence of SEQ ID NO:675 and a light chain variable domain comprising a CDR1 comprising the amino acid sequence of SEQ ID NO:678, a CDR2 comprising the amino acid sequence of SEQ ID NO:679, and a CDR3 comprising the amino acid sequence of SEQ ID NO:680.

In one embodiment, the antibody is an isolated COV_1154 antibody. As used herein, the term “COV_1154” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO: 682 and a light chain variable domain comprising the amino acid sequence of SEQ ID NO:687; or 2) a heavy chain variable domain comprising a CDR1 comprising the amino acid sequence of SEQ ID NO:683, a CDR2 comprising the amino acid sequence of SEQ ID NO:684, and a CDR3 comprising the amino acid sequence of SEQ ID NO:685 and alight chain variable domain comprising a CDR1 comprising the amino acid sequence of SEQ ID NO:688, a CDR2 comprising the amino acid sequence of SEQ ID NO:689, and a CDR3 comprising the amino acid sequence of SEQ ID NO:690.

In one embodiment, the antibody is an isolated COV_1165 antibody. As used herein, the term “COV_1165” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:692 and a light chain variable domain comprising the amino acid sequence of SEQ ID NO:697; or 2) a heavy chain variable domain comprising a CDR1 comprising the amino acid sequence of SEQ ID NO:693, a CDR2 comprising the amino acid sequence of SEQ ID NO:694, and a CDR3 comprising the amino acid sequence of SEQ ID NO:695 and a light chain variable domain comprising a CDR1 comprising the amino acid sequence of SEQ ID NO:698, a CDR2 comprising the amino acid sequence of SEQ ID NO:699, and a CDR3 comprising the amino acid sequence of SEQ ID NO:700.

In one embodiment, the antibody is an isolated COV_1166 antibody. As used herein, the term “COV_1166” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:702 and a light chain variable domain comprising the amino acid sequence of SEQ ID NO:707; or 2) a heavy chain variable domain comprising a CDR1 comprising the amino acid sequence of SEQ ID NO:703, a CDR2 comprising the amino acid sequence of SEQ ID NO:704, and a CDR3 comprising the amino acid sequence of SEQ ID NO:705 and alight chain variable domain comprising a CDR1 comprising the amino acid sequence of SEQ ID NO:708, a CDR2 comprising the amino acid sequence of SEQ ID NO:709, and a CDR3 comprising the amino acid sequence of SEQ ID NO:710.

In one embodiment, the antibody is an isolated COV_1170 antibody. As used herein, the term “COV_1170” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:712 and a light chain variable domain comprising the amino acid sequence of SEQ ID NO:717; or 2) a heavy chain variable domain comprising a CDR1 comprising the amino acid sequence of SEQ ID NO:713, a CDR2 comprising the amino acid sequence of SEQ ID NO:714, and a CDR3 comprising the amino acid sequence of SEQ ID NO:715 and alight chain variable domain comprising a CDR1 comprising the amino acid sequence of SEQ ID NO:718, a CDR2 comprising the amino acid sequence of SEQ ID NO:719, and a CDR3 comprising the amino acid sequence of SEQ ID NO:720.

In one embodiment, the antibody is an isolated COV_1172 antibody. As used herein, the term “COV_1172” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:722 and a light chain variable domain comprising the amino acid sequence of SEQ ID NO:727; or 2) a heavy chain variable domain comprising a CDR1 comprising the amino acid sequence of SEQ ID NO:723, a CDR2 comprising the amino acid sequence of SEQ ID NO:724, and a CDR3 comprising the amino acid sequence of SEQ ID NO:725 and alight chain variable domain comprising a CDR1 comprising the amino acid sequence of SEQ ID NO:728, a CDR2 comprising the amino acid sequence of SEQ ID NO:729, and a CDR3 comprising the amino acid sequence of SEQ ID NO:730.

In one embodiment, the antibody is an isolated COV_1177 antibody. As used herein, the term “COV_1177” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:732 and a light chain variable domain comprising the amino acid sequence of SEQ ID NO:737; or 2) a heavy chain variable domain comprising a CDR1 comprising the amino acid sequence of SEQ ID NO:733, a CDR2 comprising the amino acid sequence of SEQ ID NO:734, and a CDR3 comprising the amino acid sequence of SEQ ID NO:735 and alight chain variable domain comprising a CDR1 comprising the amino acid sequence of SEQ ID NO:738, a CDR2 comprising the amino acid sequence of SEQ ID NO:739, and a CDR3 comprising the amino acid sequence of SEQ ID NO:740.

In one embodiment, the antibody is an isolated COV_1184 antibody. As used herein, the term “COV_1184” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:742 and a light chain variable domain comprising the amino acid sequence of SEQ ID NO:747; or 2) a heavy chain variable domain comprising a CDR1 comprising the amino acid sequence of SEQ ID NO:743, a CDR2 comprising the amino acid sequence of SEQ ID NO:744, and a CDR3 comprising the amino acid sequence of SEQ ID NO:745 and a light chain variable domain comprising a CDR1 comprising the amino acid sequence of SEQ ID NO:748, a CDR2 comprising the amino acid sequence of SEQ ID NO:749, and a CDR3 comprising the amino acid sequence of SEQ ID NO:750.

In one embodiment, the antibody is an isolated COV_1198 antibody. As used herein, the term “COV_1198” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:752 and a light chain variable domain comprising the amino acid sequence of SEQ ID NO:757; or 2) a heavy chain variable domain comprising a CDR1 comprising the amino acid sequence of SEQ ID NO:753, a CDR2 comprising the amino acid sequence of SEQ ID NO:754, and a CDR3 comprising the amino acid sequence of SEQ ID NO:755 and alight chain variable domain comprising a CDR1 comprising the amino acid sequence of SEQ ID NO:758, a CDR2 comprising the amino acid sequence of SEQ ID NO:759, and a CDR3 comprising the amino acid sequence of SEQ ID NO:760.

In one embodiment, the antibody is an isolated COV_2032 antibody. As used herein, the term “COV_2032” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:762 and a light chain variable domain comprising the amino acid sequence of SEQ ID NO:767; or 2) a heavy chain variable domain comprising a CDR1 comprising the amino acid sequence of SEQ ID NO:763, a CDR2 comprising the amino acid sequence of SEQ ID NO:764, and a CDR3 comprising the amino acid sequence of SEQ ID NO:765 and alight chain variable domain comprising a CDR1 comprising the amino acid sequence of SEQ ID NO:768, a CDR2 comprising the amino acid sequence of SEQ ID NO:769, and a CDR3 comprising the amino acid sequence of SEQ ID NO:770.

In one embodiment, the antibody is an isolated COV_2048 antibody. As used herein, the term “COV_2048” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:772 and a light chain variable domain comprising the amino acid sequence of SEQ ID NO:777; or 2) a heavy chain variable domain comprising a CDR1 comprising the amino acid sequence of SEQ ID NO:773, a CDR2 comprising the amino acid sequence of SEQ ID NO:774, and a CDR3 comprising the amino acid sequence of SEQ ID NO:775 and alight chain variable domain comprising a CDR1 comprising the amino acid sequence of SEQ ID NO:778, a CDR2 comprising the amino acid sequence of SEQ ID NO:779, and a CDR3 comprising the amino acid sequence of SEQ ID NO:780.

In one embodiment, the antibody is an isolated COV_2055 antibody. As used herein, the term “COV_2055” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:782 and a light chain variable domain comprising the amino acid sequence of SEQ ID NO:787; or 2) a heavy chain variable domain comprising a CDR1 comprising the amino acid sequence of SEQ ID NO:783, a CDR2 comprising the amino acid sequence of SEQ ID NO:784, and a CDR3 comprising the amino acid sequence of SEQ ID NO:785 and a light chain variable domain comprising a CDR1 comprising the amino acid sequence of SEQ ID NO:788, a CDR2 comprising the amino acid sequence of SEQ ID NO:789, and a CDR3 comprising the amino acid sequence of SEQ ID NO:790.

In one embodiment, the antibody is an isolated COV_2056 antibody. As used herein, the term “COV_2056” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:792 and a light chain variable domain comprising the amino acid sequence of SEQ ID NO:797; or 2) a heavy chain variable domain comprising a CDR1 comprising the amino acid sequence of SEQ ID NO:793, a CDR2 comprising the amino acid sequence of SEQ ID NO:794, and a CDR3 comprising the amino acid sequence of SEQ ID NO:795 and alight chain variable domain comprising a CDR1 comprising the amino acid sequence of SEQ ID NO:798, a CDR2 comprising the amino acid sequence of SEQ ID NO:799, and a CDR3 comprising the amino acid sequence of SEQ ID NO:800.

In one embodiment, the antibody is an isolated COV_2064 antibody. As used herein, the term “COV_2064” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:802 and a light chain variable domain comprising the amino acid sequence of SEQ ID NO:807; or 2) a heavy chain variable domain comprising a CDR1 comprising the amino acid sequence of SEQ ID NO:803, a CDR2 comprising the amino acid sequence of SEQ ID NO:804, and a CDR3 comprising the amino acid sequence of SEQ ID NO:805 and a light chain variable domain comprising a CDR1 comprising the amino acid sequence of SEQ ID NO:808, a CDR2 comprising the amino acid sequence of SEQ ID NO:809, and a CDR3 comprising the amino acid sequence of SEQ ID NO:810.

In one embodiment, the antibody is an isolated COV_2066 antibody. As used herein, the term “COV_2066” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:812 and a light chain variable domain comprising the amino acid sequence of SEQ ID NO:817; or 2) a heavy chain variable domain comprising a CDR1 comprising the amino acid sequence of SEQ ID NO:813, a CDR2 comprising the amino acid sequence of SEQ ID NO:814, and a CDR3 comprising the amino acid sequence of SEQ ID NO:815 and a light chain variable domain comprising a CDR1 comprising the amino acid sequence of SEQ ID NO:818, a CDR2 comprising the amino acid sequence of SEQ ID NO:819, and a CDR3 comprising the amino acid sequence of SEQ ID NO: 820.

In one embodiment, the antibody is an isolated COV_2077 antibody. As used herein, the term “COV_2077” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:822 and a light chain variable domain comprising the amino acid sequence of SEQ ID NO:827; or 2) a heavy chain variable domain comprising a CDR1 comprising the amino acid sequence of SEQ ID NO:823, a CDR2 comprising the amino acid sequence of SEQ ID NO:824, and a CDR3 comprising the amino acid sequence of SEQ ID NO:825 and a light chain variable domain comprising a CDR1 comprising the amino acid sequence of SEQ ID NO:828, a CDR2 comprising the amino acid sequence of SEQ ID NO:829, and a CDR3 comprising the amino acid sequence of SEQ ID NO:830.

In one embodiment, the antibody is an isolated COV_2093 antibody. As used herein, the term “COV_2093” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:832 and a light chain variable domain comprising the amino acid sequence of SEQ ID NO:837; or 2) a heavy chain variable domain comprising a CDR1 comprising the amino acid sequence of SEQ ID NO:833, a CDR2 comprising the amino acid sequence of SEQ ID NO:834, and a CDR3 comprising the amino acid sequence of SEQ ID NO:835 and a light chain variable domain comprising a CDR1 comprising the amino acid sequence of SEQ ID NO:838, a CDR2 comprising the amino acid sequence of SEQ ID NO:839, and a CDR3 comprising the amino acid sequence of SEQ ID NO:840.

In one embodiment, the antibody is an isolated COV_2137 antibody. As used herein, the term “COV_2137” refers to a monoclonal antibody, or antigen-binding fragment thereof, that binds to the S1 NTD domain of a coronavirus, such as SARS-CoV-2, wherein the antibody comprises 1) a heavy chain variable domain comprising the amino acid sequence of SEQ ID NO:842 and a light chain variable domain comprising the amino acid sequence of SEQ ID NO: 847; or 2) a heavy chain variable domain comprising a CDR1 comprising the amino acid sequence of SEQ ID NO:843, a CDR2 comprising the amino acid sequence of SEQ ID NO:844, and a CDR3 comprising the amino acid sequence of SEQ ID NO:845 and a light chain variable domain comprising a CDR1 comprising the amino acid sequence of SEQ ID NO:848, a CDR2 comprising the amino acid sequence of SEQ ID NO:849, and a CDR3 comprising the amino acid sequence of SEQ ID NO:850; or 3) a heavy chain variable domain comprising a CDR1 comprising the amino acid sequence of SEQ ID NO:1117, a CDR2 comprising the amino acid sequence of SEQ ID NO: 1147, and a CDR3 comprising the amino acid sequence of SEQ ID NO: 1177 and a light chain variable domain comprising a CDR1 comprising the amino acid sequence of SEQ ID NO: 1207, a CDR2 comprising the amino acid sequence EVN, and a CDR3 comprising the amino acid sequence of SEQ ID NO: 1237.

In one embodiment, the antibody is an isolated COV_2143 antibody. As used herein, the term “COV_2143” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:852 and a light chain variable domain comprising the amino acid sequence of SEQ ID NO:857; or 2) a heavy chain variable domain comprising a CDR1 comprising the amino acid sequence of SEQ ID NO:853, a CDR2 comprising the amino acid sequence of SEQ ID NO:854, and a CDR3 comprising the amino acid sequence of SEQ ID NO:855 and a light chain variable domain comprising a CDR1 comprising the amino acid sequence of SEQ ID NO:858, a CDR2 comprising the amino acid sequence of SEQ ID NO:859, and a CDR3 comprising the amino acid sequence of SEQ ID NO:860.

In one embodiment, the antibody is an isolated COV_2169 antibody. As used herein, the term “COV_2169” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:862 and a light chain variable domain comprising the amino acid sequence of SEQ ID NO:867; or 2) a heavy chain variable domain comprising a CDR1 comprising the amino acid sequence of SEQ ID NO:863, a CDR2 comprising the amino acid sequence of SEQ ID NO:864, and a CDR3 comprising the amino acid sequence of SEQ ID NO:865 and a light chain variable domain comprising a CDR1 comprising the amino acid sequence of SEQ ID NO:868, a CDR2 comprising the amino acid sequence of SEQ ID NO:869, and a CDR3 comprising the amino acid sequence of SEQ ID NO: 870.

In one embodiment, the antibody is an isolated COV_2172 antibody. As used herein, the term “COV_2172” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:872 and a light chain variable domain comprising the amino acid sequence of SEQ ID NO:877; or 2) a heavy chain variable domain comprising a CDR1 comprising the amino acid sequence of SEQ ID NO:873, a CDR2 comprising the amino acid sequence of SEQ ID NO:874, and a CDR3 comprising the amino acid sequence of SEQ ID NO:875 and a light chain variable domain comprising a CDR1 comprising the amino acid sequence of SEQ ID NO:878, a CDR2 comprising the amino acid sequence of SEQ ID NO:879, and a CDR3 comprising the amino acid sequence of SEQ ID NO:880; or 3) a heavy chain variable domain comprising a CDR1 comprising the amino acid sequence of SEQ ID NO:1127, a CDR2 comprising the amino acid sequence of SEQ ID NO: 1157, and a CDR3 comprising the amino acid sequence of SEQ ID NO: 1187 and a light chain variable domain comprising a CDR1 comprising the amino acid sequence of SEQ ID NO: 1217, a CDR2 comprising the amino acid sequence DVS, and a CDR3 comprising the amino acid sequence of SEQ ID NO: 1247.

In one embodiment, the antibody is an isolated COV_2174 antibody. As used herein, the term “COV_2174” refers to a monoclonal antibody, or antigen-binding fragment thereof, that binds to the S1 RBD domain of a coronavirus, such as SARS-CoV-2, wherein the antibody comprises 1) a heavy chain variable domain comprising the amino acid sequence of SEQ ID NO:882 and a light chain variable domain comprising the amino acid sequence of SEQ ID NO:887; or 2) a heavy chain variable domain comprising a CDR1 comprising the amino acid sequence of SEQ ID NO:883, a CDR2 comprising the amino acid sequence of SEQ ID NO:884, and a CDR3 comprising the amino acid sequence of SEQ ID NO:885 and alight chain variable domain comprising a CDR1 comprising the amino acid sequence of SEQ ID NO:888, a CDR2 comprising the amino acid sequence of SEQ ID NO:889, and a CDR3 comprising the amino acid sequence of SEQ ID NO:890; or 3) a heavy chain variable domain comprising a CDR1 comprising the amino acid sequence of SEQ ID NO:1129, a CDR2 comprising the amino acid sequence of SEQ ID NO: 1159, and a CDR3 comprising the amino acid sequence of SEQ ID NO: 1189 and a light chain variable domain comprising a CDR1 comprising the amino acid sequence of SEQ ID NO: 1219, a CDR2 comprising the amino acid sequence AAS, and a CDR3 comprising the amino acid sequence of SEQ ID NO: 1249.

In one embodiment, the antibody is an isolated COV_2205 antibody. As used herein, the term “COV_2205” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:892 and a light chain variable domain comprising the amino acid sequence of SEQ ID NO:897; or 2) a heavy chain variable domain comprising a CDR1 comprising the amino acid sequence of SEQ ID NO:893, a CDR2 comprising the amino acid sequence of SEQ ID NO:894, and a CDR3 comprising the amino acid sequence of SEQ ID NO:895 and a light chain variable domain comprising a CDR1 comprising the amino acid sequence of SEQ ID NO:898, a CDR2 comprising the amino acid sequence of SEQ ID NO:899, and a CDR3 comprising the amino acid sequence of SEQ ID NO:900.

In one embodiment, the antibody is an isolated COV_2215 antibody. As used herein, the term “COV_2215” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:902 and a light chain variable domain comprising the amino acid sequence of SEQ ID NO:907; or 2) a heavy chain variable domain comprising a CDR1 comprising the amino acid sequence of SEQ ID NO:903, a CDR2 comprising the amino acid sequence of SEQ ID NO:904, and a CDR3 comprising the amino acid sequence of SEQ ID NO:905 and alight chain variable domain comprising a CDR1 comprising the amino acid sequence of SEQ ID NO:908, a CDR2 comprising the amino acid sequence of SEQ ID NO:909, and a CDR3 comprising the amino acid sequence of SEQ ID NO:910.

In one embodiment, the antibody is an isolated COV_3049 antibody. As used herein, the term “COV_3049” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:912 and a light chain variable domain comprising the amino acid sequence of SEQ ID NO:917; or 2) a heavy chain variable domain comprising a CDR1 comprising the amino acid sequence of SEQ ID NO:913, a CDR2 comprising the amino acid sequence of SEQ ID NO:914, and a CDR3 comprising the amino acid sequence of SEQ ID NO:915 and alight chain variable domain comprising a CDR1 comprising the amino acid sequence of SEQ ID NO:918, a CDR2 comprising the amino acid sequence of SEQ ID NO:919, and a CDR3 comprising the amino acid sequence of SEQ ID NO:920.

In one embodiment, the antibody is an isolated COV_3069 antibody. As used herein, the term “COV_3069” refers to a monoclonal antibody, or antigen-binding fragment thereof, that binds to the S1 domain, S1 RBD domain, S1 NTD domain, or S2 domain of a coronavirus, such as SARS-CoV-2, wherein the antibody comprises 1) a heavy chain variable domain comprising the amino acid sequence of SEQ ID NO:922 and a light chain variable domain comprising the amino acid sequence of SEQ ID NO:927; or 2) a heavy chain variable domain comprising a CDR1 comprising the amino acid sequence of SEQ ID NO:923, a CDR2 comprising the amino acid sequence of SEQ ID NO:924, and a CDR3 comprising the amino acid sequence of SEQ ID NO:925 and a light chain variable domain comprising a CDR1 comprising the amino acid sequence of SEQ ID NO:928, a CDR2 comprising the amino acid sequence of SEQ ID NO:929, and a CDR3 comprising the amino acid sequence of SEQ ID NO:930.

In one embodiment, the antibody is an isolated COV_3077 antibody. As used herein, the term “COV_3077” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:932 and a light chain variable domain comprising the amino acid sequence of SEQ ID NO:937; or 2) a heavy chain variable domain comprising a CDR1 comprising the amino acid sequence of SEQ ID NO:933, a CDR2 comprising the amino acid sequence of SEQ ID NO:934, and a CDR3 comprising the amino acid sequence of SEQ ID NO:935 and alight chain variable domain comprising a CDR1 comprising the amino acid sequence of SEQ ID NO:938, a CDR2 comprising the amino acid sequence of SEQ ID NO:939, and a CDR3 comprising the amino acid sequence of SEQ ID NO:940.

In one embodiment, the antibody is an isolated COV_3079 antibody. As used herein, the term “COV_3079” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:942 and a light chain variable domain comprising the amino acid sequence of SEQ ID NO:947; or 2) a heavy chain variable domain comprising a CDR1 comprising the amino acid sequence of SEQ ID NO:943, a CDR2 comprising the amino acid sequence of SEQ ID NO:944, and a CDR3 comprising the amino acid sequence of SEQ ID NO:945 and alight chain variable domain comprising a CDR1 comprising the amino acid sequence of SEQ ID NO:948, a CDR2 comprising the amino acid sequence of SEQ ID NO:949, and a CDR3 comprising the amino acid sequence of SEQ ID NO:950.

In one embodiment, the antibody is an isolated COV_3100 antibody. As used herein, the term “COV_3100” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:952 and a light chain variable domain comprising the amino acid sequence of SEQ ID NO:957; or 2) a heavy chain variable domain comprising a CDR1 comprising the amino acid sequence of SEQ ID NO:953, a CDR2 comprising the amino acid sequence of SEQ ID NO:954, and a CDR3 comprising the amino acid sequence of SEQ ID NO:955 and alight chain variable domain comprising a CDR1 comprising the amino acid sequence of SEQ ID NO:958, a CDR2 comprising the amino acid sequence of SEQ ID NO:959, and a CDR3 comprising the amino acid sequence of SEQ ID NO:960.

In one embodiment, the antibody is an isolated COV_3103 antibody. As used herein, the term “COV_3103” refers to a monoclonal antibody, or antigen-binding fragment thereof, that binds to the S2 domain of a coronavirus, such as SARS-CoV-2, wherein the antibody comprises 1) a heavy chain variable domain comprising the amino acid sequence of SEQ ID NO:962 and a light chain variable domain comprising the amino acid sequence of SEQ ID NO:967; or 2) a heavy chain variable domain comprising a CDR1 comprising the amino acid sequence of SEQ ID NO:963, a CDR2 comprising the amino acid sequence of SEQ ID NO:964, and a CDR3 comprising the amino acid sequence of SEQ ID NO:965 and alight chain variable domain comprising a CDR1 comprising the amino acid sequence of SEQ ID NO:968, a CDR2 comprising the amino acid sequence of SEQ ID NO:969, and a CDR3 comprising the amino acid sequence of SEQ ID NO:970.

In one embodiment, the antibody is an isolated COV_3129 antibody. As used herein, the term “COV_3129” refers to a monoclonal antibody, or antigen-binding fragment thereof, that binds to the S1 domain of a coronavirus, such as SARS-CoV-2, wherein the antibody comprises 1) a heavy chain variable domain comprising the amino acid sequence of SEQ ID NO:972 and a light chain variable domain comprising the amino acid sequence of SEQ ID NO:977; or 2) a heavy chain variable domain comprising a CDR1 comprising the amino acid sequence of SEQ ID NO:973, a CDR2 comprising the amino acid sequence of SEQ ID NO:974, and a CDR3 comprising the amino acid sequence of SEQ ID NO:975 and a light chain variable domain comprising a CDR1 comprising the amino acid sequence of SEQ ID NO:978, a CDR2 comprising the amino acid sequence of SEQ ID NO:979, and a CDR3 comprising the amino acid sequence of SEQ ID NO:980.

In one embodiment, the antibody is an isolated COV_3137 antibody. As used herein, the term “COV_3137” refers to a monoclonal antibody, or antigen-binding fragment thereof, that binds to the S1 domain of a coronavirus, such as SARS-CoV-2, wherein the antibody comprises 1) a heavy chain variable domain comprising the amino acid sequence of SEQ ID NO:982 and a light chain variable domain comprising the amino acid sequence of SEQ ID NO:987; or 2) a heavy chain variable domain comprising a CDR1 comprising the amino acid sequence of SEQ ID NO:983, a CDR2 comprising the amino acid sequence of SEQ ID NO:984, and a CDR3 comprising the amino acid sequence of SEQ ID NO:985 and alight chain variable domain comprising a CDR1 comprising the amino acid sequence of SEQ ID NO:988, a CDR2 comprising the amino acid sequence of SEQ ID NO:989, and a CDR3 comprising the amino acid sequence of SEQ ID NO:990.

In one embodiment, the antibody is an isolated COV_2038 antibody. As used herein, the term “COV_2038” refers to a monoclonal antibody, or antigen-binding fragment thereof, that binds to the NTD of a coronavirus, such as SARS-CoV-2, wherein the antibody comprises a heavy chain variable domain comprising a CDR1 comprising the amino acid sequence of SEQ ID NO:1119, a CDR2 comprising the amino acid sequence of SEQ ID NO: 1149, and a CDR3 comprising the amino acid sequence of SEQ ID NO: 1179 and a light chain variable domain comprising a CDR1 comprising the amino acid sequence of SEQ ID NO: 1209, a CDR2 comprising the amino acid sequence QDT, and a CDR3 comprising the amino acid sequence of SEQ ID NO: 1239.

Modified Antibodies

Modified versions of COV_1007, COV_1037, COV_1045, COV_1046, COV_1201, COV_2004, COV_2008, COV_2014, COV_2018, COV_2024, COV_2025, COV_2027, COV_2028, COV_2035, COV_2037, COV_2038, COV_2039, COV_2054, COV_2056, COV_2057, COV_2063, COV_2091, COV_2100, COV_2103, COV_2108, COV_2123, COV_2125, COV_2134, COV_2151, COV_2165, COV_2172, COV_2173, COV_2193, COV_2196, COV_3000, COV_3005, COV_3013, COV_3019, COV_3028, COV_3031, COV_3033, COV_3037, COV_3040, COV_3043, COV_3053, COV_3088, COV_1012, COV_1025, COV_1032, COV_1050, COV_1056, COV_1060, COV_1063, COV_1071, COV_1076, COV_1082, COV_1085, COV_1086, COV_1087, COV_1097, COV_1116, COV_1118, COV_1122, COV_1131, COV_1136, COV_1144, COV_1145, COV_1149, COV_1151, COV_1154, COV_1165, COV_1166, COV_1170, COV_1172, COV_1177, COV_1184, COV_1198, COV_2032, COV_2048, COV_2055, COV_2056, COV_2064, COV_2066, COV_2077, COV_2093, COV_2137, COV_2143, COV_2169, COV_2172, COV_2174, COV_2205, COV_2215, COV_3049, COV_3069, COV_3077, COV_3079, COV_3100, COV_3103, COV_3129, or COV_3137 antibodies are also provided. Typically, modifications to an antibody can be introduced through the nucleic acids that encode the heavy or light chain variable domains of the antibody. These modifications can include deletions, insertions, point mutations, truncations, and amino acid substitutions and addition of amino acids or non-amino acid moieties. For example, random mutagenesis of the disclosed V_{H} or V_{L} sequences can be used to generate variant V_{H} or V_{L} domains still capable of binding a coronavirus. A technique using error-prone PCR is described by Gram et al. (Proc. Nat. Acad. Sci. U.S.A. (1992) 89: 3576-3580). Another method uses direct mutagenesis of the disclosed V_{H} or V_{L} sequences. Such techniques are disclosed by Barbas et al. (Proc. Nat. Acad. Sci. U.S.A. (1994) 91: 3809-3813) and Schier et al. (J. Mol. Biol. (1996) 263: 551-567). Modifications can also be made directly to the amino acid sequence, such as by cleavage, addition of a linker molecule or addition of a detectable moiety, such as biotin, addition of a fatty acid, and the like.

In one embodiment, the antibody is a monoclonal antibody that binds to a coronavirus, such as SARS_CoV-2, and comprises 1) a heavy chain variable domain that is at least about 70%, at least about 80%, at least about 90%, at least about 95%, at least about 98% identical, or 100% identical to the amino acid sequence of the heavy chain variable domain of the COV_1007, COV_1037, COV_1045, COV_1046, COV_1201, COV_2004, COV_2008, COV_2014, COV_2018, COV_2024, COV_2025, COV_2027, COV_2028, COV_2035, COV_2037, COV_2038, COV_2039, COV_2054, COV_2056, COV_2057, COV_2063, COV_2091, COV_2100, COV_2103, COV_2108, COV_2123, COV_2125, COV_2134, COV_2151, COV_2165, COV_2172, COV_2173, COV_2193, COV_2196, COV_3000, COV_3005, COV_3013, COV_3019, COV_3028, COV_3031, COV_3033, COV_3037, COV_3040, COV_3043, COV_3053, COV_3088, COV_1012, COV_1025, COV_1032, COV_1050, COV_1056, COV_1060, COV_1063, COV_1071, COV_1076, COV_1082, COV_1085, COV_1086, COV_1087, COV_1097, COV_1116, COV_1118, COV_1122, COV_1131, COV_1136, COV_1144, COV_1145, COV_1149, COV_1151, COV_1154, COV_1165, COV_1166, COV_1170, COV_1172, COV_1177, COV_1184, COV_1198, COV_2032, COV_2048, COV_2055, COV_2056, COV_2064, COV_2066, COV_2077, COV_2093, COV_2137, COV_2143, COV_2169, COV_2172, COV_2174, COV_2205, COV_2215, COV_3049, COV_3069, COV_3077, COV_3079, COV_3100, COV_3103, COV_3129, or COV_3137 antibody as described herein, and 2) a light chain variable domain that is at least about 70%, at least about 80%, at least about 90%, at least about 95%, at least about 98% identical, or 100% identical to the amino acid sequence of the light chain variable domain of the COV_1007, COV_1037, COV_1045, COV_1046, COV_1201, COV_2004, COV_2008, COV_2014, COV_2018, COV_2024, COV_2025, COV_2027, COV_2028, COV_2035, COV_2037, COV_2038, COV_2039, COV_2054, COV_2056, COV_2057, COV_2063, COV_2091, COV_2100, COV_2103, COV_2108, COV_2123, COV_2125, COV_2134, COV_2151, COV_2165, COV_2172, COV_2173, COV_2193, COV_2196, COV_3000, COV_3005, COV_3013, COV_3019, COV_3028, COV_3031, COV_3033, COV_3037, COV_3040, COV_3043, COV_3053, COV_3088, COV_1012, COV_1025, COV_1032, COV_1050, COV_1056, COV_1060, COV_1063, COV_1071, COV_1076, COV_1082, COV_1085, COV_1086, COV_1087, COV_1097, COV_1116, COV_1118, COV_1122, COV_1131, COV_1136, COV_1144, COV_1145, COV_1149, COV_1151, COV_1154, COV_1165, COV_1166, COV_1170, COV_1172, COV_1177, COV_1184, COV_1198, COV_2032, COV_2048, COV_2055, COV_2056, COV_2064, COV_2066, COV_2077, COV_2093, COV_2137, COV_2143, COV_2169, COV_2172, COV_2174, COV_2205, COV_2215, COV_3049, COV_3069, COV_3077, COV_3079, COV_3100, COV_3103, COV_3129, or COV_3137 antibody, as described herein.

In another embodiment, the monoclonal antibody binds to a coronavirus, such as SARS_CoV-2, and comprises six CDRs (H1, H2, H3, L1, L2, and L3) that are at least about 90%, at least about 95% or at least about 98% identical to the amino acid sequences of the six CDRs (H1, H2, H3, L1, L2, and L3) of the heavy and light chain variable domains of the COV_1007, COV_1037, COV_1045, COV_1046, COV_1201, COV_2004, COV_2008, COV_2014, COV_2018, COV_2024, COV_2025, COV_2027, COV_2028, COV_2035, COV_2037, COV_2038, COV_2039, COV_2054, COV_2056, COV_2057, COV_2063, COV_2091, COV_2100, COV_2103, COV_2108, COV_2123, COV_2125, COV_2134, COV_2151, COV_2165, COV_2172, COV_2173, COV_2193, COV_2196, COV_3000, COV_3005, COV_3013, COV_3019, COV_3028, COV_3031, COV_3033, COV_3037, COV_3040, COV_3043, COV_3053, COV_3088, COV_1012, COV_1025, COV_1032, COV_1050, COV_1056, COV_1060, COV_1063, COV_1071, COV_1076, COV_1082, COV_1085, COV_1086, COV_1087, COV_1097, COV_1116, COV_1118, COV_1122, COV_1131, COV_1136, COV_1144, COV_1145, COV_1149, COV_1151, COV_1154, COV_1165, COV_1166, COV_1170, COV_1172, COV_1177, COV_1184, COV_1198, COV_2032, COV_2048, COV_2055, COV_2056, COV_2064, COV_2066, COV_2077, COV_2093, COV_2137, COV_2143, COV_2169, COV_2172, COV_2174, COV_2205, COV_2215, COV_3049, COV_3069, COV_3077, COV_3079, COV_3100, COV_3103, COV_3129, or COV_3137 antibody.

In another embodiment, the monoclonal antibody binds to a coronavirus, such as SARS_CoV-2, and comprises a heavy chain variable domain identical to the heavy chain variable domain sequences of the COV_1007, COV_1037, COV_1045, COV_1046, COV_1201, COV_2004, COV_2008, COV_2014, COV_2018, COV_2024, COV_2025, COV_2027, COV_2028, COV_2035, COV_2037, COV_2038, COV_2039, COV_2054, COV_2056, COV_2057, COV_2063, COV_2091, COV_2100, COV_2103, COV_2108, COV_2123, COV_2125, COV_2134, COV_2151, COV_2165, COV_2172, COV_2173, COV_2193, COV_2196, COV_3000, COV_3005, COV_3013, COV_3019, COV_3028, COV_3031, COV_3033, COV_3037, COV_3040, COV_3043, COV_3053, COV_3088, COV_1012, COV_1025, COV_1032, COV_1050, COV_1056, COV_1060, COV_1063, COV_1071, COV_1076, COV_1082, COV_1085, COV_1086, COV_1087, COV_1097, COV_1116, COV_1118, COV_1122, COV_1131, COV_1136, COV_1144, COV_1145, COV_1149, COV_1151, COV_1154, COV_1165, COV_1166, COV_1170, COV_1172, COV_1177, COV_1184, COV_1198, COV_2032, COV_2048, COV_2055, COV_2056, COV_2064, COV_2066, COV_2077, COV_2093, COV_2137, COV_2143, COV_2169, COV_2172, COV_2174, COV_2205, COV_2215, COV_3049, COV_3069, COV_3077, COV_3079, COV_3100, COV_3103, COV_3129, or COV_3137 antibody except for 1, up to 2, up to 3, up to 4, up to 5, up to 6, up to 7, and in certain cases, up to 10 amino acid substitutions in the CDR sequences. In another embodiment, the monoclonal antibody binds to a coronavirus, such as SARS-CoV-2, and comprises a light chain variable domain identical to the light chain variable domain sequences of the COV_1007, COV_1037, COV_1045, COV_1046, COV_1201, COV_2004, COV_2008, COV_2014, COV_2018, COV_2024, COV_2025, COV_2027, COV_2028, COV_2035, COV_2037, COV_2038, COV_2039, COV_2054, COV_2056, COV_2057, COV_2063, COV_2091, COV_2100, COV_2103, COV_2108, COV_2123, COV_2125, COV_2134, COV_2151, COV_2165, COV_2172, COV_2173, COV_2193, COV_2196, COV_3000, COV_3005, COV_3013, COV_3019, COV_3028, COV_3031, COV_3033, COV_3037, COV_3040, COV_3043, COV_3053, COV_3088, COV_1012, COV_1025, COV_1032, COV_1050, COV_1056, COV_1060, COV_1063, COV_1071, COV_1076, COV_1082, COV_1085, COV_1086, COV_1087, COV_1097, COV_1116, COV_1118, COV_1122, COV_1131, COV_1136, COV_1144, COV_1145, COV_1149, COV_1151, COV_1154, COV_1165, COV_1166, COV_1170, COV_1172, COV_1177, COV_1184, COV_1198, COV_2032, COV_2048, COV_2055, COV_2056, COV_2064, COV_2066, COV_2077, COV_2093, COV_2137, COV_2143, COV_2169, COV_2172, COV_2174, COV_2205, COV_2215, COV_3049, COV_3069, COV_3077, COV_3079, COV_3100, COV_3103, COV_3129, or COV_3137 except for 1, up to 2, up to 3, up to 4, up to 5, up to 6, up to 7, and in certain cases, up to 10 amino acid substitutions in the CDR sequences.

The specific amino acid positions that can be substituted in a CDR, as well as the donor amino acid that can be substituted into those positions can be readily determined by one of skill in the art using known methods, such as those disclosed in published U.S. Application 2006/0099204, the disclosure of which is hereby incorporated by reference in its entirety. Typically, this involves substitution of an amino acid with an amino acid having similar charge, hydrophobic, or stereochemical characteristics. More drastic substitutions in FR regions, in contrast to CDR regions, may also be made as long as they do not adversely affect (e.g., reduce affinity by more than 50% as compared to unsubstituted antibody) the binding properties of the antibody.

Modified versions of the COV_1007, COV_1037, COV_1045, COV_1046, COV_1201, COV_2004, COV_2008, COV_2014, COV_2018, COV_2024, COV_2025, COV_2027, COV_2028, COV_2035, COV_2037, COV_2038, COV_2039, COV_2054, COV_2056, COV_2057, COV_2063, COV_2091, COV_2100, COV_2103, COV_2108, COV_2123, COV_2125, COV_2134, COV_2151, COV_2165, COV_2172, COV_2173, COV_2193, COV_2196, COV_3000, COV_3005, COV_3013, COV_3019, COV_3028, COV_3031, COV_3033, COV_3037, COV_3040, COV_3043, COV_3053, COV_3088, COV_1012, COV_1025, COV_1032, COV_1050, COV_1056, COV_1060, COV_1063, COV_1071, COV_1076, COV_1082, COV_1085, COV_1086, COV_1087, COV_1097, COV_1116, COV_1118, COV_1122, COV_1131, COV_1136, COV_1144, COV_1145, COV_1149, COV_1151, COV_1154, COV_1165, COV_1166, COV_1170, COV_1172, COV_1177, COV_1184, COV_1198, COV_2032, COV_2048, COV_2055, COV_2056, COV_2064, COV_2066, COV_2077, COV_2093, COV_2137, COV_2143, COV_2169, COV_2172, COV_2174, COV_2205, COV_2215, COV_3049, COV_3069, COV_3077, COV_3079, COV_3100, COV_3103, COV_3129, or COV_3137 antibodies can also be screened to identify which mutation provides a modified antibody that retains a desired property, such as high affinity binding of the parent antibody for either coronavirus, e.g., SARS-CoV-2, and/or potent neutralizing activity.

Nucleic Acids, Cloning and Expression Systems

The present disclosure further provides isolated nucleic acids encoding the COV_1007, COV_1037, COV_1045, COV_1046, COV_1201, COV_2004, COV_2008, COV_2014, COV_2018, COV_2024, COV_2025, COV_2027, COV_2028, COV_2035, COV_2037, COV_2038, COV_2039, COV_2054, COV_2056, COV_2057, COV_2063, COV_2091, COV_2100, COV_2103, COV_2108, COV_2123, COV_2125, COV_2134, COV_2151, COV_2165, COV_2172, COV_2173, COV_2193, COV_2196, COV_3000, COV_3005, COV_3013, COV_3019, COV_3028, COV_3031, COV_3033, COV_3037, COV_3040, COV_3043, COV_3053, COV_3088, COV_1012, COV_1025, COV_1032, COV_1050, COV_1056, COV_1060, COV_1063, COV_1071, COV_1076, COV_1082, COV_1085, COV_1086, COV_1087, COV_1097, COV_1116, COV_1118, COV_1122, COV_1131, COV_1136, COV_1144, COV_1145, COV_1149, COV_1151, COV_1154, COV_1165, COV_1166, COV_1170, COV_1172, COV_1177, COV_1184, COV_1198, COV_2032, COV_2048, COV_2055, COV_2056, COV_2064, COV_2066, COV_2077, COV_2093, COV_2137, COV_2143, COV_2169, COV_2172, COV_2174, COV_2205, COV_2215, COV_3049, COV_3069, COV_3077, COV_3079, COV_3100, COV_3103, COV_3129, or COV_3137 antibodies or antigen-binding fragments thereof. The nucleic acids may comprise DNA or RNA and may be wholly or partially synthetic or recombinant. Reference to a nucleotide sequence as set out herein encompasses a DNA molecule with the specified sequence, and encompasses a RNA molecule with the specified sequence in which U is substituted for T, unless context requires otherwise.

The nucleic acids provided herein encode at least one CDR, all six CDRs (i.e., H1, H2, H3, L1, L2, and L3), a V_{H} domain, and/or a V_{L} domain of one of the COV_1007, COV_1037, COV_1045, COV_1046, COV_1201, COV_2004, COV_2008, COV_2014, COV_2018, COV_2024, COV_2025, COV_2027, COV_2028, COV_2035, COV_2037, COV, 2038, COV_2039, COV_2054, COV_2056, COV_2057, COV_2063, COV_2091, COV_2100, COV_2103, COV_2108, COV_2123, COV_2125, COV_2134, COV_2151, COV_2165, COV_2172, COV_2173, COV_2193, COV_2196, COV_3000, COV_3005, COV_3013, COV_3019, COV_3028, COV_3031, COV_3033, COV_3037, COV_3040, COV_3043, COV_3053, COV_3088, COV_1012, COV_1025, COV_1032, COV_1050, COV_1056, COV_1060, COV_1063, COV_1071, COV_1076, COV_1082, COV_1085, COV_1086, COV_1087, COV_1097, COV_1116, COV_1118, COV_1122, COV_1131, COV_1136, COV_1144, COV_1145, COV_1149, COV_1151, COV_1154, COV_1165, COV_1166, COV_1170, COV_1172, COV_1177, COV_1184, COV_1198, COV_2032, COV_2048, COV_2055, COV_2056, COV_2064, COV_2066, COV_2077, COV_2093, COV_2137, COV_2143, COV_2169, COV_2172, COV_2174, COV_2205, COV_2215, COV_3049, COV_3069, COV_3077, COV_3079, COV_3100, COV_3103, COV_3129, or COV_3137 antibodies.

For example, in some embodiments, the disclosure provides an isolated nucleic acid that encodes the heavy chain variable domain of the COV_1007 antibody, wherein the isolated nucleic acid comprises SEQ ID NO: 1 and/or the isolated nucleic acid encodes the light chain variable domain of the of the COV_1007 antibody, wherein the isolated nucleic acid comprises SEQ ID NO: 6.

In some embodiments, the disclosure provides an isolated nucleic acid that encodes the heavy chain variable domain of the COV_1037 antibody, wherein the isolated nucleic acid comprises SEQ ID NO: 11 and/or the isolated nucleic acid encodes the light chain variable domain of the of the COV_1037 antibody, wherein the isolated nucleic acid comprises SEQ ID NO: 16.

In some embodiments, the disclosure provides an isolated nucleic acid that encodes the heavy chain variable domain of the COV_1045 antibody, wherein the isolated nucleic acid comprises SEQ ID NO: 21 and/or the isolated nucleic acid encodes the light chain variable domain of the of the COV_1045 antibody, wherein the isolated nucleic acid comprises SEQ ID NO: 26.

In some embodiments, the disclosure provides an isolated nucleic acid that encodes the heavy chain variable domain of the COV_1046 antibody, wherein the isolated nucleic acid comprises SEQ ID NO: 31 and/or the isolated nucleic acid encodes the light chain variable domain of the of the COV_1046 antibody, wherein the isolated nucleic acid comprises SEQ ID NO: 36.

In some embodiments, the disclosure provides an isolated nucleic acid that encodes the heavy chain variable domain of the COV_1201 antibody, wherein the isolated nucleic acid comprises SEQ ID NO: 41 and/or the isolated nucleic acid encodes the light chain variable domain of the of the COV_1201 antibody, wherein the isolated nucleic acid comprises SEQ ID NO: 46.

In some embodiments, the disclosure provides an isolated nucleic acid that encodes the heavy chain variable domain of the COV_2004 antibody, wherein the isolated nucleic acid comprises SEQ ID NO: 51 and/or the isolated nucleic acid encodes the light chain variable domain of the of the COV_2004 antibody, wherein the isolated nucleic acid comprises SEQ ID NO: 56.

In some embodiments, the disclosure provides an isolated nucleic acid that encodes the heavy chain variable domain of the COV_2008 antibody, wherein the isolated nucleic acid comprises SEQ ID NO: 61 and/or the isolated nucleic acid encodes the light chain variable domain of the of the COV_2008 antibody, wherein the isolated nucleic acid comprises SEQ ID NO: 66.

In some embodiments, the disclosure provides an isolated nucleic acid that encodes the heavy chain variable domain of the COV_2014 antibody, wherein the isolated nucleic acid comprises SEQ ID NO: 71 and/or the isolated nucleic acid encodes the light chain variable domain of the of the COV_2014 antibody, wherein the isolated nucleic acid comprises SEQ ID NO: 76.

In some embodiments, the disclosure provides an isolated nucleic acid that encodes the heavy chain variable domain of the COV_2018 antibody, wherein the isolated nucleic acid comprises SEQ ID NO: 81 and/or the isolated nucleic acid encodes the light chain variable domain of the of the COV_2018 antibody, wherein the isolated nucleic acid comprises SEQ ID NO:86.

In some embodiments, the disclosure provides an isolated nucleic acid that encodes the heavy chain variable domain of the COV_2024 antibody, wherein the isolated nucleic acid comprises SEQ ID NO: 91 and/or the isolated nucleic acid encodes the light chain variable domain of the of the COV_2024 antibody, wherein the isolated nucleic acid comprises SEQ ID NO: 96.

In some embodiments, the disclosure provides an isolated nucleic acid that encodes the heavy chain variable domain of the COV_2025 antibody, wherein the isolated nucleic acid comprises SEQ ID NO: 101 and/or the isolated nucleic acid encodes the light chain variable domain of the of the COV_2025 antibody, wherein the isolated nucleic acid comprises SEQ ID NO: 106.

In some embodiments, the disclosure provides an isolated nucleic acid that encodes the heavy chain variable domain of the COV_2027 antibody, wherein the isolated nucleic acid comprises SEQ ID NO: 111 and/or the isolated nucleic acid encodes the light chain variable domain of the of the COV_2027 antibody, wherein the isolated nucleic acid comprises SEQ ID NO: 116.

In some embodiments, the disclosure provides an isolated nucleic acid that encodes the heavy chain variable domain of the COV_2028 antibody, wherein the isolated nucleic acid comprises SEQ ID NO: 121 and/or the isolated nucleic acid encodes the light chain variable domain of the of the COV_2028 antibody, wherein the isolated nucleic acid comprises SEQ ID NO: 126.

In some embodiments, the disclosure provides an isolated nucleic acid that encodes the heavy chain variable domain of the COV_2035 antibody, wherein the isolated nucleic acid comprises SEQ ID NO: 131 and/or the isolated nucleic acid encodes the light chain variable domain of the of the COV_2035 antibody, wherein the isolated nucleic acid comprises SEQ ID NO: 136.

In some embodiments, the disclosure provides an isolated nucleic acid that encodes the heavy chain variable domain of the COV_2037 antibody, wherein the isolated nucleic acid comprises SEQ ID NO: 141 and/or the isolated nucleic acid encodes the light chain variable domain of the of the COV_2037 antibody, wherein the isolated nucleic acid comprises SEQ ID NO: 146.

In some embodiments, the disclosure provides an isolated nucleic acid that encodes the heavy chain variable domain of the COV_2039 antibody, wherein the isolated nucleic acid comprises SEQ ID NO: 151 and/or the isolated nucleic acid encodes the light chain variable domain of the of the COV_2039 antibody, wherein the isolated nucleic acid comprises SEQ ID NO: 156.

In some embodiments, the disclosure provides an isolated nucleic acid that encodes the heavy chain variable domain of the COV_2054 antibody, wherein the isolated nucleic acid comprises SEQ ID NO: 161 and/or the isolated nucleic acid encodes the light chain variable domain of the of the COV_2054 antibody, wherein the isolated nucleic acid comprises SEQ ID NO: 166.

In some embodiments, the disclosure provides an isolated nucleic acid that encodes the heavy chain variable domain of the COV_2056 antibody, wherein the isolated nucleic acid comprises SEQ ID NO: 431 and/or the isolated nucleic acid encodes the light chain variable domain of the of the COV_2056 antibody, wherein the isolated nucleic acid comprises SEQ ID NO: 436.

In some embodiments, the disclosure provides an isolated nucleic acid that encodes the heavy chain variable domain of the COV_2057 antibody, wherein the isolated nucleic acid comprises SEQ ID NO: 171 and/or the isolated nucleic acid encodes the light chain variable domain of the of the COV_2057 antibody, wherein the isolated nucleic acid comprises SEQ ID NO: 176.

In some embodiments, the disclosure provides an isolated nucleic acid that encodes the heavy chain variable domain of the COV_2063 antibody, wherein the isolated nucleic acid comprises SEQ ID NO: 181 and/or the isolated nucleic acid encodes the light chain variable domain of the of the COV_2063 antibody, wherein the isolated nucleic acid comprises SEQ ID NO:186.

In some embodiments, the disclosure provides an isolated nucleic acid that encodes the heavy chain variable domain of the COV_2091 antibody, wherein the isolated nucleic acid comprises SEQ ID NO: 191 and/or the isolated nucleic acid encodes the light chain variable domain of the of the COV_2091 antibody, wherein the isolated nucleic acid comprises SEQ ID NO: 196.

In some embodiments, the disclosure provides an isolated nucleic acid that encodes the heavy chain variable domain of the COV_2100 antibody, wherein the isolated nucleic acid comprises SEQ ID NO: 201 and/or the isolated nucleic acid encodes the light chain variable domain of the of the COV_2100 antibody, wherein the isolated nucleic acid comprises SEQ ID NO:206.

In some embodiments, the disclosure provides an isolated nucleic acid that encodes the heavy chain variable domain of the COV_2103 antibody, wherein the isolated nucleic acid comprises SEQ ID NO: 211 and/or the isolated nucleic acid encodes the light chain variable domain of the of the COV_2103 antibody, wherein the isolated nucleic acid comprises SEQ ID NO: 216.

In some embodiments, the disclosure provides an isolated nucleic acid that encodes the heavy chain variable domain of the COV_2108 antibody, wherein the isolated nucleic acid comprises SEQ ID NO: 221 and/or the isolated nucleic acid encodes the light chain variable domain of the of the COV_2108 antibody, wherein the isolated nucleic acid comprises SEQ ID NO: 226.

In some embodiments, the disclosure provides an isolated nucleic acid that encodes the heavy chain variable domain of the COV_2123 antibody, wherein the isolated nucleic acid comprises SEQ ID NO: 231 and/or the isolated nucleic acid encodes the light chain variable domain of the of the COV_2123 antibody, wherein the isolated nucleic acid comprises SEQ ID NO: 236.

In some embodiments, the disclosure provides an isolated nucleic acid that encodes the heavy chain variable domain of the COV_2125 antibody, wherein the isolated nucleic acid comprises SEQ ID NO: 241 and/or the isolated nucleic acid encodes the light chain variable domain of the of the COV_2125 antibody, wherein the isolated nucleic acid comprises SEQ ID NO: 246.

In some embodiments, the disclosure provides an isolated nucleic acid that encodes the heavy chain variable domain of the COV_2134 antibody, wherein the isolated nucleic acid comprises SEQ ID NO: 251 and/or the isolated nucleic acid encodes the light chain variable domain of the of the COV_2134 antibody, wherein the isolated nucleic acid comprises SEQ ID NO: 256.

In some embodiments, the disclosure provides an isolated nucleic acid that encodes the heavy chain variable domain of the COV_2151 antibody, wherein the isolated nucleic acid comprises SEQ ID NO: 261 and/or the isolated nucleic acid encodes the light chain variable domain of the of the COV_2151antibody, wherein the isolated nucleic acid comprises SEQ ID NO: 266.

In some embodiments, the disclosure provides an isolated nucleic acid that encodes the heavy chain variable domain of the COV_2165 antibody, wherein the isolated nucleic acid comprises SEQ ID NO: 271 and/or the isolated nucleic acid encodes the light chain variable domain of the of the COV_2165 antibody, wherein the isolated nucleic acid comprises SEQ ID NO: 276.

In some embodiments, the disclosure provides an isolated nucleic acid that encodes the heavy chain variable domain of the COV_2172 antibody, wherein the isolated nucleic acid comprises SEQ ID NO: 441 and/or the isolated nucleic acid encodes the light chain variable domain of the of the COV_2172 antibody, wherein the isolated nucleic acid comprises SEQ ID NO: 446.

In some embodiments, the disclosure provides an isolated nucleic acid that encodes the heavy chain variable domain of the COV_2173 antibody, wherein the isolated nucleic acid comprises SEQ ID NO: 281 and/or the isolated nucleic acid encodes the light chain variable domain of the of the COV_2173 antibody, wherein the isolated nucleic acid comprises SEQ ID NO: 286.

In some embodiments, the disclosure provides an isolated nucleic acid that encodes the heavy chain variable domain of the COV_2193 antibody, wherein the isolated nucleic acid comprises SEQ ID NO: 291 and/or the isolated nucleic acid encodes the light chain variable domain of the of the COV_2193 antibody, wherein the isolated nucleic acid comprises SEQ ID NO: 296.

In some embodiments, the disclosure provides an isolated nucleic acid that encodes the heavy chain variable domain of the COV_2196 antibody, wherein the isolated nucleic acid comprises SEQ ID NO: 301 and/or the isolated nucleic acid encodes the light chain variable domain of the of the COV_2196 antibody, wherein the isolated nucleic acid comprises SEQ ID NO: 306.

In some embodiments, the disclosure provides an isolated nucleic acid that encodes the heavy chain variable domain of the COV_3000 antibody, wherein the isolated nucleic acid comprises SEQ ID NO: 311 and/or the isolated nucleic acid encodes the light chain variable domain of the of the COV_3000 antibody, wherein the isolated nucleic acid comprises SEQ ID NO: 316.

In some embodiments, the disclosure provides an isolated nucleic acid that encodes the heavy chain variable domain of the COV_3005 antibody, wherein the isolated nucleic acid comprises SEQ ID NO: 321 and/or the isolated nucleic acid encodes the light chain variable domain of the of the COV_3005 antibody, wherein the isolated nucleic acid comprises SEQ ID NO: 326.

In some embodiments, the disclosure provides an isolated nucleic acid that encodes the heavy chain variable domain of the COV_3013 antibody, wherein the isolated nucleic acid comprises SEQ ID NO: 331 and/or the isolated nucleic acid encodes the light chain variable domain of the of the COV_3013 antibody, wherein the isolated nucleic acid comprises SEQ ID NO: 336.

In some embodiments, the disclosure provides an isolated nucleic acid that encodes the heavy chain variable domain of the COV_3019 antibody, wherein the isolated nucleic acid comprises SEQ ID NO: 341 and/or the isolated nucleic acid encodes the light chain variable domain of the of the COV_3019 antibody, wherein the isolated nucleic acid comprises SEQ ID NO: 346.

In some embodiments, the disclosure provides an isolated nucleic acid that encodes the heavy chain variable domain of the COV_3028 antibody, wherein the isolated nucleic acid comprises SEQ ID NO: 351 and/or the isolated nucleic acid encodes the light chain variable domain of the of the COV_3028 antibody, wherein the isolated nucleic acid comprises SEQ ID NO: 356.

In some embodiments, the disclosure provides an isolated nucleic acid that encodes the heavy chain variable domain of the COV_3031 antibody, wherein the isolated nucleic acid comprises SEQ ID NO: 361 and/or the isolated nucleic acid encodes the light chain variable domain of the of the COV_3031 antibody, wherein the isolated nucleic acid comprises SEQ ID NO: 366.

In some embodiments, the disclosure provides an isolated nucleic acid that encodes the heavy chain variable domain of the COV_3033 antibody, wherein the isolated nucleic acid comprises SEQ ID NO: 371 and/or the isolated nucleic acid encodes the light chain variable domain of the of the COV_3033 antibody, wherein the isolated nucleic acid comprises SEQ ID NO: 376.

In some embodiments, the disclosure provides an isolated nucleic acid that encodes the heavy chain variable domain of the COV_3037 antibody, wherein the isolated nucleic acid comprises SEQ ID NO: 381 and/or the isolated nucleic acid encodes the light chain variable domain of the of the COV_3037 antibody, wherein the isolated nucleic acid comprises SEQ ID NO: 386.

In some embodiments, the disclosure provides an isolated nucleic acid that encodes the heavy chain variable domain of the COV_3040 antibody, wherein the isolated nucleic acid comprises SEQ ID NO: 391 and/or the isolated nucleic acid encodes the light chain variable domain of the of the COV_3040 antibody, wherein the isolated nucleic acid comprises SEQ ID NO: 396.

In some embodiments, the disclosure provides an isolated nucleic acid that encodes the heavy chain variable domain of the COV_3043 antibody, wherein the isolated nucleic acid comprises SEQ ID NO: 400 and/or the isolated nucleic acid encodes the light chain variable domain of the of the COV_3043 antibody, wherein the isolated nucleic acid comprises SEQ ID NO: 406.

In some embodiments, the disclosure provides an isolated nucleic acid that encodes the heavy chain variable domain of the COV_3053 antibody, wherein the isolated nucleic acid comprises SEQ ID NO: 411 and/or the isolated nucleic acid encodes the light chain variable domain of the of the COV_3053 antibody, wherein the isolated nucleic acid comprises SEQ ID NO: 416.

In some embodiments, the disclosure provides an isolated nucleic acid that encodes the heavy chain variable domain of the COV_3088 antibody, wherein the isolated nucleic acid comprises SEQ ID NO: 421 and/or the isolated nucleic acid encodes the light chain variable domain of the of the COV_3088 antibody, wherein the isolated nucleic acid comprises SEQ ID NO: 426.

In some embodiments, the disclosure provides an isolated nucleic acid that encodes the heavy chain variable domain of the COV_1012 antibody, wherein the isolated nucleic acid comprises SEQ ID NO: 451 and/or the isolated nucleic acid encodes the light chain variable domain of the of the COV_1012 antibody, wherein the isolated nucleic acid comprises SEQ ID NO: 456.

In some embodiments, the disclosure provides an isolated nucleic acid that encodes the heavy chain variable domain of the COV_1025 antibody, wherein the isolated nucleic acid comprises SEQ ID NO: 461 and/or the isolated nucleic acid encodes the light chain variable domain of the of the COV_1025 antibody, wherein the isolated nucleic acid comprises SEQ ID NO: 466.

In some embodiments, the disclosure provides an isolated nucleic acid that encodes the heavy chain variable domain of the COV_1032 antibody, wherein the isolated nucleic acid comprises SEQ ID NO: 471 and/or the isolated nucleic acid encodes the light chain variable domain of the of the COV_1032 antibody, wherein the isolated nucleic acid comprises SEQ ID NO: 476.

In some embodiments, the disclosure provides an isolated nucleic acid that encodes the heavy chain variable domain of the COV_1050 antibody, wherein the isolated nucleic acid comprises SEQ ID NO: 481 and/or the isolated nucleic acid encodes the light chain variable domain of the of the COV_1050 antibody, wherein the isolated nucleic acid comprises SEQ ID NO: 486.

In some embodiments, the disclosure provides an isolated nucleic acid that encodes the heavy chain variable domain of the COV_1056 antibody, wherein the isolated nucleic acid comprises SEQ ID NO: 491 and/or the isolated nucleic acid encodes the light chain variable domain of the of the COV_1056 antibody, wherein the isolated nucleic acid comprises SEQ ID NO: 496.

In some embodiments, the disclosure provides an isolated nucleic acid that encodes the heavy chain variable domain of the COV_1060 antibody, wherein the isolated nucleic acid comprises SEQ ID NO: 501 and/or the isolated nucleic acid encodes the light chain variable domain of the of the COV_1060 antibody, wherein the isolated nucleic acid comprises SEQ ID NO: 506.

In some embodiments, the disclosure provides an isolated nucleic acid that encodes the heavy chain variable domain of the COV_1063 antibody, wherein the isolated nucleic acid comprises SEQ ID NO: 511 and/or the isolated nucleic acid encodes the light chain variable domain of the of the COV_1063 antibody, wherein the isolated nucleic acid comprises SEQ ID NO: 516.

In some embodiments, the disclosure provides an isolated nucleic acid that encodes the heavy chain variable domain of the COV_1071 antibody, wherein the isolated nucleic acid comprises SEQ ID NO: 521 and/or the isolated nucleic acid encodes the light chain variable domain of the of the COV_1071 antibody, wherein the isolated nucleic acid comprises SEQ ID NO: 526.

In some embodiments, the disclosure provides an isolated nucleic acid that encodes the heavy chain variable domain of the COV_1076 antibody, wherein the isolated nucleic acid comprises SEQ ID NO: 531 and/or the isolated nucleic acid encodes the light chain variable domain of the of the COV_1076 antibody, wherein the isolated nucleic acid comprises SEQ ID NO: 536.

In some embodiments, the disclosure provides an isolated nucleic acid that encodes the heavy chain variable domain of the COV_1082 antibody, wherein the isolated nucleic acid comprises SEQ ID NO: 541 and/or the isolated nucleic acid encodes the light chain variable domain of the of the COV_1082 antibody, wherein the isolated nucleic acid comprises SEQ ID NO: 546.

In some embodiments, the disclosure provides an isolated nucleic acid that encodes the heavy chain variable domain of the COV_1085 antibody, wherein the isolated nucleic acid comprises SEQ ID NO: 551 and/or the isolated nucleic acid encodes the light chain variable domain of the of the COV_1085 antibody, wherein the isolated nucleic acid comprises SEQ ID NO: 556.

In some embodiments, the disclosure provides an isolated nucleic acid that encodes the heavy chain variable domain of the COV_1086 antibody, wherein the isolated nucleic acid comprises SEQ ID NO: 561 and/or the isolated nucleic acid encodes the light chain variable domain of the of the COV_1086 antibody, wherein the isolated nucleic acid comprises SEQ ID NO: 566.

In some embodiments, the disclosure provides an isolated nucleic acid that encodes the heavy chain variable domain of the COV_1087 antibody, wherein the isolated nucleic acid comprises SEQ ID NO: 571 and/or the isolated nucleic acid encodes the light chain variable domain of the of the COV_1087 antibody, wherein the isolated nucleic acid comprises SEQ ID NO: 576.

In some embodiments, the disclosure provides an isolated nucleic acid that encodes the heavy chain variable domain of the COV_1097 antibody, wherein the isolated nucleic acid comprises SEQ ID NO: 581 and/or the isolated nucleic acid encodes the light chain variable domain of the of the COV_1097 antibody, wherein the isolated nucleic acid comprises SEQ ID NO: 586.

In some embodiments, the disclosure provides an isolated nucleic acid that encodes the heavy chain variable domain of the COV_1116 antibody, wherein the isolated nucleic acid comprises SEQ ID NO: 591 and/or the isolated nucleic acid encodes the light chain variable domain of the of the COV_1116 antibody, wherein the isolated nucleic acid comprises SEQ ID NO: 596.

In some embodiments, the disclosure provides an isolated nucleic acid that encodes the heavy chain variable domain of the COV_1118 antibody, wherein the isolated nucleic acid comprises SEQ ID NO: 601 and/or the isolated nucleic acid encodes the light chain variable domain of the of the COV_1118 antibody, wherein the isolated nucleic acid comprises SEQ ID NO: 606.

In some embodiments, the disclosure provides an isolated nucleic acid that encodes the heavy chain variable domain of the COV_1122 antibody, wherein the isolated nucleic acid comprises SEQ ID NO: 611 and/or the isolated nucleic acid encodes the light chain variable domain of the of the COV_1122 antibody, wherein the isolated nucleic acid comprises SEQ ID NO: 616.

In some embodiments, the disclosure provides an isolated nucleic acid that encodes the heavy chain variable domain of the COV_1131 antibody, wherein the isolated nucleic acid comprises SEQ ID NO: 621 and/or the isolated nucleic acid encodes the light chain variable domain of the of the COV_1131 antibody, wherein the isolated nucleic acid comprises SEQ ID NO: 626.

In some embodiments, the disclosure provides an isolated nucleic acid that encodes the heavy chain variable domain of the COV_1136 antibody, wherein the isolated nucleic acid comprises SEQ ID NO: 631 and/or the isolated nucleic acid encodes the light chain variable domain of the of the COV_1136 antibody, wherein the isolated nucleic acid comprises SEQ ID NO: 636.

In some embodiments, the disclosure provides an isolated nucleic acid that encodes the heavy chain variable domain of the COV_1144 antibody, wherein the isolated nucleic acid comprises SEQ ID NO: 641 and/or the isolated nucleic acid encodes the light chain variable domain of the of the COV_1144 antibody, wherein the isolated nucleic acid comprises SEQ ID NO: 646.

In some embodiments, the disclosure provides an isolated nucleic acid that encodes the heavy chain variable domain of the COV_1145 antibody, wherein the isolated nucleic acid comprises SEQ ID NO: 651 and/or the isolated nucleic acid encodes the light chain variable domain of the of the COV_1145 antibody, wherein the isolated nucleic acid comprises SEQ ID NO: 656.

In some embodiments, the disclosure provides an isolated nucleic acid that encodes the heavy chain variable domain of the COV_1149 antibody, wherein the isolated nucleic acid comprises SEQ ID NO: 661 and/or the isolated nucleic acid encodes the light chain variable domain of the of the COV_1149 antibody, wherein the isolated nucleic acid comprises SEQ ID NO: 666.

In some embodiments, the disclosure provides an isolated nucleic acid that encodes the heavy chain variable domain of the COV_1151 antibody, wherein the isolated nucleic acid comprises SEQ ID NO: 671 and/or the isolated nucleic acid encodes the light chain variable domain of the of the COV_1151 antibody, wherein the isolated nucleic acid comprises SEQ ID NO: 676.

In some embodiments, the disclosure provides an isolated nucleic acid that encodes the heavy chain variable domain of the COV_1154 antibody, wherein the isolated nucleic acid comprises SEQ ID NO: 681 and/or the isolated nucleic acid encodes the light chain variable domain of the of the COV_1154 antibody, wherein the isolated nucleic acid comprises SEQ ID NO: 686.

In some embodiments, the disclosure provides an isolated nucleic acid that encodes the heavy chain variable domain of the COV_1165 antibody, wherein the isolated nucleic acid comprises SEQ ID NO: 691 and/or the isolated nucleic acid encodes the light chain variable domain of the of the COV_1165 antibody, wherein the isolated nucleic acid comprises SEQ ID NO: 696.

In some embodiments, the disclosure provides an isolated nucleic acid that encodes the heavy chain variable domain of the COV_1166 antibody, wherein the isolated nucleic acid comprises SEQ ID NO: 701 and/or the isolated nucleic acid encodes the light chain variable domain of the of the COV_1166 antibody, wherein the isolated nucleic acid comprises SEQ ID NO: 706.

In some embodiments, the disclosure provides an isolated nucleic acid that encodes the heavy chain variable domain of the COV_1170 antibody, wherein the isolated nucleic acid comprises SEQ ID NO: 711 and/or the isolated nucleic acid encodes the light chain variable domain of the of the COV_1170 antibody, wherein the isolated nucleic acid comprises SEQ ID NO: 716.

In some embodiments, the disclosure provides an isolated nucleic acid that encodes the heavy chain variable domain of the COV_1172 antibody, wherein the isolated nucleic acid comprises SEQ ID NO: 721 and/or the isolated nucleic acid encodes the light chain variable domain of the of the COV_1172 antibody, wherein the isolated nucleic acid comprises SEQ ID NO: 726.

In some embodiments, the disclosure provides an isolated nucleic acid that encodes the heavy chain variable domain of the COV_1177 antibody, wherein the isolated nucleic acid comprises SEQ ID NO: 731 and/or the isolated nucleic acid encodes the light chain variable domain of the of the COV_1177 antibody, wherein the isolated nucleic acid comprises SEQ ID NO: 736.

In some embodiments, the disclosure provides an isolated nucleic acid that encodes the heavy chain variable domain of the COV_1184 antibody, wherein the isolated nucleic acid comprises SEQ ID NO: 741 and/or the isolated nucleic acid encodes the light chain variable domain of the of the COV_1184 antibody, wherein the isolated nucleic acid comprises SEQ ID NO: 746.

In some embodiments, the disclosure provides an isolated nucleic acid that encodes the heavy chain variable domain of the COV_1198 antibody, wherein the isolated nucleic acid comprises SEQ ID NO: 751 and/or the isolated nucleic acid encodes the light chain variable domain of the of the COV_1198 antibody, wherein the isolated nucleic acid comprises SEQ ID NO: 756.

In some embodiments, the disclosure provides an isolated nucleic acid that encodes the heavy chain variable domain of the COV_2032 antibody, wherein the isolated nucleic acid comprises SEQ ID NO: 761 and/or the isolated nucleic acid encodes the light chain variable domain of the of the COV_2032 antibody, wherein the isolated nucleic acid comprises SEQ ID NO: 766.

In some embodiments, the disclosure provides an isolated nucleic acid that encodes the heavy chain variable domain of the COV_2048 antibody, wherein the isolated nucleic acid comprises SEQ ID NO: 771 and/or the isolated nucleic acid encodes the light chain variable domain of the of the COV_2048 antibody, wherein the isolated nucleic acid comprises SEQ ID NO: 776.

In some embodiments, the disclosure provides an isolated nucleic acid that encodes the heavy chain variable domain of the COV_2055 antibody, wherein the isolated nucleic acid comprises SEQ ID NO: 781 and/or the isolated nucleic acid encodes the light chain variable domain of the of the COV_2055 antibody, wherein the isolated nucleic acid comprises SEQ ID NO: 786.

In some embodiments, the disclosure provides an isolated nucleic acid that encodes the heavy chain variable domain of the COV_2056 antibody, wherein the isolated nucleic acid comprises SEQ ID NO: 791 and/or the isolated nucleic acid encodes the light chain variable domain of the of the COV_2056 antibody, wherein the isolated nucleic acid comprises SEQ ID NO: 796.

In some embodiments, the disclosure provides an isolated nucleic acid that encodes the heavy chain variable domain of the COV_2064 antibody, wherein the isolated nucleic acid comprises SEQ ID NO: 801 and/or the isolated nucleic acid encodes the light chain variable domain of the of the COV_2064 antibody, wherein the isolated nucleic acid comprises SEQ ID NO: 806.

In some embodiments, the disclosure provides an isolated nucleic acid that encodes the heavy chain variable domain of the COV_2066 antibody, wherein the isolated nucleic acid comprises SEQ ID NO: 811 and/or the isolated nucleic acid encodes the light chain variable domain of the of the COV_2066 antibody, wherein the isolated nucleic acid comprises SEQ ID NO: 816.

In some embodiments, the disclosure provides an isolated nucleic acid that encodes the heavy chain variable domain of the COV_2077 antibody, wherein the isolated nucleic acid comprises SEQ ID NO: 821 and/or the isolated nucleic acid encodes the light chain variable domain of the of the COV_2077 antibody, wherein the isolated nucleic acid comprises SEQ ID NO: 826.

In some embodiments, the disclosure provides an isolated nucleic acid that encodes the heavy chain variable domain of the COV_2093 antibody, wherein the isolated nucleic acid comprises SEQ ID NO: 831 and/or the isolated nucleic acid encodes the light chain variable domain of the of the COV_2093 antibody, wherein the isolated nucleic acid comprises SEQ ID NO: 836.

In some embodiments, the disclosure provides an isolated nucleic acid that encodes the heavy chain variable domain of the COV_2137 antibody, wherein the isolated nucleic acid comprises SEQ ID NO: 841 and/or the isolated nucleic acid encodes the light chain variable domain of the of the COV_2137 antibody, wherein the isolated nucleic acid comprises SEQ ID NO: 846.

In some embodiments, the disclosure provides an isolated nucleic acid that encodes the heavy chain variable domain of the COV_2143 antibody, wherein the isolated nucleic acid comprises SEQ ID NO: 851 and/or the isolated nucleic acid encodes the light chain variable domain of the of the COV_2143 antibody, wherein the isolated nucleic acid comprises SEQ ID NO: 856.

In some embodiments, the disclosure provides an isolated nucleic acid that encodes the heavy chain variable domain of the COV_2169 antibody, wherein the isolated nucleic acid comprises SEQ ID NO: 861 and/or the isolated nucleic acid encodes the light chain variable domain of the of the COV_2169 antibody, wherein the isolated nucleic acid comprises SEQ ID NO: 866.

In some embodiments, the disclosure provides an isolated nucleic acid that encodes the heavy chain variable domain of the COV_2172 antibody, wherein the isolated nucleic acid comprises SEQ ID NO: 871 and/or the isolated nucleic acid encodes the light chain variable domain of the of the COV_2172 antibody, wherein the isolated nucleic acid comprises SEQ ID NO: 876.

In some embodiments, the disclosure provides an isolated nucleic acid that encodes the heavy chain variable domain of the COV_2174 antibody, wherein the isolated nucleic acid comprises SEQ ID NO: 881 and/or the isolated nucleic acid encodes the light chain variable domain of the of the COV_2174 antibody, wherein the isolated nucleic acid comprises SEQ ID NO: 886.

In some embodiments, the disclosure provides an isolated nucleic acid that encodes the heavy chain variable domain of the COV_2205 antibody, wherein the isolated nucleic acid comprises SEQ ID NO: 891 and/or the isolated nucleic acid encodes the light chain variable domain of the of the COV_2205 antibody, wherein the isolated nucleic acid comprises SEQ ID NO: 896.

In some embodiments, the disclosure provides an isolated nucleic acid that encodes the heavy chain variable domain of the COV_2215 antibody, wherein the isolated nucleic acid comprises SEQ ID NO: 901 and/or the isolated nucleic acid encodes the light chain variable domain of the of the COV_2215 antibody, wherein the isolated nucleic acid comprises SEQ ID NO: 906.

In some embodiments, the disclosure provides an isolated nucleic acid that encodes the heavy chain variable domain of the COV_3049 antibody, wherein the isolated nucleic acid comprises SEQ ID NO: 911 and/or the isolated nucleic acid encodes the light chain variable domain of the of the COV_3049 antibody, wherein the isolated nucleic acid comprises SEQ ID NO: 916.

In some embodiments, the disclosure provides an isolated nucleic acid that encodes the heavy chain variable domain of the COV_3069 antibody, wherein the isolated nucleic acid comprises SEQ ID NO: 921 and/or the isolated nucleic acid encodes the light chain variable domain of the of the COV_3069 antibody, wherein the isolated nucleic acid comprises SEQ ID NO: 926.

In some embodiments, the disclosure provides an isolated nucleic acid that encodes the heavy chain variable domain of the COV_3077 antibody, wherein the isolated nucleic acid comprises SEQ ID NO: 931 and/or the isolated nucleic acid encodes the light chain variable domain of the of the COV_3077 antibody, wherein the isolated nucleic acid comprises SEQ ID NO: 936.

In some embodiments, the disclosure provides an isolated nucleic acid that encodes the heavy chain variable domain of the COV_3079 antibody, wherein the isolated nucleic acid comprises SEQ ID NO: 941 and/or the isolated nucleic acid encodes the light chain variable domain of the of the COV_3079 antibody, wherein the isolated nucleic acid comprises SEQ ID NO: 946.

In some embodiments, the disclosure provides an isolated nucleic acid that encodes the heavy chain variable domain of the COV_3100 antibody, wherein the isolated nucleic acid comprises SEQ ID NO: 951 and/or the isolated nucleic acid encodes the light chain variable domain of the of the COV_3100 antibody, wherein the isolated nucleic acid comprises SEQ ID NO: 956.

In some embodiments, the disclosure provides an isolated nucleic acid that encodes the heavy chain variable domain of the COV_3103 antibody, wherein the isolated nucleic acid comprises SEQ ID NO: 961 and/or the isolated nucleic acid encodes the light chain variable domain of the of the COV_3103 antibody, wherein the isolated nucleic acid comprises SEQ ID NO: 966.

In some embodiments, the disclosure provides an isolated nucleic acid that encodes the heavy chain variable domain of the COV_3129 antibody, wherein the isolated nucleic acid comprises SEQ ID NO: 971 and/or the isolated nucleic acid encodes the light chain variable domain of the of the COV_3129 antibody, wherein the isolated nucleic acid comprises SEQ ID NO: 976.

In some embodiments, the disclosure provides an isolated nucleic acid that encodes the heavy chain variable domain of the COV_3137 antibody, wherein the isolated nucleic acid comprises SEQ ID NO: 981 and/or the isolated nucleic acid encodes the light chain variable domain of the of the COV_3137 antibody, wherein the isolated nucleic acid comprises SEQ ID NO: 986.

The present disclosure also provides expression vectors (or plasmids) comprising at least one nucleic acid encoding a CDR, all six CDRs (i.e., H1, H2, H3, L1, L2, and L3), a V_{H} domain, and/or a V_{L} domain of one of the COV_1007, COV_1037, COV_1045, COV_1046, COV_1201, COV_2004, COV_2008, COV_2014, COV_2018, COV_2024, COV_2025, COV_2027, COV_2028, COV_2035, COV_2037, COV_2038, COV_2039, COV_2054, COV_2056, COV_2057, COV_2063, COV_2091, COV_2100, COV_2103, COV_2108, COV_2123, COV_2125, COV_2134, COV_2151, COV_2165, COV_2172, COV_2173, COV_2193, COV_2196, COV_3000, COV_3005, COV_3013, COV_3019, COV_3028, COV_3031, COV_3033, COV_3037, COV_3040, COV_3043, COV_3053, COV_3088, COV_1012, COV_1025, COV_1032, COV_1050, COV_1056, COV_1060, COV_1063, COV_1071, COV_1076, COV_1082, COV_1085, COV_1086, COV_1087, COV_1097, COV_1116, COV_1118, COV_1122, COV_1131, COV_1136, COV_1144, COV_1145, COV_1149, COV_1151, COV_1154, COV_1165, COV_1166, COV_1170, COV_1172, COV_1177, COV_1184, COV_1198, COV_2032, COV_2048, COV_2055, COV_2056, COV_2064, COV_2066, COV_2077, COV_2093, COV_2137, COV_2143, COV_2169, COV_2172, COV_2174, COV_2205, COV_2215, COV_3049, COV_3069, COV_3077, COV_3079, COV_3100, COV_3103, COV_3129, or COV_3137 antibodies, as well as other nucleic acid sequences useful for regulating polypeptide expression. Suitable expression vectors can be chosen or constructed, so that they contain appropriate regulatory sequences, including promoter sequences, terminator sequences, polyadenylation sequences, enhancer sequences, marker genes and other sequences as appropriate.

The expression vectors can be introduced into a host cell to produce the desired antibody. Systems for cloning and expression of a polypeptide in a variety of different host cells are well known in the art. For cells suitable for producing antibodies, see Gene Expression Systems, Academic Press, eds. Fernandez et al., 1999. Typically, the instant antibodies are expressed, e.g., in a transgenic animal (see Gene Expression Systems, Academic Press, eds. Fernandez et al., 1999), a Chinese Hamster Ovary Cell, a Human Embryonic Kidney 293T cell or in a cell described in the Examples. Any protein compatible expression system may be used to produce the disclosed antibodies.

A further aspect of the disclosure provides an isolated host cell comprising a nucleic acid (or expression vector) as disclosed herein. A still further aspect provides a method comprising introducing such nucleic acid (or expression vector) into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. The introduction of the nucleic acid into the cells may be followed by causing or allowing expression from the nucleic acid, e.g., by culturing host cells under conditions for expression of the gene. Following production by expression an antibody may be isolated and/or purified using any suitable technique, then used as appropriate.

Methods of Making Antibodies

Methods of making antibodies are described in the Examples. Numerous other methods for antibody preparation are known to those skilled in the art. For example, antibodies can also be produced using recombinant DNA methods. See, e.g., U.S. Pat. No. 4,816,567, EPO 8430268.0; EPO 85102665.8; EPO 85305604.2; PCT/GB 85/00392; EPO 85115311.4; PCT/US86/002269; and Current Trends in Monoclonal Antibody Development (Steven Shire et al., Eds. Springer, 2010), the disclosures of which are incorporated herein by reference in their entirety. Given the disclosure in this application of specific nucleic acid sequences and the V_{H} and V_{L} (or CDR) amino acid sequences encoded thereby, it is possible, using recombinant DNA techniques, to insert a nucleic acid of interest into an expression vector or otherwise express the nucleic acid of interest in a host cell to produce the desired antibody. In addition, as disclosed elsewhere in this application, modified versions of the antibodies described herein can be produced using known techniques, including, for example, random mutagenesis, error-prone PCR, and direct mutagenesis.

Monoclonal antibodies may also be produced by preparing immortalized cell lines capable of producing antibodies having desired specificity, for example against an antigen expressing a desired epitope, such as an S1 subunit, NTD, RBD or S2 subunit as disclosed in this application. Such immortalized cell lines may be produced in a variety of ways. Conveniently, a small non-human animal, such as a mouse, is hyperimmunized with the desired immunogen. The vertebrate is then sacrificed, usually several days after the final immunization, the spleen cells removed, and the spleen cells immortalized. The most common technique is fusion with a myeloma cell fusion partner, as first described by Kohler and Milstein (1975) Nature 256:495-497. Other techniques, including EBV transformation, transformation with bare DNA, e.g., oncogenes, retroviruses, etc., or any other method which provides for stable maintenance of the cell line and production of monoclonal antibodies. Specific techniques for preparing monoclonal antibodies are described in Antibodies: A Laboratory Manual, Harlow and Lane, eds., Cold Spring Harbor Laboratory, 1988, the full disclosure of which is incorporated herein by reference.

In one embodiment, the non-human animal includes at least a part of a human immunoglobulin gene. For example, it is possible to engineer transgenic mouse strains that express human heavy and light chain genes, but are incapable of expressing the endogenous mouse immunoglobulin heavy and light chain genes. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, U.S. Pat. No. 5,225,539, WO 96/34096, published Oct. 31, 1996, and PCT Application No. PCT/US96/05928, filed Apr. 29, 1996, the disclosures of which are incorporated herein by reference in their entirety.

Immortalized cell lines can be screened using standard methods, such as enzyme-linked immunosorbent assay (ELISA) or surface plasmon resonance analysis, to identify one or more hybridomas that produce an antibody that specifically binds with a specified antigen and/or epitope. Any form of the specified antigen may be used as the immunogen, e.g., recombinant antigen, naturally occurring forms, any variants or fragments thereof, as well as antigenic peptide thereof.

Another exemplary method of making antibodies includes screening protein expression libraries, e.g., phage or ribosome display libraries. Phage display technology mimics the mammalian immune system by cloning large libraries of antibody genes and selecting for binding to a desired target, such as the coronavirus epitopes disclosed in this application. Phage display is described, for example, in Ladner et al., U.S. Pat. No. 5,223,409; Smith (1985) Science 228:1315-1317; Clackson et al. (1991)Nature, 352: 624-628; Marks et al. (1991) J. Mol. Biol., 222: 581-597WO 92/18619; WO 91/17271; WO 92/20791; WO 92/15679; WO 93/01288; WO 92/01047; WO 92/09690; and WO 90/02809, the disclosures of which are incorporated herein by reference in their entirety.

Methods of Use

The antibodies described in this application that bind to a coronavirus can be used in a variety of research and medical applications. In one aspect, the disclosure provides a method of treating or preventing a coronavirus infection in a subject, comprising administering to the subject one or more of the antibodies described herein in an amount effective to treat or prevent the coronavirus infection, such as SARS—Co-V2. Subjects that can be treated with the antibodies disclosed in this application include humans and non-human mammals, including, but not limited to, non-human primates, dogs, cats, horses, cows, sheep, pigs, goats, minks, mice, rats, hamsters, and guinea pigs.

In one aspect, one or more of the antibodies described herein is used in a method of treating COVID-19. If the disease is COVID-19, the disease can be asymptomatic, mild, moderate, severe, or critical. An asymptomatic form of COVID-19 does not show any symptoms in the subject. A mild form of COVID-19 may show mild form of one or more of: tiredness, fever, cough, breathlessness after moderate exercise, sore throat, muscle ache, headache, and diarrhea. Mild form of COVID-19 may not require management of symptoms. A moderate form of COVID-19 may show moderate form of one or more of: tiredness, fever, cough, breathlessness after slight activity, sore throat, muscle ache, headache, and diarrhea. Moderate form of COVID-19 may require managing the symptoms. A severe form of COVID-19 may show of one or more of: severe tiredness, high fever, cough, breathlessness even at rest, painful breathing, loss of appetite, loss of thirst, sore throat, muscle ache, headache, diarrhea, and confusion. Severe form of COVID-19 would typically require significant intervention for managing symptoms, such as: pneumonia, hypoxemic respiratory failure, acute respiratory distress syndrome (ARDS), sepsis, septic shock, cardiomyopathy, arrhythmia, acute kidney injury, and complications from prolonged hospitalization including secondary bacterial infections, thromboembolism, gastrointestinal bleeding, and critical illness polyneuropathy/myopathy.

In another aspect, a cocktail of one or more of the antibodies described herein is used in a method of treating or preventing a coronavirus infection or disease, such as COVID-19. For example, the cocktail can include at least one first mAb or antigen-binding fragments thereof, as disclosed herein, that binds to a receptor-binding domain of the S1 subunit and at least one second mAb or antigen-binding fragments thereof, as disclosed herein, that binds to the N-terminal domain of the S1 subunit. For example, the first recombinant monoclonal antibody, or antigen-binding fragment thereof, is one of COV_2123, COV_2125, or COV_2173, or an antigen-binding fragment thereof, and the second recombinant monoclonal antibody is one of COV_2004, COV_2025, or COV_2039, or an antigen-binding fragment thereof.

The cocktail can also include at least one first mAb or antigen-binding fragments thereof, as disclosed herein, that binds to a receptor-binding domain of the S1 subunit and at least one second mAb or antigen-binding fragments thereof, as disclosed herein, that binds to the S2 subunit. The cocktail can also include at least one first mAb or antigen-binding fragments thereof, as disclosed herein, that binds to the N-terminal domain of the S1 subunit and at least one second mAb or antigen-binding fragments thereof, as disclosed herein, that binds to the S2 subunit. Or the cocktail can include at least one first mAb or antigen-binding fragments thereof, as disclosed herein, that binds to a receptor-binding domain of the S1 subunit and at least one second mAb or antigen-binding fragments thereof, as disclosed herein, that binds to the N-terminal domain of the S1 subunit, and at least one third mAb or antigen-binding fragments thereof, as disclosed herein, that binds to the S2 subunit.

Alternatively, the cocktail can include a first at least one first mAb or antigen-binding fragments thereof, as disclosed herein, that binds to a receptor-binding domain of the S1 subunit and at least one second mAb or antigen-binding fragments thereof, as disclosed herein, that binds to a receptor-binding domain of the S1 subunit, wherein the first and second mAb are different mAbs. Or the cocktail can include a first at least one first mAb or antigen-binding fragments thereof, as disclosed herein, that binds to a receptor-binding domain of the N-terminal domain of the S1 subunit and at least one second mAb or antigen-binding fragments thereof, as disclosed herein, that binds to the N-terminal domain of the S1 subunit, wherein the first and second mAb are different mAbs. Or the cocktail can include a first at least one first mAb or antigen-binding fragments thereof, as disclosed herein, that binds to a receptor-binding domain of the S2 subunit and at least one second mAb or antigen-binding fragments thereof, as disclosed herein, that binds to the S2 subunit, wherein the first and second mAb are different mAbs.

In some embodiments, one or more of the instant antibodies can be administered prophylactically before infection or in order to reduce or prevent transmission, or before any clinical indication of illness, disease or infection. In some embodiments, the one or more antibodies can be administered in a time period days before infection or before possible or presumed exposure or risk of exposure as a prophylactic. For example, one or more of the antibodies of the disclosure may be administered a day prior or before, 2 days before or prior, 3 days prior or before, 4 days prior or before, 5 days prior or before, 6 days prior or before, 7 days prior or before, a week prior or before, more than 7 days prior or before, more than a week prior or before, up to 9 days prior or before, up to 10 days prior or before expected exposure. The present antibodies may be used to provide immediate immunity, for example, to avoid an outbreak in a suitable environment, such as a nursing home, military base or hospital or to prevent transmission prior to travel (e.g., entering a plane, train, bus, etc.) or in other instances where social distancing is impractical. In some embodiments, a single administration, e.g., a single injection, may provide immediate immunity that lasts up to 6 months or longer.

In addition, one or more of the antibodies disclosed herein can be used to detect a coronavirus as described herein, such as SARS-CoV-2 in a sample. In one embodiment, the method comprises contacting one or more of the antibodies disclosed herein with the sample and analyzing the sample to detect binding of the antibody to the coronavirus in the sample, wherein binding of the antibody to the coronavirus in the sample indicates the presence of a coronavirus in the biological sample. Typically, the coronavirus detected is SARS-CoV-2. More typically, the antibodies used to detect coronavirus in a sample is one or more of COV_3053 and/or COV_3088.

In one embodiment, the sample comprises a non-biological sample, such as soil, water, or food products such as meat. In other embodiments, the sample comprises a biological sample, such as blood, serum, mucus (e.g., nasal swab), tissue, cells, urine, or stool. Such methods can be used to detect a coronavirus infection in a patient, wherein binding of the antibody to the coronavirus in a sample from the patient indicates the presence of the coronavirus infection in the patient.

Any appropriate label may be used in the detection methods and compositions described herein. A label is any molecule or composition bound to an antibody, or a secondary molecule that is conjugated thereto, and that is detectable by spectroscopic, photochemical, biochemical, immunochemical, electrical, optical or chemical means. Examples of labels, including enzymes, colloidal gold particles, colored latex particles, have been disclosed (U.S. Pat. Nos. 4,275,149; 4,313,734; 4,373,932; and 4,954,452, each incorporated by reference herein). Additional examples of useful labels include, without limitation, haptens (e.g., biotin, digoxigenin (DIG), dintrophenol (DNP), etc.), radioactive isotopes, co-factors, ligands, chemiluminescent or fluorescent agents, protein-adsorbed silver particles, protein-adsorbed iron particles, protein-adsorbed copper particles, protein-adsorbed selenium particles, protein-adsorbed sulphur particles, protein-adsorbed tellurium particles, protein-adsorbed carbon particles, and protein-coupled dye sacs. The attachment of a compound to a label can be through any means, including covalent bonds, adsorption processes, hydrophobic and/or electrostatic bonds, as in chelates and the like, or combinations of these bonds and interactions and/or may involve a linking group.

Formulations and Administration

The disclosure provides compositions comprising an antibody described herein that binds to a coronavirus as also herein described. In certain embodiments, the compositions are suitable for pharmaceutical use and administration to patients. These compositions comprise one or more of the COV_1007, COV_1037, COV_1045, COV_1046, COV_1201, COV_2004, COV_2008, COV_2014, COV_2018, COV_2024, COV_2025, COV_2027, COV_2028, COV_2035, COV_2037, COV_2038, COV_2039, COV_2054, COV_2056, COV_2057, COV_2063, COV_2091, COV_2100, COV_2103, COV_2108, COV_2123, COV_2125, COV_2134, COV_2151, COV_2165, COV_2172, COV_2173, COV_2193, COV_2196, COV_3000, COV_3005, COV_3013, COV_3019, COV_3028, COV_3031, COV_3033, COV_3037, COV_3040, COV_3043, COV_3053, COV_3088, COV_1012, COV_1025, COV_1032, COV_1050, COV_1056, COV_1060, COV_1063, COV_1071, COV_1076, COV_1082, COV_1085, COV_1086, COV_1087, COV_1097, COV_1116, COV_1118, COV_1122, COV_1131, COV_1136, COV_1144, COV_1145, COV_1149, COV_1151, COV_1154, COV_1165, COV_1166, COV_1170, COV_1172, COV_1177, COV_1184, COV_1198, COV_2032, COV_2048, COV_2055, COV_2056, COV_2064, COV_2066, COV_2077, COV_2093, COV_2137, COV_2143, COV_2169, COV_2172, COV_2174, COV_2205, COV_2215, COV_3049, COV_3069, COV_3077, COV_3079, COV_3100, COV_3103, COV_3129, or COV_3137 antibodies and a pharmaceutically acceptable excipient.

Pharmaceutically acceptable excipients include, but are not limited to a carrier or diluent, such as a gum, a starch (e.g. corn starch, pregeletanized starch), a sugar (e.g. lactose, mannitol, sucrose, dextrose), a cellulosic material (e.g. microcrystalline cellulose), an acrylate (e.g. polymethylacrylate), calcium carbonate, magnesium oxide, talc, or mixtures thereof; a binder (e.g. acacia, cornstarch, gelatin, carbomer, ethyl cellulose, guar gum, hydroxypropyl cellulose, hydroxypropyl methyl cellulose, povidone); a disintegrating agent (e.g. cornstarch, potato starch, alginic acid, silicon dioxide, croscarmelose sodium, crospovidone, guar gum, sodium starch glycolate), a buffer (e.g. Tris-HCl, acetate, phosphate) of various pH and ionic strength; and additive such as albumin or gelatin to prevent absorption to surfaces; a detergent (e.g. Tween 20, Tween 80, Pluronic F68, bile acid salts); a protease inhibitor; a surfactant (e.g. sodium lauryl sulfate); a permeation enhancer; a solubilizing agent (e.g. glycerol, polyethylene glycerol); an anti-oxidants (e.g. ascorbic acid, sodium metabisulfite, butylated hydroxyanisole); a stabilizer (e.g. hydroxypropyl cellulose, hydroxypropylmethyl cellulose); a viscosity increasing agent (e.g. carbomer, colloidal silicon dioxide, ethyl cellulose, guar gum); a sweetener (e.g. aspartame, citric acid); a preservative (e.g. Thimerosal, benzyl alcohol, parabens); a lubricant (e.g. stearic acid, magnesium stearate, polyethylene glycol, sodium lauryl sulfate); a flow-aid (e.g. colloidal silicon dioxide), a plasticizer (e.g. diethyl phthalate, triethyl citrate); an emulsifier (e.g. carbomer, hydroxypropyl cellulose, sodium lauryl sulfate); a polymer coating (e.g. poloxamers or poloxamines); a coating and film forming agent (e.g. ethyl cellulose, acrylates, polymethacrylates); an adjuvant; a pharmaceutically acceptable carrier for liquid formulations, such as an aqueous (water, alcoholic/aqueous solution, emulsion or suspension, including saline and buffered media) or non-aqueous (e.g., propylene glycol, polyethylene glycol, and injectable organic esters such as ethyl oleate) solution, suspension, emulsion or oil; and a parenteral vehicle (for subcutaneous, intravenous, intraarterial, or intramuscular injection), including but not limited to, sodium chloride solution, Ringer's dextrose, dextrose and sodium chloride, lactated Ringer's and fixed oils.

Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

A pharmaceutical composition of the disclosure is formulated to be compatible with its intended route of administration. Methods to accomplish the administration are known to those of ordinary skill in the art. This includes, for example, injections, by parenteral routes such as intravenous, intravascular, intraarterial, subcutaneous, intramuscular, intraperitoneal, intraventricular, intraepidural, or others as well as oral, nasal, ophthalmic, rectal, or topical. Sustained release administration is also specifically contemplated, by such means as depot injections or erodible implants. Localized delivery is particularly contemplated, by such means as delivery via a catheter to one or more arteries, such as the renal artery or a vessel supplying a localized site of interest.

In some embodiments, the present compositions may be formulated in nasal sprays or inhalation solutions or suspensions using approaches known and acceptable in the art and in the medical field and clinical practice. The Food and Drug Administration (FDA9 provides guideline and guidance with regard to such sprays, solutions and suspensions and spray drug products, including in Guidance for Industry documents available at fda.gov. An exemplary July 2002 Guidance for Industry document entitled Nasal Spray and Inhalation Solution, Suspension and Spray Drug Products-Chemistry, Manufacturing and Controls Documentation includes details regarding formulation components and compositions, specifications therefore, manufacturing, and closed container systems.

Nasal Sprays are drug products that contain active ingredients dissolved or suspended in a formulation, typically aqueous-based, which can contain other excipients and are intended for use by nasal inhalation. Container closure systems for nasal sprays include the container and all components that are responsible for metering, atomization, and delivery of the formulation to the patient. Nasal spray drug products contain therapeutically active ingredients (drug substances) dissolved or suspended in solutions or mixtures of excipients (e.g., preservatives, viscosity modifiers, emulsifiers, buffering agents) in nonpressurized dispensers that deliver a spray containing a metered dose of the active ingredient. The dose can be metered by the spray pump or could have been premetered during manufacture. A nasal spray unit can be designed for unit dosing or can discharge numerous metered sprays of formulation containing the drug substance. Nasal sprays are applied to the nasal cavity for local and/or systemic effects.

In some embodiments, the pharmaceutical compositions are aerosolized administration. A nebulizer is a drug delivery device used to administer medication in the form of aerosol into the respiratory tract. Nebulizers can be used for intransal and inhalation delivery of monoclonal antibodies through the mouth and nasal passage and are effective devices for delivery of monoclonal antibodies to the upper and/or lower respiratory tract. Nebulizers use oxygen, compressed air or ultrasonic power to break up medical solutions and suspensions into small aerosol droplets that can be directly inhaled from the mouthpiece of the device. In some embodiments, a metered-dose inhaler (MDI) device is used to deliver the one or more antibodies in a specific amount of medication to the lungs in the form of a short burst of aerosolized medicine that is usually self-administered by the patient via inhalation. Dry powder inhalers, which utilize micronized powder often packaged in single dose quantities in blisters or gel capsules containing the powdered medication, may also be used to deliver the one or more antibodies to the lungs. In one embodiment a subject antibody is administered to a patient by intravenous, intramuscular or subcutaneous injection. The antibody may be administered, for example, by bolus injunction or by slow infusion. The dosage may depend on the type and severity of the infection and/or on the characteristics of the individual, such as general health, age, sex, body weight and tolerance to drugs and should be adjusted, as needed, according to individual need and professional judgment. The dosage may also vary depending upon factors, such as route of administration, target site, or other therapies administered. The skilled artisan will be able to determine appropriate doses depending on these and other factors.

Toxicity and therapeutic efficacy of the composition can be determined by standard pharmaceutical procedures in cell cultures or experimental animals, e.g., determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Antibodies that exhibit large therapeutic indices may be less toxic and/or more therapeutically effective.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Example 1. Materials and Methods

### Example 1A. Human Samples

We have complied with the ethical regulations regarding these studies. These studies were approved by the Walter Reed Army Institute of Research (WRAIR) Institutional Review Board, and written informed consent was obtained from all participants. The investigators have adhered to the policies for protection of human subjects as prescribed in AR 70-25. Plasma from healthy and SARS-CoV-2 convalescent donors originated from WRAIR RV229 and RV229H studies, respectively. Other sources for convalescent plasma included StemExpress and the National Institute for Allergy and Infectious Diseases (NIAID) through its Biodefense and Emerging Infections research (BEI) repository. All convalescent donors (44% male and 56% female aged between 30-65) experienced a range of mild to severe symptoms, with blood drawn 3-7 weeks following the onset of symptoms. Donor #3, from whom monoclonal antibodies were isolated, was enrolled in the RV229H study after experiencing mild to moderate symptoms. Plasma and peripheral blood mononuclear cells (PBMC) were collected 7 weeks following a SARS-CoV-2 positive PCR test.

### Example 1B. Multiplex Antibody Binding Assay

A high-throughput bead-based antibody binding assay was performed as previously described^{51,52} with modifications to adapt to coronavirus antigens. A cocktail of 25 coronavirus antigens and 2 control proteins (HIV-1 antigens), obtained commercially (SinoBiological) or internally produced (see below), spanning spike S1 and S2 domains for all 7 human coronaviruses were covalently coupled to uniquely coded magnetic microspheres (Luminex) per manufacturer's protocol. Data was collected on a Bio-Plex®3D Suspension Array system (Bio-Rad) running xPONENT® v.4.2 (Luminex). Signal to Noise (S/N) ratio were calculated by the dividing the MFI for each sample by either Ig-depleted healthy plasma or a negative control antibody (MZ4) according to the type of sample analyzed.

### Example 1C. SARS-CoV-2 Pseudovirus Neutralization Assay

SARS-CoV-2 pseudovirions (pSV) were produced by co-transfection of HEK293T/17 cells with a pcDNA3.1 encoding SARS-CoV-2 S and an HIV-1 NL4-3 luciferase reporter plasmid (pNL4-3.Luc.R-E-, NIH AIDS Reagent Program). The S expression plasmid sequence was derived from the Wuhan Hu-1 strain (GenBank #NC_045512), which is also identical to the IL1/2020 and WA1/2020 strains. The S expression plasmid sequence was also codon optimized and modified to remove the last 18 amino acids of the cytoplasmic tail to improve S incorporation into the pseudovirions and thereby enhance infectivity. S expression plasmids for current SARS-CoV-2 VOC and VOI were similarly codon optimized, modified and included the following mutations: B.1.1.7 or Alpha, (69-70del, Yl44del, N501Y, A570D, D614G, P681H, T7181, S982A, D1118H), B.1.351 orBeta, (L18F, D80A, D215G, 241-243del, K417N, E484K, N501Y, D614G, A701V, E1195Q), B.1.617.2 or Delta, (T19R, G142D, de1156-157, R158G, L452R, T478K, D614G, P681R, D950N), P.1 or Gamma (L18F, T20N, P26S, D138Y, R190S, K417T, E484K, N501Y, D614G, H655Y,T10271) and B.1.427/429 (S131, W152C, L452R, D614G).

A D614G variant was also made from the Wuhan Hu-1 construct using the Q5 site-directed mutagenesis kit (NEB). In addition, a codon-optimized S expression plasmid encoding SARS-CoV-1 (Sino 1-11, GenBank #AY485277) was generated that incorporated a 28 amino acid C-terminal deletion to improve infectivity⁵³. Virions pseudotyped with the vesicular stomatitis virus (VSV) G protein were used as control. Infectivity and neutralization titers were determined using ACE2-expressing HEK293 target cells (Integral Molecular) in a semi-automated assay format using robotic liquid handling (Biomek NXp Beckman Coulter, as previously described¹⁸. Neutralization dose-response curves were fitted by nonlinear regression using the LabKey server, and the final titers are reported as the reciprocal of the dilution of plasma necessary to achieve 50% neutralization (ID50, 50% inhibitory dose or IC50, 50% inhibitory concentration) and 80% neutralization (ID80, 80% inhibitory dose or IC80, 80% inhibitory concentration). Assay equivalency was verified by participation in the SARS-CoV-2 Neutralizing Assay Concordance Survey (SNACS) run by the Virology Quality Assurance Program and External Quality Assurance Program Oversite Laboratory (EQAPOL) at the Duke Human Vaccine Institute, sponsored through programs supported by the National Institute of Allergy and Infectious Diseases, Division of AIDS.

### Example 1D. Sorting of SARS-CoV-2-Positive B Cells

Cryopreserved PBMCs were thawed in warm media containing benzonase, then washed with PBS and stained for viability using the Aqua Live/Dead stain (ThermoFisher). Cells were incubated at 21° C. for 30 min with a cocktail of antibodies including CD3 BV510 (BioLegend), CD4 BV510 (BD Biosciences), CD8 BV510 (BioLegend), CD14 BV510 (BioLegend), CD16 BV510 (BD Biosciences) and CD56 BV510 (BioLegend) as dump channel markers, and CD19 PE Dazzle 594 (BioLegend), CD38 BUV496 (BD Biosciences), CD27 BV605 (BioLegend), CD20 AF700 (BD Biosciences), IgD APC/Cyanine7 (BioLegend), integrin β7 PE/Cyanine7 (BD Biosciences), IgG (BioLegend), CD10 BUV395 (BD Biosciences), CD21 FITC (BioLegend), and IgM BV650 (BioLegend).

Two sorting strategies were used to maximize the number of probes used to isolate antigen-specific B cells: The first strategy utilized a stabilized SARS-CoV-2 S trimer (HexaPro¹¹) conjugated to streptavidin-APC, and the second strategy utilized a multivalent spike ferritin nanoparticle (SpFN¹⁸) displaying eight S trimers to potentially capture conformation-specific B cell receptors. SpFN was incubated with cells during primary staining, and SpFN⁺ B cell were identified by secondary staining using the MM43 monoclonal antibody (SinoBiological, #40591-MM43) conjugated to AF647 (ThermoFisher). Both strategies included SARS-CoV-2 RBD, S1, S2 (ThermoFisher) SARS-CoV S2P, RBD, and S1 to BUV737 (BD Biosciences); MERS-CoV RBD, SL, and S2 to BV421 (BioLegend); and S1+S2 from 229E, NL63, HKU1, and OC43 to BV785 (BioLegend), which were biotinylated, tetramerized, and conjugated to streptavidin-PE. Since these antigens used the same conjugated streptavidin-PE, B cell binding could not be distinguished between SARS-CoV-2 RBD, S1, and S2 using flow cytometry. Specific B cell binding by flow cytometry was determined to the stabilized trimer using conjugated APC, and SpFN using AF647 conjugated to MM43. CD19⁺ B Cells that were antigen-specific were single-cell sorted into PCR plates containing lysis buffer composed of murine RNAse inhibitor (New England Biolabs), dithiothreitol (DTT), SuperScript III First Strand Buffer (ThermoFisher), Igepal (Sigma), and carrier RNA (Qiagen) at one cell per well using a FACS ARIA (Becton Dickinson) and stored at −80° C. until subsequent reverse transcription. Analysis was performed using FlowJo 10 (BD Bioscience).

### Example 1E. Antibody Sequencing and Production

RNA from single antigen-specific B cells was reverse-transcribed using random hexamers and the SuperScriptIII kit (ThermoFisher). Antibody V (D) J genes were amplified from the cDNA by nested PCR, using the HotStar Taq DNA Polymerase kit (Qiagen) using a combination of primer sets and methods described previously³⁹. V(D)J gene assignment, somatic hypermutation and CDR3 determinations were performed using IgBlast28. Antibody variable regions were synthesized and cloned (Genscript) into CMVR expression vectors (NIH AIDS reagent program) between a murine Ig leader (GenBank DQ407610) and the constant regions of human IgG1 (GenBank AAA02914), Igκ (GenBank AKL91145) or Ig, (GenBank AAA02915). Antibodies were expressed by co-transfecting plasmids encoding paired heavy and light chains into Expi293F cells (ThermoFisher). Monoclonal antibodies were purified 4 to 5 days post-transfection using AMMAG™ protein A magnetic beads and the AMMAG™ SA purification system (Genscript), according to the manufacturer's recommendations, and buffer exchanged into phosphate-buffered saline (PBS). The purity and stability of monoclonal antibodies was assessed by SDS-PAGE and Coomassie staining in both reducing and non-reducing conditions. Control antibodies were all expressed as human IgG1 and purified from Expi293F cells, as described above.

### Example 1F. Fab Production

Freshly purified WRAIR IgGs in PBS buffer (pH 7.4) were mixed with Lys C protease (New England Biolabs) at 1:2000 (w:w) ratio. Reaction was allowed to proceed for 2-3 hours in a water bath incubator at 37° C. Digestion was assessed by SDS-PAGE and upon completion, the reaction mixture was passed through protein-A beads (0.5-1 ml beads) three times and the final flow through was assessed by SDS-PAGE for purity.

### Example 1G. Production of Recombinant Proteins

Recombinant SARS-CoV-2 proteins RBD (318-514), NTD (1-290) and S1 (1-665) were made from a synthesized full-length spike sequence (Genscript) from strain USA/IL1/2020 (GenBank #MN988713) and were cloned with C-terminal AVITAG™ (Avidity) and poly-histidine tags into the CMVR vector under the bovine prolactin leader sequence. The coding sequence for the SARS-CoV-2 (Genbank #MN908947) stabilized trimer (S-2P) was a generous gift from Jason McLellan. The S2P sequence was subcloned into the pCMVR vector with C-terminal AVITAG™ (Avidity) and poly-histidine tags. Four additional stabilizing mutations were added using the Quikchange multisite-directed mutagenesis kit (Agilent) to make the HexaPro variant with improved stability¹¹, referred to as stabilized S trimer throughout the manuscript. SARS-CoV-2 RBD constructs (331-527) were also modified to incorporate a N-terminal hexa-histidine tag were derived from the Wuhan-Hu-1 strain genome sequence (GenBank MN9089473). Subsequent RBD VOC with point mutations were generated using a modified QuikChange site-directed mutagenesis protocol (Agilent). A S-2P construct derived from SARS-CoV-1 was generated as previously described⁵⁴. Spike proteins were expressed and biotinylated as previously described⁵⁵, with mutations for B.1.1.7, B.1.351, P.1, B.1.617.2 and other variants added by QuikChange site-directed mutagenesis. ACE2-Ig, a fusion protein made by connecting the human ACE2 (Q9BYF1) extracellular domain (residues 19-611) to the constant domain of a human IgG1 was expressed and purified as described above for antibodies. All proteins were produced transiently from Expi293F or FreeStyle 293F (stabilized trimer) cells (both ThermoFisher) and purified from cell culture supernatants using Ni-NTA (Qiagen) affinity. The stabilized trimer was further purified by gel filtration on an ENRICH™ SEC 650 colunmn (Bio-Rad) and the presence of trimeric S was verified by negative stain electron microscopy. When needed, proteins were biotinylated using the BirA biotin-protein ligase kit (Avidity).

### Example 1H. Authentic SARS-CoV-2 Plaque Reduction Neutralization Test (PRNT)

Vero E6 cells (ATCC CRL-1586) maintained in Dulbecco's Modified Eagle Medium (DMEM) medium supplemented with 10% fet al bovine serum (FBS) and 2 mM L-glutamine were seeded in 6-well plates at 1×10⁶ cells per well one day prior to infection. PRNTs were performed in triplicate in a biosafety level 3 facility. Three-fold dilutions were performed for each mAb, beginning at 25 μg/ml. The dilutions were made at 2× concentrations and mixed 1:1 with 100 plaque forming units (pfu) of SARS-CoV-2 virus (isolate 2019-nCoV/Italy-INMI1, BEI NR-52284, which is 100% identical to the Wuhan Hu-1 or IL1/2020 strains). The antibody-virus mixtures were incubated at 37° C. for 1 h. The mixtures were then added to the Vero E6 monolayers, incubated for one hour at 37° C. in a humidified incubator with 5% CO², then overlaid with 0.5% agarose in serum-free minimal essential media (MEM) with 100 U/ml of penicillin-streptomycin, 0.25 μg/ml amphotericin B, and 2 mM L-glutamine. The cells were incubated for 72 hours, then fixed in 10% formaldehyde and stained with 0.5% crystal violet. The half maximal inhibitory concentration (IC50) values were determined as the concentration of antibody that resulted in a 50% reduction in number of plaques, compared to virus only control wells.

### Example 1I. Measurements of Antibody Fc Effector Functions Using Recombinant Proteins

ADCP. ADCP was measured as previously described⁵⁶ using biotinylated SARS-CoV-2 S stabilized trimer. The phagocytic score was calculated by multiplying the percentage of bead-positive cells by the geometric mean fluorescence intensity (MFI) of the bead-positive cells and dividing by 10⁴.

ADNP. Biotinylated SARS-CoV-2 stabilized trimer was incubated with yellow-green streptavidin-fluorescent beads (Molecular Probes) for 2 h at 37° C. 10 μl of a 100-fold dilution of beads-protein mixture was incubated with monoclonal antibodies as described above before addition of effector cells (50,000 cells/well). Fresh peripheral blood leukocytes from human were used as effector cells after red blood cell lysis with ACK lysing buffer (ThermoFisher Scientific). After 1 h incubation at 37° C., the cells were washed, surface stained, fixed with 4% formaldehyde solution and fluorescence was evaluated on a LSRII (BD Bioscience). Antibodies used for flow cytometry were anti-human CD3 AF700 (clone UCHT1) and anti-human CD14 APC-Cy7 (clone MϕP9; both BD Biosciences) and anti-human CD66b Pacific Blue (clone G10F5, Biolegend). The phagocytic score was calculated by multiplying the percentage of bead-positive neutrophils (SSC high, CD3-CD14-CD66+) by the geometric MFI of the bead-positive cells and dividing by 10⁴.

### Example 1J. Measurements of Antibody Fc Effector Functions Using Cell Surface-Expressed Spikes

Opsonization. SARS-CoV-2 S-expressing FREESTYLE™ 293F cells (ThermoFisher) were generated by transfection with linearized plasmid encoding a codon-optimized full-length SARS-CoV-2 S protein matching the amino acid sequence of the IL1/2020 isolate (GenBank #MN988713). Stable transfectants were single-cell sorted and selected to obtain a high-level spike surface expressing clone (293F-Spike-S2A). 293F-Spike-S 2A cells were incubated with monoclonal antibodies diluted 3-fold from 15 to 0.06 μg ml⁻⁴ for 30 min at 37° C. Cells were washed twice and stained with anti-human IgG PE (Southern Biotech). Cells were then fixed with 4% formaldehyde solution and fluorescence was evaluated on a LSRII (BD Bioscience).

Antibody dependent complement activation (ADCD). An ADCD assay was adapted from Fischinger et al.⁵⁷ Briefly, 293F-Spike-S2A cells were incubated with monoclonal antibodies as described above and washed twice and resuspended in R10 media. Cells were washed with PBS and resuspended in 200 μl of guinea pig complement (Cedarlane), which was prepared at a 1:50 dilution in Gelatin Veronal Buffer with Ca²⁺ and Mg²⁺ (Boston BioProducts). After incubation at 37° C. for 20 min, cells were washed and stained with an anti-guinea pig complement C3-FITC (polyclonal, ThermoFisher Scientific). Cells were fixed with 4% formaldehyde solution and fluorescence was evaluated on a LSRII (BD Bioscience).

### Example 1K. Epitope Binning

Epitopes of the NTD and RBD mAbs were first mapped by binding competition against each other (NTD) or against a set of control antibodies (RBD) using Biolayer interferometry (BLI) on an OCTET® RED96 instrument (ForteBio), as previously described³⁹. Antibodies were defined as competing when binding signal of the second antibody was reduced to less than 25% of its maximum binding capacity and non-competing when binding was greater than 50%. Intermediate competition was defined by binding levels of 25-50%. Control antibodies RBD-A, RBD-B and RBD-C were CC12.1, CC12.16¹³ and CR3022⁵⁸, respectively. The same approach was used to assess binding competition between NTD and RBD antibodies within the stabilized S trimer. ACE2-Ig was used like an antibody to assess the ability of NTD and RBD antibodies to block ACE2 binding to the S trimer.

### Example 1L. Biolayer Interferometry Binding Assays

Real-time interactions between purified SARS-CoV-2 proteins and antibodies were monitored on an OCTET® RED96 instrument (ForteBio) as previously described³⁹ using biotinylated SARS-CoV-2 NTD and RBD proteins as described above. After reference subtraction, apparent binding kinetic constants were determined, from at least 4 concentrations of antibody, by fitting the curves to a 1:1 binding model using the Data analysis software 10.0 (ForteBio). To assess binding to a panel of RBD mutants, HIS1K biosensors (ForteBio) were equilibrated in assay buffer (PBS) for 15 s before loading of His-tagged SARS-CoV-2 RBD, VOC RBDs, or SARS-CoV-1 RBD (30 μg ml⁻¹ diluted in PBS) for 100 s. Binding responses were measured at the end of the association step using the Data analysis software 10.0 (ForteBio). hACE2-RBD competition assays were carried out as follows: SARS-CoV-2 RBD (30 μg ml⁻¹ diluted in PBS) was immobilized on HIS1K biosensors (ForteBio) for 220 seconds. Test antibodies were allowed to bind for 200 s, followed by baseline equilibration (30 s), and then incubation with hACE2 protein (30 μg ml⁻¹) for 120 s. Percent inhibition (PI) of RBD binding to hACE2 by antibodies was determined using the equation: PI=[(ACE2 binding following RBD-antibody incubation))/(ACE2 binding)]×100. Antibody concentration was titrated from 100 μg ml⁻¹ by serial two-fold dilutions. All assays were performed at 30° C. with agitation set at 1,000 rpm.

### Example 1M. Epitope Mapping of Antibodies by Alanine Scanning

Epitope mapping was performed essentially as described previously⁵⁹ using SARS-CoV-2 (strain Wuhan-Hu-1) S protein RBD and NTD shotgun mutagenesis mutation libraries, made using a full-length expression construct for S protein. 184 residues of the RBD (between S residues 335 and 526), and 300 residues of the NTD (between residues 2 and 307) were mutated individually to alanine, and alanine residues to serine. Mutations were confirmed by DNA sequencing, and clones arrayed in 384-well plates, one mutant per well. Binding of mAbs to each mutant clone in the alanine scanning library was determined, in duplicate, by high-throughput flow cytometry. Antibody reactivity against each mutant S protein clone was calculated relative to wild-type S protein reactivity by subtracting the signal from mock-transfected controls and normalizing to the signal from wild-type S-transfected controls. Mutations within clones were identified as relevant to the mAb epitope if they did not support reactivity of the test mAb but supported reactivity of other SARS-CoV-2 antibodies. This counter-screen strategy facilitates the exclusion of S mutants that are locally misfolded or have an expression defect.

### Example 1N. X-Ray Crystallography and Structure Analysis

WRAIR-2173-RBD (15.0 mg ml⁻¹), WRAIR-2151-RBD (12.0 mg ml⁻¹), WRAIR-2057-RBD (12.0 mg ml⁻¹) and WRAIR-2125-RBD complexes (10.0 mg ml⁻¹) were screened for crystallization conditions using an Art Robbins Gryphon crystallization robot, 0.2 μl drops, and a set of 1200 conditions and observed daily using a Jan Scientific UVEX-PS. Crystals used for data collection grew in the following crystallization conditions: WRAIR-2173-RBD complex: 0.09M NPS (Sodium nitrate, Sodium phosphate dibasic, Ammonium sulfate), 0.1M buffer system 3 (Tris base and BICINE, pH 8.5), 50% precipitant mix 4 (25% v/v MPD; 25% PEG 1000; 25% w/v PEG 3350); WRAIR-2151-RBD complex: 0.1 M Sodium acetate trihydrate pH 4.6, 2.0 M Ammonium sulfate; WRAIR-2057-RBD complex: 8% v/v Tacsimate pH 5.0, 20% w/v Polyethylene glycol 3,350; WRAIR-2125-RBD complex: 0.12 M alcohol mixture (1,6-Hexanediol; 1-Butanol; 1,2-Propanediol; 2-Propanol; 1,4-Butanediol; 1,3-Propanediol), 0.1M buffer system 3 (Tris base and BICINE, pH 8.5), 50% precipitant mix 4 (25% v/v MPD; 25% PEG 1000; 25% w/v PEG 3350) and 0.1 M Manganese(II) chloride tetrahydrate.

Diffraction data were collected at Advanced Photon Source (APS) beamlines. Diffraction data for WRAIR-2125-RBD and WRAIR-2151-RBD complexes were significantly anisotropic and were corrected using the UCLA Diffraction Anisotropy Server⁶⁰. All the crystal structures described in this study were solved by molecular replacement (MR) using PHASER, and iterative model building, and refinement were performed in COOT and Phenix^{61, 62, 63} Diffraction data quality was assessed with Phenix xtriage using data output from HKL2000⁶⁴ and XDS. All structures were refined using Phenix refine with positional, global isotropic B-factor refinement and defined TLS groups. Manual model building was performed in COOT. Overall, the Ramachandran plot as determined by MOLPROBITY showed 92-95% of all residues in favored regions and 4-6% of all residues in the allowed regions. Electron density for the structures was clearly interpretable except for the heavy chain Fc1 domain of WRAIR-2151. Interactive surfaces were analyzed using PISA (www.ebi.ac.uk/pdbe/pisa/). Structure figures were prepared using PyMOL (DeLano Scientific).

### Example 1O. Negative-stain Electron Microscopy

Fab fragments and SARS-CoV-2 S-2P were mixed at a 3:1 molar ratio for 30 minutes at room temperature, followed by purification using a Superdex-200 column. Purified proteins (5-10 μg/ml) were deposited on carbon-coated copper grids and stained with 0.75% uranyl formate and imaged using a FEI T20 operating at 200 kV with an Eagle 4K CCD using SerialEM or using a Thermo Scientific Talos L120C operating at 120 kV with Thermo Scientific Ceta detector using EPU. All image processing steps were done using RELION 3.0.8⁶⁵ and cryosparc v3.2.0⁶⁶. Particles were picked either manually or using templates generated from manually picked 2D class averages. CTF estimation was done with CTFFIND 4.1.13 and used for 2D classification. 3D map reconstructions were generated using an initial reference generated from S-2P (PDB code: 6VXX) with a low pass filter of 100 Å to remove distinguishable features and ‘Cl’ symmetry. An intermediate structure model was used to create a mask to further refine the structure. Visual analysis and figure generation were performed using Chimera⁶⁷.

### Example 1P. In Vivo Protection Studies in K18-hACE2 Transgenic Mice

All research in this study involving animals was conducted in compliance with the Animal Welfare Act, and other federal statutes and regulations relating to animals and experiments involving animals and adhered to the principles stated in the Guide for the Care and Use of Laboratory Animals, NRC Publication, 1996 edition. The research protocol was approved by the Institutional Animal Care and Use Committee of the Trudeau Institute and US Army Medical Research. K18-hACE2 transgenic mice were obtained from Jackson Laboratories (Bar Harbor, ME).

Mice were housed in the animal facility of the Trudeau Institute and cared for in accordance with local, state, federal, and institutional policies in a National Institutes of Health American Association for Accreditation of Laboratory Animal Care-accredited facility. For the prophylactic protection studies, on day ˜1, groups of 15 male and female K18-hACE2 mice (8-10 weeks of age) were injected intravenously with the purified antibodies at the indicated dose. On study day 0, all mice were inoculated with 1.25 ×10⁴ PFU of SARS-CoV-2 USA-WA1/2020 via intranasal instillation, a challenge dose determined from a previous study¹⁹. In the therapeutic study, mice (8-10 weeks of age) were inoculated with SARS-CoV-2 USA-WA1/2020 24 hours prior to being injected intravenously with the indicated antibody cocktail. All mice were monitored for clinical symptoms and body weight twice daily, every 12 hours, from study day 0 to study day 14. Mice were euthanized if they displayed any signs of pain or distress as indicated by the failure to move after stimulated or inappetence, or if mice have greater than 25% weight loss compared to their study day 0 body weight. From each group, a subset (5) of mice, were sacrificed 2 days after challenge for determination of infectious virus titers in lower respiratory tract (from bronchoalveolar lavage and lung tissue) using a PRNT assay.

### Example 1Q. Evaluation of Escape and Selection of Virus Variants

For the evaluation of antibody escape ability, and generation of putative antibody escape S variants, a previously described chimeric recombinant VSV derivative (rVSV/SARS-CoV-2/GFP2E1) that encodes a SARS-CoV2 S protein in place of VSV-G, recapitulating the neutralization properties of authentic SARS-CoV-2 was prepared and passaged to generate diversity as previously described⁶⁸.

### Example 1R. Statistical Analysis

Neutralization is the geometric mean of the IC50 values calculated using 5-parameter logistic regression from at least two-independent experiments performed in triplicates (R package nplr). Non-parametric Spearman correlations were used to assess relationship between neutralization and binding or neutralization and effector function data as well as between neutralization data obtained from the pseudotyped and authentic SARS-CoV-2 neutralization assays. Two-tailed Mann-Whitney t-tests were used to verify the existence of significant differences between NTD and RBD mAbs in several binding and functional assays. In the animal studies, one-way ANOVA with Dunnett's multiple comparisons tests were used to assess significance in weight changes and viral loads across groups compared to the isotype control antibody-treated animals. Survival curves were compared individually to the isotype control antibody using a Mantel-Cox log-rank test. Fold change in binding to mutant proteins was calculated relative to the wild-type WA1/2020 spike or RBD proteins. In absence of binding, a background binding value (0.05 nm in BLI assays) was attributed. Fold change in neutralization to VOC was calculated relative to the IL1/2020 virus. Non-neutralizing mAbs were assigned the IC50 of 25 μg ml⁻¹ antibody, the mAb starting concentration in the assay. All tests, except for the 5 parameter logistic regression performed in R (version 3.6.3) and R studio (1.2.1355), were performed in Prism (version 9, GraphPad Software). Data were graphed using Prism software (version 9, GraphPad Software).

### Example 2. Results

2a. Isolation of SARS-CoV-2 Antibodies

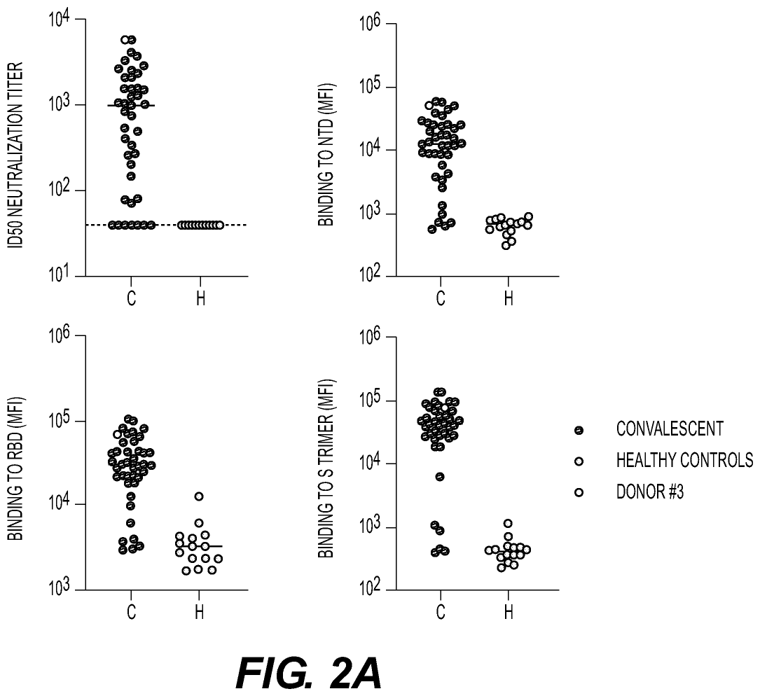

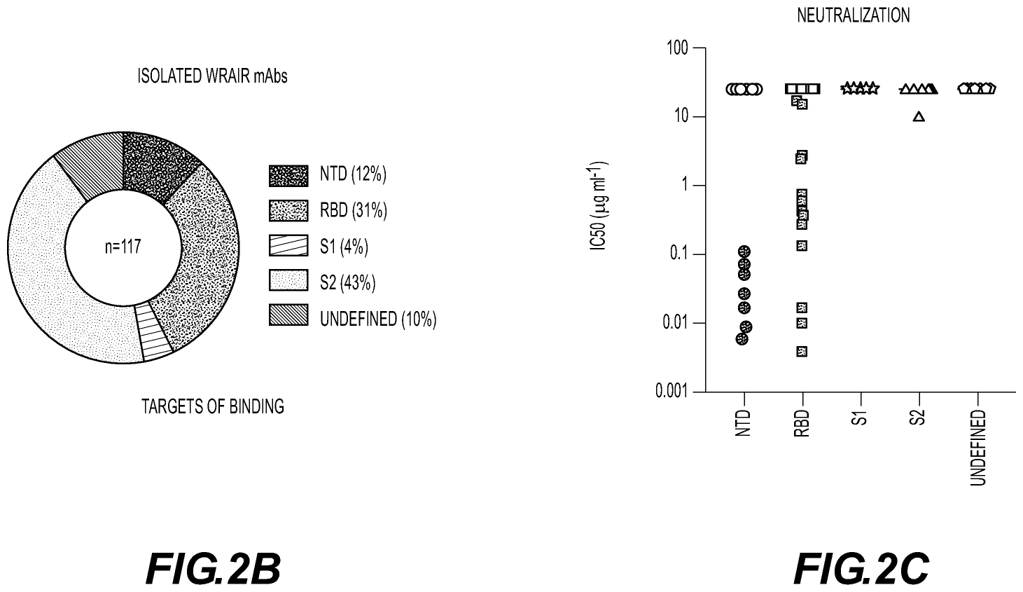

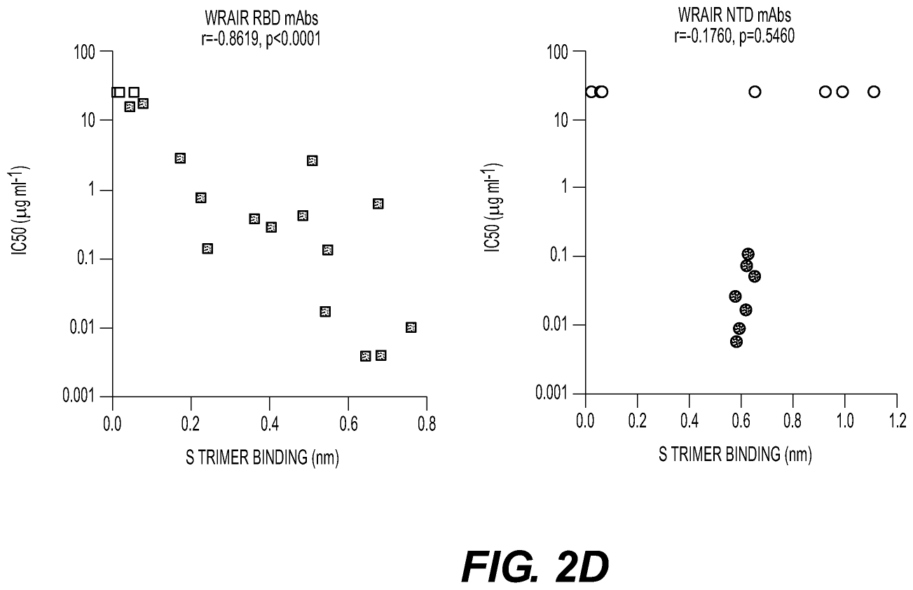

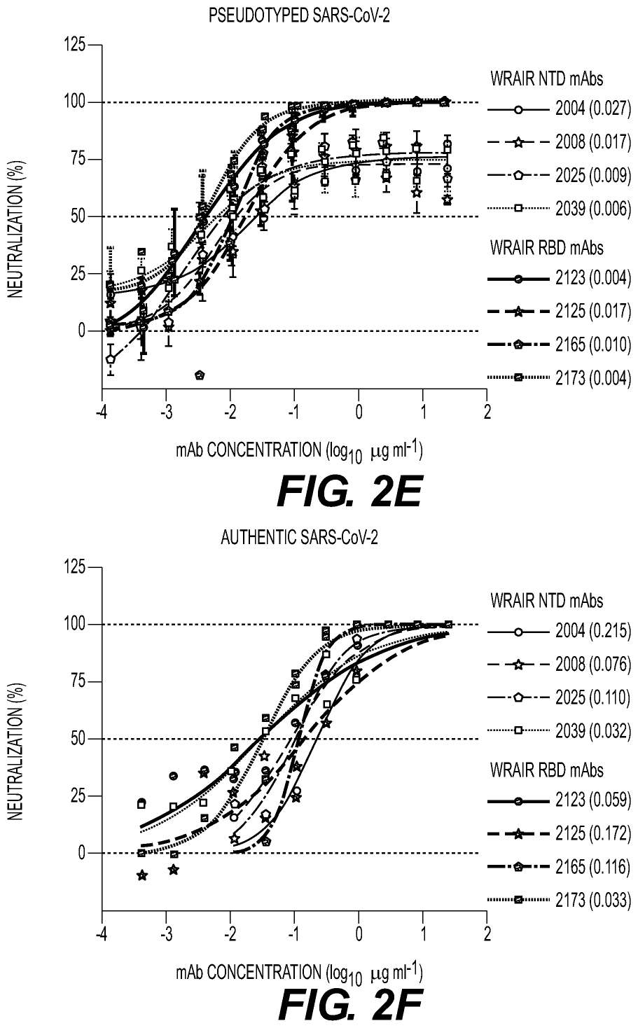

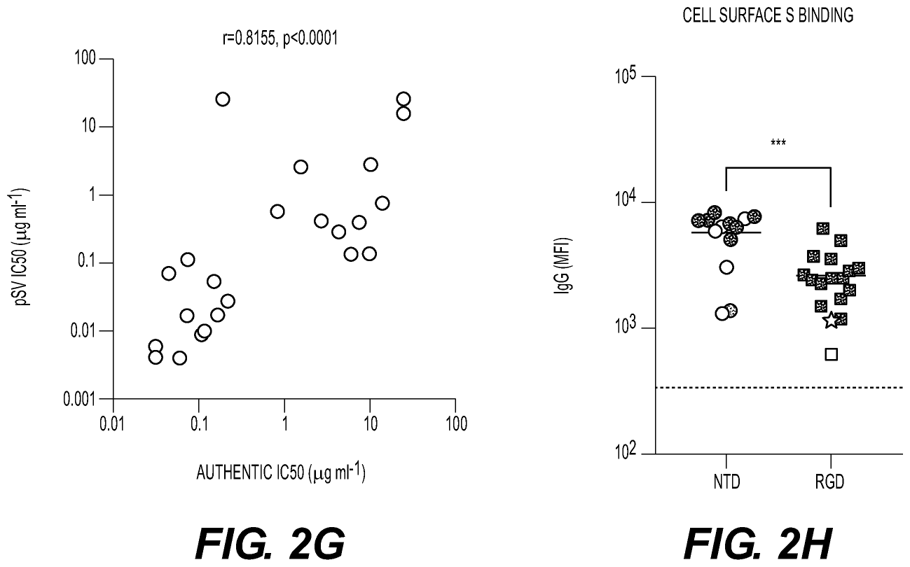

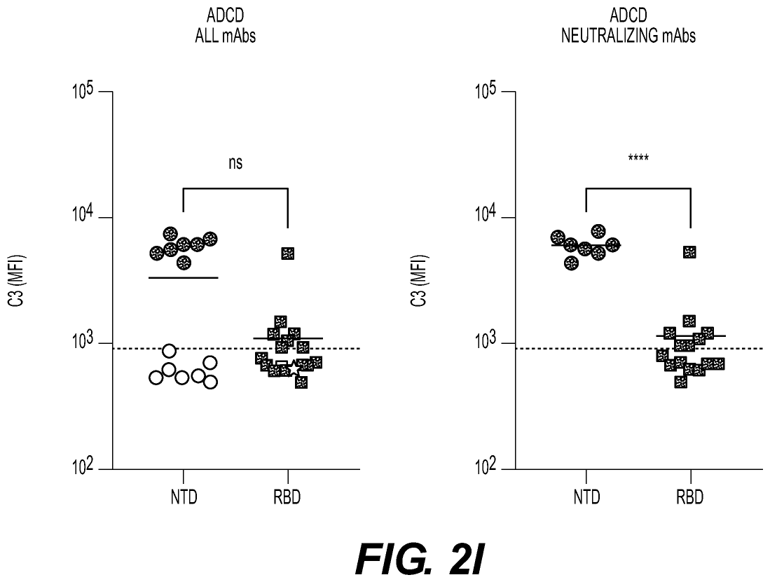

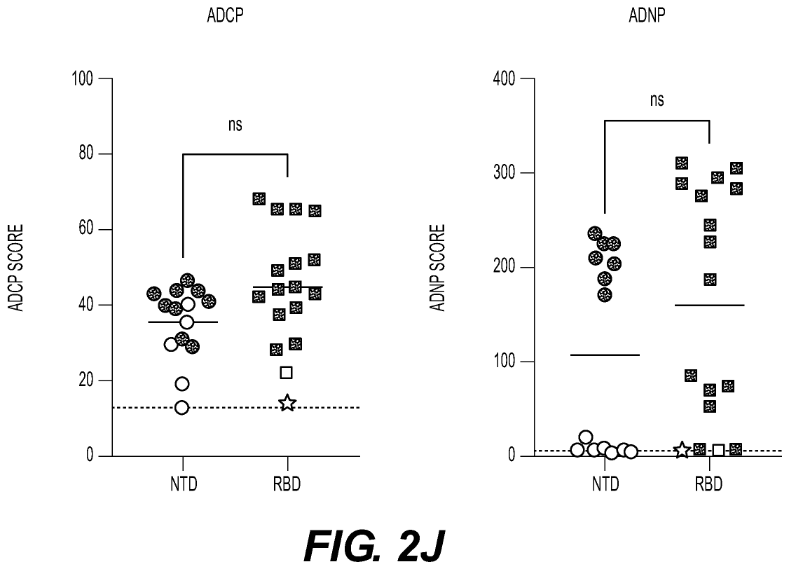

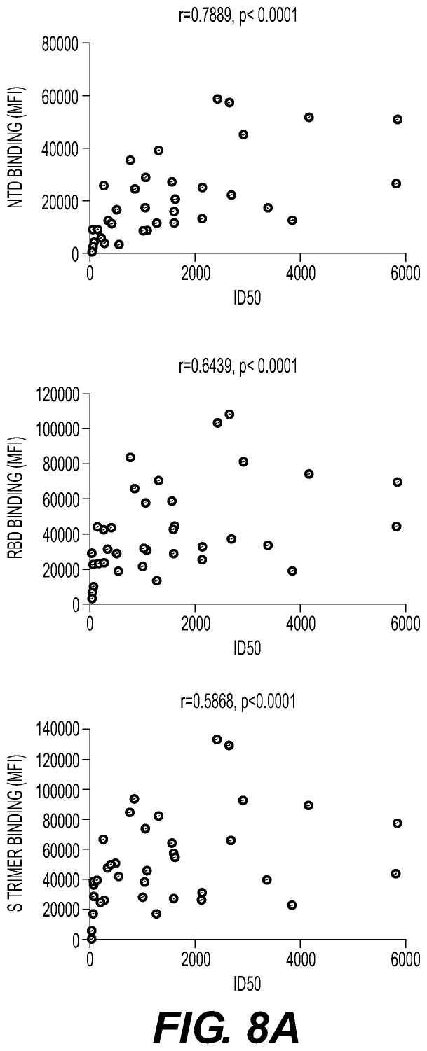

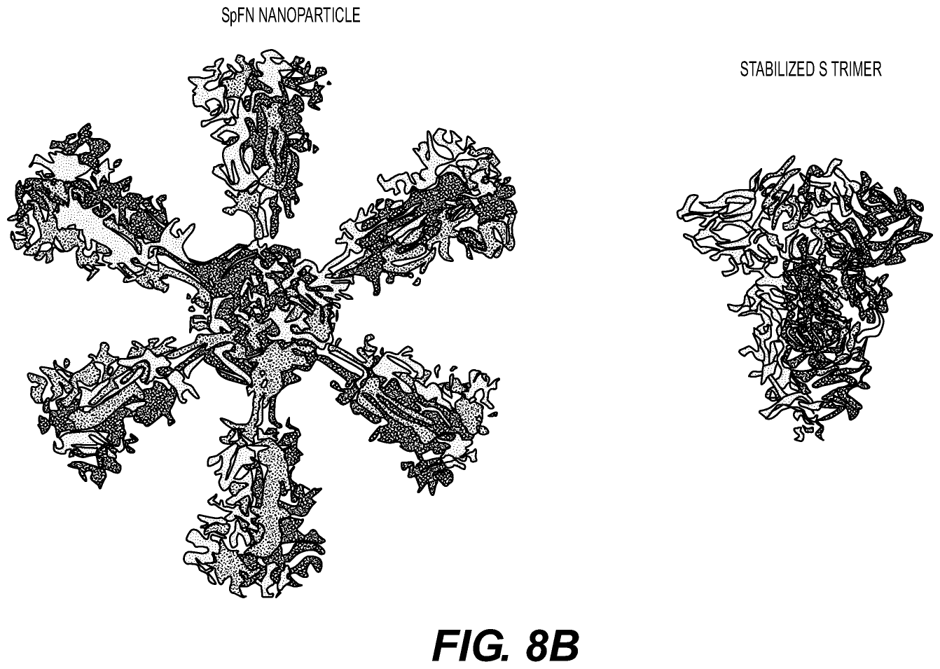

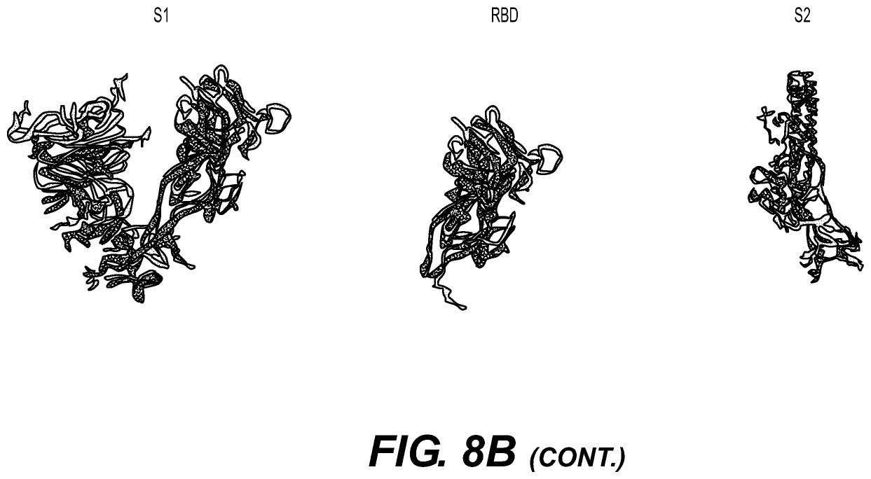

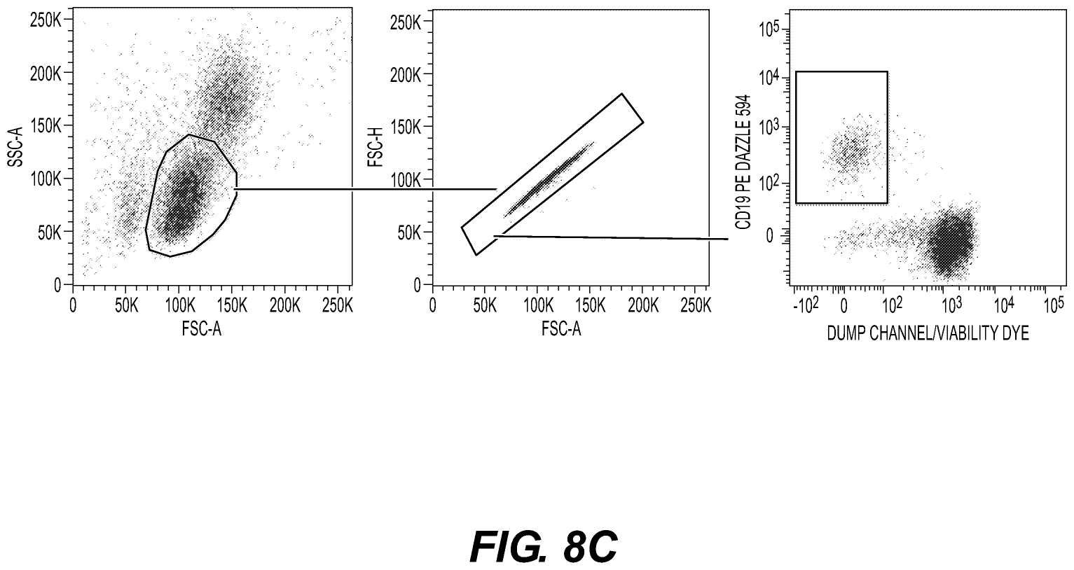

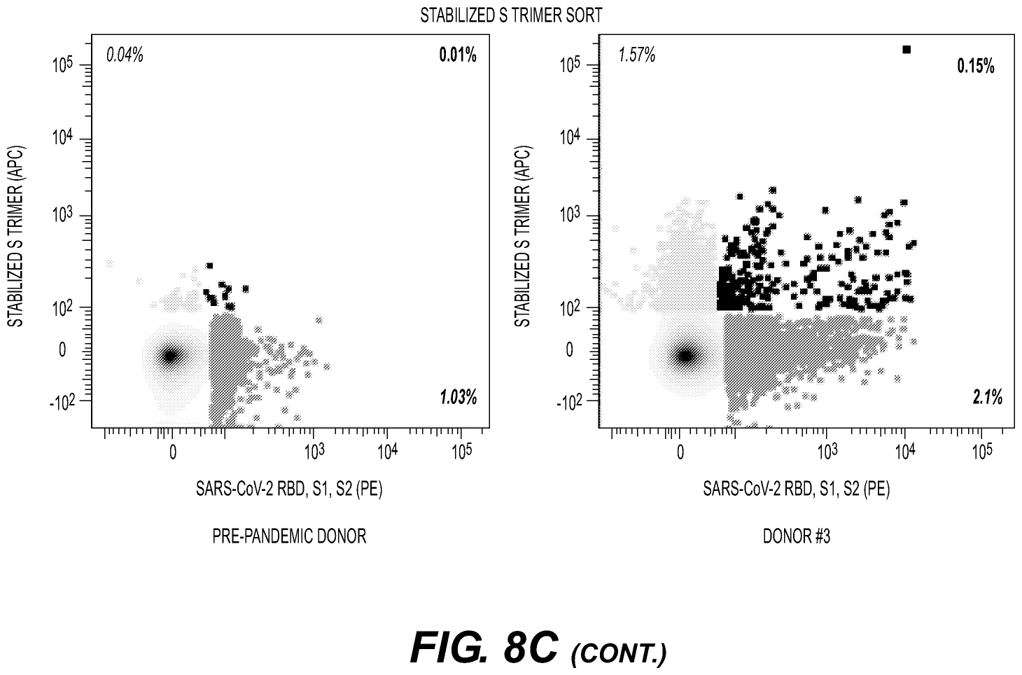

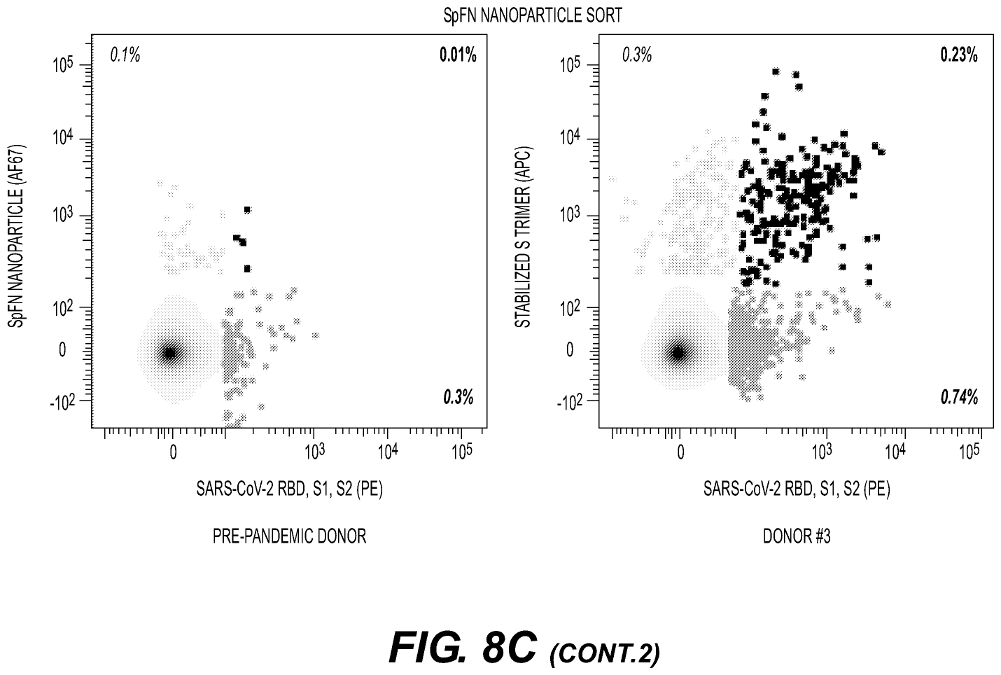

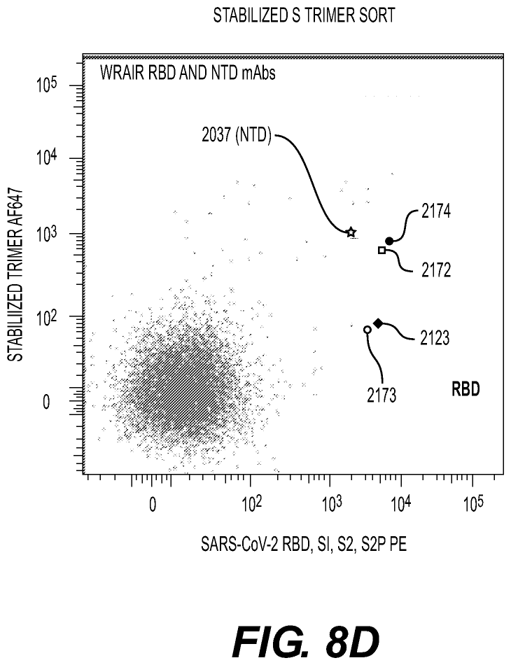

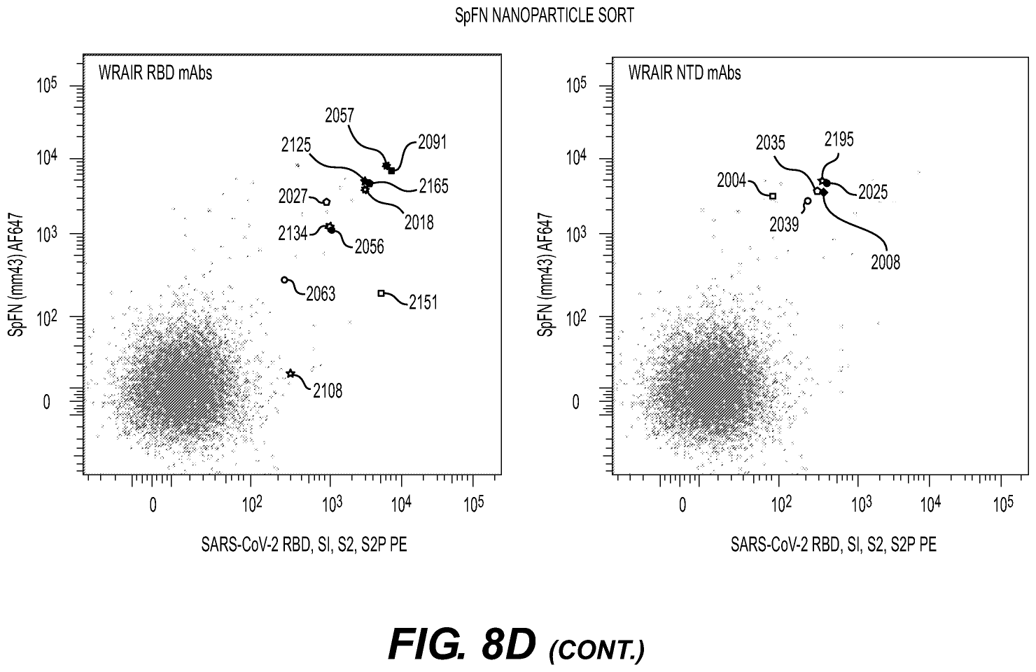

Convalescent plasma of 56 SARS-CoV-2—infected human donors, who had mild to moderate symptoms, were screened for neutralization potency. Among them, donor #3 demonstrated potent neutralization and high antibody binding to NTD, RBD, and the prefusion stabilized S trimer¹¹ (S trimer hereafter) (FIG. 2A). Binding to NTD, RBD, and the S trimer strongly correlated with plasma neutralization of pseudotyped SARS-CoV-2 virions (pSV) (FIG. 8A).

We used peripheral blood mononuclear cells (PBMCs) from donor #3 (FIG. 2A) in two independent sorting strategies to isolate SARS-CoV-2-specific CD19+B cells with a broad range of specificities. The first sorting strategy was based on high plasma neutralization against strain IL1/2020 and high magnitude binding antibodies to N-terminal domain (NTD) on the S1 subunit, the receptor-binding domain (RBD) in the S1 subunit, and Spike (S)-protein trimer (S-trimer) measured in a multiplex bead-based assay. In the second sort, the S trimer was replaced by a multivalent S ferritin nanoparticle (SpFN) displaying eight S trimers (FIG. 8B), a vaccine candidate currently in a Phase 1 clinical trial (NCT04784767)^{18, 19} SpFN was used to mimic the SARS-CoV-2 virus with the desire to isolate mAbs targeting potential conformational or quaternary epitopes. The two sorting strategies revealed complementary profiles in their ability to bind to antigen-specific B cells using flow cytometry, with a high overall frequency of SpFN and S trimer-specific B cells (FIG. 8C). The majority of potent NTD-directed neutralizing mAbs were isolated from the SpFN sort, whereas RBD neutralizing antibodies were obtained from both sorting approaches (FIG. 8D).

Antibody heavy and light chain pairs were recovered from both sorting strategies and sequenced from single-cell SARS-CoV-2 positive B cells. The nucleotide and amino acid sequence identifiers for the variable heavy chain of selected antibodies are shown in Table 1. Table 2 provides the sequence identifiers for the variable light chains. The specific germline genes (heavy V, heavy D, heavy J, light V) are also depicted in Tables 1, 2 and FIG. 2A. Percent sequence identities between the portion of each variable region sequence corresponding to germline heavy V and light V genes are also shown.

Antibodies were produced as human IgG1 in EXPI293™ cells and screened as cell culture supernatants for binding and neutralization. The mAbs were subsequently purified and tested for binding to SARS-CoV-2 subdomains, cross-reactivity to other coronaviruses and for neutralization using a pseudotyped lentivirus (pSV) neutralization assay.

2b. Binding and Cross Reactivity of Antibodies Directed Against SARS-CoV-2

As shown in Table 3, below, the majority of the mAbs bound to the S2 subunit of the SARS-CoV-2 spike protein, which may have been a result of the sorting strategy, followed by RBD and NTD, based on binding antibody assays (FIG. 2B). Eleven (11) of the selected SARS-CoV-2 antibodies bind to the NTD region of SARS-CoV-2 (Table 3). Seventeen (17) of the selected antibodies bind to the RBD region (Table 3). COV_1037 and COV_3053 bind to an epitope spanning both the NTD and RBD regions (Table 3). The remaining antibodies bind to the S2 subunit of the SARS-CoV-2 spike protein (Table 3).

Binding of the instant anti-SARS-CoV-2 antibodies against other coronaviruses including SARS-CoV01, MERS-CoV, HCoV-HKU1 HCoV-NL63 and HCoV-OC43 was also tested as described above in Example 1G. The cross-binding results are shown in Table 4. In addition to SARS-CoV-2, COV_1037, COV_1045, COV_1046, COV_2063, COV_2018, COV_3000, COV_3005, COV_3013, COV_3019, COV_3028, COV_3031, COV_3033, COV_3037, COV_3040 and COV_3043 bound to all of the coronaviruses tested (Table 4). No cross-binding was evident for COV_3053 and COV-3088 (Table 4).

2c. Neutralization and Binding Properties of Anti-NTD and Anti-RBD Antibodies

Neutralization potency of isolated mAbs segregated by subdomain was assessed from the SARS-CoV-2 (IL1/2020) lentivirus-based pseudotyped-virus (pSV) neutralization assay as discussed above. More potent neutralization activity was observed for RBD- and NTD-directed antibodies in comparison to S1 and S2 directed antibodies (FIG. 2C, IC50=50% inhibitory concentration (μg ml⁻¹)). RBD mAbs demonstrated neutralization potency ranging from subnanomolar to micromolar concentrations, whereas NTD mAbs presented a dichotomous profile being either strongly neutralizing or non-neutralizing (FIG. 2C). RBD mAbs revealed a strong correlation between neutralization potency and binding magnitude to the S trimer (FIG. 2D). In contrast, binding to the S trimer did not correlate with neutralization by NTD-targeting mAbs. All NTD neutralizing mAbs displayed intermediate binding to the S trimer, whereas binding responses observed with non-neutralizing NTD mAbs were either high or absent, revealing three distinct binding profiles (FIG. 2D).

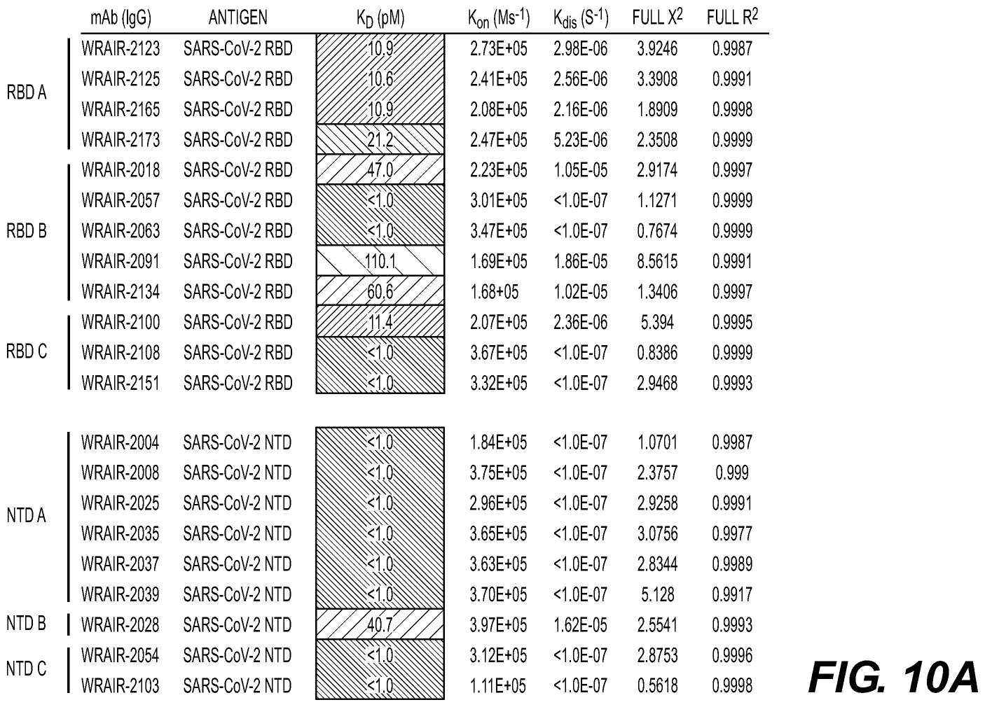

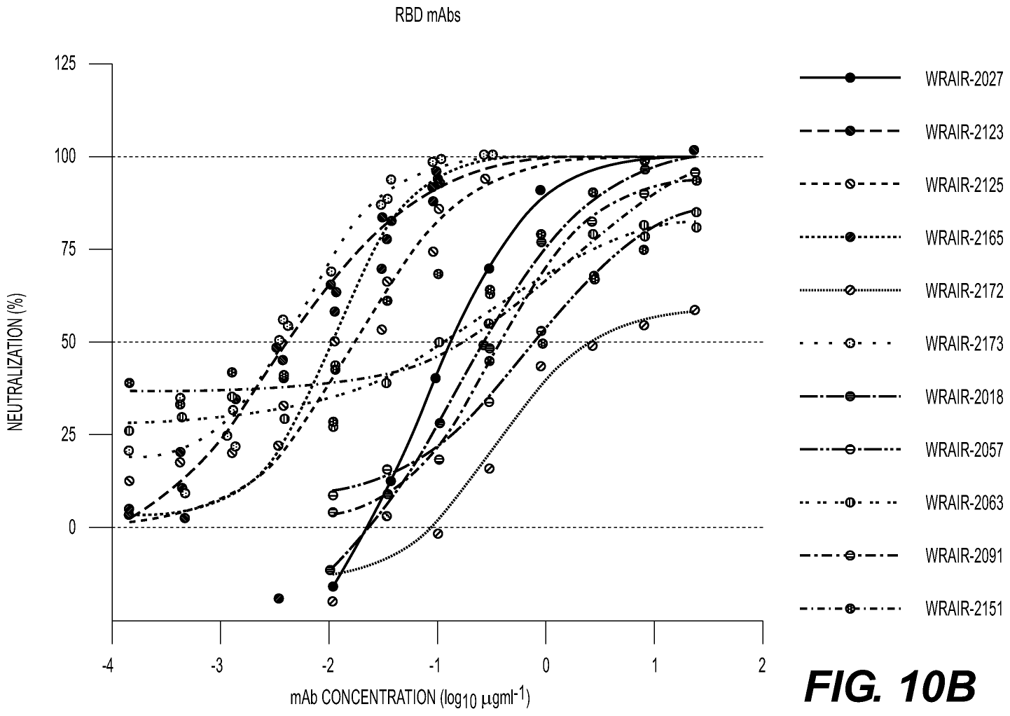

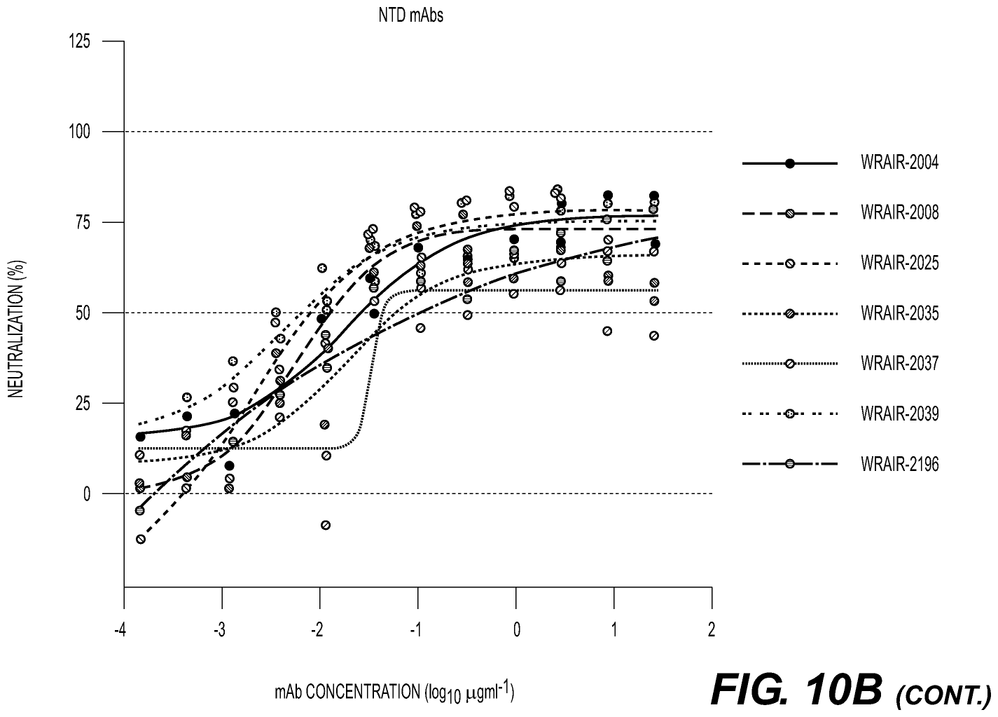

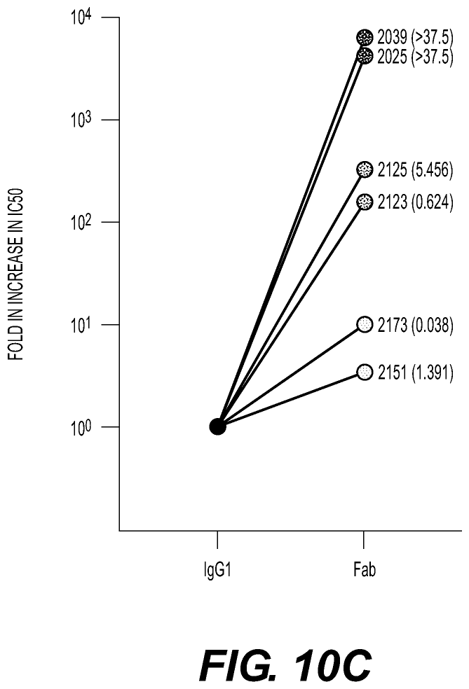

In view of the greater neutralization potency of NTD- and RBD-directed antibodies, we further characterized these antibodies. FIGS. 10B and 10C depicts neutralization curves of anti-NTD and anti-RBD antibodies against the SARS-CoV-2 (IL/2020 strain), which were obtained in the pSV neutralization assay. Three-fold dilutions of antibodies were incubated with HIV-1 virus-like particles pseudotyped with SARS-CoV-2 D18 spike protein and bearing a luciferase reporter gene. The virus-antibody mixtures were tested for their ability to infect HEK293-ACE2 cells by measuring luciferase signal in cell lysates 48 hours post-infection. Mean IC50 and IC80 (ug/mL) values are shown in Table 3. The IC50 values for the tested antibodies ranged from 0-25 μg/ml with antibodies COV_1201, COV_2004, COV_2008, COV-2025, COV_2035, COV_2037, COV_2039, COV_2125, COV_2123, and COV_2173 demonstrating neutralization in the nanomolar range.

Dissociation constants (K_{D}) were determined assessed as described above in Example 1D for selected NTD- and RBD-directed antibodies. As shown in Table 3, two (2) of the seven (7) anti-RBD antibodies tested, COV_2057 and COV_2063, exhibited K_{D} values of <1 pM. As shown in Table 3, Eight (8) of the ten (10) anti-NTD antibodies tested exhibited K_{D} values of <1 pM, i.e., COV_2008, COV_2025, COV_2035, COV_2037, COV_2039, COV_2004, COV_2054 and COV_2103.

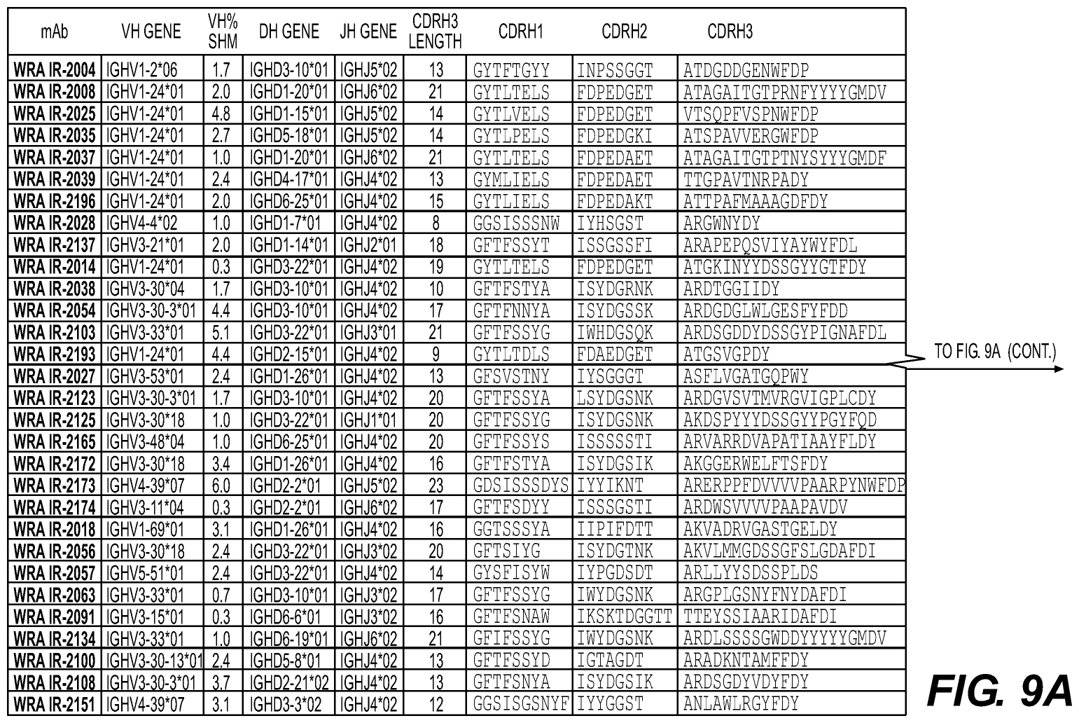

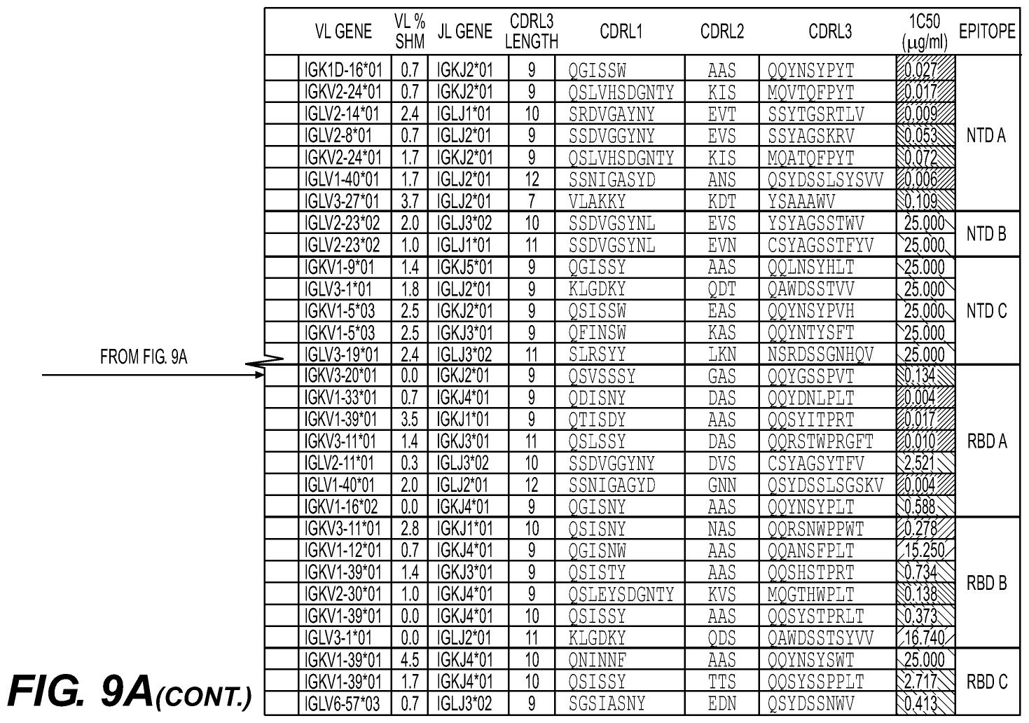

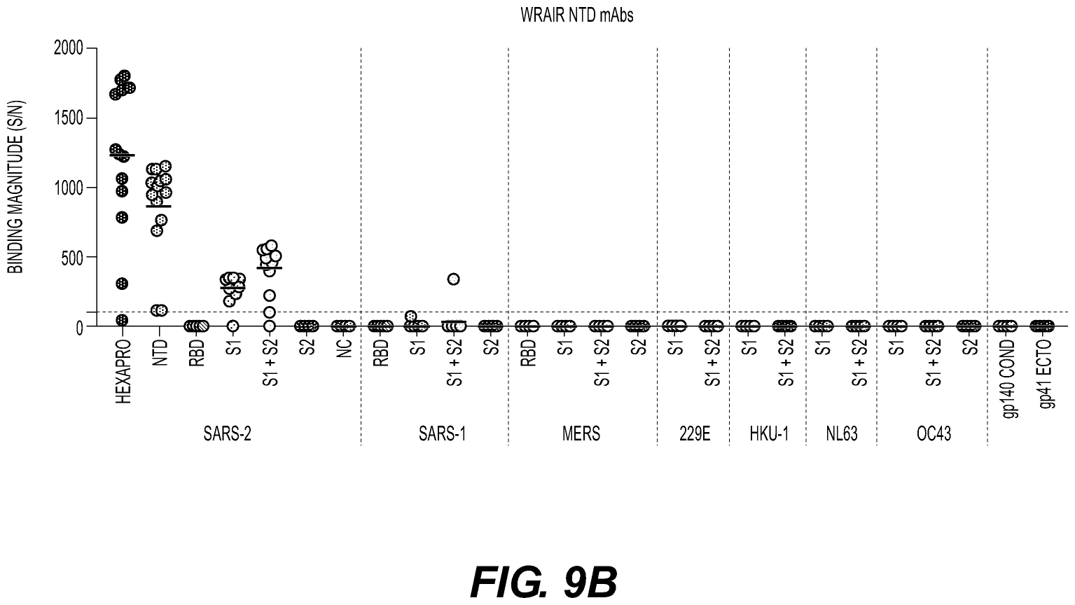

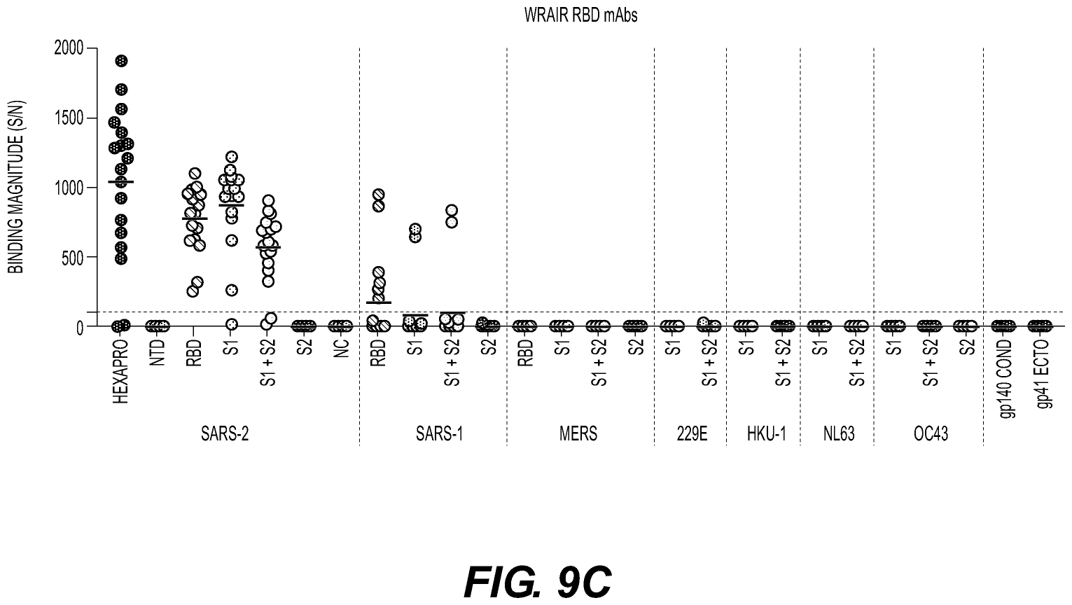

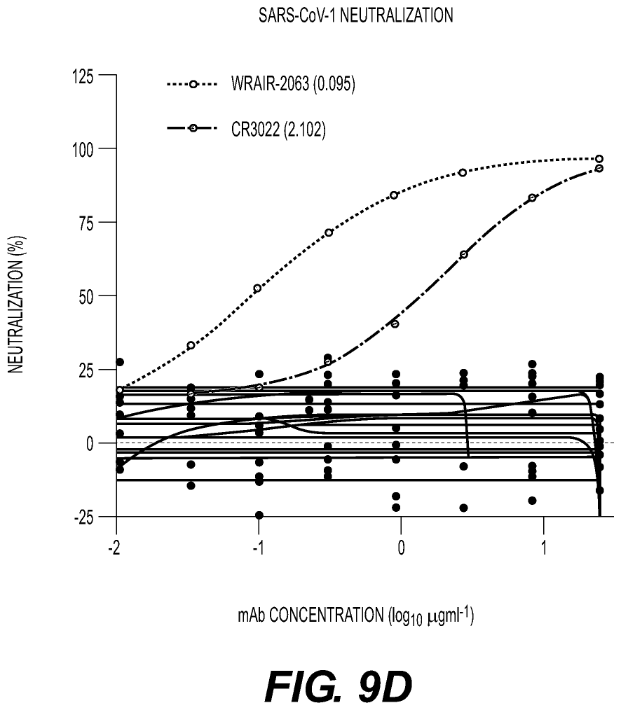

Binding cross-reactivity across human alpha and beta coronaviruses demonstrated that isolated NTD mAbs were SARS-CoV-2 specific, whereas a few RBD mAbs cross-reacted with SARS-CoV-1 (FIG. 9B, C, Table 4). Of these, WRAIR-2063 was able to potently neutralize SARS-CoV-1 with an IC50 of 95 ng ml⁻¹ (FIG. 9d).

2d. Comparison of Neutralization Using Pseudotyped and Authentic SARS-CoV-2 Virus Neutralization Assays

We next compared the neutralization potency of specific anti-NTD and anti-RBD mAbs in pseudotyped virus neutralization assays with authentic SARS-CoV-2 virus neutralization assays using strains IL1/2020 and INMI1/2020, respectively, which share an identical S sequence. NTD mAbs displayed potent neutralization in both assays with the notable difference that neutralization curves reached a plateau around 75% neutralization in the pseudotyped assay, while the same NTD mAbs were able to fully neutralize authentic SARS-CoV-2 (FIGS. 2E, 2F). A significant correlation between results obtained from both assays was observed (FIG. 2G). Per the pseudotyped neutralization assay, all NTD mAbs demonstrated IC50 below 100 ng ml⁻¹, with COV-2039 and COV-2025 being the most potent at 6 and 9 ng ml⁻¹, respectively (FIG. 2E, F, FIG. 10B and Table 3). RBD-directed antibodies displayed typical sigmoidal curves in both assays and a much wider range of IC50s spanning over several orders of magnitude (FIG. 2E,2F, FIG. 10B). MAbs COV-2173 and COV-2123 were the most potent with identical IC50 values of 4 ng ml⁻¹, followed by WRAIR-2165 (10 ng ml⁻¹) and WRAIR-2125 (17 ng ml⁻¹). When tested as Fabs, COV NTD mAbs no longer neutralized the pseudotyped virus, suggesting that bivalent binding and/or the presence of the Fc domain in the IgG1 format is important for pSV neutralization (FIG. 10C). Fab versions of COV RBD mAbs such as COV-2173 and COV-2151 retained most of their potency but others, like COV-2123 and COV-2125, showed markedly reduced activity by 2- or 3-order of magnitude, likely reflecting differences in their mechanism of action (FIG. 10C).

2e Fc Effector Function

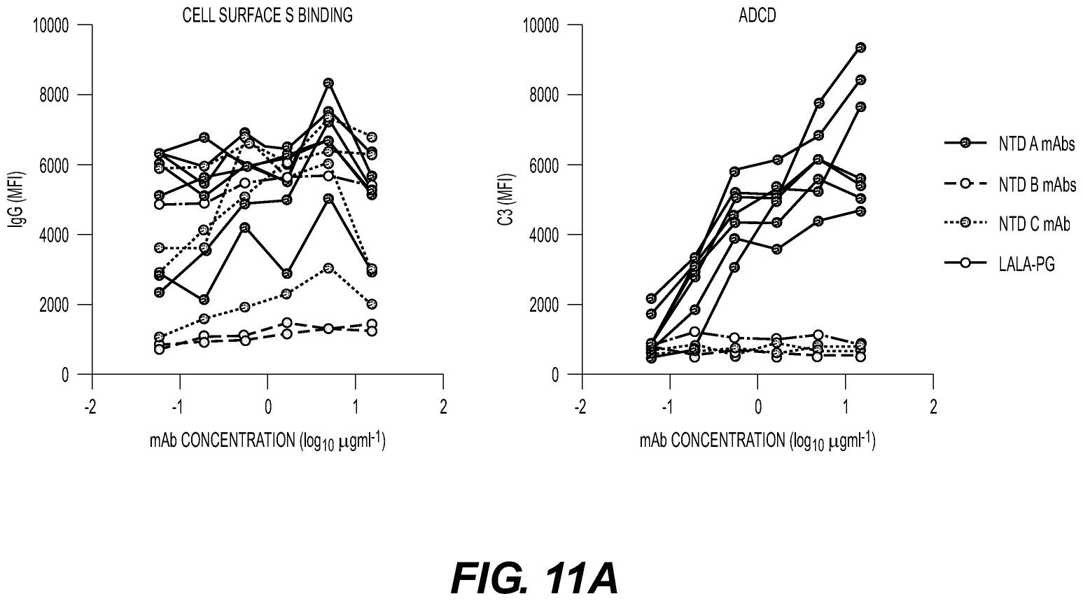

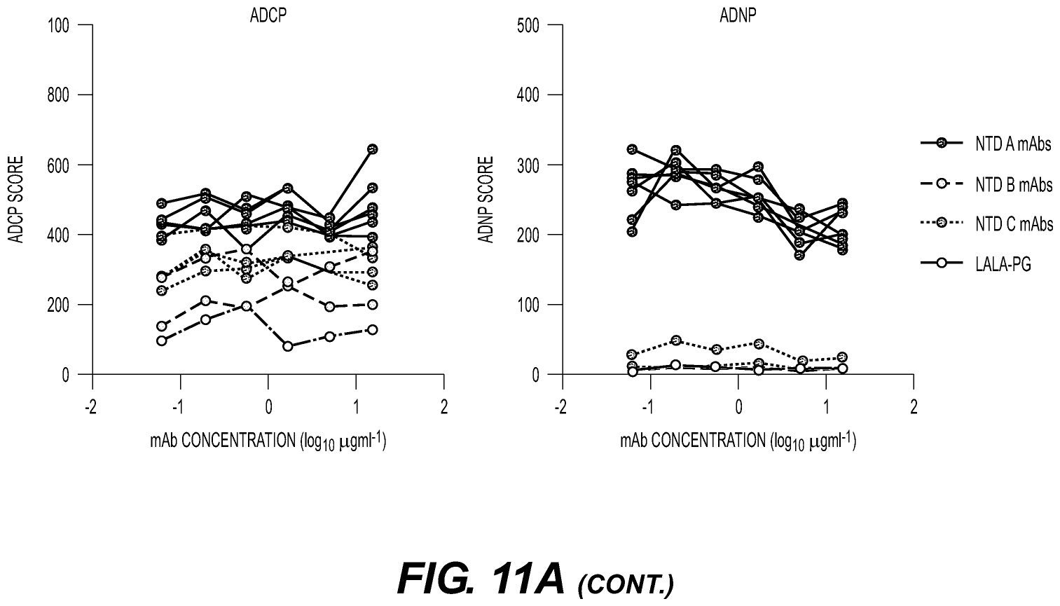

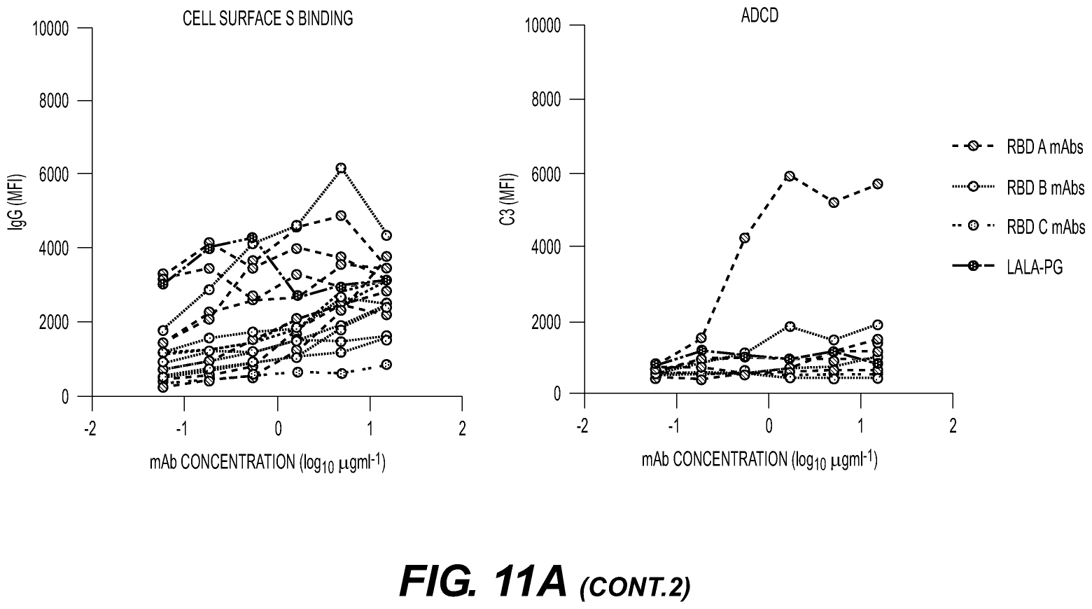

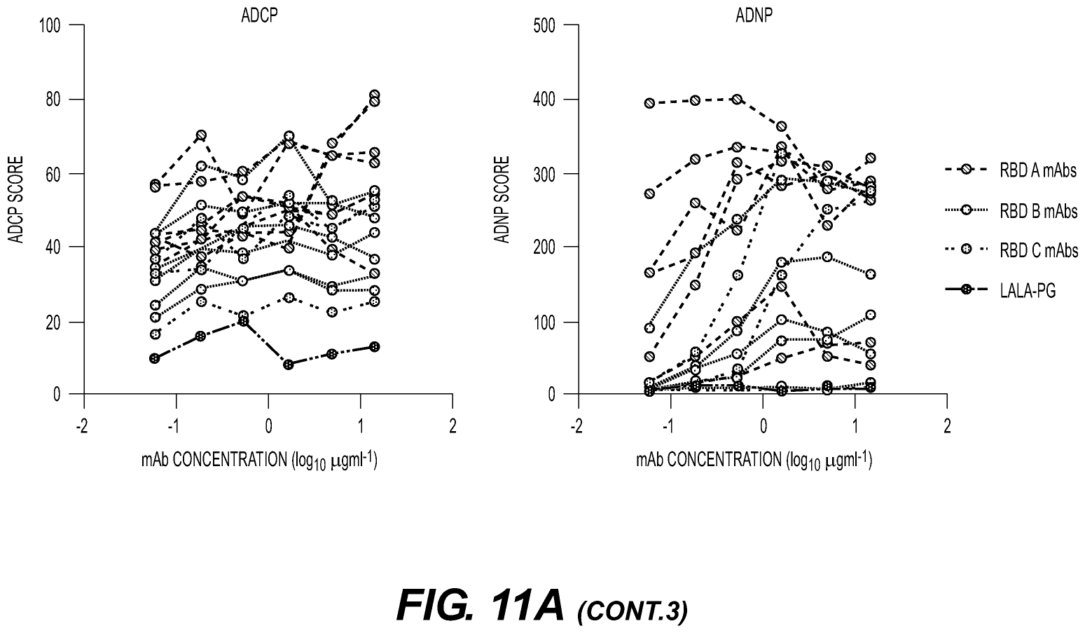

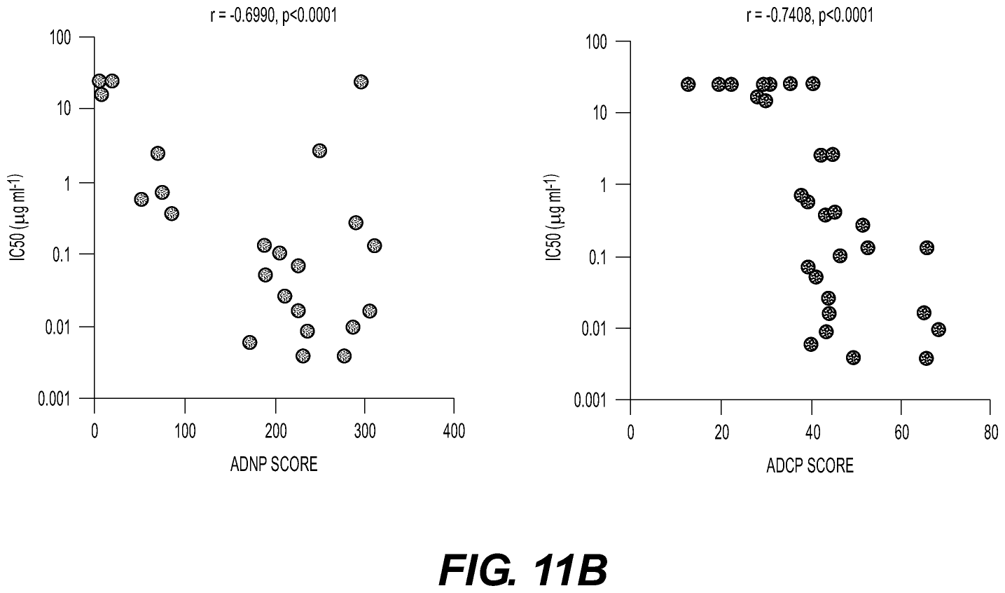

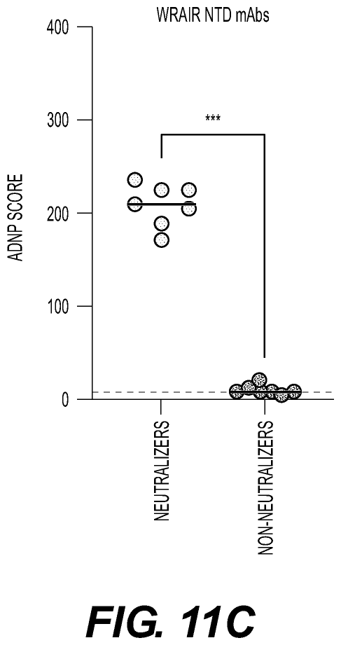

In addition to neutralization activity, Fc effector functions have also been shown to play a role in protection against SARS-CoV-2 in vivo. Therefore, we investigated the ability of our NTD and RBD mAbs, all expressed as IgG1, to promote Fc effector functions (FIG. 11A). We first observed that COV NTD mAbs, inclusive of non-neutralizing mAbs, were significantly better than COV RBD mAbs at mediating opsonization of cells expressing S at their surface (FIG. 2H), a prerequisite for any Fc-effector activities against virus-infected cells. Among NTD mAbs, binding to cell surface Spike was associated with complement recruitment for the neutralizing mAbs only, indicating that non-neutralizing NTD epitopes may not be compatible with Antibody Dependent Complement Deposition, (ADCD) (FIG. 2I). Only one RBD mAb (COV-2165) was able to recruit complement to the level of NTD mAbs and as such, neutralizing NTD mAbs displayed significantly higher ADCD activity than RBD neutralizing mAbs (FIG. 2I). When looking at phagocytic activities with monocytes (Antibody Dependent Cellular Phagocytosis, ADCP) and neutrophils (Antibody Dependent Neutrophil Phagocytosis, ADNP) using a S-expressing 293F cell line whereas phagocytic activities were determined using the stabilized S trimer, COV NTD and RBD mAbs performed equally well, with higher scores significantly correlating with neutralization activity (FIG. 2J and FIG. 111B). However, neutralizing NTD mAbs were significantly better at mediating ADNP compared to non-neutralizing mAbs (FIG. 11C). Taken together, we identified potent neutralizing antibodies directed to the NTD and RBD domains of the Spike on SARS-CoV-2 that are able to mediate Fc effector functions, with the former also demonstrating a unique and strong ability to promote complement deposition.

### Example 3. Epitope Characterization NTD-Targeting mAbs

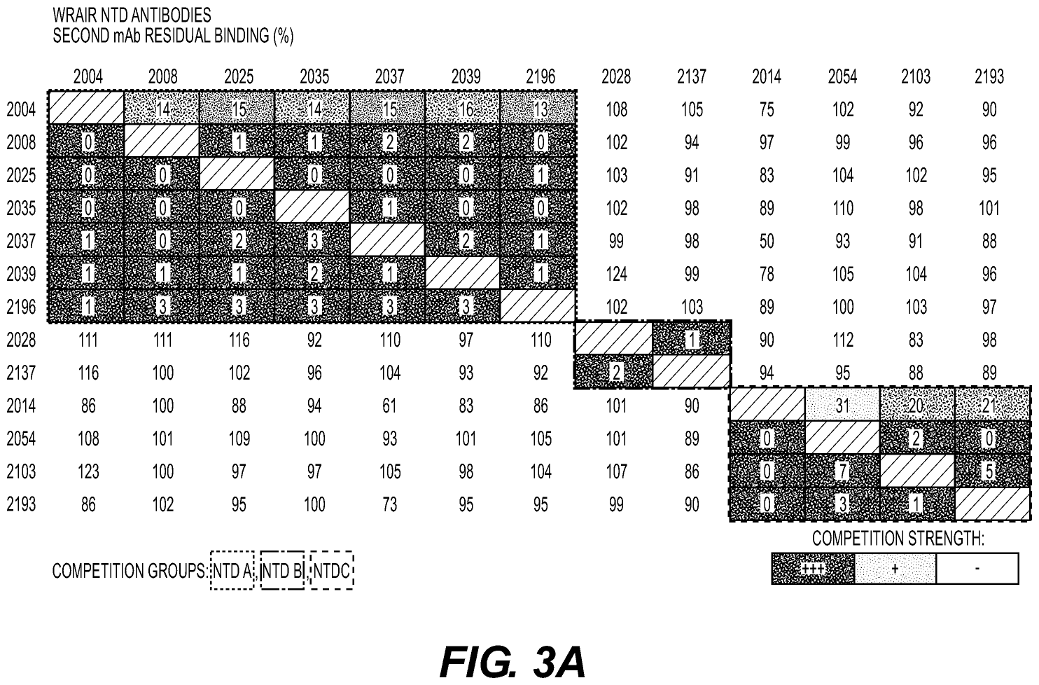

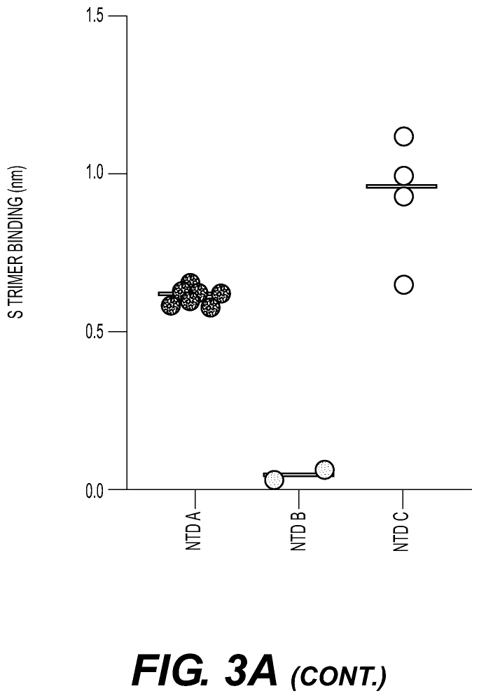

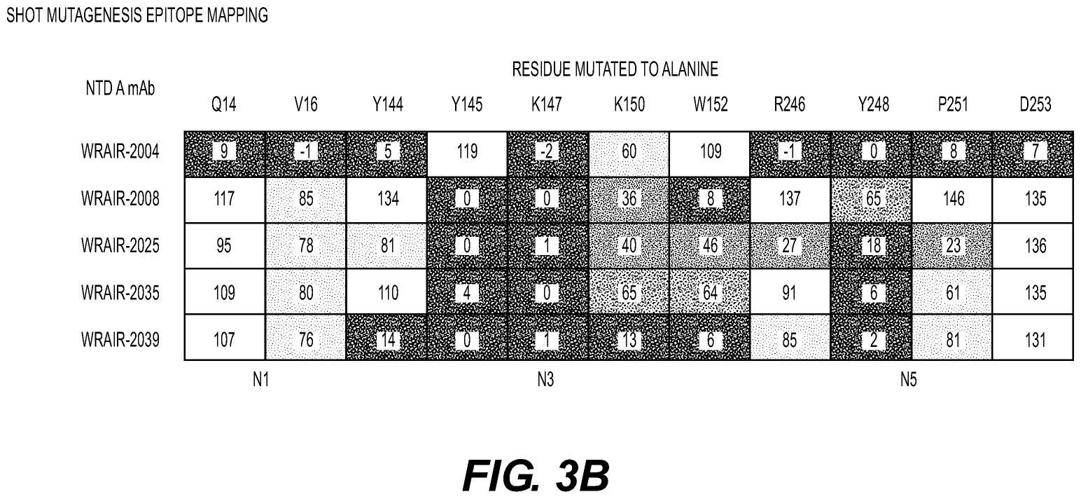

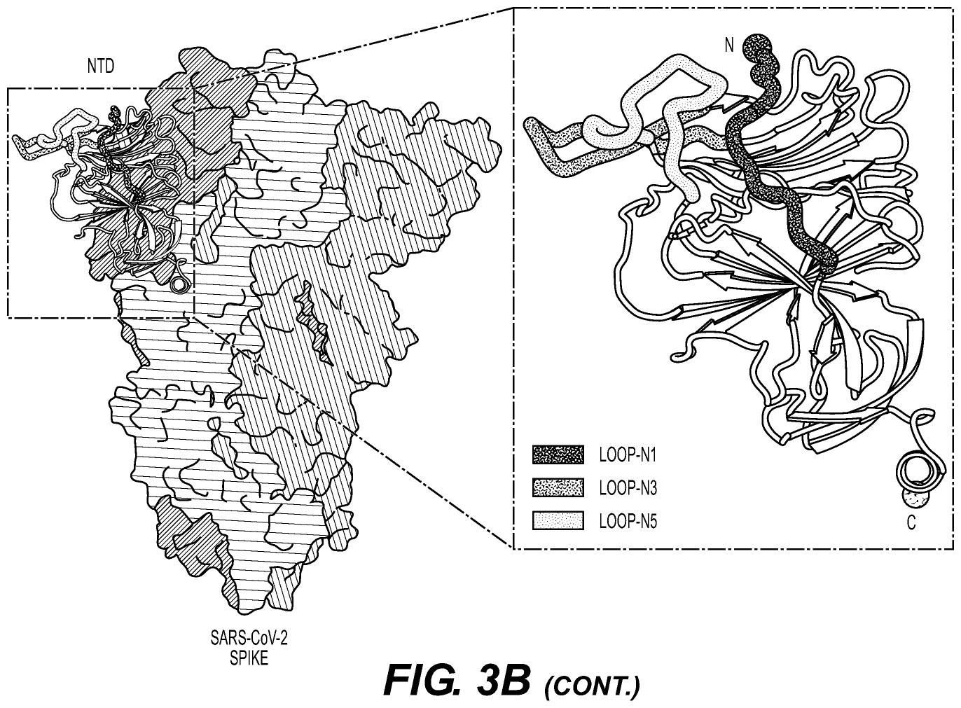

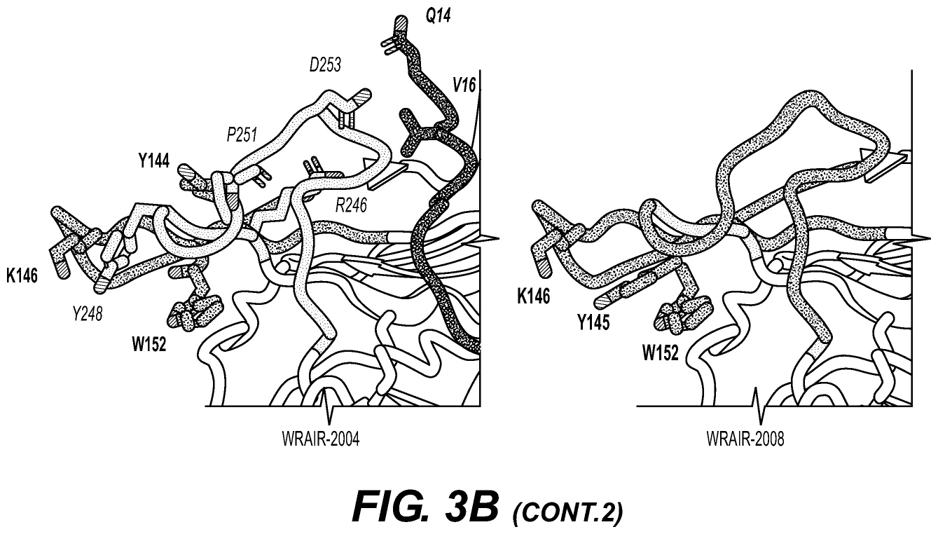

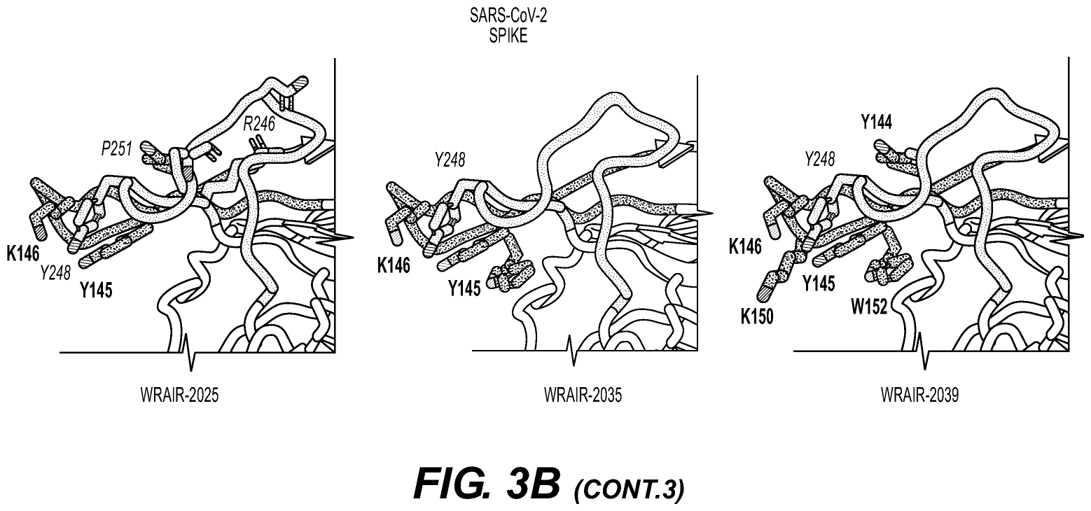

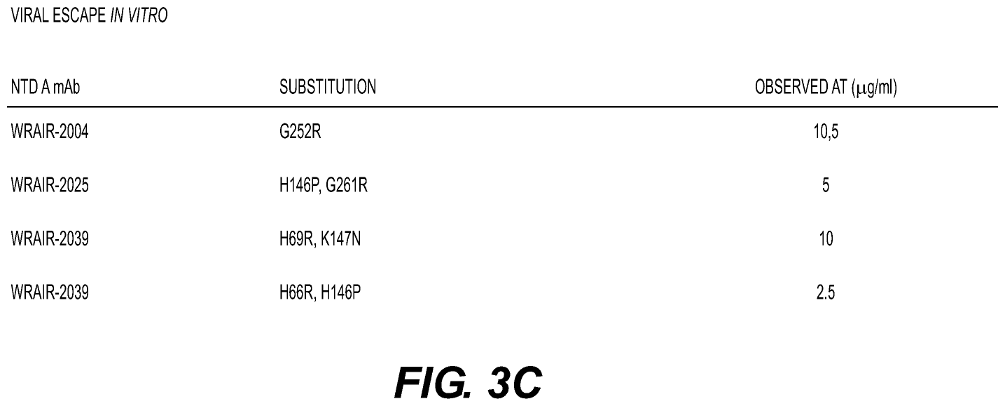

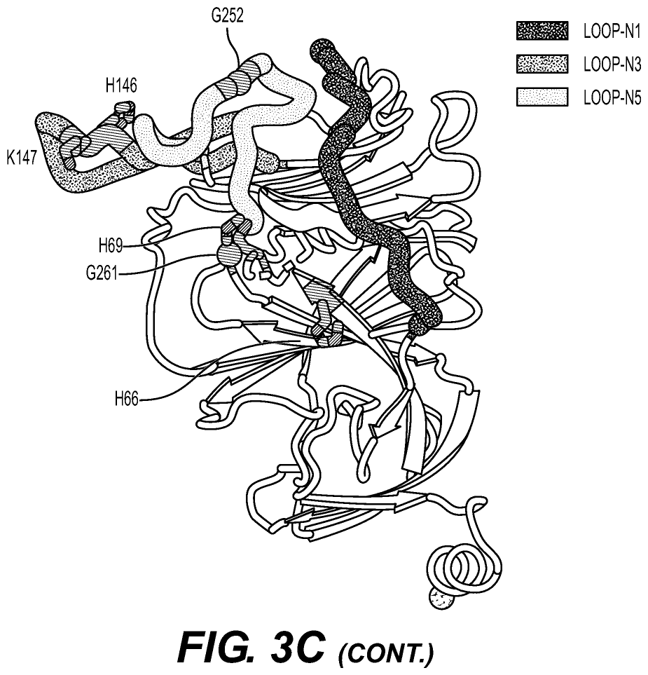

We next used a biolayer interferometry (BLI) competition binding assay as a first step to delineate the antigenic sites targeted by these mAbs (FIG. 3A). WRAIR NTD mAbs fell into three distinct groups; all neutralizing antibodies clustered into one group (NTD A), while non-neutralizing antibodies clustered into two groups (NTD B and C) that differed by their ability to bind the S trimer. While NTD C mAbs bound strongly to the S trimer, NTD B mAbs only interacted with the isolated NTD domain, likely recognizing a cryptic epitope hidden in the ‘closed’ prefusion S trimer (FIG. 3A). Notably, many NTD A neutralizing antibodies used heavy chain IGHV1-24 (FIG. 9A), similar to previous mAbs isolated in several convalescent donors^{8,20,26,27}—such as 4A8²⁶, 1-87⁸ and CM25²⁷. Secondly, to further characterize the epitopes targeted by the NTD neutralizing antibodies, we mapped epitopes using a shotgun mutagenesis platform, which measures loss of binding. Despite variations in their antibody complementarity determining region (CDR) H3 lengths and sequences (FIG. 9A), binding of the V_{H}1-24-derived NTD neutralizing mAbs was affected by mutations in the N3 (Y145, K147) and/or N5 (R246, Y248) loops within the previously characterized NTD antigenic supersite^{7,8}. The epitope of NTD mAb WRAIR-2004 (VH1-2 gene) was more extensive with the inclusion of residues in N1 (Q14, V16), in addition to residues in N3 (Y144, K147) and N5 (R246, Y248, P251 and D253) (FIG. 3b). These results were further confirmed by growing a recombinant VSV, encoding SARS-CoV-2 S, in vitro in the presence of NTD neutralizing antibodies (FIG. 3C). All selected viral variants had substitutions in N3 and/or N5 loops at the same position or in the vicinity of the residues identified by the shotgun mutagenesis approach (FIG. 3C). Overall, we identified 3 non-competing groups of NTD-directed antibodies, with NTD A mAbs demonstrating high affinity and neutralization potency.

### Example 4. Structural Determination of RBD-Targeting Antibodies

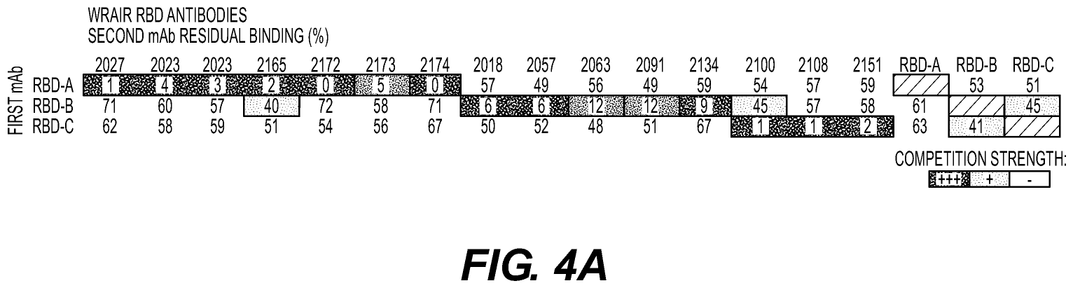

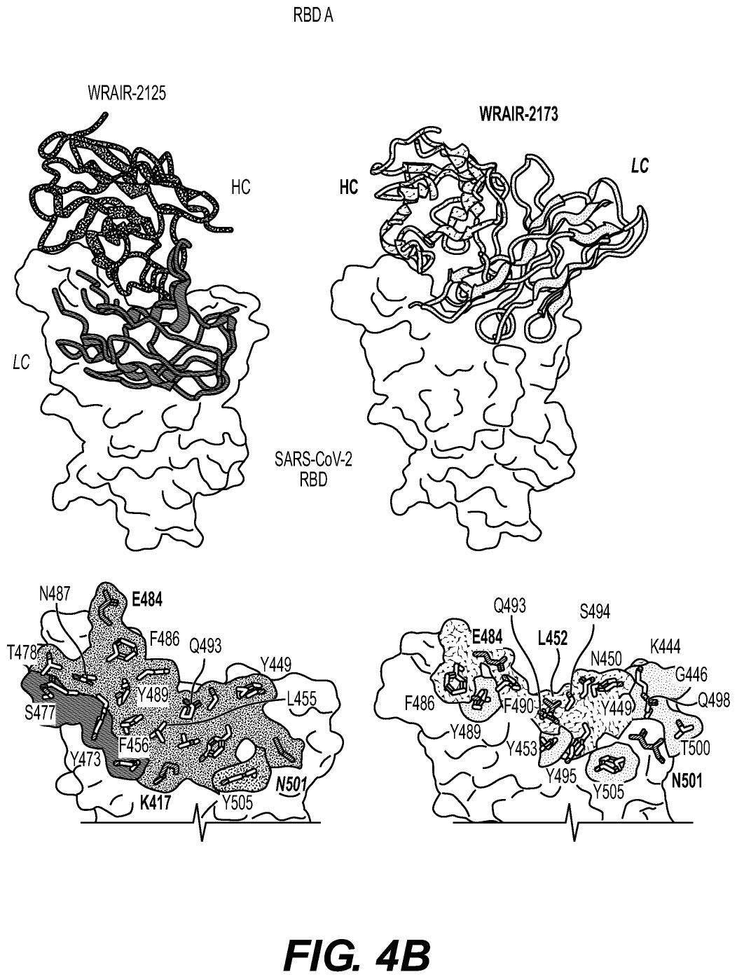

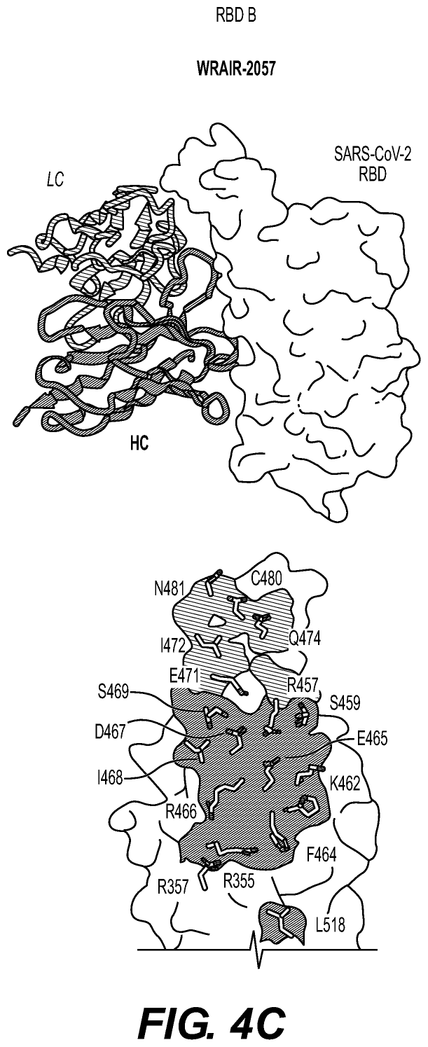

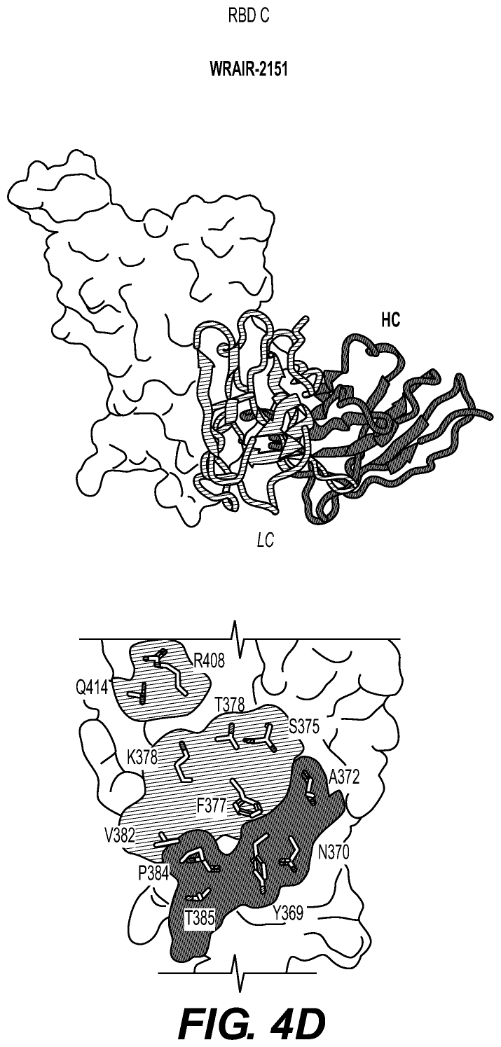

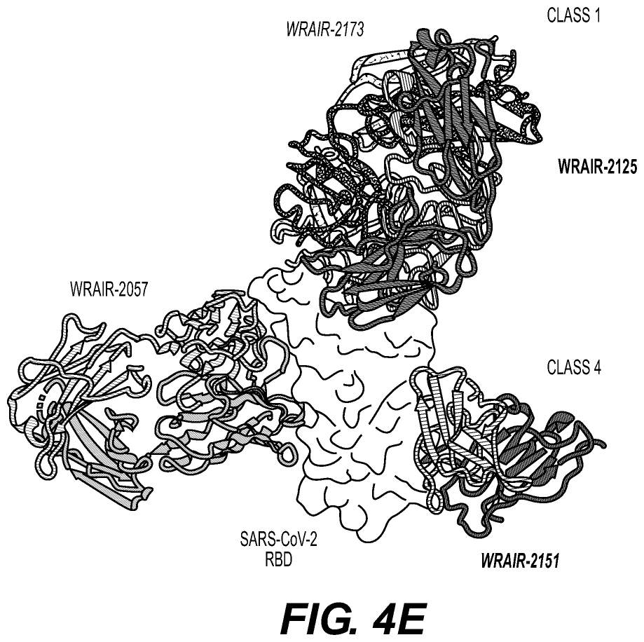

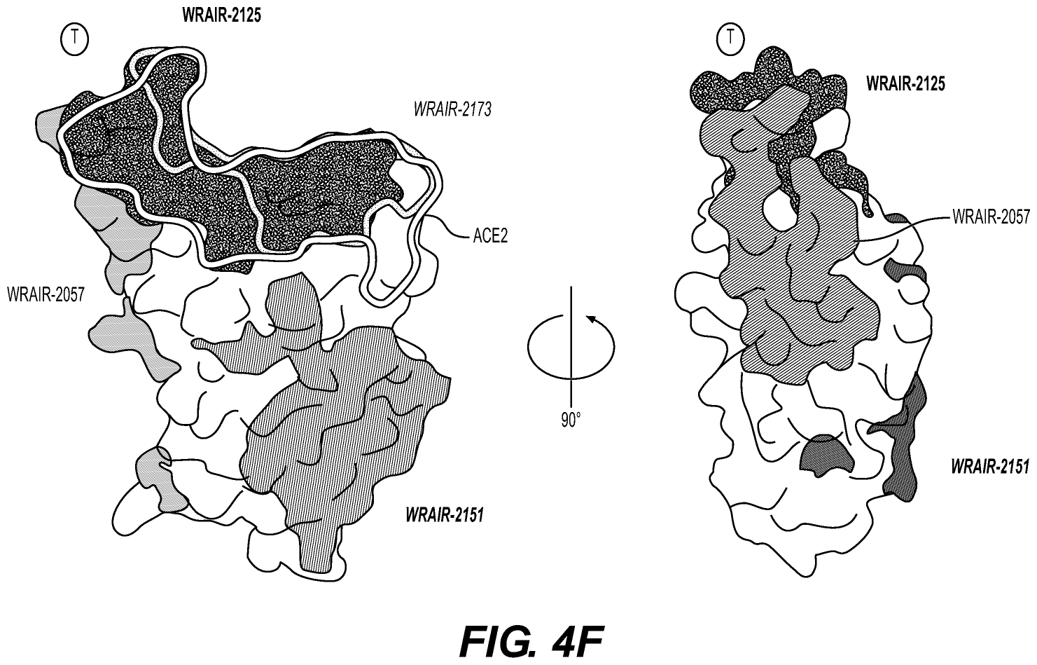

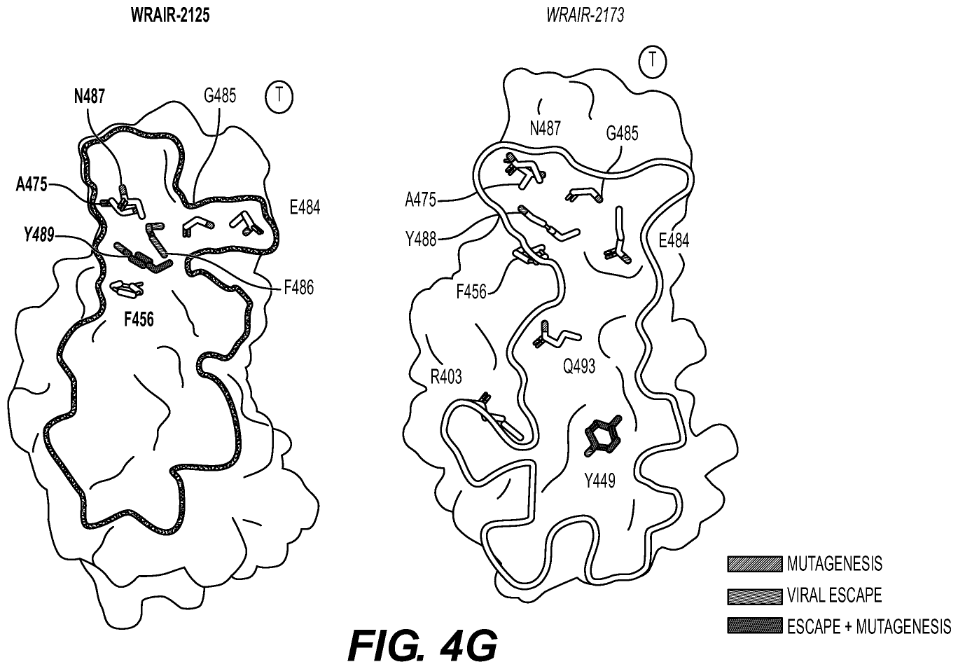

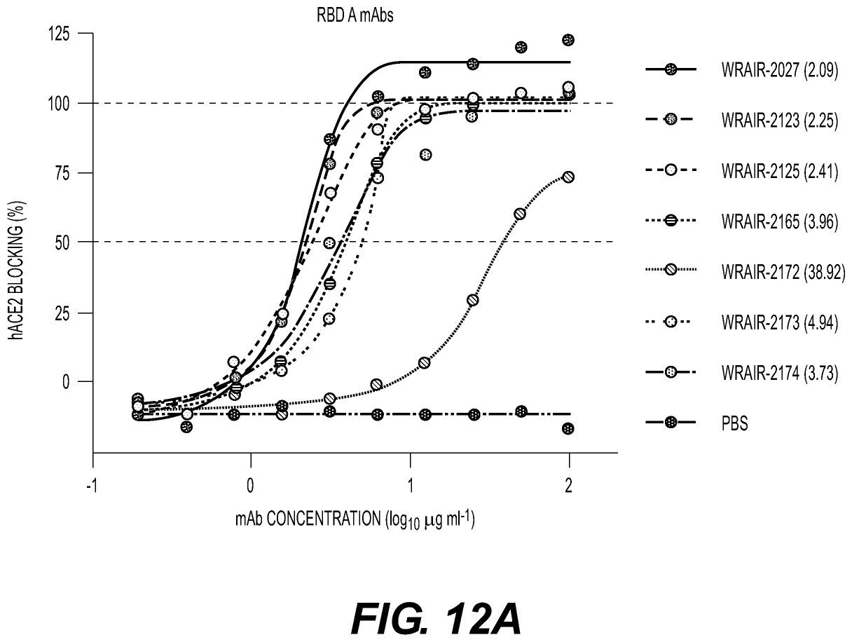

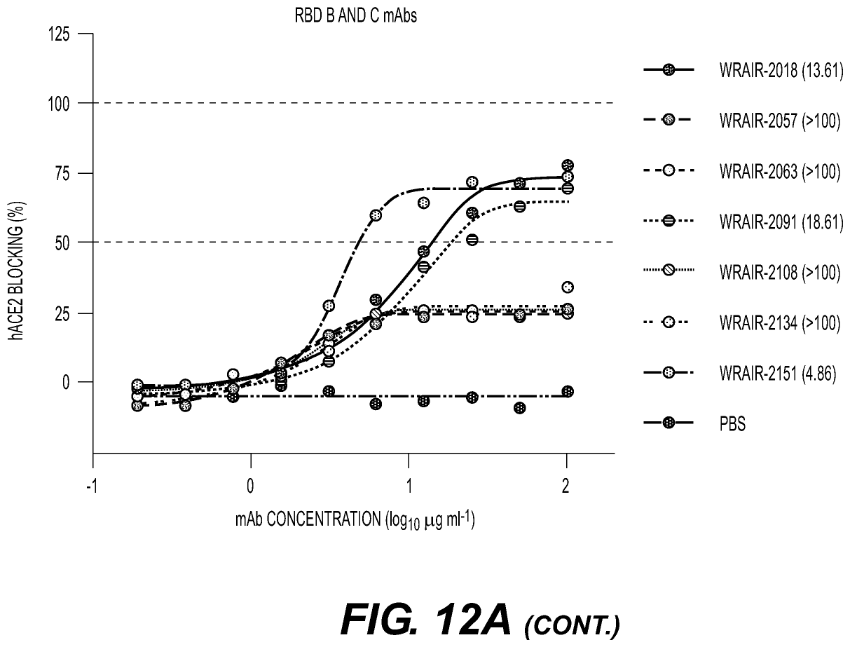

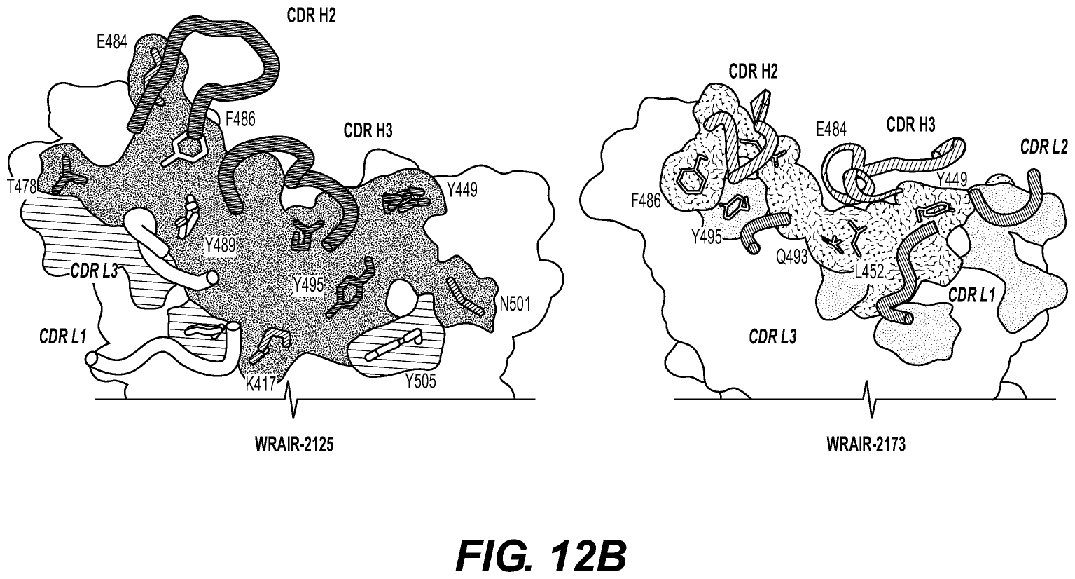

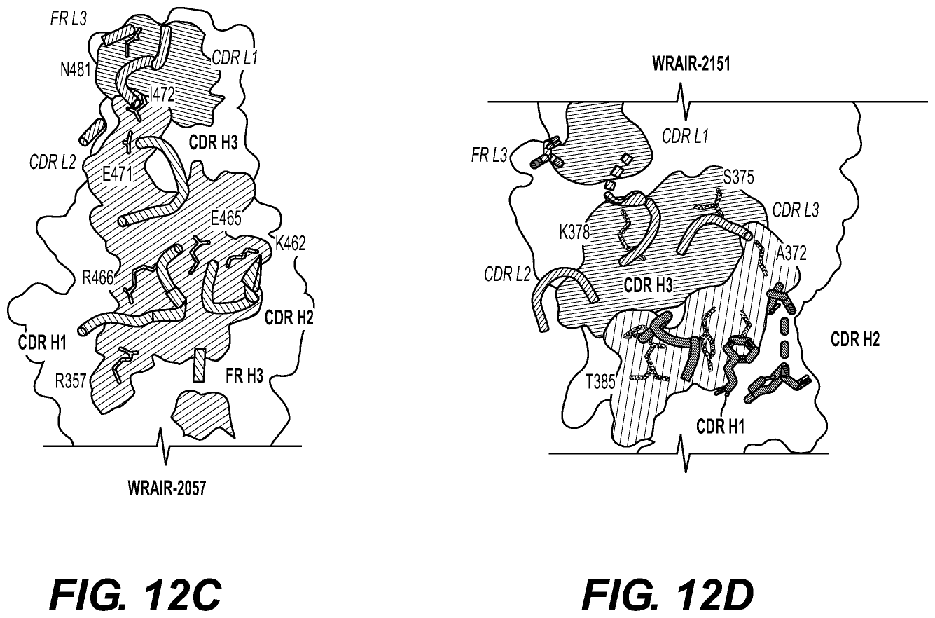

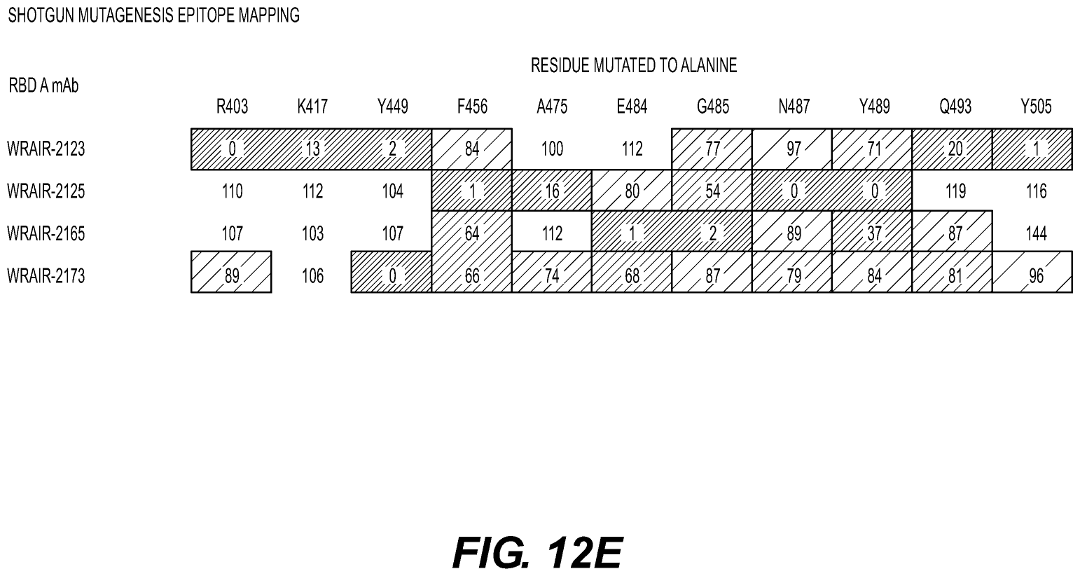

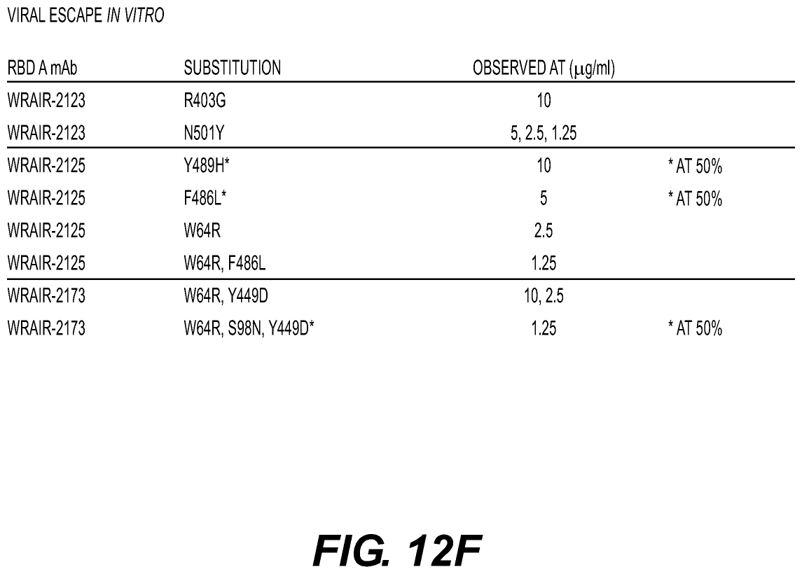

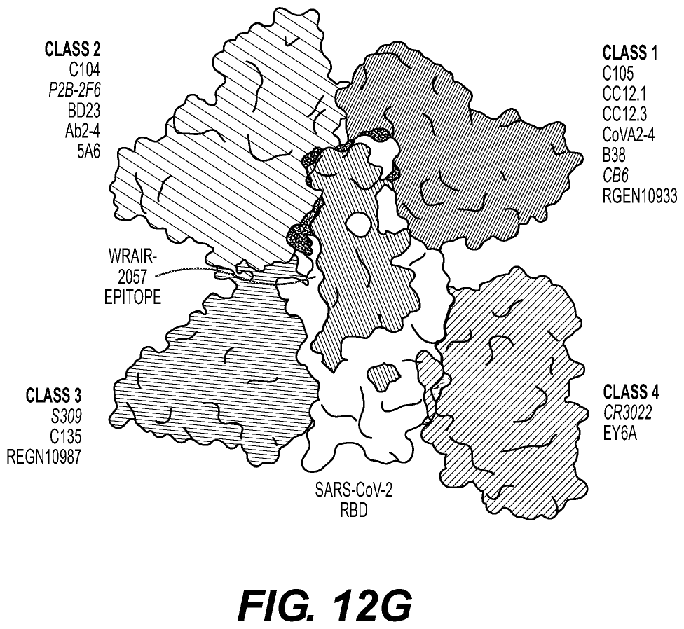

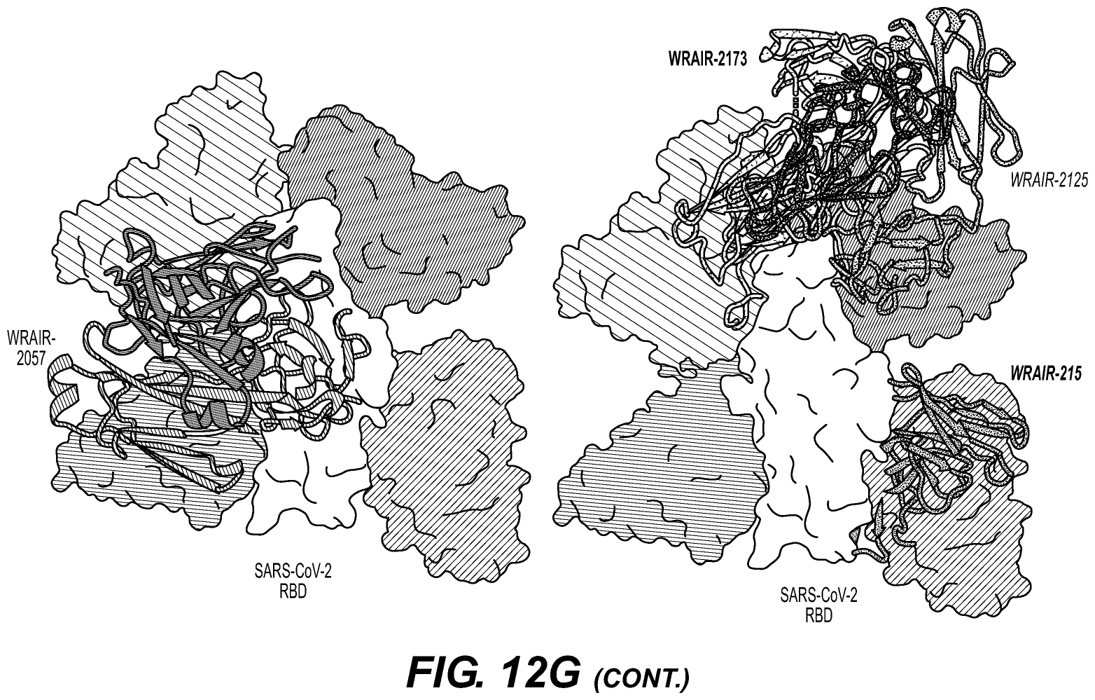

To gain insights into the epitopes targeted by the RBD neutralizing mAbs, we conducted similar binding antibody competitions as described above. Based on their competition with previously described mAbs CC12.1, CC12.16 and CR3022^{13,28} WRAIR RBD neutralizing mAbs segregated into 3 distinct groups: RBD-A, B, and C, respectively (FIG. 4A). The most potent neutralizing mAbs belonged to the RBD-A group, which encompassed previously defined RBD mAb classes 1 and 2 that compete strongly with ACE2²⁹ (FIG. 12A). To understand the structural basis of RBD recognition, crystal structures of representative group A, B and C mAbs in complex with RBD were determined (FIG. 4B-E, FIG. 12B-D). Crystal structures of group A potent neutralizing antibodies WRAIR-2125 and -2173 in complex with SARS-CoV-2 RBD were analyzed to a final resolution of 3.77 Å and 2.2 Å, respectively. Both group A mAbs target the ACE2 binding site with overlapping, but distinct epitopes (FIG. 4B,E,Fand FIG. 12B).

WRAIR-2173 forms extensive interactions across the entire length of the hACE2 receptor binding region whereas WRAIR-2125 is focused to one side and engages fewer RBD residues (FIG. 4B and FIG. 12B). The WRAIR-2125 epitope buries greater than 890 Å² of surface area with heavy and light chains contributing 65% and 35% of total buried surface area (BSA), respectively and is primarily based on CDR H2-3 and CDR L1 and L3 interactions. This includes antibody hydrophobic CDR H2-3 residues V50, Y58, Y99, P100G and CDR L1-3 residues Y32, Y92 and 193, which stack against a hydrophobic patch of the RBD ACE2 binding site (L455, F456, Y473, F486 and Y489).

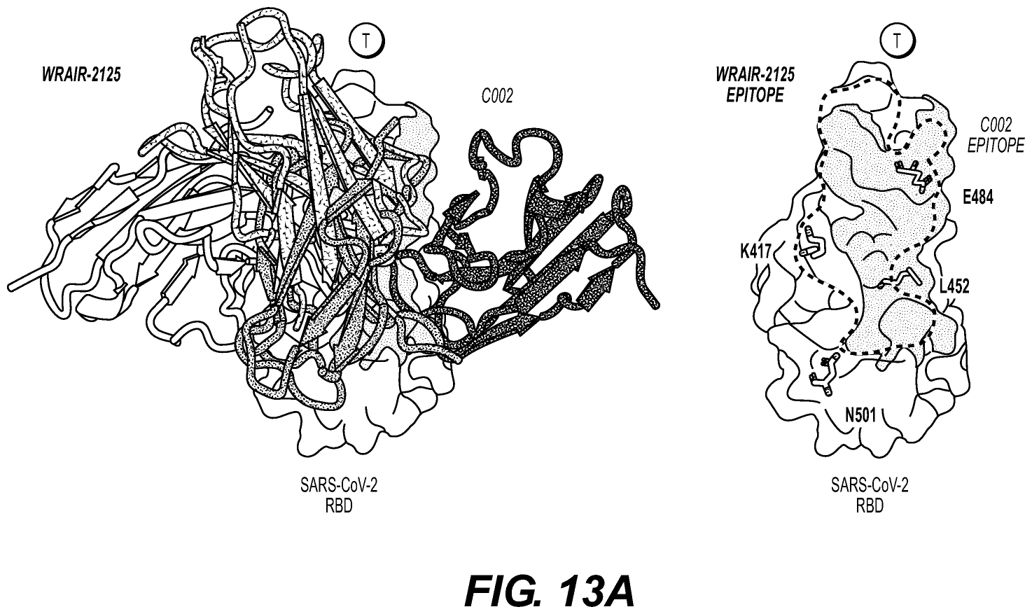

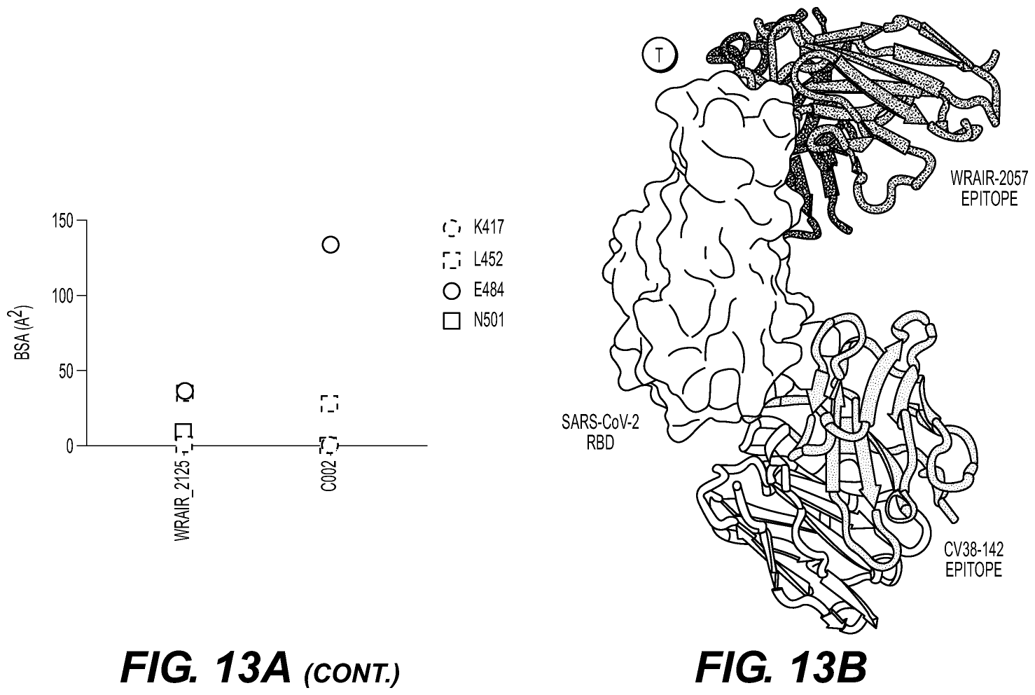

WRAIR-2173 mAb epitope is >900 Å² with heavy and light chains contributing ˜65% and 35% of total BSA, respectively. WRAIR-2173 recognition of SARS-CoV-2 RBD is also based primarily on CDR H2-3 and CDR L1-3 (FIG. 4B and FIG. 12B). The CDR H2 and H3 loops cover about 200 Å² and more than 400 Å² of the RBD interface, respectively. CDR H2 residues K55, N56, T57, and Y58 interact with RBD residues 483-486 while CDR H3 recognition involves extensive hydrophobic contacts using CDR H3 residues P98-Y100J to interact with RBD residues K444, Y449, N450, L452, and Q493-Y495. Both WRAIR-2125 and -2173 form strong interactions with RBD F486 overlapping with RBD-hACE2 contacts (FIG. 4B,F,G). Shotgun mutagenesis-based epitope mapping experiments confirmed the hACE2 binding site as the target for RBD A antibodies and identified F486, N487 and Y489 as relevant residues of the WRAIR-2125 epitope, while WRAIR-2173 binding was only moderately affected by mutations at these sites (FIG. 4F and FIG. 12E). Viral escape experiments also identified F486L and Y489H as escape mutations for WRAIR-2125 and Y449D for WRAIR-2173, each in agreement with the structural and epitope mapping data (FIG. 4F and FIG. 12F). Based on the structural superimposition with representative antibodies from previously defined classes WRAIR-2125 and WRAIR-2173 are grouped into Class-1 type mAbs (FIG. 4G). While WRAIR-2125 shares heavy and light chain germline genes with a previously reported mAb, C002^{14,29}, both mAbs have dissimilar CDR H3 sequences and target different epitopes on the RBD (FIG. 13A).

Representative group B mAb WRAIR-2057 binds to a unique epitope located on the “side” of the RBD molecule, distal from the ACE2 binding site (FIG. 4C, E, F and FIGS. 12C and 12G). Antibodies that target the RBD-B epitope have been seen in other convalescent donor samples^{13,30}, but to our knowledge, this is the first high-resolution structure reported. The epitope covers BSA of 855 Å² with heavy and light chains contributing 72.5% and 27.5% of total BSA, respectively. WRAIR-2057 recognition of SARS-CoV-2 RBD is primarily based on CDR H1-3 and CDR L1 (FIG. 4C and FIG. 12C. Heavy chain interactions form a total of 6 hydrogen bonds and 3 salt-bridges with the RBD along with a set of CDR H1 and H3 hydrophobic residues involved in major contacts, while light chain contacts are primarily mediated by CDR L1 and L2. WRAIR-2057 shares heavy (IGVH5-51) and light (IGKV1-39) chain germline gene usage with SARS-CoV-2 mAb CV38-142³¹. However, these antibodies have distinct non-overlapping epitopes (FIG. 13A,C).

Representative group C mAb WRAIR-2151 binds to the previously defined CR3022 epitope on the RBD^{28,32} (FIG. 4D-F, FIG. 12G), burying more than 670 Å² with heavy and light chains contributing 37.5% and 62.5% of the total BSA, respectively. WRAIR-2151 recognition of SARS-CoV-2 RBD is primarily based on CDR H2-3 and CDR L1-3 (FIG. 4D, FIG. 12D. Overall contacts are mediated by both hydrophobic and hydrophilic residues (FIG. 4D, FIG. 13A). In summary, we determined the molecular determinants of four RBD-directed neutralizing antibodies belonging to three different classes each with distinct features that bind to SARS-CoV-2.

### Example 5. Efficacy of NTD- and RBD-Directed Antibodies In Vivo

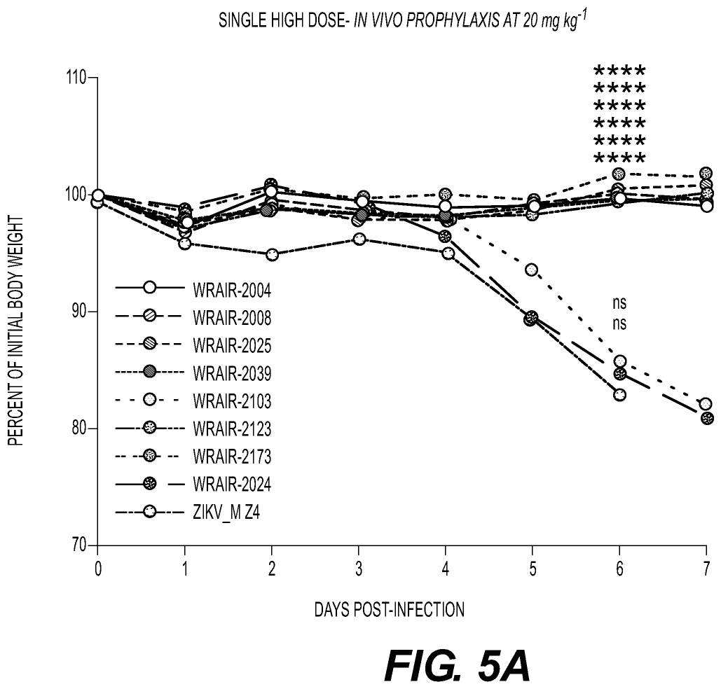

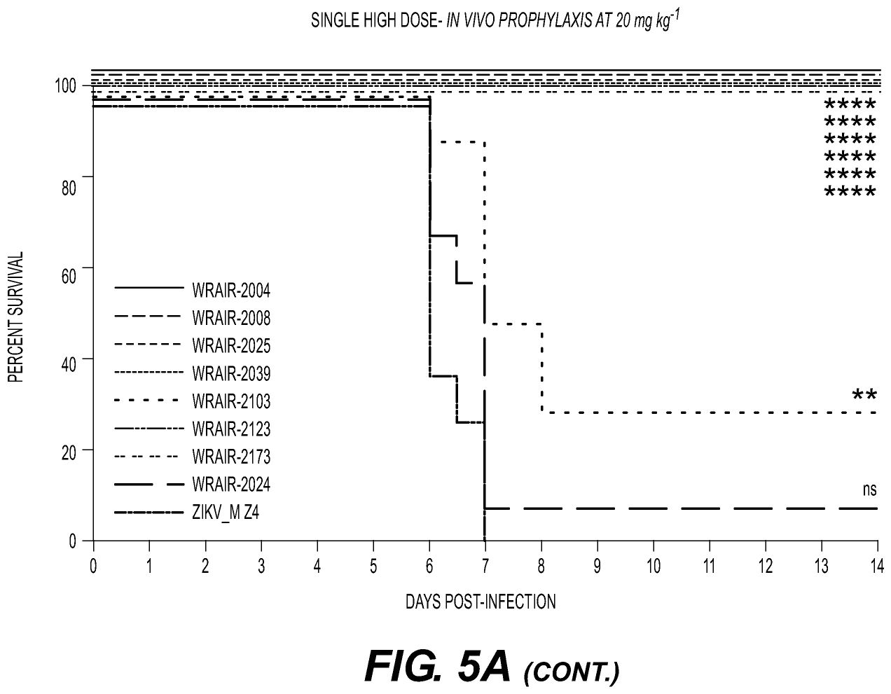

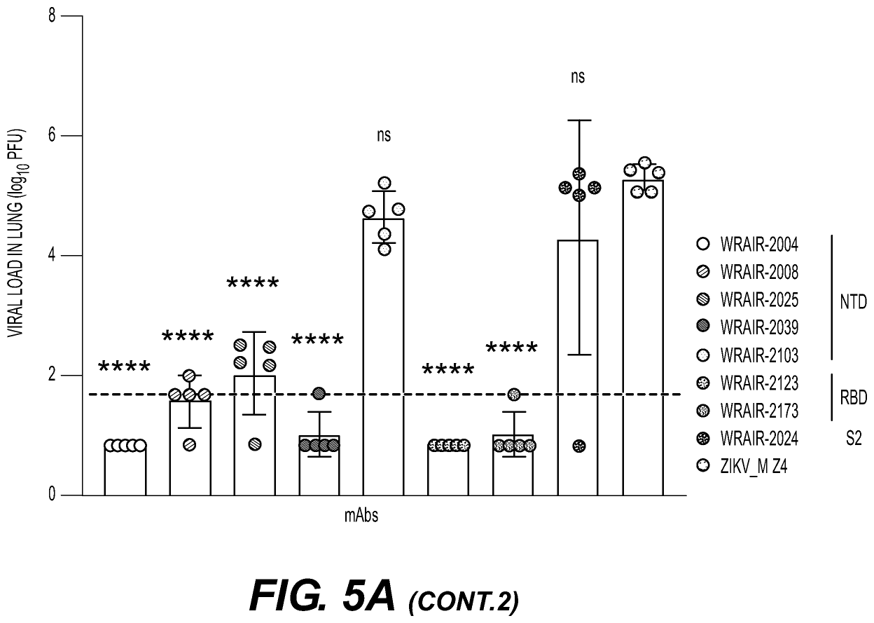

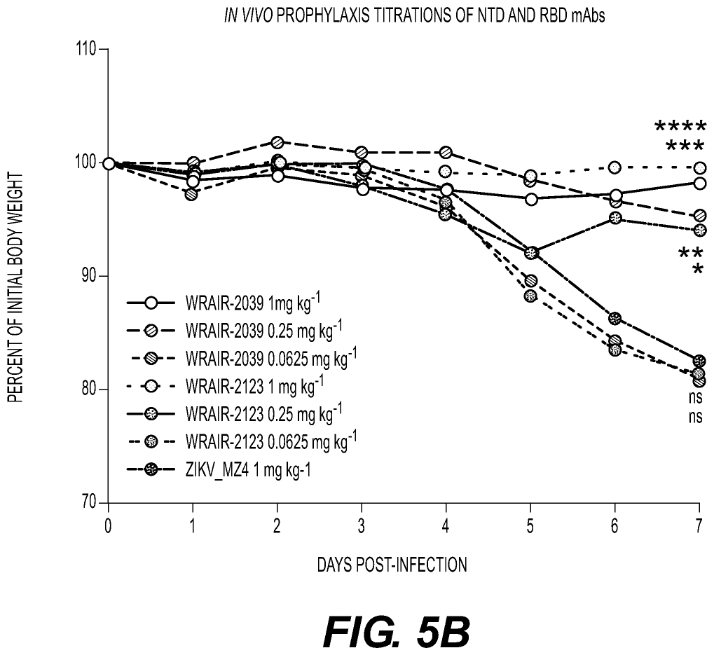

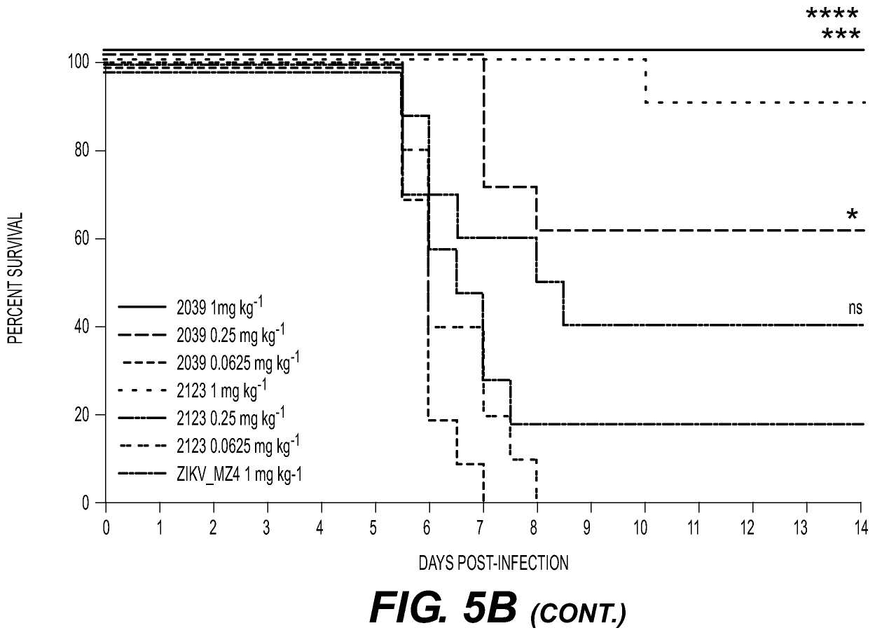

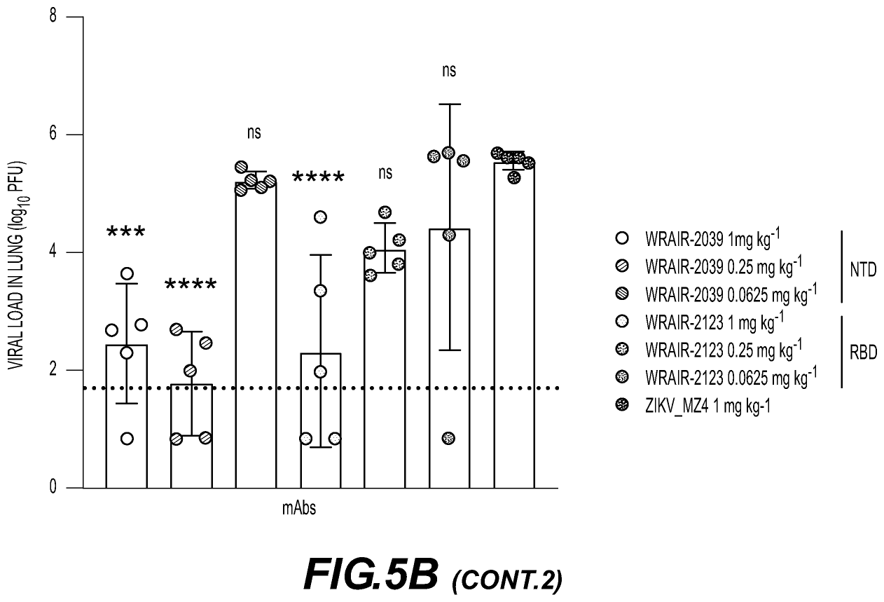

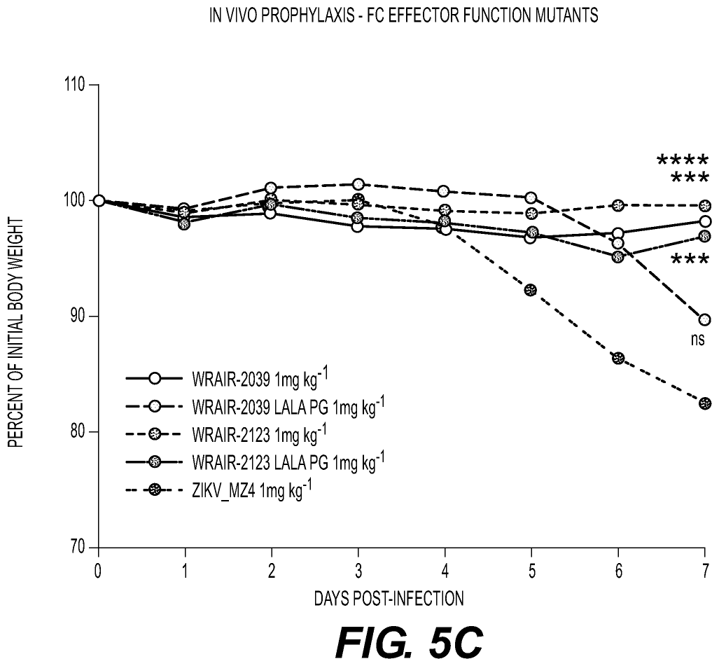

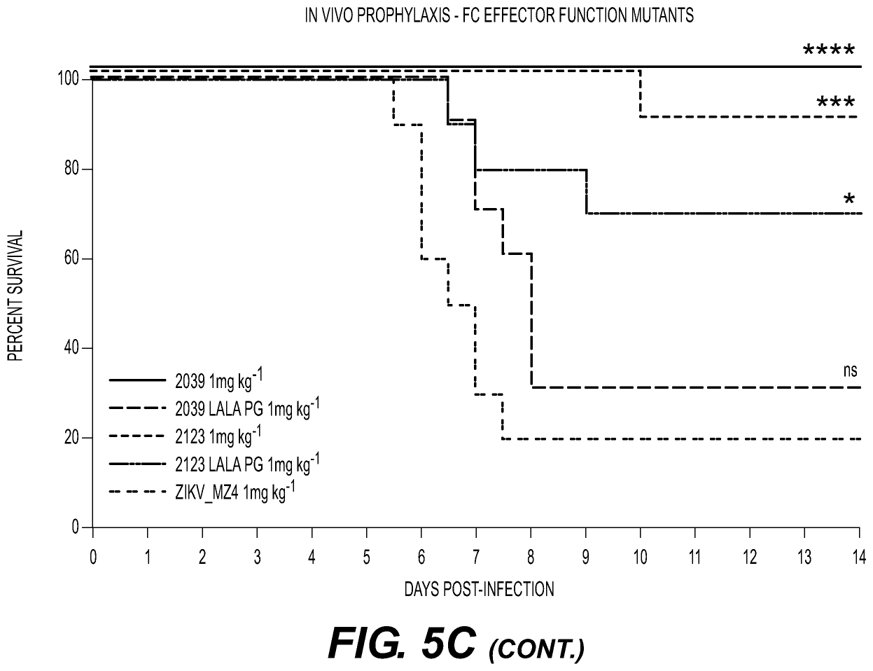

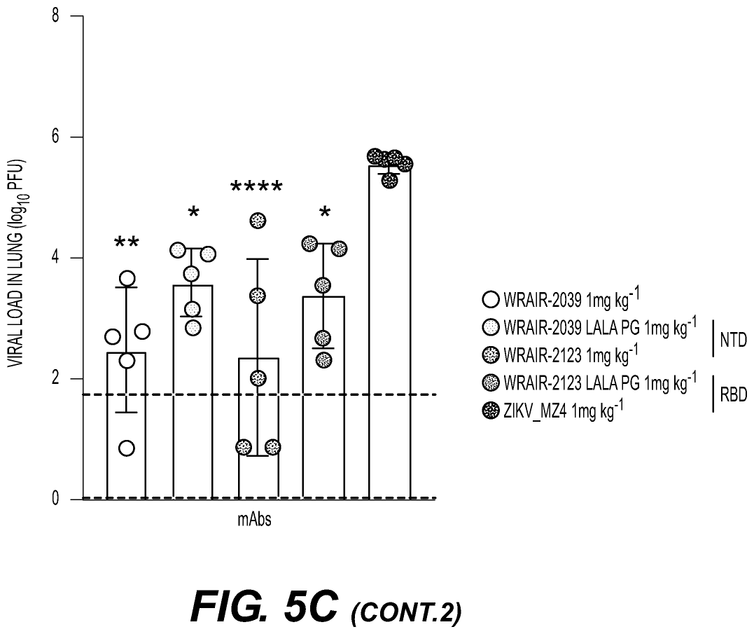

We next determined whether WRAIR NTD and RBD mAbs could confer protection in vivo with a series of experiments using the lethal K18-hACE2 transgenic SARS-CoV-2 mouse model^{33,34}. To assess protection provided by prophylaxis, mAbs were infused intravenously 24 hours prior to intranasal challenge with an 80% lethal dose of SARS-CoV-2 (1.25×10⁴ PFU WA1/2020). Using a high dose of 400 μg (20 mg kg⁻¹) of either NTD or RBD neutralizing mAbs provided complete protection (FIG. 5A). In contrast, S2-targeting mAb WRAIR-2024 and NTD non-neutralizing mAb WRAIR-2103 did not prevent infection or death at the same concentration of 20 mg kg⁻¹ (FIG. 5A), suggesting that targeting neutralization epitopes is beneficial for in vivo protection.

To determine the minimal protective dose for prophylactic protection, we next titrated the passively administered potent neutralizing mAbs WRAIR-2039 (NTD) and WRAIR-2123 (RBD) until protection was lost (FIG. 5B). Remarkably, a 5 μg (0.25 mg kg⁻¹) dose of the NTD mAb WRAIR-2039 used alone was sufficient to suppress viral replication in the lungs, confirming the high potency of NTD-directed mAbs in vivo, whereas the lowest dose where protection was observed was 1 mg kg⁻¹ for RBD mAb WRAIR-2123 (FIG. 5B).

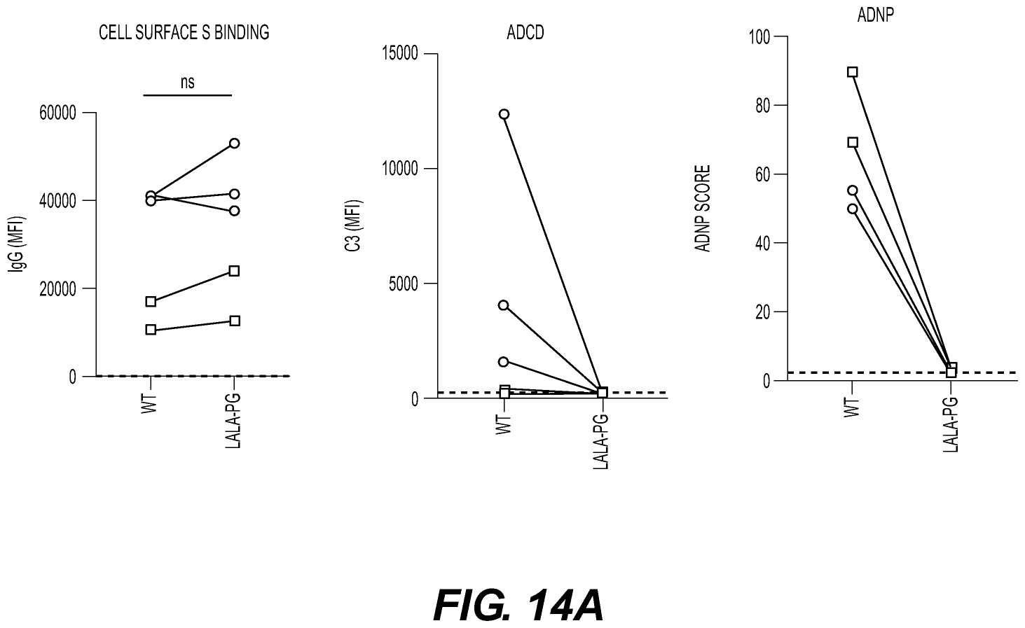

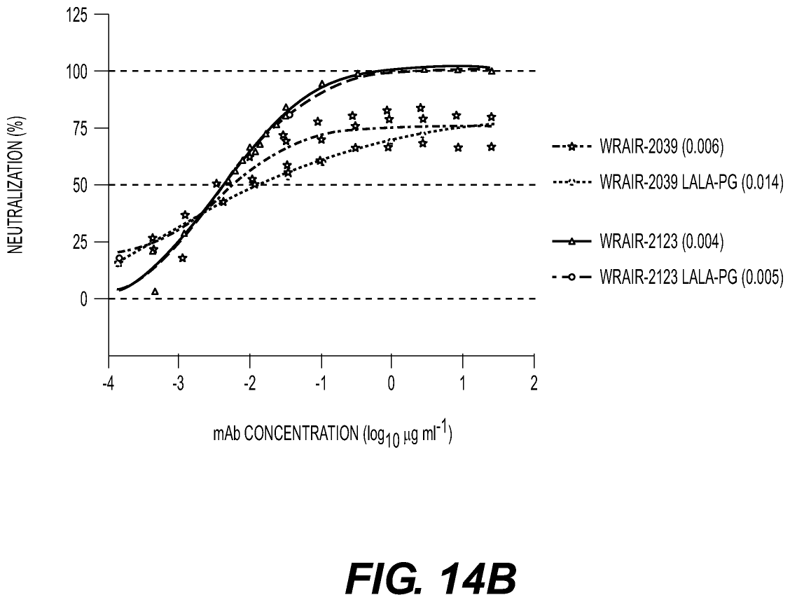

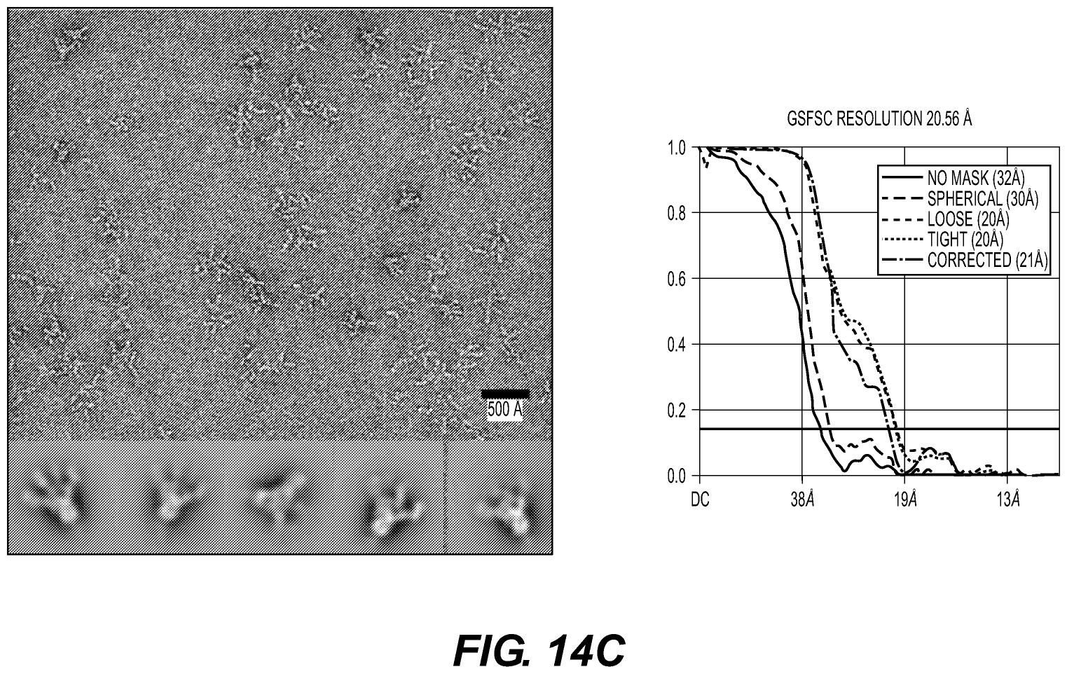

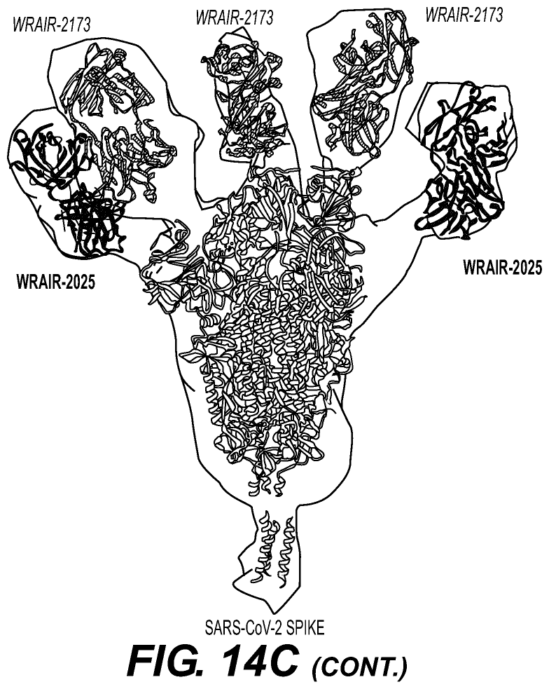

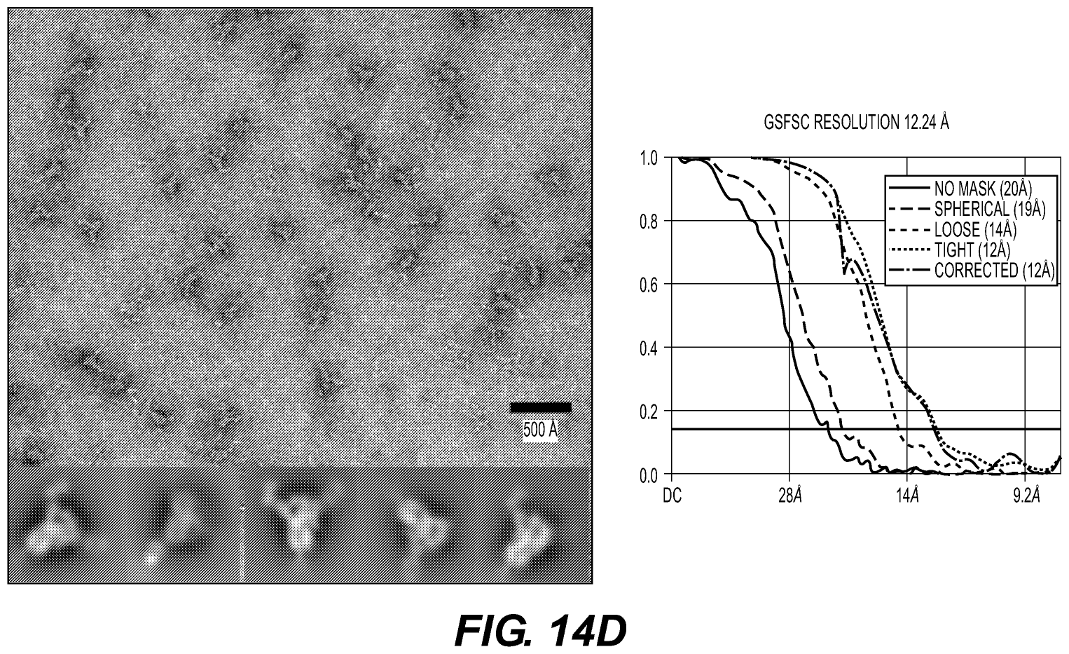

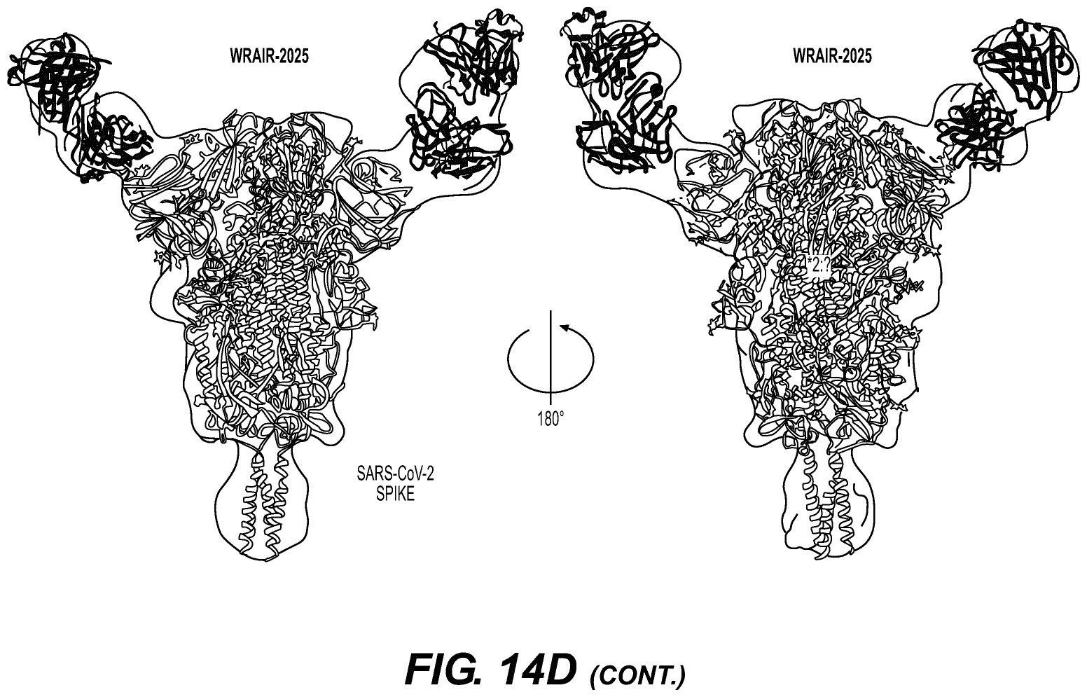

Since NTD and RBD mAbs displayed a wide range of Fc effector functions in vitro, with NTD neutralizing mAbs unique to their class in demonstrating high ADCD activity (FIG. 2H), we sought to examine whether the in vivo potency observed could be explained by engagement of Fe effector functions. RBD mAb WRAIR-2123 and NTD mAb WRAIR-2039 and were modified to harbor a triple mutation (LALA-PG)³⁵ ablating all Fe effector functions, while maintaining binding to cell surface expressed S and potent neutralization (FIG. 14A,B). When tested in vivo for prophylactic protection following passive transfer, the RBD mAb WRAIR-2123 LALA-PG mutant revealed partial protection at the 20 μg (1 mg kg¹) dose, with over half of the animals surviving infection (FIG. 5C). The requirement of Fc effector functions for in vivo protection was more pronounced for the NTD WRAIR-2039 LALA-PG mAb, where most of the animals succumbed to infection by day 8, with modest suppression of viral load in the lungs (FIG. 5C).

### Example 6. Compatibility of Neutralizing NTD and RBD Antibodies

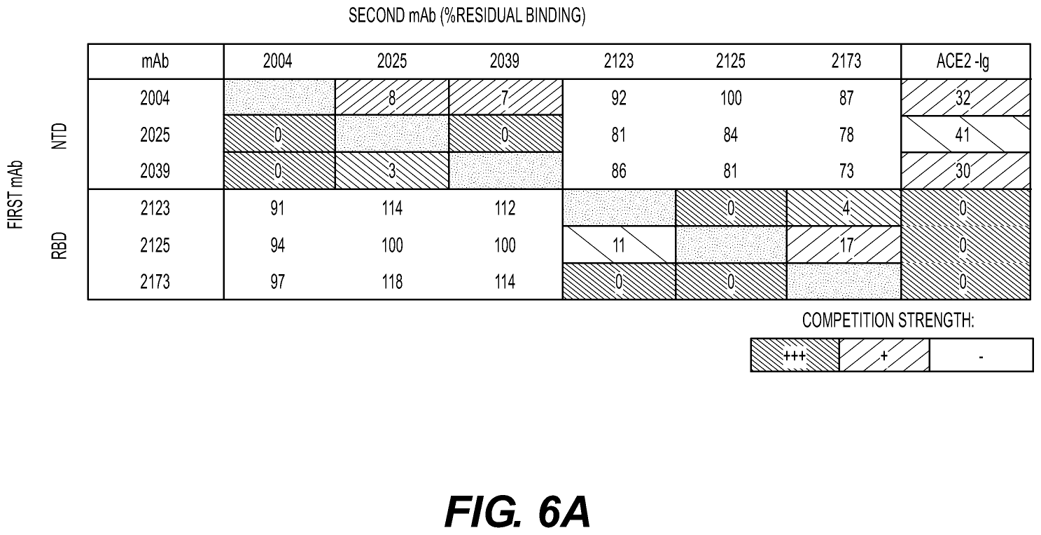

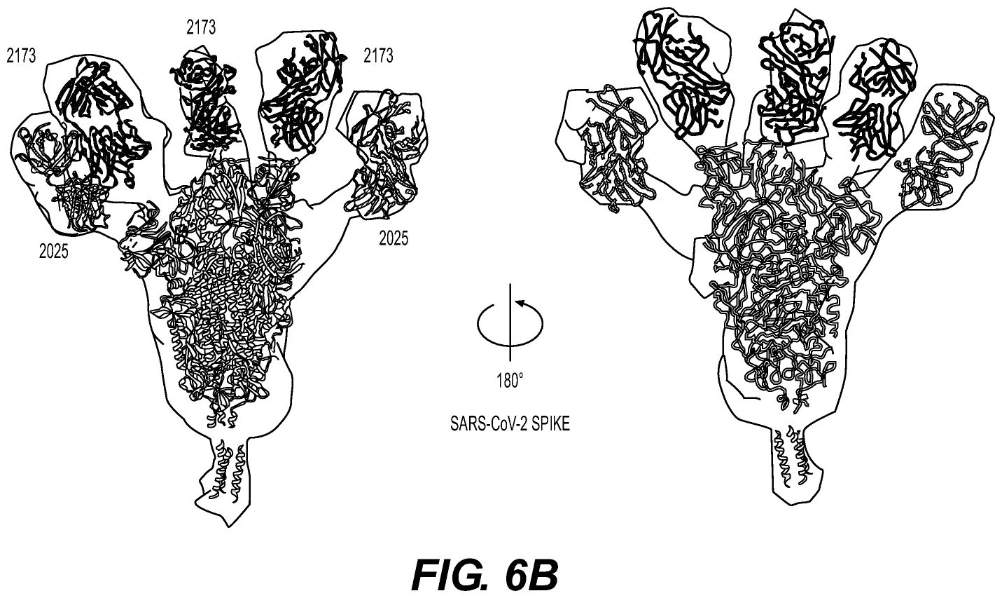

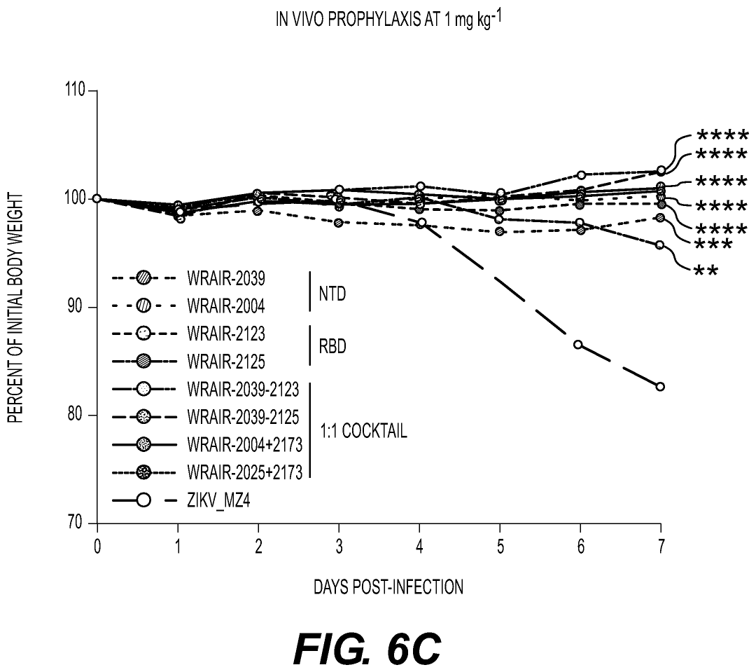

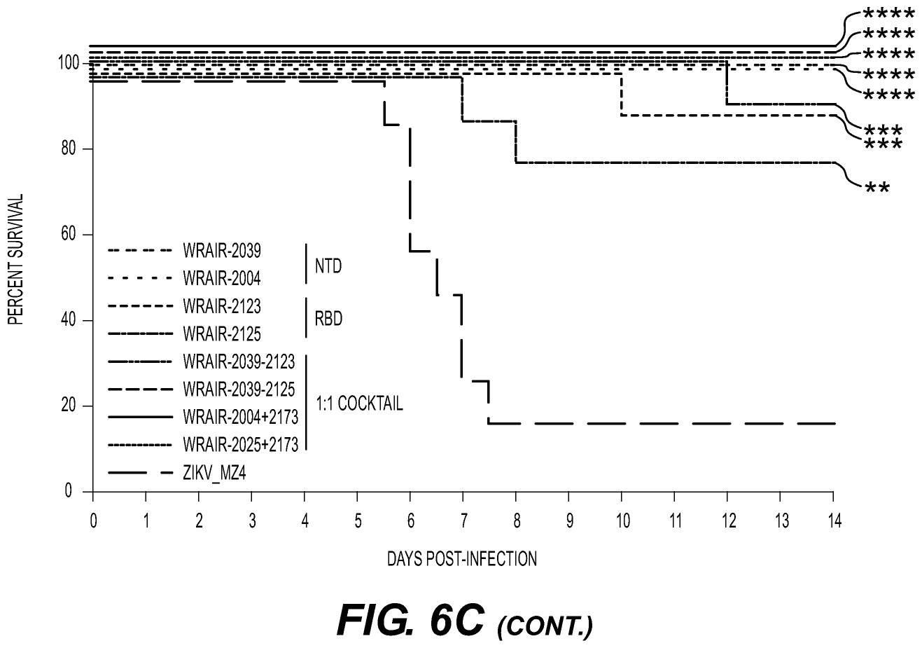

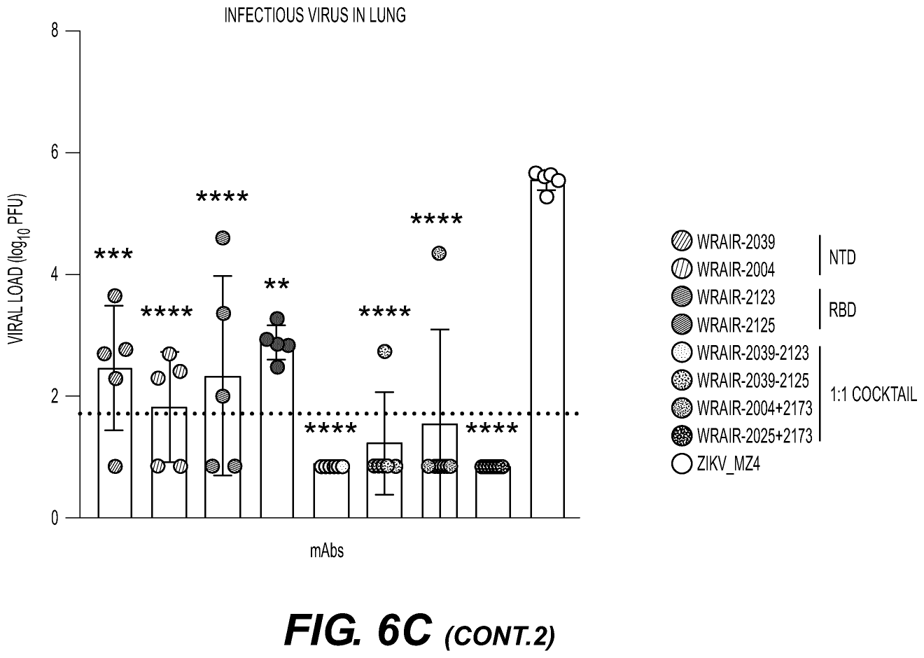

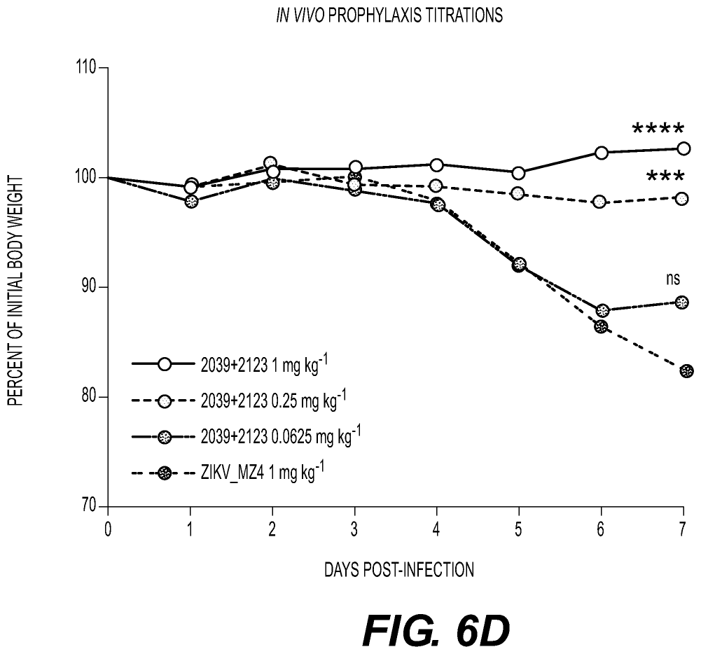

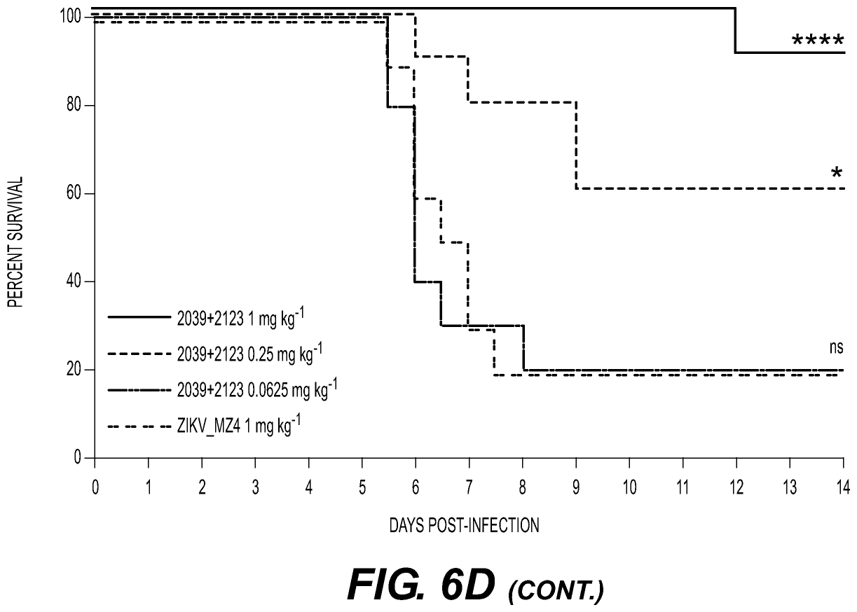

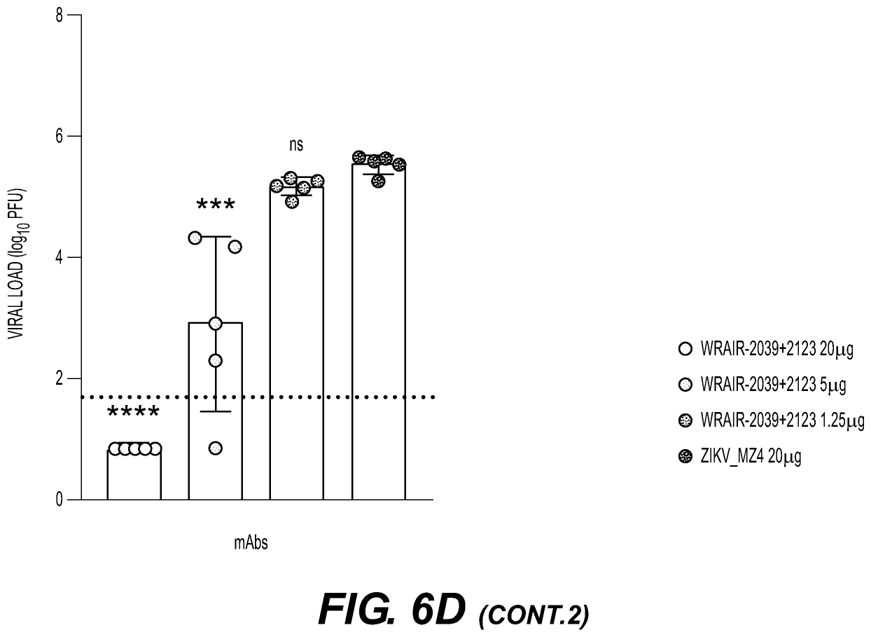

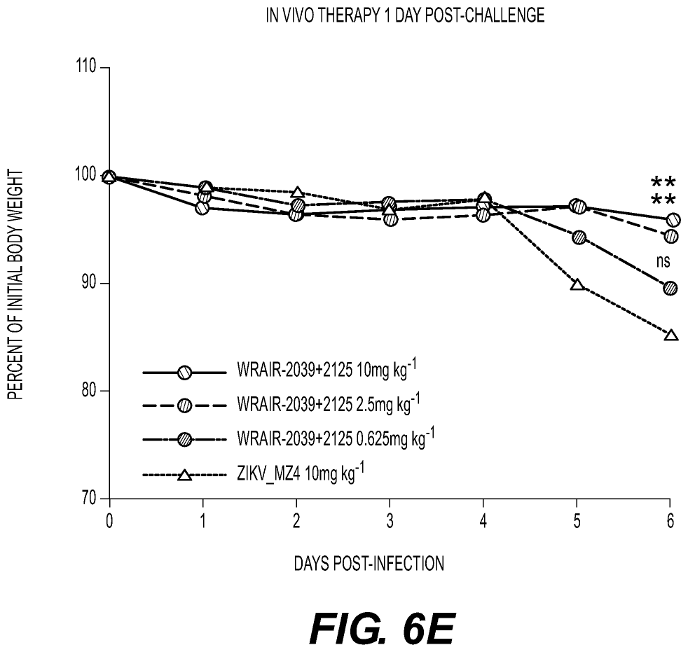

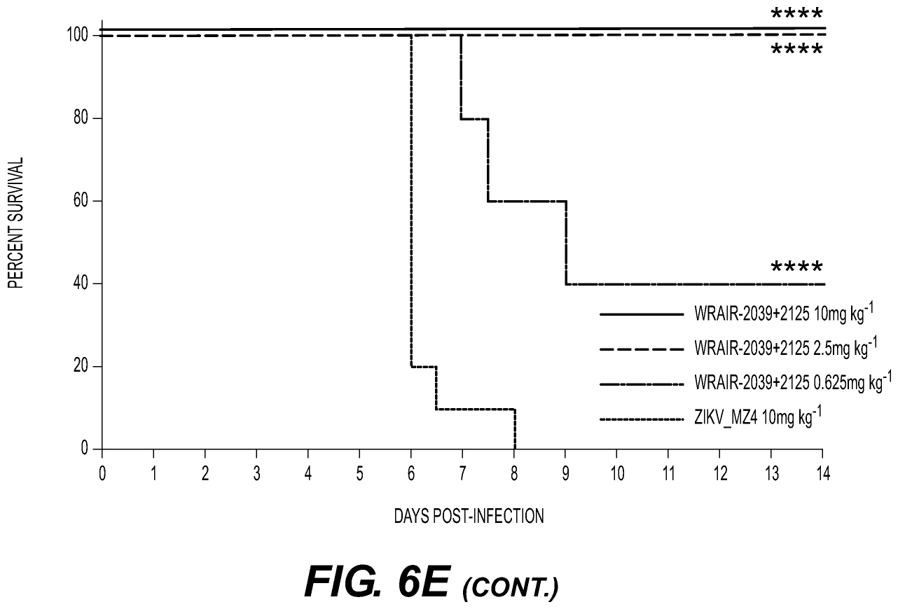

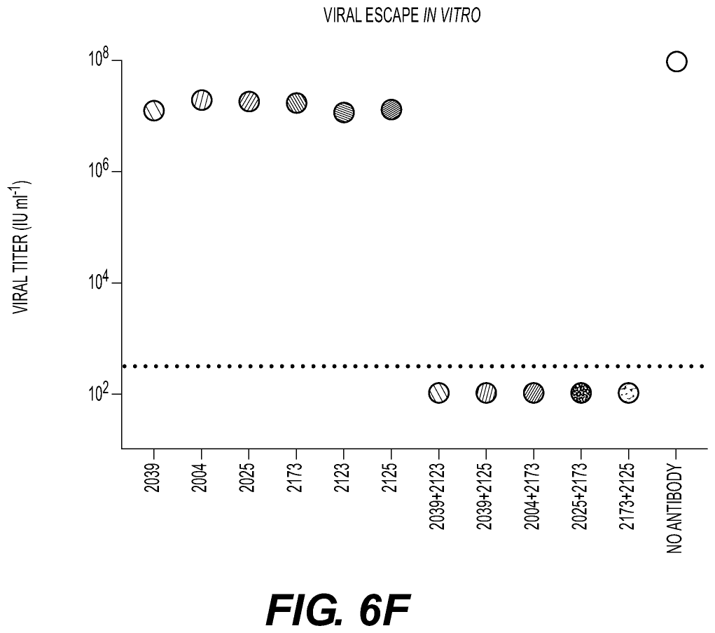

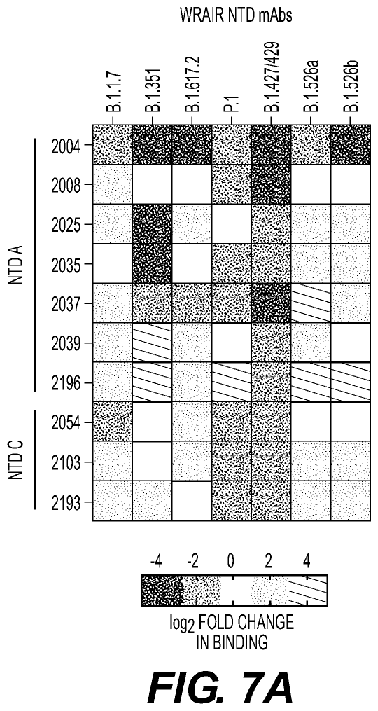

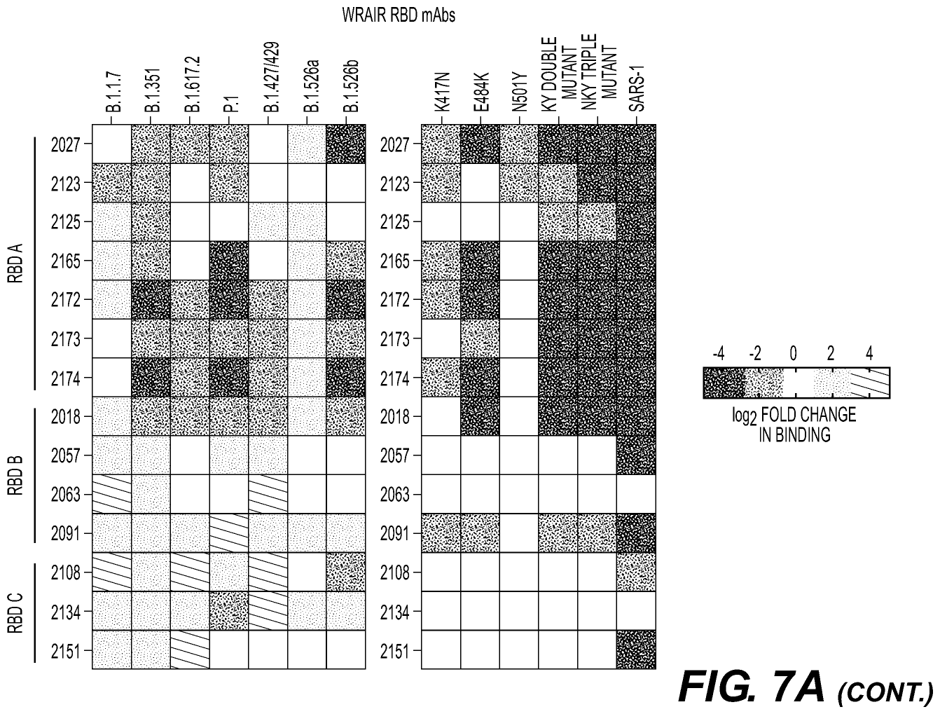

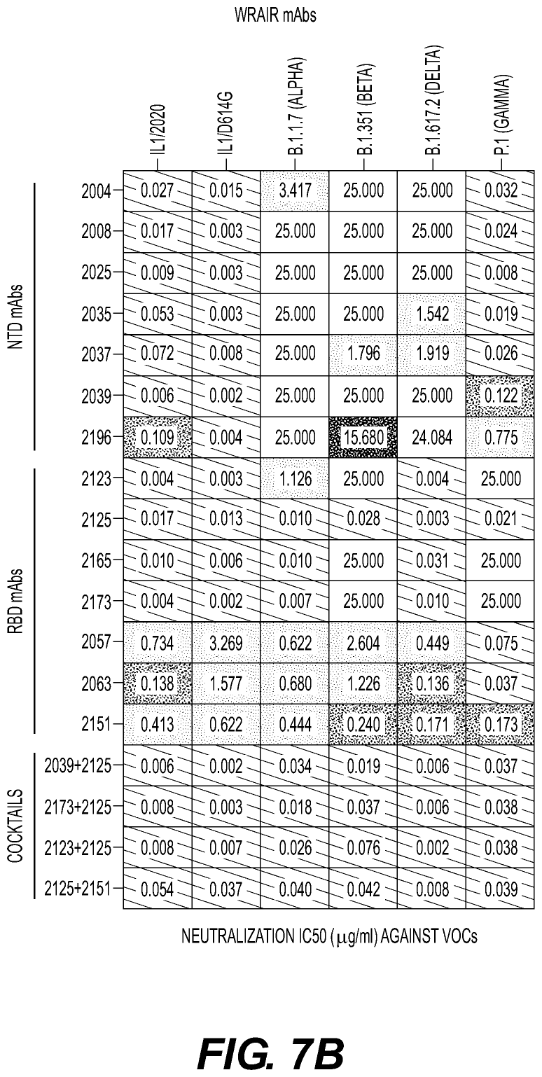

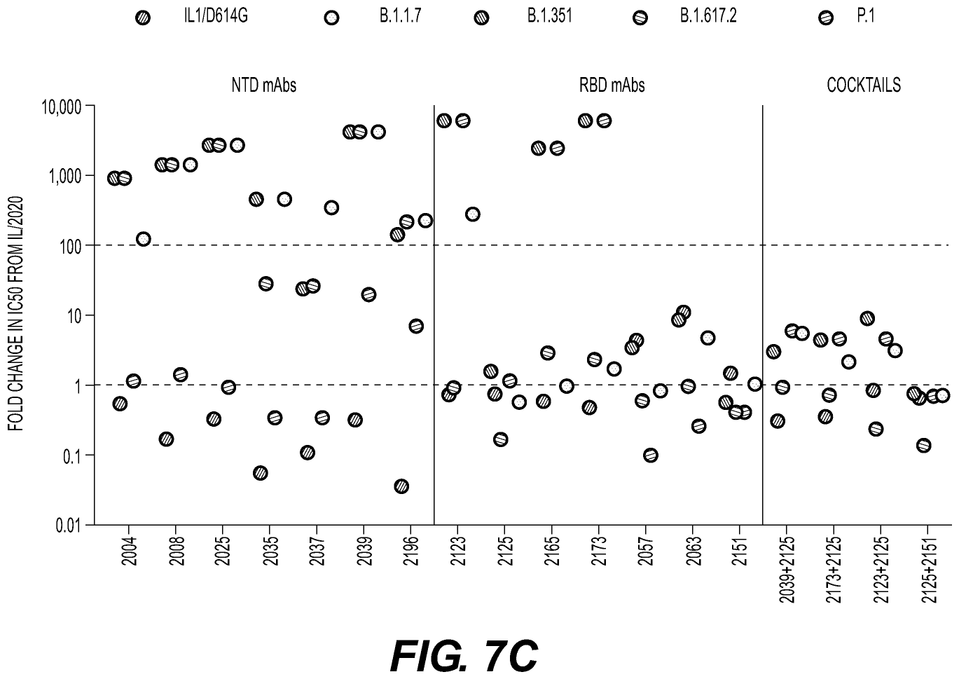

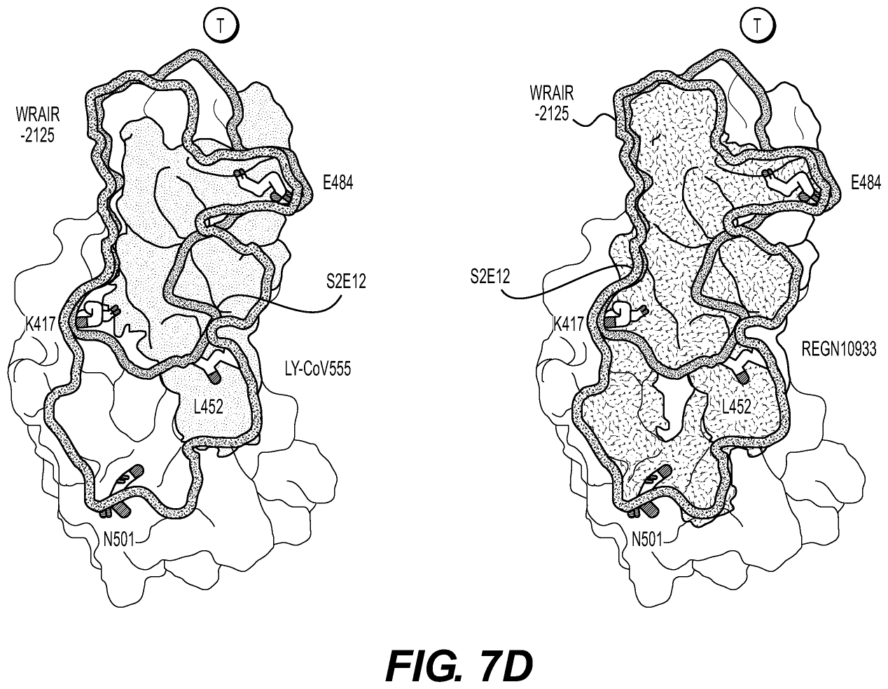

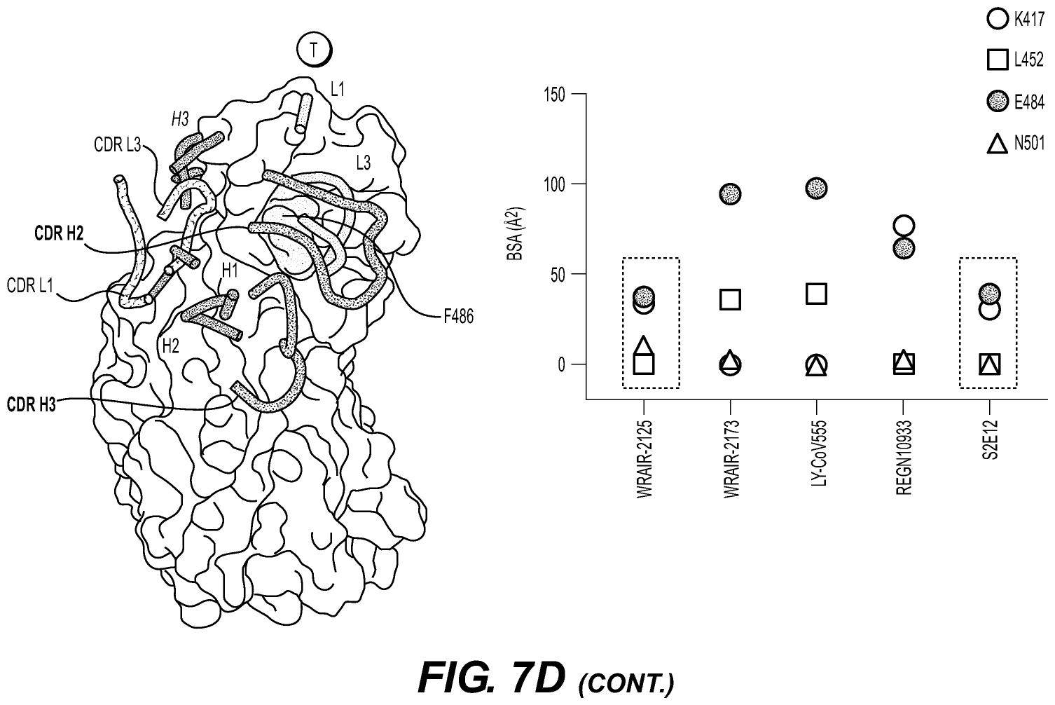

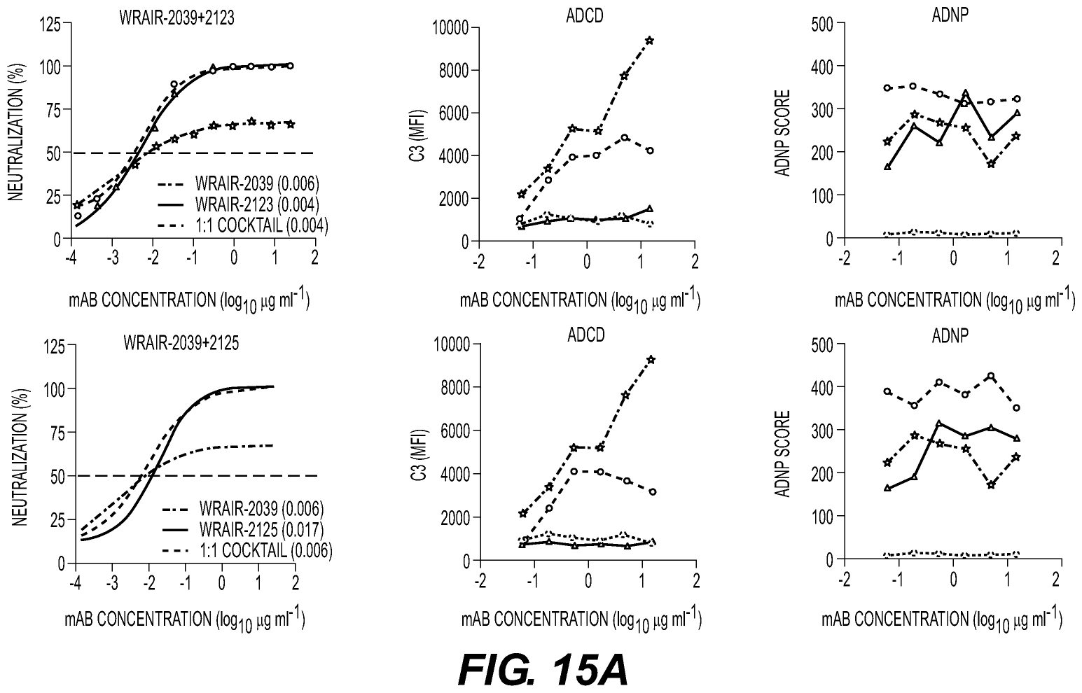

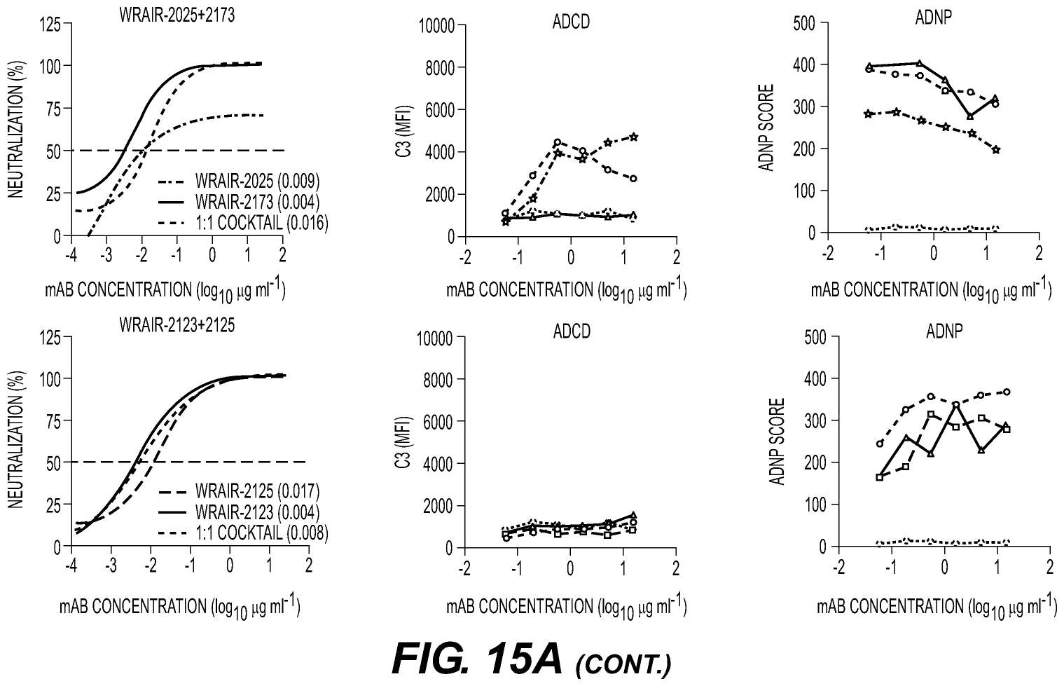

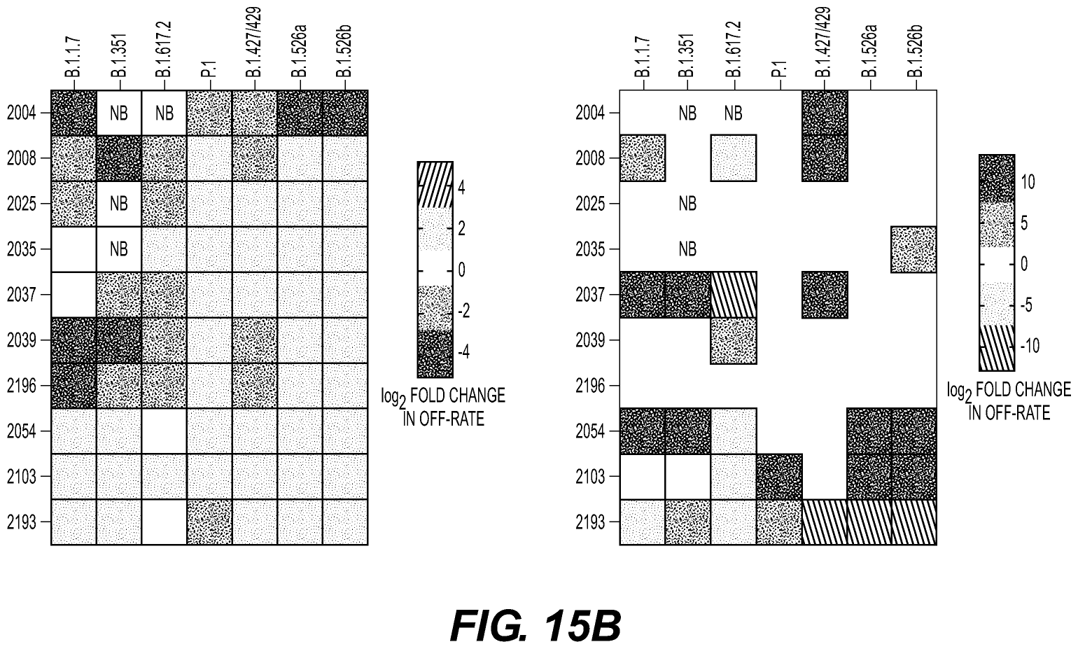

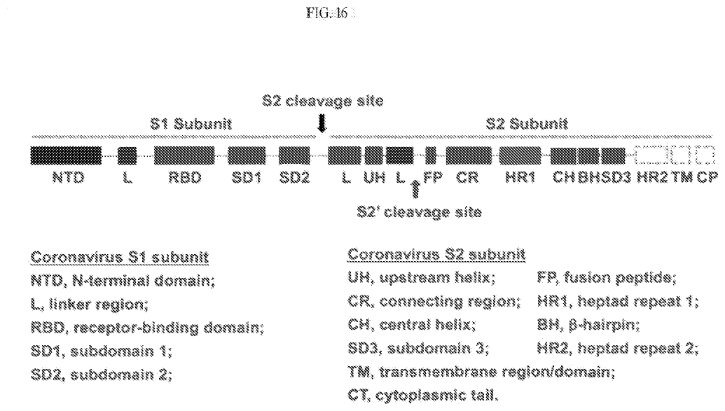

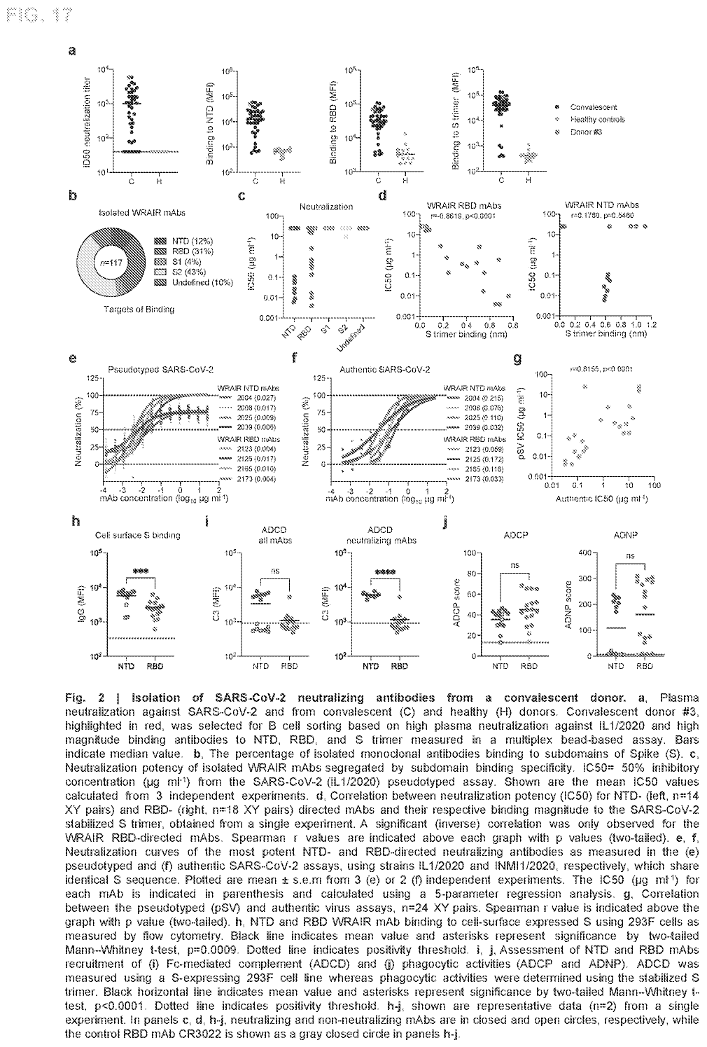

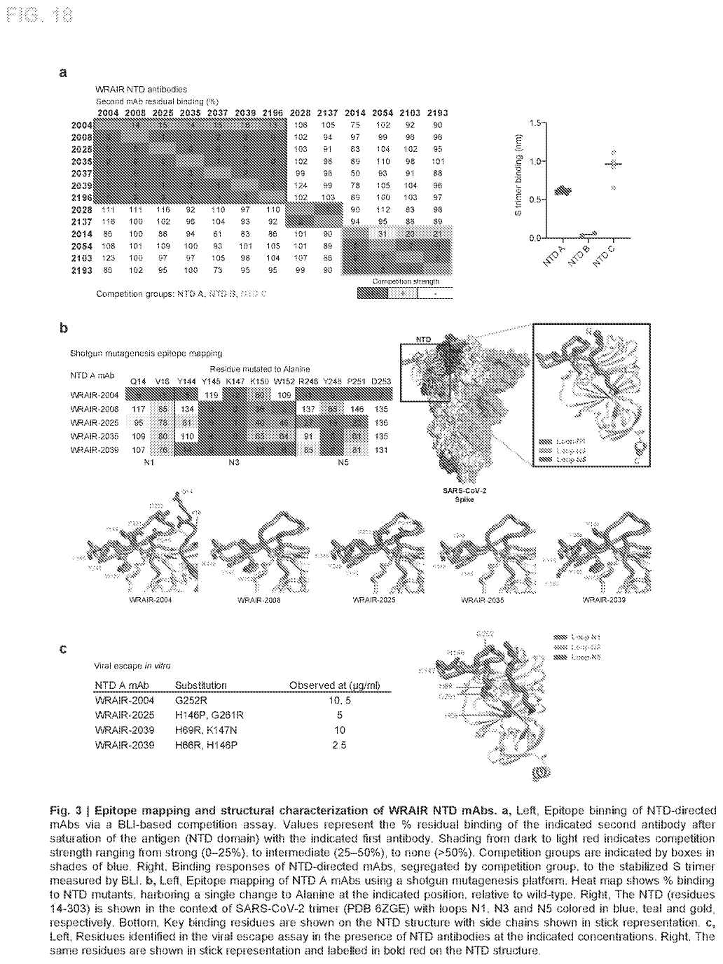

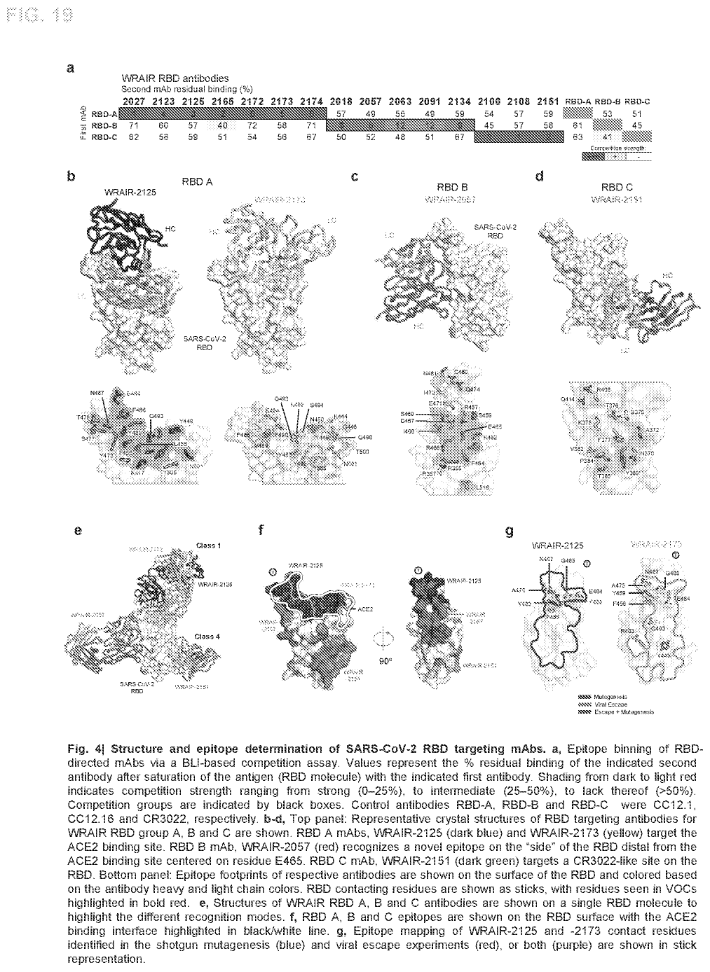

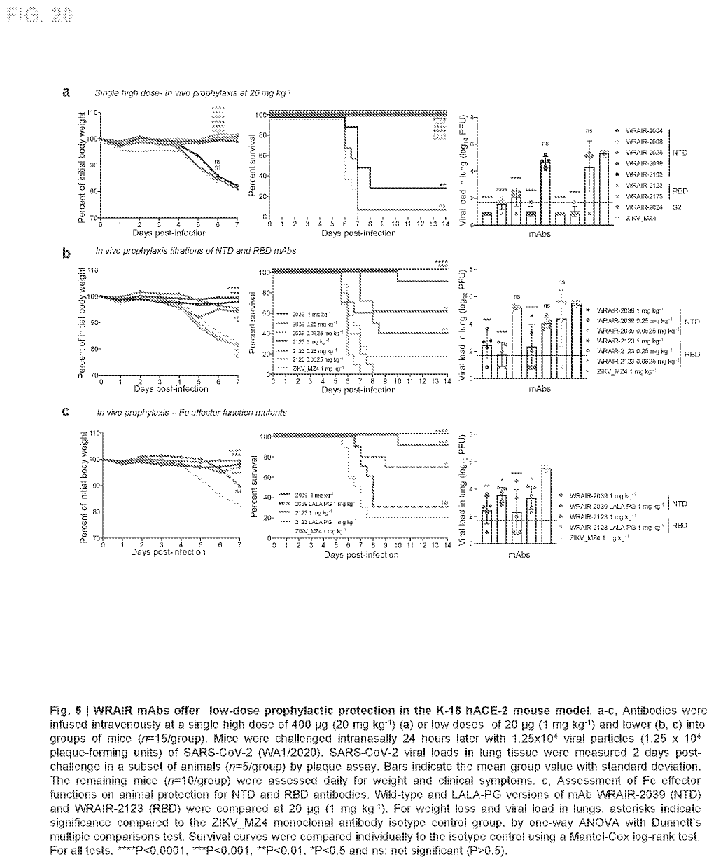

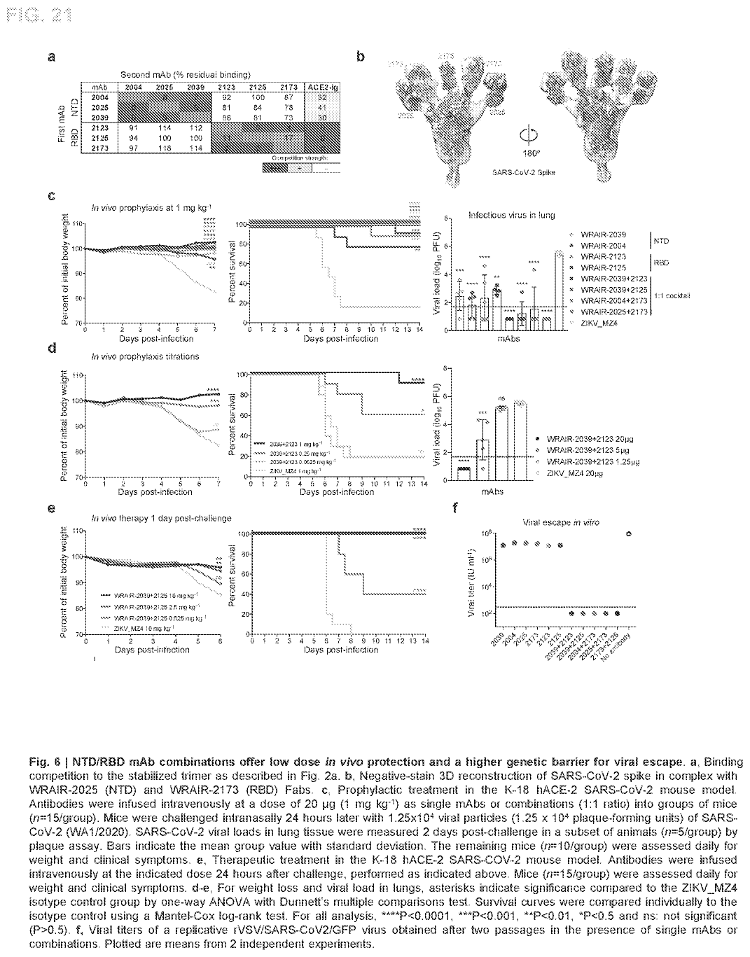

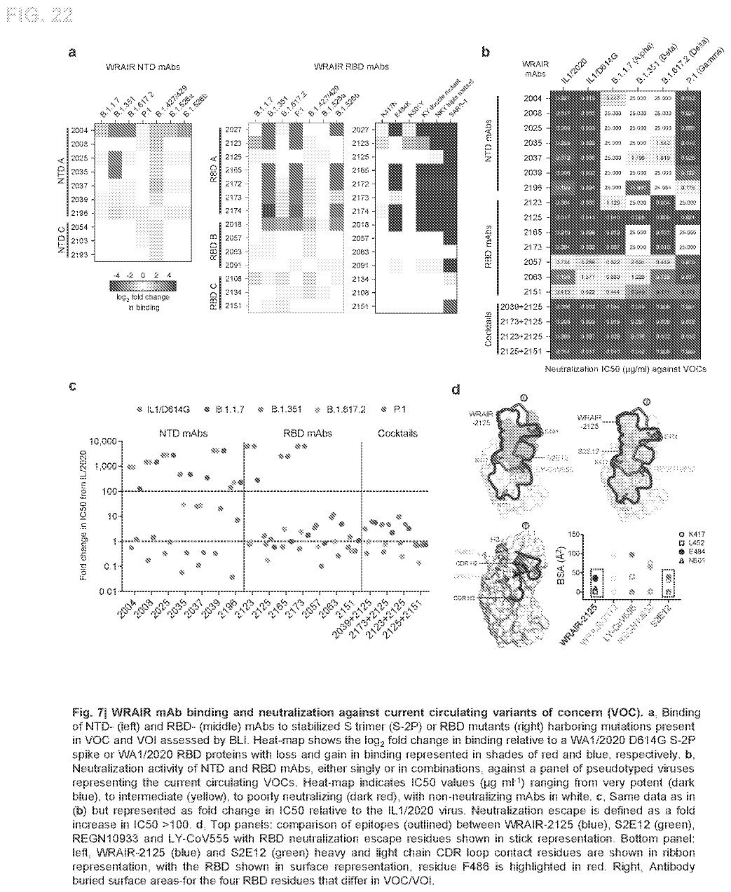

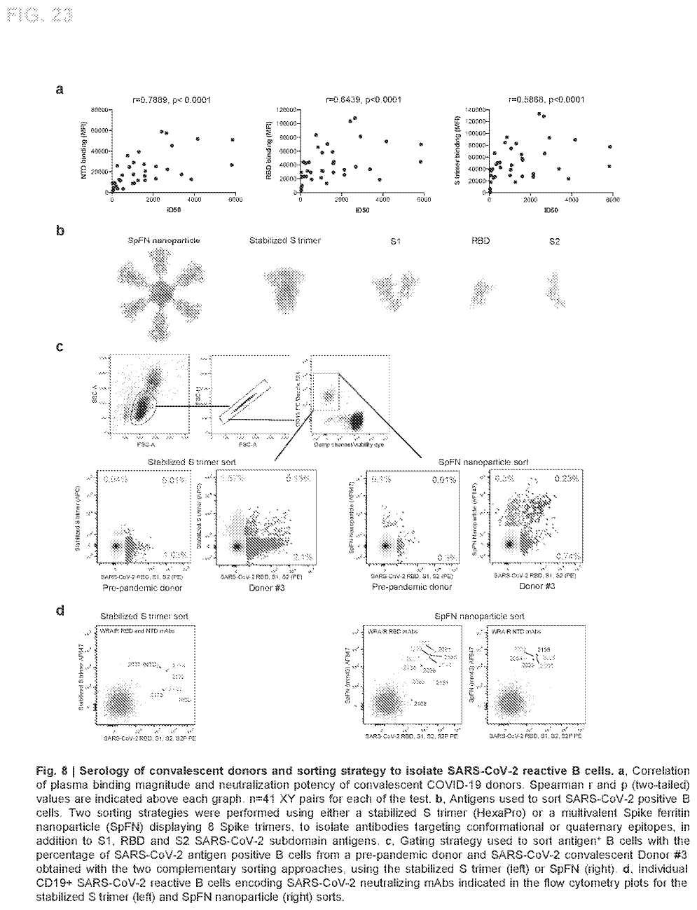

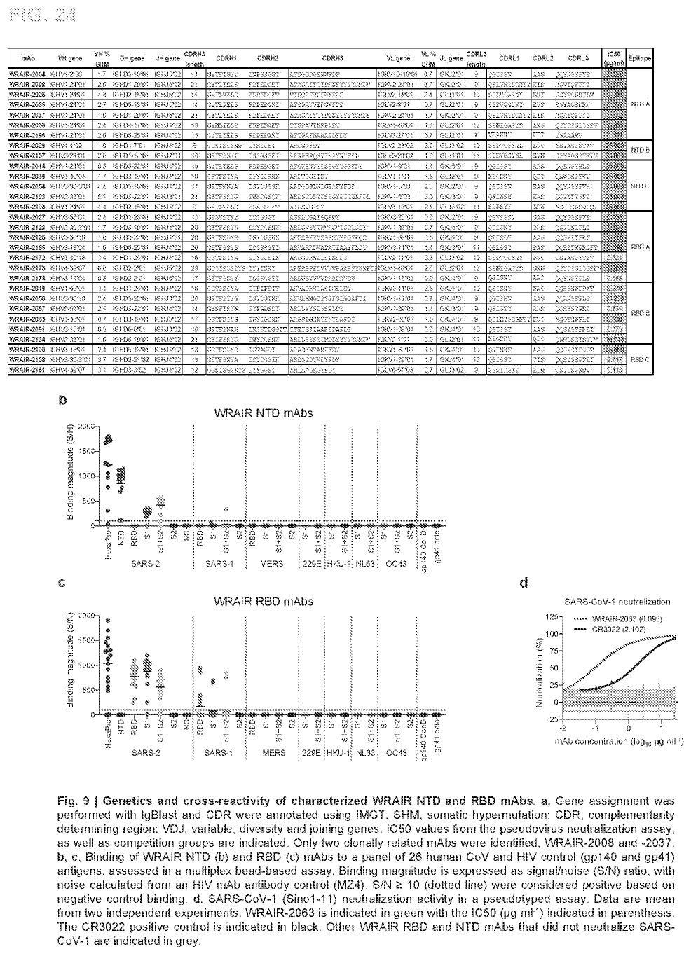

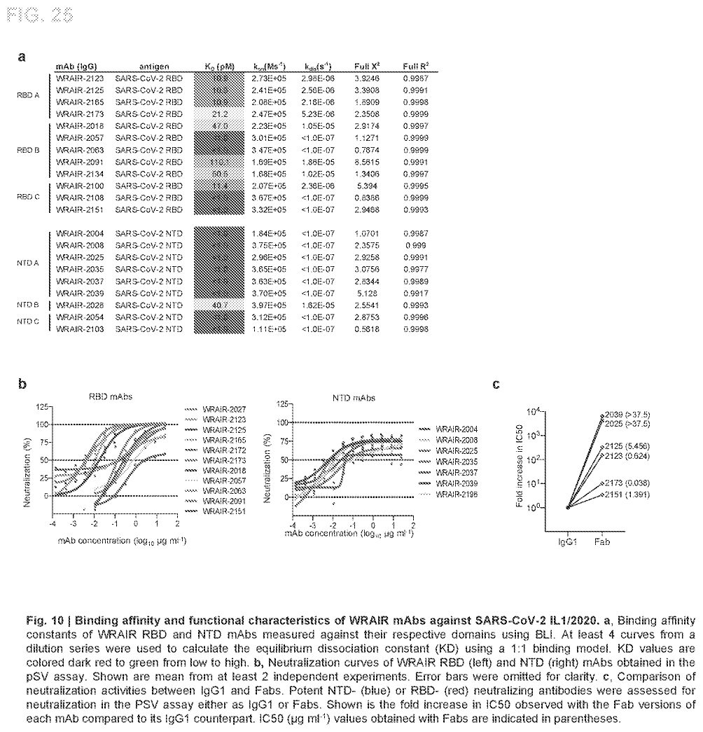

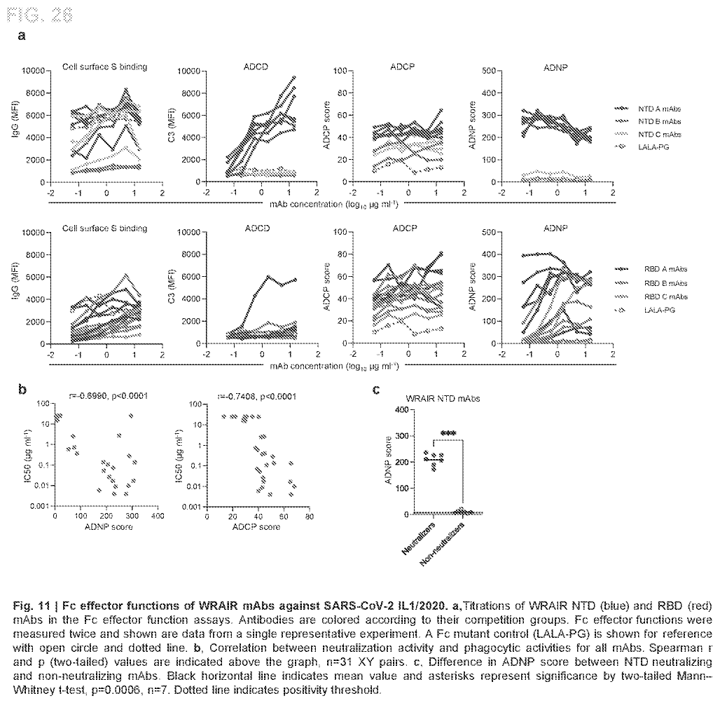

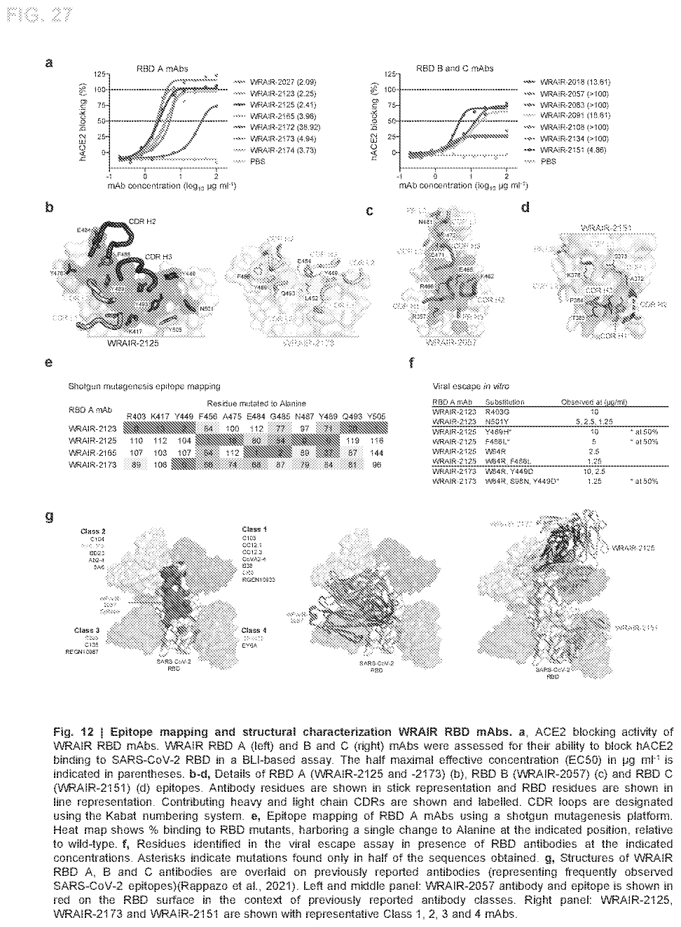

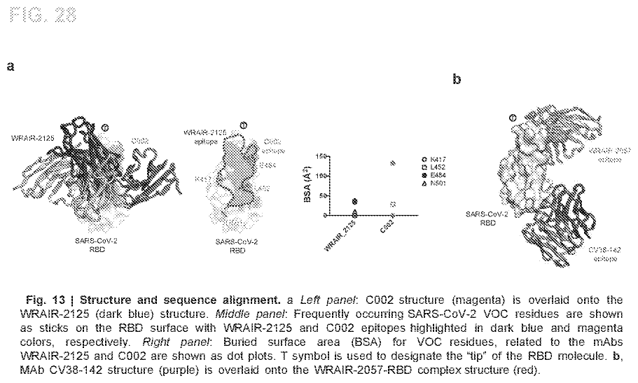

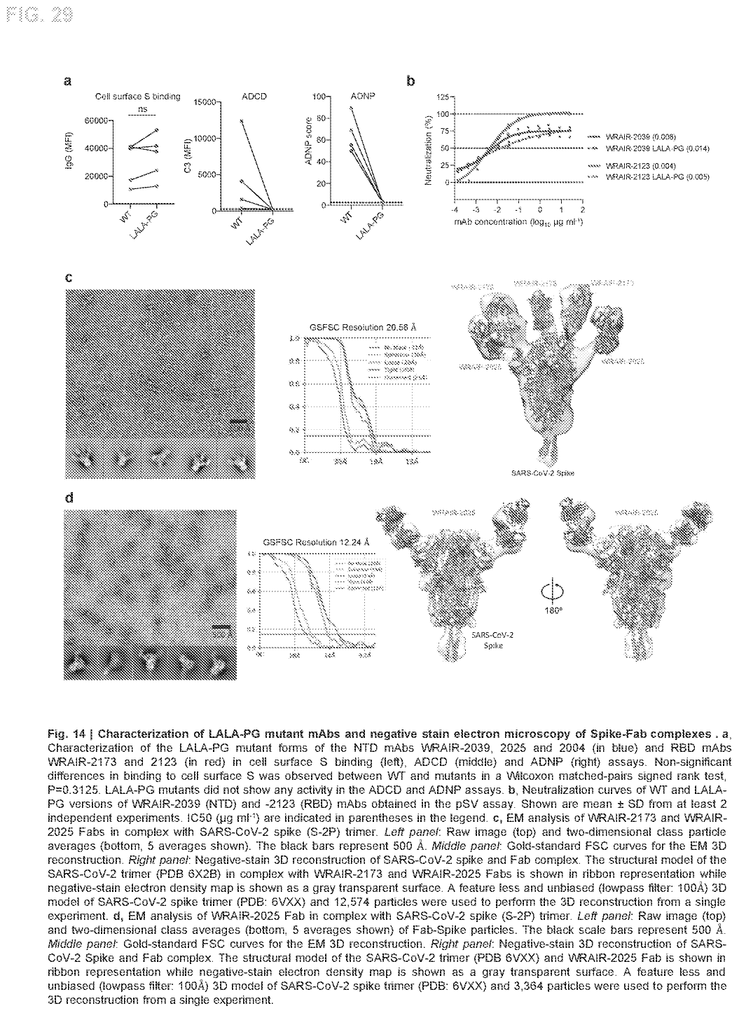

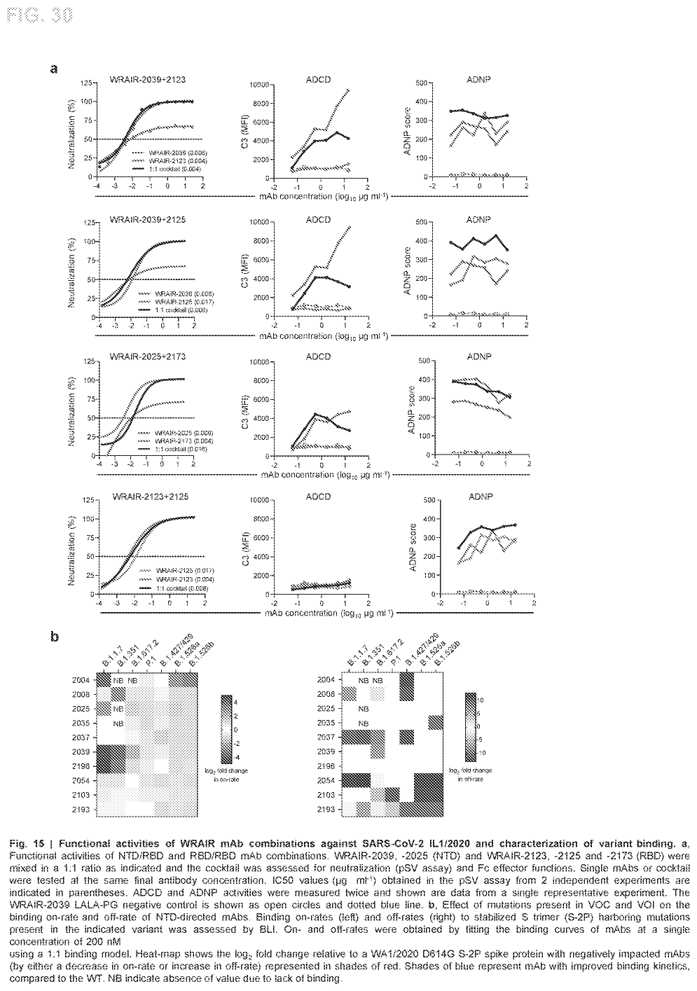

Combining mAbs targeting different sites on the surface of the viral Spike could offer advantages by increasing both breadth and potency through additive or synergistic mechanisms that can impact both neutralization and Fc effector functions, and possibly mitigate the risk for viral escape. To assess the compatibility of our potent neutralizing NTD and RBD mAbs, we first performed competition experiments with the stabilized S trimer. As expected, the NTD mAbs competed against each other, but they did not prevent binding of COV RBD mAbs to the stabilized S trimer (FIG. 7A). Similar results were obtained when competing COV group A RBD mAbs, where they competed against each other, but could bind simultaneously with COV neutralizing NTD mAbs (FIG. 7A). Modest inhibition of ACE2 binding was observed with the NTD mAbs, likely through steric hindrance through their light chain and/or Fc domains, as reported for MERS-CoV²⁸. However, group A RBD mAbs all fully blocked ACE2 recognition (FIG. 7A). Negative stain electron microscopy (EM) imaging confirmed that COV-2025 and-2173 were indeed able to engage the S trimer concomitantly, albeit with different stoichiometry (FIG. 6B). Two copies of the NTD-directed mAb COV-2025 were observed for the majority of the complexes whereas all three RBD subdomains of the Spike were occupied by COV-2173 (FIG. 7A, 6B. FIG. 14C-D). To verify that combining NTD and RBD mAbs would not be detrimental to their neutralization activity, we analyzed several combinations of the most potent COV mAbs in the SARS-CoV-2 pseudotyped assay. We found no evidence of interference between the two classes of mAbs as all combinations tested showed additive effects, indicating that NTD/RBD mAb cocktails would offer a potent dual target approach (FIG. 15A), Likewise, additive effects were also observed for Fc effector functions of NTD/RBD nAb combinations, particularly with respect to ADCD and ADNP (FIG. 15A).

Next, we determined whether WRAIR NTD and RBD mAb combinations could confer protection in vivo with a series of experiments using the lethal K18-hACE2 transgenic SARS-CoV-2 mouse as described above. To assess protection provided by prophylaxis, potently neutralizing NTD and RBD mAbs were administered either singly or as a 1:1 combination at a low dose of 20 μg (1 mg kg⁻¹). K18-hACE2 mice treated with these single or dual mAb combinations did not show any clinical signs of illness over the course post-challenge follow-up, while weight loss was observed from day 5 in control animals that received the isotype control mAb (ZIKV_MZ4³⁹) (FIG. 6c, left). By day 7, animals in the control group succumbed to SARS-CoV-2 infection (FIG. 6C, (CONT.)). High infectious virus titer levels were found in lung homogenates, measured at the peak of viral replication, two days post-infection (FIG. 6c, CONT2.). While all mAb-treated groups exhibited significantly lower viral titers in the lungs compared to the isotype control group, all animals treated with the mAb combinations demonstrated undetectable virus in the lungs, with the exception of 2 mice (FIG. 6c, CONT2.). In contrast, low levels of replicating virus were found in mice that received a single mAb at 1 mg kg⁻¹ (FIG. 6c, CONT2.), supporting a role of enhanced protection by combination mAbs targeting two different sites on the Spike surface.

To determine the minimal protective dose for prophylactic protection for a combination of WRAIR-2039 (NTD) and WRAIR-2123 (RBD), we next titrated the passively administered potent neutralizing mAbs until protection was lost (FIG. 6D). In a 1:1 combination, WRAIR-2039 (NTD) and WRAIR-2123 (RBD), provided suppression of viral replication in the lungs at a low dose of 5 μg (0.25 mg kg⁻¹), where each mAb used at 2.5 μg dose or 0.125 mg kg⁻¹ (FIG. 6D). In addition to prophylaxis, we assessed whether NTD- and RBD-targeting mAb combinations could provide therapeutic benefit, one day after challenge in the same K-18 mice model. A dose-titration experiment revealed that 50 μg (2.5 mg kg⁻¹) of the NTD mAb WRAIR-2039 in combination with RBD mAb WRAIR-2125 was fully protective, with partial protection (4/10 animals) observed at the 12.5 μg (0.625 mg kg⁻¹) dose (FIG. 6E), demonstrating high potency of mAb combinations in both prophylactic and therapeutic challenge models.

Targeting 2 different sites on the Spike surface may also prevent the emergence of antibody resistant viral variants. To test this hypothesis, we cultivated rVSV/SARS-CoV2/GFP in the presence of single NTD and RBD mAbs, and subsequently selected for resistant viral populations that replicated to high levels, as expected (FIG. 6F). In contrast, when dual combinations containing NTD and RBD mAbs were used at the same total concentration (10 μg ml¹) as was used for the individual mAbs, no infectious rVSV/SARS-CoV2/GFP was recovered (FIG. 6F). Thus, consistent with previous observations³⁶, S mutations can be readily acquired causing escape from individual antibodies, but mAb combinations that target distinct epitopes present a higher genetic barrier to viral escape. Collectively, NTD and RBD mAb combinations demonstrate complementary antibody functions, enhanced in vivo protection, and provide higher resistance to viral escape.

### Example 7. Neutralization of Variants of SARS-CoV-2 Virus

The emergence of several viral variants of concern (VOCs) threatens current preventative and therapeutic strategies using SARS-CoV-2 neutralizing mAbs. To evaluate the activity of WRAIR mAbs against VOCs, we first assessed binding against a set of S trimers harboring mutations found in circulating VOC (Alpha, Beta, Delta, and Gamma strains) and two variants of interest (VOI) (B.1.427/429 and B.1.526a/b). NTD mAbs showed up to 8-fold reduced binding to B.1.351 (Beta) and 2- to 3-fold to B.1.427/429, but most retained binding to B.1.1.7 (Alpha), B.1.617.2 (Delta) and P.1 (Gamma) (FIG. 7A). However, even when binding was detected, NTD mAbs exhibited altered binding kinetics to B.1.1.7, B.1.351 and B.1.617.2 S trimers, manifested by slower association (decrease in on-rate) and/or faster dissociation (increase in off-rate) (FIG. 15B). RBD mAbs were tested against the same panel of S variants. For RBD A mAbs, loss of binding was largely driven by the E484K mutation, especially when combined with other RBD residue changes such as K417N/T and N501Y (found in the B.1.351 (Beta) and P.1 (Gamma) variants) (FIG. 7A,CONT., left). Binding to RBD proteins harboring those 3 mutations, both individually and in combinations, confirmed these results (FIG. 7A, CONT., right). Among potent neutralizing mAbs, RBD mAb WRAIR-2125 retained binding to all VOC tested, while RBD mAb WRAIR-2173 binding was ablated by the combined double and triple mutations found in VOCs such as B.1.351 and P.1 (FIG. 7a, CONT., right).

As expected, binding of RBD mAbs from competition groups B and C were less affected by these mutations as their epitopes lie outside of the ACE2 binding interface (FIG. 3). Neutralizing RBD B mAbWRAIR-2063 bound equally well to all WT and mutant proteins, including SARS-CoV-1 (Sino 1-11) RBD (FIG. 7a, CONT., right). We next performed pSV neutralization assays against a panel of SARS-CoV-2 strains encompassing the original virus and circulating VOC. Several mutations such as 69-70del and Y144del (B.1.1.7), 241-243del (B.1.351) or 156-157del (B.1.617.2) conferred SARS-CoV-2 resistance to NTD-mediated neutralization (FIG. 7B,C). As a result, most WRAIR NTD neutralizing mAbs lost their activity against pseudotyped B.1.1.7 (Alpha), B.1.351 (Beta) and B.1.617.2 (Delta), but, interestingly, retained intact potency against P.1 (Gamma), indicating that the mutations present in the NTD of this variant are not as disruptive (FIG. 7B,C). However, both WRAIR-2035 and-2037 retained modest neutralizing activity against B.1.617.2 (Delta), while the latter also neutralized B.1.351 (Beta). For the WRAIR RBD mAbs, several remained highly potent against the B.1.1.7 Alpha variant, which harbor a single RBD mutation, at position N501Y. Similarly, the mutations L452R and T478K present in the B.1.617.2 (Delta) did not significantly impact the neutralization activity of the most potent RBD mAbs such as WRAIR-2123 and-2125, which both displayed IC50 value of 3-4 ng ml¹ against this currently dominating variant. Other variants such as B.1.351 (Beta) and P.1 (Gamma), which combine mutations K417N/T, E484K and N501Y, escaped pSV neutralization from most RBD A mAbs, including three of the most potent WRAIR mAbs, WRAIR-2123, -2165 and -2173. Remarkably, and in agreement with its ability to bind to S trimers harboring mutations found in VOC, WRAIR-2125 was the only RBD A mAb able to potently neutralize all VOC (FIG. 7B,C). RBD mAbs targeting epitopes outside of the ACE2 binding interface, such as WRAIR-2151, were also able to neutralize all SARS-CoV-2 strains tested, albeit less potently than WRAIR-2125 (FIG. 7B,C). In addition, antibody combinations comprising of WRAIR-2125 and either the NTD mAb WRAIR-2039 or the RBD mAbs WRAIR-2123, -2173 or -2151 demonstrated potent neutralization across all VOC (FIG. 7B,C). Taken together, multiple sets of residue mutations and deletions impact antibody binding and neutralization. However, remarkably, WRAIR-2125, retained potent neutralization activities against all VOC either alone or in combination with NTD or other RBD mAbs.

### Example 8. COV-2125

COV-2125 shares heavy (IGHV3-30*18) and light (IGKV1-39*01) chain usage with previously reported mAbs P17 and C002 (Yao, H. et al. (2021) Cell Res 31, 25-36; Barnes, C. O. et al. (2020) Nature 588, 682-687). However, COV-2125 binding mode is very different from C002, which relies heavily on E484 while COV-2125 is centered around F486 (FIG. 13). Unlike COV-2125, approved first generation mAbs REGN10933 and LY-CoV555 also rely heavily on residues frequently mutated in VOC (FIG. 7D). COV-2125 has reduced BSA interaction with E484 but also K417 residues, explaining its ability to resist neutralization escape by VOCs (FIG. 7D; FIG. 13). In this regard, the binding mode of COV-2125 shares more resemblance to a class of F486-targeting IGHV1-58/IGKV3-20-derived mAbs belonging to a public clonotype identified in multiple donors (Tortorici, M. A. et al. (2020) Science 370, 950-957; Dong, J. et al. (2021) bioRxiv; Wang, L. et al. (2021) bioRxiv). Similarly to COV-2125, these mAbs are potent neutralizers of SARS-CoV-2 and retained high potency across all VOC (Chen, R. E. et al. (2021) Nature medicine). In order to understand the neutralization coverage of COV-2125-like mAbs against SARS-CoV-2 variants, we compared the epitopes of COV-2125 with one of this IGHV1-58/IGKV3-20 mAb, S2E12 (Tortorici, M. A. et al. (2020) Science 370, 950-957), with COV-2173 and emergency use authorized antibodies, REGN10933 and LY-CoV555 (Wang, L. et al. (2021) bioRxiv; Hansen, J. et al. (2020) Science 369, 1010-1014; Gottlieb, R. L. et al. (2021) JAMA 325, 632-644).

Structural analysis revealed that REGN10933, tightly contacted with variant residues K417 and E484 burying a total surface area of 77 Å² and 64.81 Å², respectively, and made a weak contact with residue N501. Antibody LY-COV555, formed a very strong contact with residue E484 burying a total interfacial area of 97.3 Å² (FIG. 7D). In contrast, COV-2125 and S2E12 are heavily shifted towards one side of the RBD epitope encircling a minimal ACE2 epitope, thereby weakly contacting residues K417 (BSA 44.0 Å² for 2125 and 30.3 Å² for S2E12) and E484 (BSA 34.7 Å² for 2125 and 39.4 Å² for S2E12), COV-2173 mAb forms a strong contact with residue E484 (BSA 94.7 Å²) and minor contacts with N501 (BSA 3.6 Å²) (FIG. 7d). These structural data highlight the advantages of the COV-2173 and -2125 epitopes that can potentially be exploited for next generation vaccine development and therapeutic use.

In this study, we isolated potent neutralizing monoclonal antibodies targeting the NTD supersite and RBD on the surface of the viral Spike glycoprotein, adding to the current arsenal of potent neutralizing antibodies described (Barnes, C. O. et al. (2020) Nature). In addition to neutralization, both NTD and RBD-targeting mAbs were capable of mediating Fc effectors functions, with a unique ability of NTD neutralizing mAbs to leverage complement deposition. Since these NTD and RBD mAbs do not compete for binding to the S trimer, several combinations of NTD and RBD mAbs were tested for neutralization and in vivo protection. Cocktails of NTD and RBD mAbs demonstrated additive effects on viral neutralization and Fc effector functions in vitro and yielded potent in vivo prophylactic and therapeutic protection. Prophylactic sterilizing protection was observed at a low dose of 20 μg (1 mg kg⁻¹), and partial protection at a 5 μg (0.25 mg kg⁻¹) dose, while therapeutic protection was provided at 2.5 mg kg⁻¹. Prophylactic in vivo protection by NTD-, but not RBD-targeting mAbs, required an intact IgG Fc domain, underlining the importance of Fc effector functions for NTD-targeting mAbs in mediating protection. Along with ADNP, engagement of complement (ADCD) was associated with survival of Covid-19, and collaboration between Fab and Fc effector functions has been shown to be beneficial for vaccine-elicited protection (Gorman, M. J., et al. (2021) bioRxiv).

Structural analyses revealed these mAbs targeted several epitopes within the NTD supersite and the RBD. COV NTD mAbs share epitope similarities to other IGHV1-24 mAbs described previously (Chi, X. et al. (2020) Science 369, 650-655), with COV-2039, -2025 and -004 (using IGHV1-2) among the most potent of the class. RBD mAbs COV-2173 and -2123 are most similar to RBD class 1 mAbs targeting the receptor binding motif (RBM). COV-2151 largely overlaps with class 3 S309 and CR3022 epitopes, and as such, displayed broad efficacy against SARS-CoV-2 strains. COV-2057 targets a unique RBD epitope on the opposite side of RBD. These mAbs originated from multiple different B cell lineages, indicating that SARS-CoV-2 infection was able to induce neutralizing antibodies through multiple genetic pathways, including public lineages such as IGVH1-24 and IGVH3-53 for NTD and RBD antibodies, respectively. Consistent with previous studies, the majority of isolated SARS-CoV-2 mAbs were close to germline sequences, in agreement with the observation that germline encoded residues play a significant role in binding of several potent neutralizing antibodies across multiple classes (Yuan, M. et al. (2020) Science 369, 1119-1123).

As VOC such as B.1.1.7, B.1.351 and P.1, harboring multiple mutations in both N-terminal (NTD) and Receptor Binding (RBD) domains, continue to escape first-generation monoclonal antibody therapeutics (Madhi, S. A. et al. (2021) The New England journal of medicine; Zhou, D. et al. (2021) Cell), there is a need for prophylactic and therapeutic mAbs with broad and potent activity against all circulating SARS-CoV-2 strains. Most studies have largely focused on RBD and NTD antibodies separately, with only a few describing potential advantages of combining the two classes of mAbs as a two- or even three-mAb combinations to provide additional coverage (Suryadevara, N. et al. (2021) Cell; Sun, Y. et al. (2021) Cell Res 31, 597-600). Although COV mAbs retained binding activities against current circulating VOC, SARS-CoV-2 variants B.1.1.7, B.1.351 and B.1.427/429 mostly evaded neutralization by NTD mAbs, and multiple mutations present within the B.1.351 strain affected neutralization by some of the most potent COV RBD mAbs. Remarkably, RBD mAb COV-2125, which targets residue F486 in RBD, demonstrated potent neutralizing activity against all SARS-CoV-2 VOC tested and, in combination with NTD and other RBD mAbs, was able to prevent viral escape. COV-2125 targets a minimal epitope required for ACE2 engagement. Combined, these data demonstrate that NTD/RBD mAb cocktails offer potent protection in vivo and broader coverage across VOC, offering advantages over monoclonal antibodies restricted to targeting only than 1 site of vulnerability on the viral surface.

TABLE 1
Structural Characteristics of the variable heavy chains of the of SARS-CoV-2 Antibodies

| | | | | | Nucleotide | Amino Acid |
|---|---|---|---|---|---|---|
| | | Heavy V | | | Sequence, | Sequence, |
| Antibody | Heavy V | Gene | Heavy D | Heavy J | Variable | Variable |
| Name | Gene | Identity % | Gene | Gene | Heavy Chain | Heavy Chain |
| COV_1007 | IGHV2-26 | 98.66 | IGHD5-12 | IGHJ4 | SEQ ID | SEQ ID |
| | | | | | NO: 1 | NO: 2 |
| COV_1037 | IGHV3-33 | 100 | IGHD2-12 | IGHJ6 | SEQ ID | SEQ ID |
| | | | | | NO: 11 | NO: 12 |
| COV_1045 | IGHV1-46 | 84.75 | IGHD3/OR15-3A; | IGHJ4 | SEQ ID | SEQ ID |
| | | | IGHD3/OR15-3B | | NO: 21 | NO: 22 |
| COV_1046 | IGHV1-46 | 89.15 | IGHD3/OR15-3A; | IGHJ4 | SEQ ID | SEQ ID |
| | | | IGHD3/OR15-3B | | NO: 31 | NO: 32 |
| COV_1201 | IGHV1-2 | 98.99 | IGHD3-10 | IGHJ6 | SEQ ID | SEQ ID |
| | | | | | NO: 41 | NO: 42 |
| COV_2004 | IGHV1-2*06 | 98.31 | IGHD3-10*01 | IGHJ5*02 | SEQ ID | SEQ ID |
| | | | | | NO: 51 | NO: 52 |
| COV_2008 | IGHV1-24*01 | 97.97 | IGHD1-20*01 | IGHJ6*02 | SEQ ID | SEQ ID |
| | | | | | NO: 61 | NO: 62 |
| COV_2014 | IGHV1-24*01 | 99.66 | IGHD3-22*01 | IGHJ4*02 | SEQ ID | SEQ ID |
| | | | | | NO: 71 | NO: 72 |
| COV_2018 | IGHV1-69*01; | 96.92 | IGHD1-26*01 | IGHJ4*02 | SEQ ID | SEQ ID |
| | IGHV1-69D | | | | NO: 81 | NO: 82 |
| COV_2024 | IGHV1-69; | 96.96 | IGHD5-12 | IGHJ4*02 | SEQ ID | SEQ ID |
| | IGHV1-69D | | | | NO: 91 | NO: 92 |
| COV_2025 | IGHV1-24*01 | 95.22 | IGHD2-15*01 | IGHJ5*02 | SEQ ID | SEQ ID |
| | | | | | NO: 101 | NO: 102 |
| COV_2027 | IGHV3-53*01 | 97.59 | IGHD1-26*01 | IGHJ4*02 | SEQ ID | SEQ ID |
| | | | | | NO: 111 | NO: 112 |
| COV_2028 | IGHV4-4*02 | 98.64 | IGHD1-7 | IGHJ4*02 | SEQ ID | SEQ ID |
| | | | | | NO: 121 | NO: 122 |
| COV_2035 | IGHV1-24*01 | 97.28 | IGHD5-18*01 | IGHJ5*02 | SEQ ID | SEQ ID |
| | | | | | NO: 131 | NO: 132 |
| COV_2037 | IGHV1-24*01 | 98.98 | IGHD1-20*01 | IGHJ6*02 | SEQ ID | SEQ ID |
| | | | | | NO: 141 | NO: 142 |
| COV_2039 | IGHV1-24*01 | 97.63 | IGHD4-17*01 | IGHJ4*02 | SEQ ID | SEQ ID |
| | | | | | NO: 151 | NO: 152 |
| COV_2054 | IGHV3-30-3*01 | 95.59 | IGHD3-10*01 | IGHJ4*02 | SEQ ID | SEQ ID |
| | | | | | NO: 161 | NO: 162 |
| COV_2056 | IGHV3-30*18; | 97.62 | IGHD3-22*01 | IGHJ3*02 | SEQ ID | SEQ ID |
| | IGHV3-30-5 | | | | NO: 431 | NO: 432 |
| COV_2057 | IGHV5-51*01 | 97.63 | IGHD3-22*01 | IGHJ4*02 | SEQ ID | SEQ ID |
| | | | | | NO: 171 | NO: 172 |
| COV_2063 | IGHV3-33*01 | 99.32 | IGHD3-10*01 | IGHJ3*02 | SEQ ID | SEQ ID |
| | | | | | NO: 181 | NO: 182 |
| COV_2091 | IGHV3-15*01 | 99.67 | IGHD6-6*01 | IGHJ3*02 | SEQ ID | SEQ ID |
| | | | | | NO: 191 | NO: 192 |
| COV_2100 | IGHV3-30-3*01 | 98.63 | IGHD3-3*01; | IGHJ4*02 | SEQ ID | SEQ ID |
| | | | IGHD5-5 | | NO: 201 | NO: 202 |
| COV_2103 | IGHV3-33*01 | 94.92 | IGHD3-22*01 | IGHJ3*01 | SEQ ID | SEQ ID |
| | | | | | NO: 211 | NO: 212 |
| COV_2108 | IGHV3-30-3*01 | 96.27 | IGHD2-21*02 | IGHJ4*02 | SEQ ID | SEQ ID |
| | | | | | NO: 221 | NO: 222 |
| COV_2123 | IGHV3-30-3*01 | 98.31 | IGHD3-10*01 | IGHJ4*02 | SEQ ID | SEQ ID |
| | | | | | NO: 231 | NO: 232 |
| COV_2125 | IGHV3-30*18; | 98.64 | IGHD3-22*01 | IGHJ1*01 | SEQ ID | SEQ ID |
| | IGHV3-30-5 | | | | NO: 241 | NO: 242 |
| COV_2134 | IGHV3-33*01 | 98.98 | IGHD6-19*01 | IGHJ6*02 | SEQ ID | SEQ ID |
| | | | | | NO: 251 | NO: 252 |
| COV_2151 | IGHV4-39*07 | 96.95 | IGHD3-3*02 | IGHJ4*02 | SEQ ID | SEQ ID |
| | | | | | NO: 261 | NO: 262 |
| COV_2165 | IGHV3-48*04 | 98.98 | IGHD6-25*01 | IGHJ4*02 | SEQ ID | SEQ ID |
| | | | | | NO: 271 | NO: 272 |
| COV_2172 | IGHV3-30*18; | 98.26 | IGHD1-26*01 | IGHJ4*02 | SEQ ID | SEQ ID |
| | IGHV3-30-5 | | | | NO: 441 | NO: 442 |
| COV_2173 | IGHV4-39 | 93.98 | IGHD2-2*01 | IGHJ5*02 | SEQ ID | SEQ ID |
| | | | | | NO: 281 | NO: 282 |
| COV_2193 | IGHV1-24*01 | 95.58 | IGHD2-15*01 | IGHJ4*02 | SEQ ID | SEQ ID |
| | | | | | NO: 291 | NO: 292 |
| COV_2196 | IGHV1-24*01 | 97.96 | IGHD6-25*01 | IGHJ4*02 | SEQ ID | SEQ ID |
| | | | | | NO: 301 | NO: 302 |
| COV_3000 | IGHV3-49 | 94.04 | IGHD3-3 | IGHJ4 | SEQ ID | SEQ ID |
| | | | | | NO: 311 | NO: 312 |
| COV_3005 | IGHV3-23; | 82.77 | IGHD6-6 | IGHJ4 | SEQ ID | SEQ ID |
| | IGHV3-23D | | | | NO: 321 | NO: 322 |
| COV_3013 | IGHV3-49 | 92.72 | IGHD3-3 | IGHJ3 | SEQ ID | SEQ ID |
| | | | | | NO: 331 | NO: 332 |
| COV_3019 | IGHV3-49 | 91.39 | IGHD5/OR15-5A; | IGHJ43 | SEQ ID | SEQ ID |
| | | | IGHD5/OR15-5B | | NO: 341 | NO: 342 |
| COV_3028 | IGHV3-49 | 91.06 | IGHD5/OR15-5A; | IGHJ3 | SEQ ID | SEQ ID |
| | | | IGHD5/OR15-5B | | NO: 351 | NO: 352 |
| COV_3031 | IGHV3-49 | 94.37 | IGHD2-2 | IGHJ3 | SEQ ID | SEQ ID |
| | | | | | NO: 361 | NO: 362 |
| COV_3033 | IGHV3-49 | 84.44 | IGHD2-2 | IGHJ3 | SEQ ID | SEQ ID |
| | | | | | NO: 371 | NO: 372 |
| COV_3037 | IGHV3-49 | 93.71 | IGHD3-3 | IGHJ3 | SEQ ID | SEQ ID |
| | | | | | NO: 381 | NO: 382 |
| COV_3040 | IGHV3-49 | 93.02 | IGHD3-3 | IGHJ3 | SEQ ID | SEQ ID |
| | | | | | NO: 391 | NO: 392 |
| COV_3043 | IGHV3-49 | 93.05 | IGHD2/OR15-2A; | IGHJ3 | SEQ ID | SEQ ID |
| | | | IGHD2/OR15-2B | | NO: 401 | NO: 402 |
| COV_3053 | IGHV3-49 | 89.74 | IGHD3-10 | IGHJ3 | SEQ ID | SEQ ID |
| | | | | | NO: 411 | NO: 412 |
| COV_3088 | IGHV3-13 | 100 | IGHD6-19 | IGHJ4 | SEQ ID | SEQ ID |
| | | | | | NO: 421 | NO: 422 |
| CoV_1012 | IGHV4-4 | 97.95 | IGHD1-26 | IGHJ3 | SEQ ID | SEQ ID |
| | | | | | NO: 451 | NO: 452 |
| CoV_1025 | IGHV3-30-3 | 97.97 | IGHD3-10 | IGHJ6 | SEQ ID | SEQ ID |
| | | | | | NO: 461 | NO: 462 |
| CoV_1032 | IGHV4-59 | 98.97 | IGHD3-10 | IGHJ4 | SEQ ID | SEQ ID |
| | | | | | NO: 471 | NO: 472 |
| CoV_1050 | IGHV3-30-3 | 96.93 | IGHD3-22 | IGHJ1; IGHJ4; | SEQ ID | SEQ ID |
| | | | | IGHJ5 | NO: 481 | NO: 482 |
| CoV_1056 | IGHV3-30; | 92.83 | IGHD2-21 | IGHJ5 | SEQ ID | SEQ ID |
| | IGHV3-30-5; | | | | NO: 491 | NO: 492 |
| | IGHV3-33 | | | | | |
| CoV_1060 | IGHV5-51 | 97.28 | IGHD2-8 | IGHJ6 | SEQ ID | SEQ ID |
| | | | | | NO: 501 | NO: 502 |
| CoV_1063 | IGHV3-7 | 85.76 | IGHD2-2; | IGHJ4; IGHJ5 | SEQ ID | SEQ ID |
| | | | IGHD2/OR15-2A; | | NO: 511 | NO: 512 |
| | | | IGHD2/OR15-2B | | | |
| CoV_1071 | IGHV1-46 | 85.08 | IGHD3/OR15-3A; | IGHJ4 | SEQ ID | SEQ ID |
| | | | IGHD3/OR15-3B | | NO: 521 | NO: 522 |
| CoV_1076 | IGHV3-11 | 92.86 | IGHD7-27 | IGHJ4; IGHJ5 | SEQ ID | SEQ ID |
| | | | | | NO: 531 | NO: 532 |
| CoV_1082 | IGHV3-48 | 87.38 | IGHD3-3; | IGHJ5 | SEQ ID | SEQ ID |
| | | | IGHD3-9 | | NO: 541 | NO: 542 |
| CoV_1085 | IGHV3-33 | 97.3 | IGHD6-19 | IGHJ4 | SEQ ID | SEQ ID |
| | | | | | NO: 551 | NO: 552 |
| CoV_1086 | IGHV3-23; | 91.55 | IGHD6-19 | IGHJ4 | SEQ ID | SEQ ID |
| | IGHV3-23D | | | | NO: 561 | NO: 562 |
| CoV_1087 | IGHV1-46 | 87.8 | IGHD3/OR15-3A; | IGHJ4 | SEQ ID | SEQ ID |
| | | | IGHD3/OR15-3B | | NO: 571 | NO: 572 |
| CoV_1097 | IGHV4-59 | 98.63 | IGHD2-8 | IGHJ6 | SEQ ID | SEQ ID |
| | | | | | NO: 581 | NO: 582 |
| CoV_1116 | IGHV3-21 | 98.31 | IGHD6-6 | IGHJ6 | SEQ ID | SEQ ID |
| | | | | | NO: 591 | NO: 592 |
| CoV_1118 | IGHV3-21 | 97.64 | IGHD3-22 | IGHJ4 | SEQ ID | SEQ ID |
| | | | | | NO: 601 | NO: 602 |
| CoV_1122 | IGHV4-59 | 98.97 | IGHD3-22 | IGHJ6 | SEQ ID | SEQ ID |
| | | | | | NO: 611 | NO: 612 |
| CoV_1131 | IGHV4-59 | 98.29 | IGHD3-10 | IGHJ3 | SEQ ID | SEQ ID |
| | | | | | NO: 621 | NO: 622 |
| CoV_1136 | IGHV3-21 | 96.96 | IGHD3-22 | IGHJ4 | SEQ ID | SEQ ID |
| | | | | | NO: 631 | NO: 632 |
| CoV_1144 | IGHV3-7 | 96.61 | IGHD2-8 | IGHJ4 | SEQ ID | SEQ ID |
| | | | | | NO: 641 | NO: 642 |
| CoV_1145 | IGHV3-33 | 99.32 | IGHD1-26 | IGHJ4 | SEQ ID | SEQ ID |
| | | | | | NO: 651 | NO: 652 |
| CoV_1149 | IGHV3-30; | 100 | IGHD5-18; | IGHJ4; IGHJ5 | SEQ ID | SEQ ID |
| | IGHV3-30-5 | | IGHD5-5 | | NO: 661 | NO: 662 |
| CoV_1151 | IGHV4-34 | 96.59 | IGHD4-17 | IGHJ6 | SEQ ID | SEQ ID |
| | | | | | NO: 671 | NO: 672 |
| CoV_1154 | IGHV3-21 | 96.62 | IGHD3-22 | IGHJ4 | SEQ ID | SEQ ID |
| | | | | | NO: 681 | NO: 682 |
| CoV_1165 | IGHV3-33 | 91.89 | IGHD2/OR15-2A; | IGHJ4 | SEQ ID | SEQ ID |
| | | | IGHD2/OR15-2B | | NO: 691 | NO: 692 |
| CoV_1166 | IGHV3-7 | 97.97 | IGHD1-26 | IGHJ3 | SEQ ID | SEQ ID |
| | | | | | NO: 701 | NO: 702 |
| CoV_1170 | IGHV3-30-3 | 98.98 | IGHD1-14 | IGHJ5 | SEQ ID | SEQ ID |
| | | | | | NO: 711 | NO: 712 |
| CoV_1172 | IGHV4-59 | 97.95 | IGHD2-15 | IGHJ3 | SEQ ID | SEQ ID |
| | | | | | NO: 721 | NO: 722 |
| CoV_1177 | IGHV3-33 | 100 | IGHD6-19 | IGHJ4 | SEQ ID | SEQ ID |
| | | | | | NO: 731 | NO: 732 |
| CoV_1184 | IGHV4-34 | 99.32 | IGHD3-22 | IGHJ5 | SEQ ID | SEQ ID |
| | | | | | NO: 741 | NO: 742 |
| CoV_1198 | IGHV3-64D | 90.66 | IGHD4-11; | IGHJ4 | SEQ ID | SEQ ID |
| | | | IGHD4-4 | | NO: 751 | NO: 752 |
| CoV_2032 | IGHV3-74 | 97.3 | IGHD2-8 | IGHJ4 | SEQ ID | SEQ ID |
| | | | | | NO: 761 | NO: 762 |
| CoV_2048 | IGHV4-39 | 96.66 | IGHD4-17 | IGHJ4 | SEQ ID | SEQ ID |
| | | | | | NO: 771 | NO: 772 |
| CoV_2055 | IGHV3-30*018; | 97.96 | IGHD3-22*01/ | IGHJ4*02 | SEQ ID | SEQ ID |
| | IGHV3-30-5 | | OR15-2A; | | NO: 781 | NO: 782 |
| | | | IGHD2/OR15-2B | | | |
| CoV_2056 | IGHV3-30; | 97.62 | IGHD3-22 | IGHJ3 | SEQ ID | SEQ ID |
| | IGHV3-30-5 | | | | NO: 791 | NO: 792 |
| CoV_2064 | IGHV3-74 | 93.2 | IGHD3-9 | IGHJ6 | SEQ ID | SEQ ID |
| | | | | | NO: 801 | NO: 802 |
| CoV_2066 | IGHV4-38-2 | 86.78 | IGHD6-13; | IGHJ4 | SEQ ID | SEQ ID |
| | | | IGHD6-19; | | NO: 811 | NO: 812 |
| | | | IGHD6-25 | | | |
| CoV_2077 | IGHV4-59 | 99.66 | IGHD1-7 | IGHJ4 | SEQ ID | SEQ ID |
| | | | | | NO: 821 | NO: 822 |
| CoV_2093 | IGHV3-7 | 96.28 | IGHD3-3 | IGHJ6 | SEQ ID | SEQ ID |
| | | | | | NO: 831 | NO: 832 |
| CoV_2137 | IGHV3-21*01 | 97.97 | IGHD1-14*01 | IGHJ2*01 | SEQ ID | SEQ ID |
| | | | | | NO: 841 | NO: 842 |
| CoV_2143 | IGHV3-30; | 96.94 | IGHD3-22 | IGHJ3 | SEQ ID | SEQ ID |
| | IGHV3-30-3 | | | | NO: 851 | NO: 852 |
| CoV_2169 | IGHV3-30-3 | 96.18 | IGHD3-22 | IGHJ3 | SEQ ID | SEQ ID |
| | | | | | NO: 861 | NO: 862 |
| CoV_2172 | IGHV3-30; | 96.26 | IGHD1-26 | IGHJ4 | SEQ ID | SEQ ID |
| | IGHV3-30-5 | | | | NO: 871 | NO: 872 |
| CoV_2174 | IGHV3-11*04 | 99.66 | IGHD2-2*01 | IGHJ6*02 | SEQ ID | SEQ ID |
| | | | | | NO: 881 | NO: 882 |
| CoV_2205 | IGHV3-11 | 96.94 | IGHD3-9 | IGHJ4 | SEQ ID | SEQ ID |
| | | | | | NO: 891 | NO: 892 |
| CoV_2215 | IGHV3-15 | 99 | IGHD3-22 | IGHJ3 | SEQ ID | SEQ ID |
| | | | | | NO: 901 | NO: 902 |
| CoV_3049 | IGHV3-49 | 92.72 | IGHD2-15; | IGHJ3 | SEQ ID | SEQ ID |
| | | | IGHD2-2 | | NO: 911 | NO: 912 |
| CoV_3069 | IGHV3-21 | 95.25 | IGHD3-22 | IGHJ4 | SEQ ID | SEQ ID |
| | | | | | NO: 921 | NO: 922 |
| CoV_3077 | IGHV4-38-2 | 100 | IGHD1-1; | IGHJ3 | SEQ ID | SEQ ID |
| | | | IGHD2-15; | | NO: 931 | NO: 932 |
| | | | IGHD2-2; | | | |
| | | | IGHD2-8; | | | |
| | | | IGHD2/OR15-2A; | | | |
| | | | IGHD2/OR15-2B; | | | |
| | | | IGHD5-24 | | | |
| CoV_3079 | IGHV3-30-3 | 100 | IGHD1-26 | IGHJ6 | SEQ ID | SEQ ID |
| | | | | | NO: 941 | NO: 942 |
| CoV_3100 | IGHV3-49 | 91.39 | IGHD2-2 | IGHJ3 | SEQ ID | SEQ ID |
| | | | | | NO: 951 | NO: 952 |
| CoV_3103 | IGHV3-9 | 86.78 | IGHD3-10 | IGHJ6 | SEQ ID | SEQ ID |
| | | | | | NO: 961 | NO: 962 |
| CoV_3129 | IGHV4-34 | 92.23 | IGHD2-8 | IGHJ4 | SEQ ID | SEQ ID |
| | | | | | NO: 971 | NO: 972 |
| CoV_3137 | IGHV3-33 | 83.88 | IGHD6-19 | IGHJ4 | SEQ ID | SEQ ID |
| | | | | | NO: 981 | NO: 982 |

TABLE 2
Structural Characteristics of the variable
light chain of the of SARS-CoV-2 Antibodies

| | | | | Nucleotide | Amino Acid |
|---|---|---|---|---|---|
| | | Light V | | Sequence, | Sequence, |
| Antibody | Light V | Gene | Light J | Variable | Variable |
| Name | Gene | % Identity | Gene | Light Chain | Light Chain |
| COV_1007 | IGKV1D-12 | 98.23 | IGKJ4 | SEQ ID NO: 6 | SEQ ID NO: 7 |
| COV_1037 | IGKV2-28; | 100 | IGKJ3 | SEQ ID NO: 16 | SEQ ID NO: 17 |
| | IGKV2D-28 | | | | |
| COV_1045 | IGKV2-24 | 95.02 | IGKJ1 | SEQ ID NO: 26 | SEQ ID NO: 27 |
| COV_1046 | IGKV2-24 | 96.68 | IGKJ1 | SEQ ID NO: 36 | SEQ ID NO: 37 |
| COV_1201 | IGLV2-14 | 99.32 | IGLJ1 | SEQ ID NO: 46 | SEQ ID NO: 47 |
| COV_2004 | IGKV1D-16 | 99.3 | IGKJ2 | SEQ ID NO: 56 | SEQ ID NO: 57 |
| COV_2008 | IGKV2-24 | 99.33 | IGKJ2 | SEQ ID NO: 66 | SEQ ID NO: 67 |
| COV_2014 | IGKV1-9 | 98.25 | IGKJ5 | SEQ ID NO: 76 | SEQ ID NO: 77 |
| COV_2018 | IGKV3-11 | 97.21 | IGKJ1 | SEQ ID NO: 86 | SEQ ID NO: 87 |
| COV_2024 | IGKV3-11 | 98.26 | IGKJ1 | SEQ ID NO: 96 | SEQ ID NO: 97 |
| COV_2025 | IGLV2-14 | 97.64 | IGLJ1 | SEQ ID NO: 106 | SEQ ID NO: 107 |
| COV_2027 | IGKV3-20 | 100 | IGKJ2 | SEQ ID NO: 116 | SEQ ID NO: 117 |
| COV_2028 | IGLV2-23 | 97.97 | IGLJ3 | SEQ ID NO: 126 | SEQ ID NO: 127 |
| COV_2035 | IGLV2-8 | 98.97 | IGLJ2; | SEQ ID NO: 136 | SEQ ID NO: 137 |
| | | | IGLJ3 | | |
| COV_2037 | IGKV2-24 | 98.33 | IGKJ2 | SEQ ID NO: 146 | SEQ ID NO: 147 |
| COV_2039 | IGLV1-40 | 98.33 | IGLJ2; | SEQ ID NO: 156 | SEQ ID NO: 157 |
| | | | IGLJ3 | | |
| COV_2054 | IGKV1-5 | 97.51 | IGKJ2 | SEQ ID NO: 166 | SEQ ID NO: 167 |
| COV_2056 | IGKV1-12 | 99.3 | IGKJ4 | SEQ ID NO: 436 | SEQ ID NO: 437 |
| COV_2057 | IGKV1-39; | 98.25 | IGKJ3 | SEQ ID NO: 176 | SEQ ID NO: 177 |
| | IGKV1D-39 | | | | |
| COV_2063 | IGKV2-30 | 99 | IGKJ4 | SEQ ID NO: 186 | SEQ ID NO: 187 |
| COV_2091 | IGKV1-39; | 100 | IGKJ4 | SEQ ID NO: 196 | SEQ ID NO: 197 |
| | IGKV1D-39 | | | | |
| COV_2100 | IGKV1-39; | 96.48 | IGKJ4 | SEQ ID NO: 206 | SEQ ID NO: 207 |
| | IGKV1D-39 | | | | |
| COV_2103 | IGKV1-5 | 97.54 | IGKJ3 | SEQ ID NO: 216 | SEQ ID NO: 217 |
| COV_2108 | IGKV1-39; | 98.26 | IGKJ4 | SEQ ID NO: 226 | SEQ ID NO: 227 |
| | IGKV1D-39 | | | | |
| COV_2123 | IGKV1-33; | 98.95 | IGKJ4 | SEQ ID NO: 236 | SEQ ID NO: 237 |
| | IGKV1D-33 | | | | |
| COV_2125 | IGKV1-39; | 96.5 | IGKJ1 | SEQ ID NO: 246 | SEQ ID NO: 247 |
| | IGKV1D-39 | | | | |
| COV_2134 | IGLV3-1 | 99.65 | IGLJ2; | SEQ ID NO: 256 | SEQ ID NO: 257 |
| | | | IGLJ3 | | |
| COV_2151 | IGLV6-57 | 98.98 | IGLJ3 | SEQ ID NO: 266 | SEQ ID NO: 267 |
| COV_2165 | IGKV3-11 | 98.6 | IGKJ3 | SEQ ID NO: 276 | SEQ ID NO: 277 |
| COV_2172 | IGLV2-11 | 100 | IGLJ3 | SEQ ID NO: 446 | SEQ ID NO: 447 |
| COV_2173 | IGLV1-40 | 97.99 | IGLJ2; | SEQ ID NO: 286 | SEQ ID NO: 287 |
| | | | IGLJ3 | | |
| COV_2193 | IGLV3-19 | 97.58 | IGLJ3 | SEQ ID NO: 296 | SEQ ID NO: 297 |
| COV_2196 | IGLV3-27 | 95.94 | IGLJ3 | SEQ ID NO: 306 | SEQ ID NO: 307 |
| COV_3000 | IGKV2D-29 | 93.36 | IGKJ1 | SEQ ID NO: 316 | SEQ ID NO: 317 |
| COV_3005 | IGKV1-17 | 89.2 | IGKJ1 | SEQ ID NO: 326 | SEQ ID NO: 327 |
| COV_3013 | IGKV2D-29 | 95.35 | IGKJ1 | SEQ ID NO: 336 | SEQ ID NO: 337 |
| COV_3019 | IGKV2D-29 | 91.36 | IGKJ1 | SEQ ID NO: 346 | SEQ ID NO: 347 |
| COV_3028 | IGKV2D-29 | 89.7 | IGKJ1 | SEQ ID NO: 356 | SEQ ID NO: 357 |
| COV_3031 | IGKV2D-29 | 93.36 | IGKJ1 | SEQ ID NO: 366 | SEQ ID NO: 367 |
| COV_3033 | IGKV2D-29 | 90.37 | IGKJ1 | SEQ ID NO: 376 | SEQ ID NO: 377 |
| COV_3037 | IGKV2D-29 | 92.36 | IGKJ1 | SEQ ID NO: 386 | SEQ ID NO: 387 |
| COV_3040 | IGKV2-29; | 93.36 | IGKJ1 | SEQ ID NO: 396 | SEQ ID NO: 397 |
| | IGKV2D-29 | | | | |
| COV_3043 | IGKV2D-29 | 92.36 | IGKJ1 | SEQ ID NO: 406 | SEQ ID NO: 407 |
| COV_3053 | IGKV4-1 | 93.42 | IGKJ1 | SEQ ID NO: 416 | SEQ ID NO: 417 |
| COV_3088 | IGLV3-21 | 98.96 | IGLJ3 | SEQ ID NO: 426 | SEQ ID NO: 427 |
| CoV_1012 | IGLV1-47 | 99.66 | IGLJ2; | SEQ ID NO: 456 | SEQ ID NO: 457 |
| | | | IGLJ3 | | |
| CoV_1025 | IGKV1-39; | 100 | IGKJ4 | SEQ ID NO: 466 | SEQ ID NO: 467 |
| | IGKV1D-39 | | | | |
| CoV_1032 | IGKV1-39; | 95.32 | IGKJ1 | SEQ ID NO: 476 | SEQ ID NO: 477 |
| | IGKV1D-39 | | | | |
| CoV_1050 | IGLV3-25 | 93.62 | IGLJ3 | SEQ ID NO: 486 | SEQ ID NO: 487 |
| CoV_1056 | IGLV7-46 | 94.86 | IGLJ1 | SEQ ID NO: 496 | SEQ ID NO: 497 |
| CoV_1060 | IGLV2-14 | 97.98 | IGLJ3 | SEQ ID NO: 506 | SEQ ID NO: 507 |
| CoV_1063 | IGKV1-27 | 88.73 | IGKJ1 | SEQ ID NO: 516 | SEQ ID NO: 517 |
| CoV_1071 | IGKV2-24 | 95.02 | IGKJ1 | SEQ ID NO: 526 | SEQ ID NO: 527 |
| CoV_1076 | IGKV4-1 | 95.36 | IGKJ4 | SEQ ID NO: 536 | SEQ ID NO: 537 |
| CoV_1082 | IGKV2-24 | 94.02 | IGKJ1 | SEQ ID NO: 546 | SEQ ID NO: 547 |
| CoV_1085 | IGLV1-47 | 99.32 | IGLJ7 | SEQ ID NO: 556 | SEQ ID NO: 557 |
| CoV_1086 | IGKV2-24 | 97.67 | IGKJ2 | SEQ ID NO: 566 | SEQ ID NO: 567 |
| CoV_1087 | IGKV2-24 | 96.35 | IGKJ1 | SEQ ID NO: 576 | SEQ ID NO: 577 |
| CoV_1097 | IGLV1-40 | 97.99 | IGLJ3 | SEQ ID NO: 586 | SEQ ID NO: 587 |
| CoV_1116 | IGKV1-39; | 98.26 | IGKJ4 | SEQ ID NO: 596 | SEQ ID NO: 597 |
| | IGKV1D-39 | | | | |
| CoV_1118 | IGKV1-5 | 98.24 | IGKJ1 | SEQ ID NO: 606 | SEQ ID NO: 607 |
| CoV_1122 | IGLV1-44 | 98.99 | IGLJ3 | SEQ ID NO: 616 | SEQ ID NO: 617 |
| CoV_1131 | IGKV1-39; | 98.6 | IGKJ1 | SEQ ID NO: 626 | SEQ ID NO: 627 |
| | IGKV1D-39 | | | | |
| CoV_1136 | IGKV1-5 | 97.54 | IGKJ1 | SEQ ID NO: 636 | SEQ ID NO: 637 |
| CoV_1144 | IGKV1-39; | 98.24 | IGKJ1 | SEQ ID NO: 646 | SEQ ID NO: 647 |
| | IGKV1D-39 | | | | |
| CoV_1145 | IGLV3-10 | 98.97 | IGLJ3 | SEQ ID NO: 656 | SEQ ID NO: 657 |
| CoV_1149 | IGLV2-14 | 100 | IGLJ2; | SEQ ID NO: 666 | SEQ ID NO: 667 |
| | | | IGLJ3 | | |
| CoV_1151 | IGKV3-20 | 98.26 | IGKJ1 | SEQ ID NO: 676 | SEQ ID NO: 677 |
| CoV_1154 | IGKV1-5 | 97.89 | IGKJ1 | SEQ ID NO: 686 | SEQ ID NO: 687 |
| CoV_1165 | IGLV2-11 | 96.24 | IGLJ2; | SEQ ID NO: 696 | SEQ ID NO: 697 |
| | | | IGLJ3 | | |
| CoV_1166 | IGKV1-39; | 99.65 | IGKJ1 | SEQ ID NO: 706 | SEQ ID NO: 707 |
| | IGKV1D-39 | | | | |
| CoV_1170 | IGKV1-9 | 100 | IGKJ3 | SEQ ID NO: 716 | SEQ ID NO: 717 |
| CoV_1172 | IGKV1-39; | 98.24 | IGKJ3 | SEQ ID NO: 726 | SEQ ID NO: 727 |
| | IGKV1D-39 | | | | |
| CoV_1177 | IGLV3-21 | 100 | IGLJ3 | SEQ ID NO: 736 | SEQ ID NO: 737 |
| CoV_1184 | IGKV3-15 | 100 | IGKJ5 | SEQ ID NO: 746 | SEQ ID NO: 747 |
| CoV_1198 | IGKV3-15 | 97.54 | IGKJ2 | SEQ ID NO: 756 | SEQ ID NO: 757 |
| CoV_2032 | IGLV3-1 | 98.6 | IGLJ3 | SEQ ID NO: 766 | SEQ ID NO: 767 |
| CoV_2048 | IGLV2-11 | 99.66 | IGLJ2; | SEQ ID NO: 776 | SEQ ID NO: 777 |
| | | | IGLJ3 | | |
| CoV_2055 | IGKV2-30 | 100 | IGKJ5 | SEQ ID NO: 786 | SEQ ID NO: 787 |
| CoV_2056 | IGKV1-12 | 99.3 | IGKJ4 | SEQ ID NO: 796 | SEQ ID NO: 797 |
| CoV_2064 | IGKV1-5 | 92.96 | IGKJ2 | SEQ ID NO: 806 | SEQ ID NO: 807 |
| CoV_2066 | IGKV4-1 | 97.33 | IGKJ3 | SEQ ID NO: 816 | SEQ ID NO: 817 |
| CoV_2077 | IGKV1-39; | 99.3 | IGKJ2 | SEQ ID NO: 826 | SEQ ID NO: 827 |
| | IGKV1D-39 | | | | |
| CoV_2093 | IGKV1-39; | 97.9 | IGKJ4 | SEQ ID NO: 836 | SEQ ID NO: 837 |
| | IGKV1D-39 | | | | |
| CoV_2137 | IGLV2-23 | 98.99 | IGLJ1 | SEQ ID NO: 846 | SEQ ID NO: 847 |
| CoV_2143 | IGLV1-40 | 98.99 | IGLJ3 | SEQ ID NO: 856 | SEQ ID NO: 857 |
| CoV_2169 | IGKV3-20 | 98.24 | IGKJ2 | SEQ ID NO: 866 | SEQ ID NO: 867 |
| CoV_2172 | IGLV2-11 | 100 | IGLJ3 | SEQ ID NO: 876 | SEQ ID NO: 877 |
| CoV_2174 | IGKV1-16 | 99.65 | IGKJ4 | SEQ ID NO: 886 | SEQ ID NO: 887 |
| CoV_2205 | IGLV1-44 | 99.32 | IGLJ2; | SEQ ID NO: 896 | SEQ ID NO: 897 |
| | | | IGLJ3 | | |
| CoV_2215 | IGLV3-10 | 100 | IGLJ2; | SEQ ID NO: 906 | SEQ ID NO: 907 |
| | | | IGLJ3 | | |
| CoV_3049 | IGKV2-29; | 91.69 | IGKJ1 | SEQ ID NO: 916 | SEQ ID NO: 917 |
| | IGKV2D-29 | | | | |
| CoV_3069 | IGKV1-5 | 96.5 | IGKJ1 | SEQ ID NO: 926 | SEQ ID NO: 927 |
| CoV_3077 | IGKV1-5 | 99.65 | IGKJ4 | SEQ ID NO: 936 | SEQ ID NO: 937 |
| CoV_3079 | IGKV1-39; | 99.65 | IGKJ4 | SEQ ID NO: 946 | SEQ ID NO: 947 |
| | IGKV1D-39 | | | | |
| CoV_3100 | IGKV2D-29 | 92.03 | IGKJ1 | SEQ ID NO: 956 | SEQ ID NO: 957 |
| CoV_3103 | IGKV3-20 | 94.08 | IGKJ2 | SEQ ID NO: 966 | SEQ ID NO: 967 |
| CoV_3129 | IGKV3-11 | 94.01 | IGKJ5 | SEQ ID NO: 976 | SEQ ID NO: 977 |
| CoV_3137 | IGLV2-8 | 91.13 | IGLJ3 | SEQ ID NO: 986 | SEQ ID NO: 987 |

TABLE 3
SARS-CoV-2 Antibody Properties

| | | | KD(pM) | KD(pM) | SARS- |
|---|---|---|---|---|---|
| | Mean | Mean | SARS- | SARS- | CoV-2 |
| Antibody | IC50 | IC80 | CoV2- | CoV2- | Binding |
| Name | (μg/ml) | (μg/ml) | RBD | NTD | Epitope |
| COV_1007 | 9.00 | 25.0 | | | NTD |
| COV_1037 | 25.00 | 25.0 | | | NTD, RBD |
| COV_1045 | 25.00 | 25.0 | | | S2 |
| COV_1046 | 25.00 | 25.0 | | | S2 |
| COV_1201 | 0.09 | 0.30 | | | RBD |
| COV_2004 | 0.04 | 3.64 | | <1.0 | NTD |
| COV_2008 | 0.02 | 0.31 | | <1.0 | NTD |
| COV_2014 | 25.00 | 25.00 | | | NTD |
| COV_2018 | 0.31 | 1.27 | 47.0 | | RBD |
| COV_2024 | 9.59 | 25.0 | | | S2 |
| COV_2025 | 0.02 | 0.49 | | <1.0 | NTD |
| COV_2027 | 0.16 | 0.45 | | | RBD |
| COV_2028 | 25.00 | 25.00 | | 40.7 | NTD |
| COV_2035 | 0.04 | 25.00 | | <1.0 | NTD |
| COV_2037 | 0.07 | 25.00 | | <1.0 | NTD |
| COV_2039 | 0.01 | 1.80 | | <1.0 | NTD |
| COV_2054 | 25.00 | 25.00 | | <1.0 | NTD |
| COV_2056 | 13.20 | 25.00 | | | RBD |
| COV_2057 | 0.97 | 8.99 | <1.0 | | RBD |
| COV_2063 | 0.10 | 7.03 | <1.0 | | RBD |
| COV_2091 | 0.39 | 1.86 | 110.1 | | RBD |
| COV_2100 | 23.03 | 25.00 | 11.4 | | RBD |
| COV_2103 | 25.00 | 25.00 | | <1.0 | NTD |
| COV_2108 | 13.51 | 15.82 | <1.0 | | RBD |
| COV_2123 | 0.00 | 0.03 | 10.9 | | RBD |
| COV_2125 | 0.02 | 0.11 | 10.6 | | RBD |
| COV_2134 | 16.12 | 25.00 | 60.6 | | RBD |
| COV_2151 | 0.12 | 1.21 | <1.0 | | RBD |
| COV_2165 | 0.02 | 0.04 | 10.9 | | RBD |
| COV_2172 | 3.73 | 25.00 | | | RBD |
| COV_2173 | 0.00 | 0.01 | 21.2 | | RBD |
| COV_2193 | 25.00 | 25.0 | | | NTD |
| COV_2196 | 0.02 | 23.37 | | | NTD |
| COV_3000 | 25.00 | 25.00 | | | S2 |
| COV_3005 | 25.00 | 25.00 | | | S2 |
| COV_3013 | 25.00 | 25.00 | | | S2 |
| COV_3019 | 25.00 | 25.00 | | | S2 |
| COV_3028 | 25.00 | 25.00 | | | S2 |
| COV_3031 | 25.00 | 25.00 | | | S2 |
| COV_3033 | 25.00 | 25.00 | | | S2 |
| COV_3037 | 25.00 | 25.00 | | | S2 |
| COV_3040 | 25.00 | 25.00 | | | S2 |
| COV_3043 | 25.00 | 25.00 | | | S2 |
| COV_3053 | | | | | NTD, RBD |
| COV_3088 | 25.00 | 25.0 | | | RBD |
| CoV_1012 | 25 | 25 | | | S2 |
| CoV_1025 | | | | | S2 |
| CoV_1032 | 2.501 | 25 | | | S2 |
| CoV_1050 | 25 | 25 | | | S2 |
| CoV_1056 | 25.00 | 25.00 | | | S2 |
| CoV_1060 | 25.00 | 25.00 | | | S2 |
| CoV_1063 | 25.00 | 25.00 | | | S2 |
| CoV_1071 | 25.00 | 25.00 | | | S2 |
| CoV_1076 | 25.00 | 25.00 | | | S2 |
| CoV_1082 | 25.00 | 25.00 | | | S2 |
| CoV_1085 | | | | | S2 |
| CoV_1086 | 25.00 | 25.00 | | | S2 |
| CoV_1087 | 25.00 | 25.00 | | | S2 |
| CoV_1097 | | | | | S2 |
| CoV_1116 | 6.99 | 25.00 | | | S2 |
| CoV_1118 | | | | | S2 |
| CoV_1122 | | | | | S2 |
| CoV_1131 | 25.00 | 25.00 | | | S2 |
| CoV_1136 | | | | | S2 |
| CoV_1144 | 25.00 | 25.00 | | | S2 |
| CoV_1145 | | | | | S2 |
| CoV_1149 | 25.00 | 25.00 | | | S2 |
| CoV_1151 | 25.00 | 25.00 | | | S2 |
| CoV_1154 | | | | | S2 |
| CoV_1165 | | | | | S2 |
| CoV_1166 | 25.00 | 25.00 | | | S2 |
| CoV_1170 | 0.20 | 25.00 | | | S2 |
| CoV_1172 | 25.00 | 25.00 | | | S2 |
| CoV_1177 | | | | | S2 |
| CoV_1184 | | | | | S2 |
| CoV_1198 | 25.00 | 25.00 | | | S2 |
| CoV_2032 | | | | | S2 |
| CoV_2048 | 20.31 | 25.00 | | | S2 |
| CoV_2055 | | | | | S2 |
| CoV_2056 | 7.46 | 25.00 | | | RBD |
| CoV_2064 | 25.00 | 25.00 | | | S2 |
| CoV_2066 | 25.00 | 25.00 | | | S2 |
| CoV_2077 | 25.00 | 25.00 | | | S2 |
| CoV_2093 | 25.00 | 25.00 | | | S2 |
| CoV_2137 | | | | | NTD |
| CoV_2143 | 25.00 | 25.00 | | | S2 |
| CoV_2169 | | | | | S2 |
| CoV_2172 | 3.987 | 25 | | | RBD |
| CoV_2174 | 0.502 | 2.036 | | | RBD |
| CoV_2205 | | | | | S2 |
| CoV_2215 | | | | | S2 |
| CoV_3049 | | | | | S2 |
| CoV_3069 | | | | | |
| CoV_3077 | 25 | 25 | | | S2 |
| CoV_3079 | 25 | 25 | | | S2 |
| CoV_3100 | | | | | S2 |
| CoV_3103 | | | | | S2 |
| CoV_3129 | | | | | S1 |
| CoV_3137 | | | | | S2 |

TABLE 4
Cross Binding Ability of the SARS-CoV-2 Antibodies

| | Cross- | Cross- | Cross- | Cross- | Cross- | Cross- |
|---|---|---|---|---|---|---|
| Antibody | binding for | binding for | binding for | binding for | binding for | binding for |
| Name | SARS-CoV-1 | MERS-CoV | HCoV-229E | HCoV-HKU1 | HCoV-NL63 | HCoV-OC43 |
| COV_1007 | No | Yes | No | Yes | No | No |
| COV_1037 | Yes | Yes | Yes | Yes | Yes | Yes |
| COV_1045 | Yes | Yes | Yes | Yes | Yes | Yes |
| COV_1046 | Yes | Yes | Yes | Yes | Yes | Yes |
| COV_1201 | No | No | No | No | No | No |
| COV_2004 | No | No | No | No | No | Yes |
| COV_2008 | No | Yes | No | No | No | No |
| COV_2014 | No | No | No | No | No | No |
| COV_2018 | No | Yes | No | Yes | No | No |
| COV_2024 | Yes | Yes | Yes | Yes | Yes | No |
| COV_2025 | No | Yes | No | No | No | No |
| COV_2027 | No | Yes | No | No | No | No |
| COV_2028 | No | Yes | No | No | No | No |
| COV_2035 | No | Yes | No | Yes | No | No |
| COV_2037 | No | Yes | No | No | No | No |
| COV_2039 | No | Yes | No | No | No | No |
| COV_2054 | No | Yes | No | No | No | No |
| COV_2057 | No | Yes | No | No | No | No |
| COV_2063 | Yes | Yes | Yes | Yes | Yes | Yes |
| COV_2091 | No | Yes | No | No | No | No |
| COV_2100 | Yes | Yes | No | No | No | No |
| COV_2103 | No | Yes | No | No | No | No |
| COV_2108 | Yes | Yes | Yes | Yes | Yes | Yes |
| COV_2123 | Yes | Yes | Yes | No | No | No |
| COV_2125 | No | Yes | No | No | No | No |
| COV_2134 | Yes | Yes | No | No | No | No |
| COV_2151 | Yes | Yes | Yes | Yes | Yes | No |
| COV_2056 | Yes | Yes | Yes | Yes | Yes | No |
| COV_2165 | No | Yes | Yes | Yes | Yes | No |
| COV_2173 | No | Yes | No | No | No | No |
| COV_2173 | Yes | Yes | Yes | Yes | Yes | No |
| COV_2193 | Yes | Yes | No | No | No | No |
| COV_2196 | No | Yes | No | No | No | No |
| COV_3000 | Yes | Yes | Yes | Yes | Yes | Yes |
| COV_3005 | Yes | Yes | Yes | Yes | Yes | Yes |
| COV_3013 | Yes | Yes | Yes | Yes | Yes | Yes |
| COV_3019 | Yes | Yes | Yes | Yes | Yes | Yes |
| COV_3028 | Yes | Yes | Yes | Yes | Yes | Yes |
| COV_3031 | Yes | Yes | Yes | Yes | Yes | Yes |
| COV_3033 | Yes | Yes | Yes | Yes | Yes | Yes |
| COV_3037 | Yes | Yes | Yes | Yes | Yes | Yes |
| COV_3040 | Yes | Yes | Yes | Yes | Yes | Yes |
| COV_3043 | Yes | Yes | Yes | Yes | Yes | Yes |
| COV_3053 | No | No | No | No | No | No |
| COV_3088 | No | No | No | No | No | No |
| CoV_1012 | Yes | Yes | No | Yes | Yes | Yes |
| CoV_1025 | Yes | No | No | No | No | No |
| CoV_1032 | Yes | No | No | Yes | Yes | Yes |
| CoV_1050 | No | No | No | No | No | No |
| CoV_1056 | Yes | Yes | Yes | Yes | Yes | Yes |
| CoV_1060 | Yes | Yes | Yes | Yes | Yes | Yes |
| CoV_1063 | No | Yes | Yes | Yes | Yes | Yes |
| CoV_1071 | Yes | Yes | Yes | Yes | Yes | Yes |
| CoV_1076 | Yes | Yes | Yes | Yes | Yes | Yes |
| CoV_1082 | Yes | Yes | Yes | Yes | Yes | Yes |
| CoV_1085 | Yes | Yes | Yes | Yes | Yes | Yes |
| CoV_1086 | Yes | Yes | Yes | Yes | Yes | Yes |
| CoV_1087 | Yes | Yes | Yes | Yes | Yes | Yes |
| CoV_1097 | Yes | Yes | Yes | Yes | Yes | Yes |
| CoV_1116 | Yes | No | No | No | Yes | Yes |
| CoV_1118 | No | No | No | No | No | No |
| CoV_1122 | Yes | No | No | No | No | No |
| CoV_1131 | Yes | No | No | No | Yes | Yes |
| CoV_1136 | No | No | No | No | No | No |
| CoV_1144 | Yes | No | No | No | No | No |
| CoV_1145 | No | No | No | No | No | No |
| CoV_1149 | No | No | No | No | No | No |
| CoV_1151 | Yes | No | No | No | No | No |
| CoV_1154 | No | No | No | No | No | No |
| CoV_1165 | Yes | Yes | Yes | Yes | Yes | Yes |
| CoV_1166 | Yes | No | No | No | No | No |
| CoV_1170 | Yes | No | No | No | No | No |
| CoV_1172 | Yes | No | No | No | No | No |
| CoV_1177 | No | No | No | No | No | No |
| CoV_1184 | No | No | No | No | No | No |
| CoV_1198 | Yes | Yes | Yes | Yes | Yes | Yes |
| CoV_2032 | Yes | No | No | No | No | No |
| CoV_2048 | Yes | No | No | No | No | No |
| CoV_2055 | Yes | No | No | Yes | Yes | Yes |
| CoV_2056 | No | No | No | No | No | No |
| CoV_2064 | Yes | No | No | No | Yes | Yes |
| CoV_2066 | Yes | Yes | Yes | Yes | Yes | Yes |
| CoV_2077 | Yes | No | No | No | No | No |
| CoV_2093 | Yes | No | No | No | No | No |
| CoV_2137 | No | No | No | No | No | No |
| CoV_2143 | Yes | No | No | No | No | No |
| CoV_2169 | No | No | No | No | No | No |
| CoV_2172 | No | No | No | No | No | No |
| CoV_2174 | No | No | No | No | No | No |
| CoV_2205 | No | No | No | No | No | No |
| CoV_2215 | No | No | No | No | No | No |
| CoV_3049 | Yes | Yes | Yes | Yes | Yes | Yes |
| CoV_3069 | No | No | No | No | No | No |
| CoV_3077 | No | No | No | No | No | No |
| CoV_3079 | Yes | No | No | No | No | No |
| CoV_3100 | Yes | Yes | Yes | Yes | Yes | Yes |
| CoV_3103 | No | No | No | No | Yes | Yes |
| CoV_3129 | No | No | No | No | No | No |
| CoV_3137 | Yes | Yes | Yes | Yes | Yes | Yes |

### REFERENCES

1. Gottlieb, R. L. et al. Effect of Bamlanivimab as Monotherapy or in Combination With Etesevimab on Viral Load in Patients With Mild to Moderate COVID-19: A Randomized Clinical Trial. JAMA 325, 632-644 (2021).
2. O'Brien, M. P. et al. Subcutaneous REGEN-COV Antibody Combination to Prevent Covid-19. The New England journal of medicine 385, 1184-1195 (2021).
3. Starr, T. N. et al. SARS-CoV-2 RBD antibodies that maximize breadth and resistance to escape. Nature 597, 97-102 (2021).
4. Dong, J. et al. Genetic and structural basis for SARS-CoV-2 variant neutralization by a two-antibody cocktail. Nat Microbiol 6, 1233-1244 (2021).
5. Martinez, D. R. et al. Prevention and therapy of SARS-CoV-2 and the B.1.351 variant in mice. Cell reports 36, 109450 (2021).
6. Hoffmann, M. et al. SARS-CoV-2 Cell Entry Depends on ACE2 and TMPRSS2 and Is Blocked by a Clinically Proven Protease Inhibitor. Cell 181, 271-280 e278 (2020).
7. McCallum, M. et al. N-terminal domain antigenic mapping reveals a site of vulnerability for SARS-CoV-2. Cell 184, 2332-2347 e2316 (2021).
8. Cerutti, G. et al. Potent SARS-CoV-2 neutralizing antibodies directed against spike N-terminal domain target a single supersite. Cell host & microbe 29, 819-833 e817 (2021).
9. Chen, R. E. et al. Resistance of SARS-CoV-2 variants to neutralization by monoclonal and serum-derived polyclonal antibodies. Nature medicine 27, 717-726 (2021).
10. Planas, D. et al. Reduced sensitivity of SARS-CoV-2 variant Delta to antibody neutralization. Nature 596, 276-280 (2021).
11. Hsieh, C. L. et al. Structure-based design of prefusion-stabilized SARS-CoV-2 spikes. Science 369, 1501-1505 (2020).
12. Ju, B. et al. Human neutralizing antibodies elicited by SARS-CoV-2 infection. Nature 584, 115-119 (2020).
13. Rogers, T. F. et al. Isolation of potent SARS-CoV-2 neutralizing antibodies and protection from disease in a small animal model. Science 369, 956-963 (2020).
14. Robbiani, D. F. et al. Convergent antibody responses to SARS-CoV-2 in convalescent individuals. Nature 584, 437-442 (2020).
15. Zost, S. J. et al. Potently neutralizing and protective human antibodies against SARS-CoV-2. Nature 584, 443-449 (2020).
16. Tortorici, M. A. et al. Ultrapotent human antibodies protect against SARS-CoV-2 challenge via multiple mechanisms. Science 370, 950-957 (2020).
17. Cao, Y. et al. Potent Neutralizing Antibodies against SARS-CoV-2 Identified by High-Throughput Single-Cell Sequencing of Convalescent Patients' B Cells. Cell 182, 73-84 e16 (2020).
18. Joyce, M. G. et al. Efficacy of a Broadly Neutralizing SARS-CoV-2 Ferritin Nanoparticle Vaccine in Nonhuman Primates. bioRxiv htpps://doi.org/10.1101/2021.03.24.436523. (2021).
19. Joyce, M. G. et al. SARS-CoV-2 ferritin nanoparticle vaccines elicit broad SARS coronavirus immunogenicity. bioRxiv htpps://doi.org/10.1101/2021.05.09.443331. (2021).
20. Liu, L. et al. Potent neutralizing antibodies against multiple epitopes on SARS-CoV-2 spike. Nature 584, 450-456 (2020).
21. Sholukh, A. M. et al. Evaluation of cell-based and surrogate SARS-CoV-2 neutralization assays. J Clin Microbiol 59, e0052721 (2021).
22. Suryadevara, N. et al. Neutralizing and protective human monoclonal antibodies recognizing the N-terminal domain of the SARS-CoV-2 spike protein. Cell 184, 2316-2331 e2315 (2021).
23. Winkler, E. S. et al. Human neutralizing antibodies against SARS-CoV-2 require intact Fc effector functions for optimal therapeutic protection. Cell 184, 1804-1820 e1816 (2021).
24. Schafer, A. et al. Antibody potency, effector function, and combinations in protection and therapy for SARS-CoV-2 infection in vivo. The Journal of experimental medicine 218, e20201993 (2021).
25. Ullah, I. et al. Live imaging of SARS-CoV-2 infection in mice reveals that neutralizing antibodies require Fc function for optimal efficacy. Immunity 54, 2143-2158 e2115 (2021).
26. Chi, X. et al. A neutralizing human antibody binds to the N-terminal domain of the Spike protein of SARS-CoV-2. Science 369, 650-655 (2020).
27. Voss, W. N. et al. Prevalent, protective, and convergent IgG recognition of SARS-CoV-2 non-RBD spike epitopes. Science 372, 1108-1112 (2021).
28. Joyce, M. G. et al. A Cryptic Site of Vulnerability on the Receptor Binding Domain of the SARS-CoV-2 Spike Glycoprotein. bioRxiv https://doi.org/10.1101/2020.03.15.992883 (2020).
29. Barnes, C. O. et al. SARS-CoV-2 neutralizing antibody structures inform therapeutic strategies. Nature 588, 682-687 (2020).
30. Dejnirattisai, W. et al. The antigenic anatomy of SARS-CoV-2 receptor binding domain. Cell 184, 2183-2200 e2122 (2021).
31. Liu, H. et al. A combination of cross-neutralizing antibodies synergizes to prevent SARS-CoV-2 and SARS-CoV pseudovirus infection. Cell host & microbe 29, 806-818 e806 (2021).
32. Yuan, M. et al. A highly conserved cryptic epitope in the receptor binding domains of SARS-CoV-2 and SARS-CoV. Science 368, 630-633 (2020).
33. Winkler, E. S. et al. SARS-CoV-2 infection of human ACE2-transgenic mice causes severe lung inflammation and impaired function. Nat Immunol21, 1327-1335 (2020).
34. Oladunni, F. S. et al. Lethality of SARS-CoV-2 infection in K18 human angiotensin-converting enzyme 2 transgenic mice. Nat Commun 11, 6122 (2020).
35. Lo, M. et al. Effector-attenuating Substitutions That Maintain Antibody Stability and Reduce Toxicity in Mice. The Journal of biological chemistry 292, 3900-3908 (2017).
36. Weisblum, Y. et al. Escape from neutralizing antibodies by SARS-CoV-2 spike protein variants. Elife 9, e61312 (2020).
37. Baum, A. et al. Antibody cocktail to SARS-CoV-2 spike protein prevents rapid mutational escape seen with individual antibodies. Science 369, 1014-1018 (2020).
38. Zhou, H. et al. Structural definition of a neutralization epitope on the N-terminal domain of MERS-CoV spike glycoprotein. Nat Commun 10, 3068 (2019).
39. Dussupt, V. et al. Potent Zika and dengue cross-neutralizing antibodies induced by Zika vaccination in a dengue-experienced donor. Nature medicine 26, 228-235 (2020).
40. Wang, L. et al. Ultrapotent antibodies against diverse and highly transmissible SARS-CoV-2 variants. Science 373, eabhl766 (2021).
41. Hansen, J. et al. Studies in humanized mice and convalescent humans yield a SARS-CoV-2 antibody cocktail. Science 369, 1010-1014 (2020).
42. Andreano, E. et al. Extremely potent human monoclonal antibodies from COVID-19 convalescent patients. Cell 184, 1821-1835 (2021).
43. Jones, B. E. et al. The neutralizing antibody, LY-CoV555, protects against SARS-CoV-2 infection in nonhuman primates. Science translational medicine 13, eabfl906 (2021).
44. Sun, Y. et al. Structure-based development of three- and four-antibody cocktails against SARS-CoV-2 via multiple mechanisms. Cell Res 31, 597-600 (2021).
45. Yan, R. et al. Structural basis for bivalent binding and inhibition of SARS-CoV-2 infection by human potent neutralizing antibodies. Cell Res 31, 517-525 (2021).
46. Atyeo, C. et al. Distinct Early Serological Signatures Track with SARS-CoV-2 Survival. Immunity 53, 524-532 e524 (2020).
47. Gorman, M. J. et al. Fab and Fc contribute to maximal protection against SARS-CoV-2 following NVX-CoV2373 subunit vaccine with Matrix-M vaccination. Cell Rep Med 2, 100405 (2021).
48. Yuan, M. et al. Structural basis of a shared antibody response to SARS-CoV-2. Science 369, 1119-1123 (2020).
49. Zhang, L. et al. A proof of concept for neutralizing antibody-guided vaccine design against SARS-CoV-2. Natl Sci Rev., nwab053 (2021).
50. Shi, R. et al. A human neutralizing antibody targets the receptor-binding site of SARS-CoV-2. Nature 584, 120-124 (2020).
51. Brown, E. P. et al. High-throughput, multiplexed IgG subclassing of antigen-specific antibodies from clinical samples. Journal of immunological methods 386, 117-123 (2012).
52. Tomaras, G. D. et al. Initial B-cell responses to transmitted human immunodeficiency virus type 1: virion-binding immunoglobulin M (IgM) and IgG antibodies followed by plasma anti-gp41 antibodies with ineffective control of initial viremia. Journal of virology 82, 12449-12463 (2008).
53. Moore, M. J. et al. Retroviruses pseudotyped with the severe acute respiratory syndrome coronavirus spike protein efficiently infect cells expressing angiotensin-converting enzyme 2. Journal of virology 78, 10628-10635 (2004).
54. Kirchdoerfer, R. N. et al. Stabilized coronavirus spikes are resistant to conformational changes induced by receptor recognition or proteolysis. Scientific reports 8, 15701 (2018).
55. Zhou, T. et al. Structure-Based Design with Tag-Based Purification and In-Process Biotinylation Enable Streamlined Development of SARS-CoV-2 Spike Molecular Probes. Cell reports 33, 108322 (2020).
56. Ackerman, M. E. et al. A robust, high-throughput assay to determine the phagocytic activity of clinical antibody samples. Journal of immunological methods 366, 8-19 (2011).
57. Fischinger, S. et al. A high-throughput, bead-based, antigen-specific assay to assess the ability of antibodies to induce complement activation. Journal of immunological methods 473, 112630 (2019).
58. ter Meulen, J. et al. Human monoclonal antibody combination against SARS coronavirus: synergy and coverage of escape mutants. PLoS Med3, e237 (2006).
59. Davidson, E. & Doranz, B. J. A high-throughput shotgun mutagenesis approach to mapping B-cell antibody epitopes. Immunology 143, 13-20 (2014).
60. Strong, M. et al. Toward the structural genomics of complexes: crystal structure of a PE/PPE protein complex from Mycobacterium tuberculosis. Proceedings of the National Academy of Sciences of the United States ofAmerica 103, 8060-8065 (2006).
61. McCoy, A. J. et al. Phaser crystallographic software. JAppl Crystallogr 40, 658-674 (2007).
62. Emsley, P. & Cowtan, K. Coot: model-building tools for molecular graphics. Acta Crystallogr D Biol Crystallogr 60, 2126-2132 (2004).
63. Adams, P. D., Mustyakimov, M., Afonine, P. V. & Langan, P. Generalized X-ray and neutron crystallographic analysis: more accurate and complete structures for biological macromolecules. Acta Crystallogr D Biol Crystallogr 65, 567-573 (2009).
64. Otwinowski, Z. & Minor, W. Processing of X-ray diffraction data collected in oscillation mode. Methods Enzymol 276, 307-326 (1997).
65. Scheres, S. H. RELION: implementation of a Bayesian approach to cryo-EM structure determination. J Struct Biol 180, 519-530 (2012).
66. Punjani, A., Rubinstein, J. L., Fleet, D. J. & Brubaker, M. A. cryoSPARC: algorithms for rapid unsupervised cryo-EM structure determination. Nat Methods 14, 290-296 (2017).
67. Pettersen, E. F. et al. UCSF Chimera-a visualization system for exploratory research and analysis. J Comput Chem 25, 1605-1612 (2004).

1. An isolated recombinant monoclonal antibody, or an antigen-binding fragment thereof, that binds to a coronavirus, wherein said antibody comprises a heavy chain variable domain and a light chain variable domain,
(1) wherein the heavy chain variable domain comprises a complementarity determining region 1 (CDR1) comprising the amino acid sequence of SEQ ID NO:3; a CDR2 comprising the amino acid sequence of SEQ ID NO:4; and a CDR3 comprising the amino acid sequence of SEQ ID NO:5; and wherein the light chain variable domain comprises: a CDR1 comprising the amino acid sequence of SEQ ID NO:8; a CDR2 comprising the amino acid sequence of SEQ ID NO:9; and a CDR3 comprising the amino acid sequence of SEQ ID NO:10;
(2) wherein the heavy chain variable domain comprises a CDR1 comprising the amino acid sequence of SEQ ID NO:13; a CDR2 comprising the amino acid sequence of SEQ ID NO:14; and a CDR3 comprising the amino acid sequence of SEQ ID NO:15; and wherein the light chain variable domain comprises: a CDR1 comprising the amino acid sequence of SEQ ID NO:18; a CDR2 comprising the amino acid sequence of SEQ ID NO:19; and a CDR3 comprising the amino acid sequence of SEQ ID NO:20;
(3) wherein the heavy chain variable domain comprises a CDR1 comprising the amino acid sequence of SEQ ID NO:23; a CDR2 comprising the amino acid sequence of SEQ ID NO:24; and a CDR3 comprising the amino acid sequence of SEQ ID NO:25; and wherein the light chain variable domain comprises: a CDR1 comprising the amino acid sequence of SEQ ID NO:28; a CDR2 comprising the amino acid sequence of SEQ ID NO:29; and a CDR3 comprising the amino acid sequence of SEQ ID NO:30;
(4) wherein the heavy chain variable domain comprises a CDR1 comprising the amino acid sequence of SEQ ID NO:33; a CDR2 comprising the amino acid sequence of SEQ ID NO:34; and a CDR3 comprising the amino acid sequence of SEQ ID NO:35; and wherein the light chain variable domain comprises: a CDR1 comprising the amino acid sequence of SEQ ID NO:38; a CDR2 comprising the amino acid sequence of SEQ ID NO:39; and a CDR3 comprising the amino acid sequence of SEQ ID NO:40;
(5) wherein the heavy chain variable domain comprises a CDR1 comprising the amino acid sequence of SEQ ID NO:43; a CDR2 comprising the amino acid sequence of SEQ ID NO:44; and a CDR3 comprising the amino acid sequence of SEQ ID NO:45; and wherein the light chain variable domain comprises: a CDR1 comprising the amino acid sequence of SEQ ID NO:48; a CDR2 comprising the amino acid sequence of SEQ ID NO:49; and a CDR3 comprising the amino acid sequence of SEQ ID NO:50;
(6) wherein the heavy chain variable domain comprises a CDR1 comprising the amino acid sequence of SEQ ID NO:53; a CDR2 comprising the amino acid sequence of SEQ ID NO:54; and a CDR3 comprising the amino acid sequence of SEQ ID NO:55; and wherein the light chain variable domain comprises: a CDR1 comprising the amino acid sequence of SEQ ID NO:58; a CDR2 comprising the amino acid sequence of SEQ ID NO:59; and a CDR3 comprising the amino acid sequence of SEQ ID NO:60;
(7) wherein the heavy chain variable domain comprises a CDR1 comprising the amino acid sequence of SEQ ID NO:63; a CDR2 comprising the amino acid sequence of SEQ ID NO:64; and a CDR3 comprising the amino acid sequence of SEQ ID NO:65; and wherein the light chain variable domain comprises: a CDR1 comprising the amino acid sequence of SEQ ID NO:68; a CDR2 comprising the amino acid sequence of SEQ ID NO:69; and a CDR3 comprising the amino acid sequence of SEQ ID NO:70;
(8) wherein the heavy chain variable domain comprises a CDR1 comprising the amino acid sequence of SEQ ID NO:73; a CDR2 comprising the amino acid sequence of SEQ ID NO:74; and a CDR3 comprising the amino acid sequence of SEQ ID NO:75; and wherein the light chain variable domain comprises: a CDR1 comprising the amino acid sequence of SEQ ID NO:78; a CDR2 comprising the amino acid sequence of SEQ ID NO:79; and a CDR3 comprising the amino acid sequence of SEQ ID NO:80;
(9) wherein the heavy chain variable domain comprises a CDR1 comprising the amino acid sequence of SEQ ID NO:83; a CDR2 comprising the amino acid sequence of SEQ ID NO:84; and a CDR3 comprising the amino acid sequence of SEQ ID NO:85; and wherein the light chain variable domain comprises: a CDR1 comprising the amino acid sequence of SEQ ID NO:88; a CDR2 comprising the amino acid sequence of SEQ ID NO:89; and a CDR3 comprising the amino acid sequence of SEQ ID NO:90;
(10) wherein the heavy chain variable domain comprises a CDR1 comprising the amino acid sequence of SEQ ID NO:93; a CDR2 comprising the amino acid sequence of SEQ ID NO:94; and a CDR3 comprising the amino acid sequence of SEQ ID NO:95; and wherein the light chain variable domain comprises: a CDR1 comprising the amino acid sequence of SEQ ID NO:98; a CDR2 comprising the amino acid sequence of SEQ ID NO:99; and a CDR3 comprising the amino acid sequence of SEQ ID NO:100;
(11) wherein the heavy chain variable domain comprises a CDR1 comprising the amino acid sequence of SEQ ID NO:103; a CDR2 comprising the amino acid sequence of SEQ ID NO:104; and a CDR3 comprising the amino acid sequence of SEQ ID NO:105; and wherein the light chain variable domain comprises: a CDR1 comprising the amino acid sequence of SEQ ID NO:108; a CDR2 comprising the amino acid sequence of SEQ ID NO:109; and a CDR3 comprising the amino acid sequence of SEQ ID NO:110;
(12) wherein the heavy chain variable domain comprises a CDR1 comprising the amino acid sequence of SEQ ID NO:113; a CDR2 comprising the amino acid sequence of SEQ ID NO:114; and a CDR3 comprising the amino acid sequence of SEQ ID NO:115; and wherein the light chain variable domain comprises: a CDR1 comprising the amino acid sequence of SEQ ID NO:118; a CDR2 comprising the amino acid sequence of SEQ ID NO:119; and a CDR3 comprising the amino acid sequence of SEQ ID NO:120;
(13) wherein the heavy chain variable domain comprises a CDR1 comprising the amino acid sequence of SEQ ID NO:123; a CDR2 comprising the amino acid sequence of SEQ ID NO:124; and a CDR3 comprising the amino acid sequence of SEQ ID NO:125; and wherein the light chain variable domain comprises: a CDR1 comprising the amino acid sequence of SEQ ID NO:128; a CDR2 comprising the amino acid sequence of SEQ ID NO:129; and a CDR3 comprising the amino acid sequence of SEQ ID NO:130;
(14) wherein the heavy chain variable domain comprises a CDR1 comprising the amino acid sequence of SEQ ID NO:133; a CDR2 comprising the amino acid sequence of SEQ ID NO:134; and a CDR3 comprising the amino acid sequence of SEQ ID NO:135; and wherein the light chain variable domain comprises: a CDR1 comprising the amino acid sequence of SEQ ID NO:138; a CDR2 comprising the amino acid sequence of SEQ ID NO:139; and a CDR3 comprising the amino acid sequence of SEQ ID NO:140;
(15) wherein the heavy chain variable domain comprises a CDR1 comprising the amino acid sequence of SEQ ID NO:143; a CDR2 comprising the amino acid sequence of SEQ ID NO:144; and a CDR3 comprising the amino acid sequence of SEQ ID NO:145; and wherein the light chain variable domain comprises: a CDR1 comprising the amino acid sequence of SEQ ID NO:148; a CDR2 comprising the amino acid sequence of SEQ ID NO149; and a CDR3 comprising the amino acid sequence of SEQ ID NO:150;
(16) wherein the heavy chain variable domain comprises a CDR1 comprising the amino acid sequence of SEQ ID NO:153; a CDR2 comprising the amino acid sequence of SEQ ID NO:154; and a CDR3 comprising the amino acid sequence of SEQ ID NO:155; and wherein the light chain variable domain comprises: a CDR1 comprising the amino acid sequence of SEQ ID NO:158; a CDR2 comprising the amino acid sequence of SEQ ID NO:159; and a CDR3 comprising the amino acid sequence of SEQ ID NO:160;
(17) wherein the heavy chain variable domain comprises a CDR1 comprising the amino acid sequence of SEQ ID NO:163; a CDR2 comprising the amino acid sequence of SEQ ID NO:164; and a CDR3 comprising the amino acid sequence of SEQ ID NO:165; and wherein the light chain variable domain comprises: a CDR1 comprising the amino acid sequence of SEQ ID NO:168; a CDR2 comprising the amino acid sequence of SEQ ID NO:169; and a CDR3 comprising the amino acid sequence of SEQ ID NO:170;
(18) wherein the heavy chain variable domain comprises a CDR1 comprising the amino acid sequence of SEQ ID NO:173; a CDR2 comprising the amino acid sequence of SEQ ID NO:174; and a CDR3 comprising the amino acid sequence of SEQ ID NO:175; and wherein the light chain variable domain comprises: a CDR1 comprising the amino acid sequence of SEQ ID NO:178; a CDR2 comprising the amino acid sequence of SEQ ID NO:179; and a CDR3 comprising the amino acid sequence of SEQ ID NO:180;
(19) wherein the heavy chain variable domain comprises a CDR1 comprising the amino acid sequence of SEQ ID NO:183; a CDR2 comprising the amino acid sequence of SEQ ID NO:184; and a CDR3 comprising the amino acid sequence of SEQ ID NO:185; and wherein the light chain variable domain comprises: a CDR1 comprising the amino acid sequence of SEQ ID NO:188; a CDR2 comprising the amino acid sequence of SEQ ID NO:189; and a CDR3 comprising the amino acid sequence of SEQ ID NO:190;
(20) wherein the heavy chain variable domain comprises a CDR1 comprising the amino acid sequence of SEQ ID NO:193; a CDR2 comprising the amino acid sequence of SEQ ID NO:194; and a CDR3 comprising the amino acid sequence of SEQ ID NO:195; and wherein the light chain variable domain comprises: a CDR1 comprising the amino acid sequence of SEQ ID NO:198; a CDR2 comprising the amino acid sequence of SEQ ID NO:199; and a CDR3 comprising the amino acid sequence of SEQ ID NO:200;
(21) wherein the heavy chain variable domain comprises a CDR1 comprising the amino acid sequence of SEQ ID NO:203; a CDR2 comprising the amino acid sequence of SEQ ID NO:204; and a CDR3 comprising the amino acid sequence of SEQ ID NO:205; and wherein the light chain variable domain comprises: a CDR1 comprising the amino acid sequence of SEQ ID NO:208; a CDR2 comprising the amino acid sequence of SEQ ID NO:209; and a CDR3 comprising the amino acid sequence of SEQ ID NO:210;
(22) wherein the heavy chain variable domain comprises a CDR1 comprising the amino acid sequence of SEQ ID NO:213; a CDR2 comprising the amino acid sequence of SEQ ID NO:214; and a CDR3 comprising the amino acid sequence of SEQ ID NO:215; and wherein the light chain variable domain comprises: a CDR1 comprising the amino acid sequence of SEQ ID NO:218; a CDR2 comprising the amino acid sequence of SEQ ID NO:219; and a CDR3 comprising the amino acid sequence of SEQ ID NO:220;
(23) wherein the heavy chain variable domain comprises a CDR1 comprising the amino acid sequence of SEQ ID NO:223; a CDR2 comprising the amino acid sequence of SEQ ID NO:224; and a CDR3 comprising the amino acid sequence of SEQ ID NO:225; and wherein the light chain variable domain comprises: a CDR1 comprising the amino acid sequence of SEQ ID NO:228; a CDR2 comprising the amino acid sequence of SEQ ID NO:229; and a CDR3 comprising the amino acid sequence of SEQ ID NO:230;
(24) wherein the heavy chain variable domain comprises a CDR1 comprising the amino acid sequence of SEQ ID NO:233; a CDR2 comprising the amino acid sequence of SEQ ID NO:234; and a CDR3 comprising the amino acid sequence of SEQ ID NO:235; and wherein the light chain variable domain comprises: a CDR1 comprising the amino acid sequence of SEQ ID NO:238; a CDR2 comprising the amino acid sequence of SEQ ID NO:239; and a CDR3 comprising the amino acid sequence of SEQ ID NO:240;
(25) wherein the heavy chain variable domain comprises a CDR1 comprising the amino acid sequence of SEQ ID NO:243; a CDR2 comprising the amino acid sequence of SEQ ID NO:244; and a CDR3 comprising the amino acid sequence of SEQ ID NO:245; and wherein the light chain variable domain comprises: a CDR1 comprising the amino acid sequence of SEQ ID NO:248; a CDR2 comprising the amino acid sequence of SEQ ID NO:249; and a CDR3 comprising the amino acid sequence of SEQ ID NO:250;
(26) wherein the heavy chain variable domain comprises a CDR1 comprising the amino acid sequence of SEQ ID NO:253; a CDR2 comprising the amino acid sequence of SEQ ID NO:254; and a CDR3 comprising the amino acid sequence of SEQ ID NO:255; and wherein the light chain variable domain comprises: a CDR1 comprising the amino acid sequence of SEQ ID NO:258; a CDR2 comprising the amino acid sequence of SEQ ID NO:259; and a CDR3 comprising the amino acid sequence of SEQ ID NO:260;
(27) wherein the heavy chain variable domain comprises a CDR1 comprising the amino acid sequence of SEQ ID NO:263; a CDR2 comprising the amino acid sequence of SEQ ID NO:264; and a CDR3 comprising the amino acid sequence of SEQ ID NO:265; and wherein the light chain variable domain comprises: a CDR1 comprising the amino acid sequence of SEQ ID NO:268; a CDR2 comprising the amino acid sequence of SEQ ID NO:269; and a CDR3 comprising the amino acid sequence of SEQ ID NO:270;
(28) wherein the heavy chain variable domain comprises a CDR1 comprising the amino acid sequence of SEQ ID NO:273; a CDR2 comprising the amino acid sequence of SEQ ID NO:274; and a CDR3 comprising the amino acid sequence of SEQ ID NO:275; and wherein the light chain variable domain comprises: a CDR1 comprising the amino acid sequence of SEQ ID NO:278; a CDR2 comprising the amino acid sequence of SEQ ID NO:279; and a CDR3 comprising the amino acid sequence of SEQ ID NO:280;
(29) wherein the heavy chain variable domain comprises a CDR1 comprising the amino acid sequence of SEQ ID NO:283; a CDR2 comprising the amino acid sequence of SEQ ID NO:284; and a CDR3 comprising the amino acid sequence of SEQ ID NO:285; and wherein the light chain variable domain comprises: a CDR1 comprising the amino acid sequence of SEQ ID NO:288; a CDR2 comprising the amino acid sequence of SEQ ID NO:289; and a CDR3 comprising the amino acid sequence of SEQ ID NO:290;
(30) wherein the heavy chain variable domain comprises a CDR1 comprising the amino acid sequence of SEQ ID NO:293; a CDR2 comprising the amino acid sequence of SEQ ID NO:294; and a CDR3 comprising the amino acid sequence of SEQ ID NO:295; and wherein the light chain variable domain comprises: a CDR1 comprising the amino acid sequence of SEQ ID NO:298; a CDR2 comprising the amino acid sequence of SEQ ID NO:299; and a CDR3 comprising the amino acid sequence of SEQ ID NO:300;
(31) wherein the heavy chain variable domain comprises a CDR1 comprising the amino acid sequence of SEQ ID NO:303; a CDR2 comprising the amino acid sequence of SEQ ID NO:304; and a CDR3 comprising the amino acid sequence of SEQ ID NO:305; and wherein the light chain variable domain comprises: a CDR1 comprising the amino acid sequence of SEQ ID NO:308; a CDR2 comprising the amino acid sequence of SEQ ID NO:309; and a CDR3 comprising the amino acid sequence of SEQ ID NO:310;
(32) wherein the heavy chain variable domain comprises a CDR1 comprising the amino acid sequence of SEQ ID NO:313; a CDR2 comprising the amino acid sequence of SEQ ID NO:314; and a CDR3 comprising the amino acid sequence of SEQ ID NO:315; and wherein the light chain variable domain comprises: a CDR1 comprising the amino acid sequence of SEQ ID NO:318; a CDR2 comprising the amino acid sequence of SEQ ID NO:319; and a CDR3 comprising the amino acid sequence of SEQ ID NO:320;
(33) wherein the heavy chain variable domain comprises a CDR1 comprising the amino acid sequence of SEQ ID NO:323; a CDR2 comprising the amino acid sequence of SEQ ID NO:324; and a CDR3 comprising the amino acid sequence of SEQ ID NO:325; and wherein the light chain variable domain comprises: a CDR1 comprising the amino acid sequence of SEQ ID NO:328; a CDR2 comprising the amino acid sequence of SEQ ID NO:329; and a CDR3 comprising the amino acid sequence of SEQ ID NO:330;
(34) wherein the heavy chain variable domain comprises a CDR1 comprising the amino acid sequence of SEQ ID NO:333; a CDR2 comprising the amino acid sequence of SEQ ID NO:334; and a CDR3 comprising the amino acid sequence of SEQ ID NO:335; and wherein the light chain variable domain comprises: a CDR1 comprising the amino acid sequence of SEQ ID NO:338; a CDR2 comprising the amino acid sequence of SEQ ID NO:339; and a CDR3 comprising the amino acid sequence of SEQ ID NO:340;
(35) wherein the heavy chain variable domain comprises a CDR1 comprising the amino acid sequence of SEQ ID NO:343; a CDR2 comprising the amino acid sequence of SEQ ID NO:344; and a CDR3 comprising the amino acid sequence of SEQ ID NO:345; and wherein the light chain variable domain comprises: a CDR1 comprising the amino acid sequence of SEQ ID NO:348; a CDR2 comprising the amino acid sequence of SEQ ID NO:349; and a CDR3 comprising the amino acid sequence of SEQ ID NO:350;
(36) wherein the heavy chain variable domain comprises a CDR1 comprising the amino acid sequence of SEQ ID NO:353; a CDR2 comprising the amino acid sequence of SEQ ID NO:354; and a CDR3 comprising the amino acid sequence of SEQ ID NO:355; and wherein the light chain variable domain comprises: a CDR1 comprising the amino acid sequence of SEQ ID NO:358; a CDR2 comprising the amino acid sequence of SEQ ID NO:359; and a CDR3 comprising the amino acid sequence of SEQ ID NO:360;
(37) wherein the heavy chain variable domain comprises a CDR1 comprising the amino acid sequence of SEQ ID NO:363; a CDR2 comprising the amino acid sequence of SEQ ID NO:364; and a CDR3 comprising the amino acid sequence of SEQ ID NO:365; and wherein the light chain variable domain comprises: a CDR1 comprising the amino acid sequence of SEQ ID NO:368; a CDR2 comprising the amino acid sequence of SEQ ID NO:369; and a CDR3 comprising the amino acid sequence of SEQ ID NO:370;
(38) wherein the heavy chain variable domain comprises a CDR1 comprising the amino acid sequence of SEQ ID NO:373; a CDR2 comprising the amino acid sequence of SEQ ID NO:374; and a CDR3 comprising the amino acid sequence of SEQ ID NO:375; and wherein the light chain variable domain comprises: a CDR1 comprising the amino acid sequence of SEQ ID NO:378; a CDR2 comprising the amino acid sequence of SEQ ID NO:379; and a CDR3 comprising the amino acid sequence of SEQ ID NO:380;
(39) wherein the heavy chain variable domain comprises a CDR1 comprising the amino acid sequence of SEQ ID NO:383; a CDR2 comprising the amino acid sequence of SEQ ID NO:384; and a CDR3 comprising the amino acid sequence of SEQ ID NO:385; and wherein the light chain variable domain comprises: a CDR1 comprising the amino acid sequence of SEQ ID NO:388; a CDR2 comprising the amino acid sequence of SEQ ID NO:389; and a CDR3 comprising the amino acid sequence of SEQ ID NO:390;
(40) wherein the heavy chain variable domain comprises a CDR1 comprising the amino acid sequence of SEQ ID NO:393; a CDR2 comprising the amino acid sequence of SEQ ID NO:394; and a CDR3 comprising the amino acid sequence of SEQ ID NO:395; and wherein the light chain variable domain comprises: a CDR1 comprising the amino acid sequence of SEQ ID NO:398; a CDR2 comprising the amino acid sequence of SEQ ID NO:399; and a CDR3 comprising the amino acid sequence of SEQ ID NO:400;
(41) wherein the heavy chain variable domain comprises a CDR1 comprising the amino acid sequence of SEQ ID NO:403; a CDR2 comprising the amino acid sequence of SEQ ID NO:404; and a CDR3 comprising the amino acid sequence of SEQ ID NO:405; and wherein the light chain variable domain comprises: a CDR1 comprising the amino acid sequence of SEQ ID NO:408; a CDR2 comprising the amino acid sequence of SEQ ID NO:409; and a CDR3 comprising the amino acid sequence of SEQ ID NO:410;
(42) wherein the heavy chain variable domain comprises a CDR1 comprising the amino acid sequence of SEQ ID NO:413; a CDR2 comprising the amino acid sequence of SEQ ID NO:414; and a CDR3 comprising the amino acid sequence of SEQ ID NO:415; and wherein the light chain variable domain comprises: a CDR1 comprising the amino acid sequence of SEQ ID NO:418; a CDR2 comprising the amino acid sequence of SEQ ID NO:419; and a CDR3 comprising the amino acid sequence of SEQ ID NO:420;
(43) wherein the heavy chain variable domain comprises a CDR1 comprising the amino acid sequence of SEQ ID NO:423; a CDR2 comprising the amino acid sequence of SEQ ID NO:424; and a CDR3 comprising the amino acid sequence of SEQ ID NO:425; and wherein the light chain variable domain comprises: a CDR1 comprising the amino acid sequence of SEQ ID NO:428; a CDR2 comprising the amino acid sequence of SEQ ID NO:429; and a CDR3 comprising the amino acid sequence of SEQ ID NO:430;
(44) wherein the heavy chain variable domain comprises a CDR1 comprising the amino acid sequence of SEQ ID NO:433; a CDR2 comprising the amino acid sequence of SEQ ID NO:434; and a CDR3 comprising the amino acid sequence of SEQ ID NO:435; and wherein the light chain variable domain comprises: a CDR1 comprising the amino acid sequence of SEQ ID NO:438; a CDR2 comprising the amino acid sequence of SEQ ID NO:439; and a CDR3 comprising the amino acid sequence of SEQ ID NO:440;
(45) wherein the heavy chain variable domain comprises a CDR1 comprising the amino acid sequence of SEQ ID NO:443; a CDR2 comprising the amino acid sequence of SEQ ID NO:444; and a CDR3 comprising the amino acid sequence of SEQ ID NO:445; and wherein the light chain variable domain comprises: a CDR1 comprising the amino acid sequence of SEQ ID NO:448; a CDR2 comprising the amino acid sequence of SEQ ID NO:449; and a CDR3 comprising the amino acid sequence of SEQ ID NO:450;
(46) wherein the heavy chain variable domain comprises a CDR1 comprising the amino acid sequence of SEQ ID NO:453; a CDR2 comprising the amino acid sequence of SEQ ID NO:454; and a CDR3 comprising the amino acid sequence of SEQ ID NO:455; and wherein the light chain variable domain comprises: a CDR1 comprising the amino acid sequence of SEQ ID NO:458; a CDR2 comprising the amino acid sequence of SEQ ID NO:459; and a CDR3 comprising the amino acid sequence of SEQ ID NO:460;
(47) wherein the heavy chain variable domain comprises a CDR1 comprising the amino acid sequence of SEQ ID NO:463; a CDR2 comprising the amino acid sequence of SEQ ID NO:464; and a CDR3 comprising the amino acid sequence of SEQ ID NO:465; and wherein the light chain variable domain comprises: a CDR1 comprising the amino acid sequence of SEQ ID NO:468; a CDR2 comprising the amino acid sequence of SEQ ID NO:469; and a CDR3 comprising the amino acid sequence of SEQ ID NO:470;
(48) wherein the heavy chain variable domain comprises a CDR1 comprising the amino acid sequence of SEQ ID NO:473; a CDR2 comprising the amino acid sequence of SEQ ID NO:474; and a CDR3 comprising the amino acid sequence of SEQ ID NO:475; and wherein the light chain variable domain comprises: a CDR1 comprising the amino acid sequence of SEQ ID NO:478; a CDR2 comprising the amino acid sequence of SEQ ID NO:479; and a CDR3 comprising the amino acid sequence of SEQ ID NO:480;
(49) wherein the heavy chain variable domain comprises a CDR1 comprising the amino acid sequence of SEQ ID NO:483; a CDR2 comprising the amino acid sequence of SEQ ID NO:484; and a CDR3 comprising the amino acid sequence of SEQ ID NO:485; and wherein the light chain variable domain comprises: a CDR1 comprising the amino acid sequence of SEQ ID NO:488; a CDR2 comprising the amino acid sequence of SEQ ID NO:489; and a CDR3 comprising the amino acid sequence of SEQ ID NO:490;
(50) wherein the heavy chain variable domain comprises a CDR1 comprising the amino acid sequence of SEQ ID NO:493; a CDR2 comprising the amino acid sequence of SEQ ID NO:494; and a CDR3 comprising the amino acid sequence of SEQ ID NO:495; and wherein the light chain variable domain comprises: a CDR1 comprising the amino acid sequence of SEQ ID NO:498; a CDR2 comprising the amino acid sequence of SEQ ID NO:499; and a CDR3 comprising the amino acid sequence of SEQ ID NO:500;
(51) wherein the heavy chain variable domain comprises a CDR1 comprising the amino acid sequence of SEQ ID NO:503; a CDR2 comprising the amino acid sequence of SEQ ID NO:504; and a CDR3 comprising the amino acid sequence of SEQ ID NO:505; and wherein the light chain variable domain comprises: a CDR1 comprising the amino acid sequence of SEQ ID NO:508; a CDR2 comprising the amino acid sequence of SEQ ID NO:509; and a CDR3 comprising the amino acid sequence of SEQ ID NO:510;
(52) wherein the heavy chain variable domain comprises a CDR1 comprising the amino acid sequence of SEQ ID NO:513; a CDR2 comprising the amino acid sequence of SEQ ID NO:514; and a CDR3 comprising the amino acid sequence of SEQ ID NO:515; and wherein the light chain variable domain comprises: a CDR1 comprising the amino acid sequence of SEQ ID NO:518; a CDR2 comprising the amino acid sequence of SEQ ID NO:519; and a CDR3 comprising the amino acid sequence of SEQ ID NO:520;
(53) wherein the heavy chain variable domain comprises a CDR1 comprising the amino acid sequence of SEQ ID NO:523; a CDR2 comprising the amino acid sequence of SEQ ID NO:524; and a CDR3 comprising the amino acid sequence of SEQ ID NO:525; and wherein the light chain variable domain comprises: a CDR1 comprising the amino acid sequence of SEQ ID NO:528; a CDR2 comprising the amino acid sequence of SEQ ID NO:529; and a CDR3 comprising the amino acid sequence of SEQ ID NO:530;
(54) wherein the heavy chain variable domain comprises a CDR1 comprising the amino acid sequence of SEQ ID NO:533; a CDR2 comprising the amino acid sequence of SEQ ID NO:534; and a CDR3 comprising the amino acid sequence of SEQ ID NO:535; and wherein the light chain variable domain comprises: a CDR1 comprising the amino acid sequence of SEQ ID NO:538; a CDR2 comprising the amino acid sequence of SEQ ID NO:539; and a CDR3 comprising the amino acid sequence of SEQ ID NO:540;
(55) wherein the heavy chain variable domain comprises a CDR1 comprising the amino acid sequence of SEQ ID NO:543; a CDR2 comprising the amino acid sequence of SEQ ID NO:544; and a CDR3 comprising the amino acid sequence of SEQ ID NO:545; and wherein the light chain variable domain comprises: a CDR1 comprising the amino acid sequence of SEQ ID NO:548; a CDR2 comprising the amino acid sequence of SEQ ID NO:549; and a CDR3 comprising the amino acid sequence of SEQ ID NO:550;
(56) wherein the heavy chain variable domain comprises a CDR1 comprising the amino acid sequence of SEQ ID NO:553; a CDR2 comprising the amino acid sequence of SEQ ID NO:554; and a CDR3 comprising the amino acid sequence of SEQ ID NO:555; and wherein the light chain variable domain comprises: a CDR1 comprising the amino acid sequence of SEQ ID NO:558; a CDR2 comprising the amino acid sequence of SEQ ID NO:559; and a CDR3 comprising the amino acid sequence of SEQ ID NO:560;
(57) wherein the heavy chain variable domain comprises a CDR1 comprising the amino acid sequence of SEQ ID NO:563; a CDR2 comprising the amino acid sequence of SEQ ID NO:564; and a CDR3 comprising the amino acid sequence of SEQ ID NO:565; and wherein the light chain variable domain comprises: a CDR1 comprising the amino acid sequence of SEQ ID NO:568; a CDR2 comprising the amino acid sequence of SEQ ID NO:569; and a CDR3 comprising the amino acid sequence of SEQ ID NO:570;
(58) wherein the heavy chain variable domain comprises a CDR1 comprising the amino acid sequence of SEQ ID NO:573; a CDR2 comprising the amino acid sequence of SEQ ID NO:574; and a CDR3 comprising the amino acid sequence of SEQ ID NO:575; and wherein the light chain variable domain comprises: a CDR1 comprising the amino acid sequence of SEQ ID NO:578; a CDR2 comprising the amino acid sequence of SEQ ID NO:579; and a CDR3 comprising the amino acid sequence of SEQ ID NO:580;
(59) wherein the heavy chain variable domain comprises a CDR1 comprising the amino acid sequence of SEQ ID NO:583; a CDR2 comprising the amino acid sequence of SEQ ID NO:584; and a CDR3 comprising the amino acid sequence of SEQ ID NO:585; and wherein the light chain variable domain comprises: a CDR1 comprising the amino acid sequence of SEQ ID NO:588; a CDR2 comprising the amino acid sequence of SEQ ID NO:589; and a CDR3 comprising the amino acid sequence of SEQ ID NO:590;
(60) wherein the heavy chain variable domain comprises a CDR1 comprising the amino acid sequence of SEQ ID NO:593; a CDR2 comprising the amino acid sequence of SEQ ID NO:594; and a CDR3 comprising the amino acid sequence of SEQ ID NO:595; and wherein the light chain variable domain comprises: a CDR1 comprising the amino acid sequence of SEQ ID NO:598; a CDR2 comprising the amino acid sequence of SEQ ID NO:599; and a CDR3 comprising the amino acid sequence of SEQ ID NO:600;
(61) wherein the heavy chain variable domain comprises a CDR1 comprising the amino acid sequence of SEQ ID NO:603; a CDR2 comprising the amino acid sequence of SEQ ID NO:604; and a CDR3 comprising the amino acid sequence of SEQ ID NO:605; and wherein the light chain variable domain comprises: a CDR1 comprising the amino acid sequence of SEQ ID NO:608; a CDR2 comprising the amino acid sequence of SEQ ID NO:609; and a CDR3 comprising the amino acid sequence of SEQ ID NO:610;
(62) wherein the heavy chain variable domain comprises a CDR1 comprising the amino acid sequence of SEQ ID NO:613; a CDR2 comprising the amino acid sequence of SEQ ID NO:614; and a CDR3 comprising the amino acid sequence of SEQ ID NO:615; and wherein the light chain variable domain comprises: a CDR1 comprising the amino acid sequence of SEQ ID NO:618; a CDR2 comprising the amino acid sequence of SEQ ID NO:619; and a CDR3 comprising the amino acid sequence of SEQ ID NO:620;
(63) wherein the heavy chain variable domain comprises a CDR1 comprising the amino acid sequence of SEQ ID NO:623; a CDR2 comprising the amino acid sequence of SEQ ID NO:624; and a CDR3 comprising the amino acid sequence of SEQ ID NO:625; and wherein the light chain variable domain comprises: a CDR1 comprising the amino acid sequence of SEQ ID NO:628; a CDR2 comprising the amino acid sequence of SEQ ID NO:629; and a CDR3 comprising the amino acid sequence of SEQ ID NO:630;
(64) wherein the heavy chain variable domain comprises a CDR1 comprising the amino acid sequence of SEQ ID NO:633; a CDR2 comprising the amino acid sequence of SEQ ID NO:634; and a CDR3 comprising the amino acid sequence of SEQ ID NO:635; and wherein the light chain variable domain comprises: a CDR1 comprising the amino acid sequence of SEQ ID NO:638; a CDR2 comprising the amino acid sequence of SEQ ID NO:639; and a CDR3 comprising the amino acid sequence of SEQ ID NO:640;
(65) wherein the heavy chain variable domain comprises a CDR1 comprising the amino acid sequence of SEQ ID NO:643; a CDR2 comprising the amino acid sequence of SEQ ID NO:644; and a CDR3 comprising the amino acid sequence of SEQ ID NO:645; and wherein the light chain variable domain comprises: a CDR1 comprising the amino acid sequence of SEQ ID NO:648; a CDR2 comprising the amino acid sequence of SEQ ID NO:649; and a CDR3 comprising the amino acid sequence of SEQ ID NO:650;
(66) wherein the heavy chain variable domain comprises a CDR1 comprising the amino acid sequence of SEQ ID NO:653; a CDR2 comprising the amino acid sequence of SEQ ID NO:654; and a CDR3 comprising the amino acid sequence of SEQ ID NO:655; and wherein the light chain variable domain comprises: a CDR1 comprising the amino acid sequence of SEQ ID NO:658; a CDR2 comprising the amino acid sequence of SEQ ID NO:659; and a CDR3 comprising the amino acid sequence of SEQ ID NO:660;
(67) wherein the heavy chain variable domain comprises a CDR1 comprising the amino acid sequence of SEQ ID NO:663; a CDR2 comprising the amino acid sequence of SEQ ID NO:664; and a CDR3 comprising the amino acid sequence of SEQ ID NO:665; and wherein the light chain variable domain comprises: a CDR1 comprising the amino acid sequence of SEQ ID NO:668; a CDR2 comprising the amino acid sequence of SEQ ID NO:669; and a CDR3 comprising the amino acid sequence of SEQ ID NO:670;
(68) wherein the heavy chain variable domain comprises a CDR1 comprising the amino acid sequence of SEQ ID NO:673; a CDR2 comprising the amino acid sequence of SEQ ID NO:674; and a CDR3 comprising the amino acid sequence of SEQ ID NO:675; and wherein the light chain variable domain comprises: a CDR1 comprising the amino acid sequence of SEQ ID NO:678; a CDR2 comprising the amino acid sequence of SEQ ID NO:679; and a CDR3 comprising the amino acid sequence of SEQ ID NO:680;
(69) wherein the heavy chain variable domain comprises a CDR1 comprising the amino acid sequence of SEQ ID NO:683; a CDR2 comprising the amino acid sequence of SEQ ID NO:684; and a CDR3 comprising the amino acid sequence of SEQ ID NO:685; and wherein the light chain variable domain comprises: a CDR1 comprising the amino acid sequence of SEQ ID NO:688; a CDR2 comprising the amino acid sequence of SEQ ID NO:689; and a CDR3 comprising the amino acid sequence of SEQ ID NO:690;
(70) wherein the heavy chain variable domain comprises a CDR1 comprising the amino acid sequence of SEQ ID NO:693; a CDR2 comprising the amino acid sequence of SEQ ID NO:694; and a CDR3 comprising the amino acid sequence of SEQ ID NO:695; and wherein the light chain variable domain comprises: a CDR1 comprising the amino acid sequence of SEQ ID NO:698; a CDR2 comprising the amino acid sequence of SEQ ID NO:699; and a CDR3 comprising the amino acid sequence of SEQ ID NO:700;
(71) wherein the heavy chain variable domain comprises a CDR1 comprising the amino acid sequence of SEQ ID NO:703; a CDR2 comprising the amino acid sequence of SEQ ID NO:704; and a CDR3 comprising the amino acid sequence of SEQ ID NO:705; and wherein the light chain variable domain comprises: a CDR1 comprising the amino acid sequence of SEQ ID NO:708; a CDR2 comprising the amino acid sequence of SEQ ID NO:709; and a CDR3 comprising the amino acid sequence of SEQ ID NO:710;
(72) wherein the heavy chain variable domain comprises a CDR1 comprising the amino acid sequence of SEQ ID NO:713; a CDR2 comprising the amino acid sequence of SEQ ID NO:714; and a CDR3 comprising the amino acid sequence of SEQ ID NO:715; and wherein the light chain variable domain comprises: a CDR1 comprising the amino acid sequence of SEQ ID NO:718; a CDR2 comprising the amino acid sequence of SEQ ID NO:719; and a CDR3 comprising the amino acid sequence of SEQ ID NO:720;
(73) wherein the heavy chain variable domain comprises a CDR1 comprising the amino acid sequence of SEQ ID NO:723; a CDR2 comprising the amino acid sequence of SEQ ID NO:724; and a CDR3 comprising the amino acid sequence of SEQ ID NO:725; and wherein the light chain variable domain comprises: a CDR1 comprising the amino acid sequence of SEQ ID NO:728; a CDR2 comprising the amino acid sequence of SEQ ID NO:729; and a CDR3 comprising the amino acid sequence of SEQ ID NO:730;
(74) wherein the heavy chain variable domain comprises a CDR1 comprising the amino acid sequence of SEQ ID NO:733; a CDR2 comprising the amino acid sequence of SEQ ID NO:734; and a CDR3 comprising the amino acid sequence of SEQ ID NO:735; and wherein the light chain variable domain comprises: a CDR1 comprising the amino acid sequence of SEQ ID NO:738; a CDR2 comprising the amino acid sequence of SEQ ID NO:739; and a CDR3 comprising the amino acid sequence of SEQ ID NO:740;
(75) wherein the heavy chain variable domain comprises a CDR1 comprising the amino acid sequence of SEQ ID NO:743; a CDR2 comprising the amino acid sequence of SEQ ID NO:744; and a CDR3 comprising the amino acid sequence of SEQ ID NO:745; and wherein the light chain variable domain comprises: a CDR1 comprising the amino acid sequence of SEQ ID NO:748; a CDR2 comprising the amino acid sequence of SEQ ID NO:749; and a CDR3 comprising the amino acid sequence of SEQ ID NO:750;
(76) wherein the heavy chain variable domain comprises a CDR1 comprising the amino acid sequence of SEQ ID NO:753; a CDR2 comprising the amino acid sequence of SEQ ID NO:754; and a CDR3 comprising the amino acid sequence of SEQ ID NO:755; and wherein the light chain variable domain comprises: a CDR1 comprising the amino acid sequence of SEQ ID NO:758; a CDR2 comprising the amino acid sequence of SEQ ID NO:759; and a CDR3 comprising the amino acid sequence of SEQ ID NO:760;
(77) wherein the heavy chain variable domain comprises a CDR1 comprising the amino acid sequence of SEQ ID NO:763; a CDR2 comprising the amino acid sequence of SEQ ID NO:764; and a CDR3 comprising the amino acid sequence of SEQ ID NO:765; and wherein the light chain variable domain comprises: a CDR1 comprising the amino acid sequence of SEQ ID NO:768; a CDR2 comprising the amino acid sequence of SEQ ID NO:769; and a CDR3 comprising the amino acid sequence of SEQ ID NO:770;
(78) wherein the heavy chain variable domain comprises a CDR1 comprising the amino acid sequence of SEQ ID NO:773; a CDR2 comprising the amino acid sequence of SEQ ID NO:774; and a CDR3 comprising the amino acid sequence of SEQ ID NO:775; and wherein the light chain variable domain comprises: a CDR1 comprising the amino acid sequence of SEQ ID NO:778; a CDR2 comprising the amino acid sequence of SEQ ID NO:779; and a CDR3 comprising the amino acid sequence of SEQ ID NO:780;
(79) wherein the heavy chain variable domain comprises a CDR1 comprising the amino acid sequence of SEQ ID NO:783; a CDR2 comprising the amino acid sequence of SEQ ID NO:784; and a CDR3 comprising the amino acid sequence of SEQ ID NO:785; and wherein the light chain variable domain comprises: a CDR1 comprising the amino acid sequence of SEQ ID NO:788; a CDR2 comprising the amino acid sequence of SEQ ID NO:789; and a CDR3 comprising the amino acid sequence of SEQ ID NO:790;
(80) wherein the heavy chain variable domain comprises a CDR1 comprising the amino acid sequence of SEQ ID NO:793; a CDR2 comprising the amino acid sequence of SEQ ID NO:794; and a CDR3 comprising the amino acid sequence of SEQ ID NO:795; and wherein the light chain variable domain comprises: a CDR1 comprising the amino acid sequence of SEQ ID NO:798; a CDR2 comprising the amino acid sequence of SEQ ID NO:799; and a CDR3 comprising the amino acid sequence of SEQ ID NO:800;
(81) wherein the heavy chain variable domain comprises a CDR1 comprising the amino acid sequence of SEQ ID NO:803; a CDR2 comprising the amino acid sequence of SEQ ID NO:804; and a CDR3 comprising the amino acid sequence of SEQ ID NO:805; and wherein the light chain variable domain comprises: a CDR1 comprising the amino acid sequence of SEQ ID NO:808; a CDR2 comprising the amino acid sequence of SEQ ID NO:809; and a CDR3 comprising the amino acid sequence of SEQ ID NO:810;
(82) wherein the heavy chain variable domain comprises a CDR1 comprising the amino acid sequence of SEQ ID NO:813; a CDR2 comprising the amino acid sequence of SEQ ID NO:814; and a CDR3 comprising the amino acid sequence of SEQ ID NO:815; and wherein the light chain variable domain comprises: a CDR1 comprising the amino acid sequence of SEQ ID NO:818; a CDR2 comprising the amino acid sequence of SEQ ID NO:819; and a CDR3 comprising the amino acid sequence of SEQ ID NO:820;
(83) wherein the heavy chain variable domain comprises a CDR1 comprising the amino acid sequence of SEQ ID NO:823; a CDR2 comprising the amino acid sequence of SEQ ID NO:824; and a CDR3 comprising the amino acid sequence of SEQ ID NO:825; and wherein the light chain variable domain comprises: a CDR1 comprising the amino acid sequence of SEQ ID NO:828; a CDR2 comprising the amino acid sequence of SEQ ID NO:829; and a CDR3 comprising the amino acid sequence of SEQ ID NO:830;
(84) wherein the heavy chain variable domain comprises a CDR1 comprising the amino acid sequence of SEQ ID NO:833; a CDR2 comprising the amino acid sequence of SEQ ID NO:834; and a CDR3 comprising the amino acid sequence of SEQ ID NO:835; and wherein the light chain variable domain comprises: a CDR1 comprising the amino acid sequence of SEQ ID NO:838; a CDR2 comprising the amino acid sequence of SEQ ID NO:839; and a CDR3 comprising the amino acid sequence of SEQ ID NO:840;
(85) wherein the heavy chain variable domain comprises a CDR1 comprising the amino acid sequence of SEQ ID NO:843; a CDR2 comprising the amino acid sequence of SEQ ID NO:844; and a CDR3 comprising the amino acid sequence of SEQ ID NO:845; and wherein the light chain variable domain comprises: a CDR1 comprising the amino acid sequence of SEQ ID NO:848; a CDR2 comprising the amino acid sequence of SEQ ID NO:849; and a CDR3 comprising the amino acid sequence of SEQ ID NO:850;
(86) wherein the heavy chain variable domain comprises a CDR1 comprising the amino acid sequence of SEQ ID NO:853; a CDR2 comprising the amino acid sequence of SEQ ID NO:854; and a CDR3 comprising the amino acid sequence of SEQ ID NO:855; and wherein the light chain variable domain comprises: a CDR1 comprising the amino acid sequence of SEQ ID NO:858; a CDR2 comprising the amino acid sequence of SEQ ID NO:859; and a CDR3 comprising the amino acid sequence of SEQ ID NO:860;
(87) wherein the heavy chain variable domain comprises a CDR1 comprising the amino acid sequence of SEQ ID NO:863; a CDR2 comprising the amino acid sequence of SEQ ID NO:864; and a CDR3 comprising the amino acid sequence of SEQ ID NO:865; and wherein the light chain variable domain comprises: a CDR1 comprising the amino acid sequence of SEQ ID NO:868; a CDR2 comprising the amino acid sequence of SEQ ID NO:869; and a CDR3 comprising the amino acid sequence of SEQ ID NO:870;
(88) wherein the heavy chain variable domain comprises a CDR1 comprising the amino acid sequence of SEQ ID NO:873; a CDR2 comprising the amino acid sequence of SEQ ID NO:874; and a CDR3 comprising the amino acid sequence of SEQ ID NO:875; and wherein the light chain variable domain comprises: a CDR1 comprising the amino acid sequence of SEQ ID NO:878; a CDR2 comprising the amino acid sequence of SEQ ID NO:879; and a CDR3 comprising the amino acid sequence of SEQ ID NO:880;
(89) wherein the heavy chain variable domain comprises a CDR1 comprising the amino acid sequence of SEQ ID NO:883; a CDR2 comprising the amino acid sequence of SEQ ID NO:884; and a CDR3 comprising the amino acid sequence of SEQ ID NO:885; and wherein the light chain variable domain comprises: a CDR1 comprising the amino acid sequence of SEQ ID NO:888; a CDR2 comprising the amino acid sequence of SEQ ID NO:889; and a CDR3 comprising the amino acid sequence of SEQ ID NO:890;
(90) wherein the heavy chain variable domain comprises a CDR1 comprising the amino acid sequence of SEQ ID NO:893; a CDR2 comprising the amino acid sequence of SEQ ID NO:894; and a CDR3 comprising the amino acid sequence of SEQ ID NO:895; and wherein the light chain variable domain comprises: a CDR1 comprising the amino acid sequence of SEQ ID NO:898; a CDR2 comprising the amino acid sequence of SEQ ID NO:899; and a CDR3 comprising the amino acid sequence of SEQ ID NO:900;
(91) wherein the heavy chain variable domain comprises a CDR1 comprising the amino acid sequence of SEQ ID NO:903; a CDR2 comprising the amino acid sequence of SEQ ID NO:904; and a CDR3 comprising the amino acid sequence of SEQ ID NO:905; and wherein the light chain variable domain comprises: a CDR1 comprising the amino acid sequence of SEQ ID NO:908; a CDR2 comprising the amino acid sequence of SEQ ID NO:909; and a CDR3 comprising the amino acid sequence of SEQ ID NO:910;
(92) wherein the heavy chain variable domain comprises a CDR1 comprising the amino acid sequence of SEQ ID NO:913; a CDR2 comprising the amino acid sequence of SEQ ID NO:914; and a CDR3 comprising the amino acid sequence of SEQ ID NO:915; and wherein the light chain variable domain comprises: a CDR1 comprising the amino acid sequence of SEQ ID NO:918; a CDR2 comprising the amino acid sequence of SEQ ID NO:919; and a CDR3 comprising the amino acid sequence of SEQ ID NO:920;
(93) wherein the heavy chain variable domain comprises a CDR1 comprising the amino acid sequence of SEQ ID NO:923; a CDR2 comprising the amino acid sequence of SEQ ID NO:924; and a CDR3 comprising the amino acid sequence of SEQ ID NO:925; and wherein the light chain variable domain comprises: a CDR1 comprising the amino acid sequence of SEQ ID NO:928; a CDR2 comprising the amino acid sequence of SEQ ID NO:929; and a CDR3 comprising the amino acid sequence of SEQ ID NO:930;
(94) wherein the heavy chain variable domain comprises a CDR1 comprising the amino acid sequence of SEQ ID NO:933; a CDR2 comprising the amino acid sequence of SEQ ID NO:934; and a CDR3 comprising the amino acid sequence of SEQ ID NO:935; and wherein the light chain variable domain comprises: a CDR1 comprising the amino acid sequence of SEQ ID NO:938; a CDR2 comprising the amino acid sequence of SEQ ID NO:939; and a CDR3 comprising the amino acid sequence of SEQ ID NO:940;
(95) wherein the heavy chain variable domain comprises a CDR1 comprising the amino acid sequence of SEQ ID NO:943; a CDR2 comprising the amino acid sequence of SEQ ID NO:944; and a CDR3 comprising the amino acid sequence of SEQ ID NO:945; and wherein the light chain variable domain comprises: a CDR1 comprising the amino acid sequence of SEQ ID NO:948; a CDR2 comprising the amino acid sequence of SEQ ID NO:949; and a CDR3 comprising the amino acid sequence of SEQ ID NO:950;
(96) wherein the heavy chain variable domain comprises a CDR1 comprising the amino acid sequence of SEQ ID NO:953; a CDR2 comprising the amino acid sequence of SEQ ID NO:954; and a CDR3 comprising the amino acid sequence of SEQ ID NO:955; and wherein the light chain variable domain comprises: a CDR1 comprising the amino acid sequence of SEQ ID NO:958; a CDR2 comprising the amino acid sequence of SEQ ID NO:959; and a CDR3 comprising the amino acid sequence of SEQ ID NO:960;
(97) wherein the heavy chain variable domain comprises a CDR1 comprising the amino acid sequence of SEQ ID NO:963; a CDR2 comprising the amino acid sequence of SEQ ID NO:964; and a CDR3 comprising the amino acid sequence of SEQ ID NO:965; and wherein the light chain variable domain comprises: a CDR1 comprising the amino acid sequence of SEQ ID NO:968; a CDR2 comprising the amino acid sequence of SEQ ID NO:969; and a CDR3 comprising the amino acid sequence of SEQ ID NO:970;
(98) wherein the heavy chain variable domain comprises a CDR1 comprising the amino acid sequence of SEQ ID NO:973; a CDR2 comprising the amino acid sequence of SEQ ID NO:974; and a CDR3 comprising the amino acid sequence of SEQ ID NO:975; and wherein the light chain variable domain comprises: a CDR1 comprising the amino acid sequence of SEQ ID NO:978; a CDR2 comprising the amino acid sequence of SEQ ID NO:979; and a CDR3 comprising the amino acid sequence of SEQ ID NO:980;
(99) wherein the heavy chain variable domain comprises a CDR1 comprising the amino acid sequence of SEQ ID NO:983; a CDR2 comprising the amino acid sequence of SEQ ID NO:984; and a CDR3 comprising the amino acid sequence of SEQ ID NO:985; and wherein the light chain variable domain comprises: a CDR1 comprising the amino acid sequence of SEQ ID NO:988; a CDR2 comprising the amino acid sequence of SEQ ID NO:989; and a CDR3 comprising the amino acid sequence of SEQ ID NO:990;
(100) wherein the heavy chain variable domain comprises a complementarity determining region 1 (CDR1) comprising the amino acid sequence of SEQ ID NO:1109, a CDR2 comprising the amino acid sequence of SEQ ID NO:1139, and a CDR3 comprising the amino acid sequence of SEQ ID NO:1169; and wherein the light chain variable domain comprises a CDR1 comprising the amino acid sequence of SEQ ID NO:1199, a CDR2 comprising the amino acid sequence AAS, and a CDR3 comprising the amino acid sequence of SEQ ID NO:1229;
(101) wherein the heavy chain variable domain comprises a CDR1 comprising the amino acid sequence of SEQ ID NO:1110, a CDR2 comprising the amino acid sequence of SEQ ID NO: 1140, and a CDR3 comprising the amino acid sequence of SEQ ID NO: 1170; wherein the light chain variable domain comprises a CDR1 comprising the amino acid sequence of SEQ ID NO: 1200, a CDR2 comprising the amino acid sequence KIS, and a CDR3 comprising the amino acid sequence of SEQ ID NO: 1230;
(102) wherein the heavy chain variable domain comprises a CDR1 comprising the amino acid sequence of SEQ ID NO:1111, a CDR2 comprising the amino acid sequence of SEQ ID NO: 1141, and a CDR3 comprising the amino acid sequence of SEQ ID NO: 1171; wherein the light chain variable domain comprises a CDR1 comprising the amino acid sequence of SEQ ID NO: 1201, a CDR2 comprising the amino acid sequence EVT, and a CDR3 comprising the amino acid sequence of SEQ ID NO: 1231;
(103) wherein the heavy chain variable domain comprises a CDR1 comprising the amino acid sequence of SEQ ID NO:1112, a CDR2 comprising the amino acid sequence of SEQ ID NO: 1142, and a CDR3 comprising the amino acid sequence of SEQ ID NO: 1172; wherein the light chain variable domain comprises a CDR1 comprising the amino acid sequence of SEQ ID NO: 1202, a CDR2 comprising the amino acid sequence EVS, and a CDR3 comprising the amino acid sequence of SEQ ID NO: 1232;
(104) wherein the heavy chain variable domain comprises a CDR1 comprising the amino acid sequence of SEQ ID NO:1113, a CDR2 comprising the amino acid sequence of SEQ ID NO: 1143, and a CDR3 comprising the amino acid sequence of SEQ ID NO: 1173; wherein the light chain variable domain comprises a CDR1 comprising the amino acid sequence of SEQ ID NO: 1203, a CDR2 comprising the amino acid sequence KIS, and a CDR3 comprising the amino acid sequence of SEQ ID NO: 1233;
(105) wherein the heavy chain variable domain comprises a CDR1 comprising the amino acid sequence of SEQ ID NO:1114, a CDR2 comprising the amino acid sequence of SEQ ID NO: 1144, and a CDR3 comprising the amino acid sequence of SEQ ID NO: 1174; wherein the light chain variable domain comprises a CDR1 comprising the amino acid sequence of SEQ ID NO: 1204, a CDR2 comprising the amino acid sequence ANS, and a CDR3 comprising the amino acid sequence of SEQ ID NO: 1234;
(106) wherein the heavy chain variable domain comprises a CDR1 comprising the amino acid sequence of SEQ ID NO:1115, a CDR2 comprising the amino acid sequence of SEQ ID NO: 1145, and a CDR3 comprising the amino acid sequence of SEQ ID NO: 1175; wherein the light chain variable domain comprises a CDR1 comprising the amino acid sequence of SEQ ID NO: 1205, a CDR2 comprising the amino acid sequence KDT, and a CDR3 comprising the amino acid sequence of SEQ ID NO: 1235;
(107) wherein the heavy chain variable domain comprises a CDR1 comprising the amino acid sequence of SEQ ID NO:1116, a CDR2 comprising the amino acid sequence of SEQ ID NO: 1146, and a CDR3 comprising the amino acid sequence of SEQ ID NO: 1176; wherein the light chain variable domain comprises a CDR1 comprising the amino acid sequence of SEQ ID NO: 1206, a CDR2 comprising the amino acid sequence EVS, and a CDR3 comprising the amino acid sequence of SEQ ID NO: 1236;
(108) wherein the heavy chain variable domain comprises a CDR1 comprising the amino acid sequence of SEQ ID NO:1117, a CDR2 comprising the amino acid sequence of SEQ ID NO: 1147, and a CDR3 comprising the amino acid sequence of SEQ ID NO: 1177; wherein the light chain variable domain comprises a CDR1 comprising the amino acid sequence of SEQ ID NO: 1207, a CDR2 comprising the amino acid sequence EVN, and a CDR3 comprising the amino acid sequence of SEQ ID NO: 1237;
(109) wherein the heavy chain variable domain comprises a CDR1 comprising the amino acid sequence of SEQ ID NO:1118, a CDR2 comprising the amino acid sequence of SEQ ID NO: 1148, and a CDR3 comprising the amino acid sequence of SEQ ID NO: 1178; wherein the light chain variable domain comprises a CDR1 comprising the amino acid sequence of SEQ ID NO: 1208, a CDR2 comprising the amino acid sequence AAS, and a CDR3 comprising the amino acid sequence of SEQ ID NO: 1238;
(110) wherein the heavy chain variable domain comprises a CDR1 comprising the amino acid sequence of SEQ ID NO:1119, a CDR2 comprising the amino acid sequence of SEQ ID NO: 1149, and a CDR3 comprising the amino acid sequence of SEQ ID NO: 1179; wherein the light chain variable domain comprises a CDR1 comprising the amino acid sequence of SEQ ID NO: 1209, a CDR2 comprising the amino acid sequence QDT, and a CDR3 comprising the amino acid sequence of SEQ ID NO: 1239;
(111) wherein the heavy chain variable domain comprises a CDR1 comprising the amino acid sequence of SEQ ID NO:1120, a CDR2 comprising the amino acid sequence of SEQ ID NO: 1150, and a CDR3 comprising the amino acid sequence of SEQ ID NO: 1180 and wherein the light chain variable domain comprises: a CDR1 comprising the amino acid sequence of SEQ ID NO: 1210, a CDR2 comprising the amino acid sequence EAS, and a CDR3 comprising the amino acid sequence of SEQ ID NO: 1240;
(112) wherein the heavy chain variable domain comprises a CDR1 comprising the amino acid sequence of SEQ ID NO:1121, a CDR2 comprising the amino acid sequence of SEQ ID NO: 1151, and a CDR3 comprising the amino acid sequence of SEQ ID NO: 1181 and wherein the light chain variable domain comprises: a CDR1 comprising the amino acid sequence of SEQ ID NO: 1211, a CDR2 comprising the amino acid sequence KAS, and a CDR3 comprising the amino acid sequence of SEQ ID NO: 1241;
(113) wherein the heavy chain variable domain comprises a CDR1 comprising the amino acid sequence of SEQ ID NO:1122, a CDR2 comprising the amino acid sequence of SEQ ID NO: 1152, and a CDR3 comprising the amino acid sequence of SEQ ID NO: 1182 and wherein the light chain variable domain comprises: a CDR1 comprising the amino acid sequence of SEQ ID NO: 1212, a CDR2 comprising the amino acid sequence LKN, and a CDR3 comprising the amino acid sequence of SEQ ID NO: 1242;
(114) wherein the heavy chain variable domain comprises a CDR1 comprising the amino acid sequence of SEQ ID NO:1123, a CDR2 comprising the amino acid sequence of SEQ ID NO: 1153, and a CDR3 comprising the amino acid sequence of SEQ ID NO: 1183 and wherein the light chain variable domain comprises: a CDR1 comprising the amino acid sequence of SEQ ID NO: 1213, a CDR2 comprising the amino acid sequence GAS, and a CDR3 comprising the amino acid sequence of SEQ ID NO: 1243;
(115) wherein the heavy chain variable domain comprises a CDR1 comprising the amino acid sequence of SEQ ID NO:1124, a CDR2 comprising the amino acid sequence of SEQ ID NO: 1154, and a CDR3 comprising the amino acid sequence of SEQ ID NO: 1184 and wherein the light chain variable domain comprises: a CDR1 comprising the amino acid sequence of SEQ ID NO: 1214, a CDR2 comprising the amino acid sequence DAS, and a CDR3 comprising the amino acid sequence of SEQ ID NO: 1244;
(116) wherein the heavy chain variable domain comprises a CDR1 comprising the amino acid sequence of SEQ ID NO:1125, a CDR2 comprising the amino acid sequence of SEQ ID NO: 1155, and a CDR3 comprising the amino acid sequence of SEQ ID NO: 1185 and wherein the light chain variable domain comprises: a CDR1 comprising the amino acid sequence of SEQ ID NO: 1215, a CDR2 comprising the amino acid sequence AAS, and a CDR3 comprising the amino acid sequence of SEQ ID NO: 1245;
(117) wherein the heavy chain variable domain comprises a CDR1 comprising the amino acid sequence of SEQ ID NO:1126, a CDR2 comprising the amino acid sequence of SEQ ID NO: 1156, and a CDR3 comprising the amino acid sequence of SEQ ID NO: 1186 and wherein the light chain variable domain comprises: a CDR1 comprising the amino acid sequence of SEQ ID NO: 1216, a CDR2 comprising the amino acid sequence DAS, and a CDR3 comprising the amino acid sequence of SEQ ID NO: 1246;
(118) wherein the heavy chain variable domain comprises a CDR1 comprising the amino acid sequence of SEQ ID NO:1127, a CDR2 comprising the amino acid sequence of SEQ ID NO: 1157, and a CDR3 comprising the amino acid sequence of SEQ ID NO: 1187 and wherein the light chain variable domain comprises: a CDR1 comprising the amino acid sequence of SEQ ID NO: 1217, a CDR2 comprising the amino acid sequence DVS, and a CDR3 comprising the amino acid sequence of SEQ ID NO: 1247;
(119) wherein the heavy chain variable domain comprises a CDR1 comprising the amino acid sequence of SEQ ID NO:1128, a CDR2 comprising the amino acid sequence of SEQ ID NO: 1158, and a CDR3 comprising the amino acid sequence of SEQ ID NO: 1188 and wherein the light chain variable domain comprises: a CDR1 comprising the amino acid sequence of SEQ ID NO: 1218, a CDR2 comprising the amino acid sequence GNN, and a CDR3 comprising the amino acid sequence of SEQ ID NO: 1248;
(120) wherein the heavy chain variable domain comprises a CDR1 comprising the amino acid sequence of SEQ ID NO:1129, a CDR2 comprising the amino acid sequence of SEQ ID NO: 1159, and a CDR3 comprising the amino acid sequence of SEQ ID NO: 1189 and wherein the light chain variable domain comprises: a CDR1 comprising the amino acid sequence of SEQ ID NO: 1219, a CDR2 comprising the amino acid sequence AAS, and a CDR3 comprising the amino acid sequence of SEQ ID NO: 1249;
(121) wherein the heavy chain variable domain comprises a CDR1 comprising the amino acid sequence of SEQ ID NO:1130, a CDR2 comprising the amino acid sequence of SEQ ID NO: 1160, and a CDR3 comprising the amino acid sequence of SEQ ID NO: 1190 and wherein the light chain variable domain comprises: a CDR1 comprising the amino acid sequence of SEQ ID NO: 1220, a CDR2 comprising the amino acid sequence NAS, and a CDR3 comprising the amino acid sequence of SEQ ID NO: 1250;
(122) wherein the heavy chain variable domain comprises a CDR1 comprising the amino acid sequence of SEQ ID NO:1131, a CDR2 comprising the amino acid sequence of SEQ ID NO: 1161, and a CDR3 comprising the amino acid sequence of SEQ ID NO: 1191 and wherein the light chain variable domain comprises: a CDR1 comprising the amino acid sequence of SEQ ID NO: 1221, a CDR2 comprising the amino acid sequence AAS, and a CDR3 comprising the amino acid sequence of SEQ ID NO: 1251;
(123) wherein the heavy chain variable domain comprises a CDR1 comprising the amino acid sequence of SEQ ID NO:1132, a CDR2 comprising the amino acid sequence of SEQ ID NO: 1162, and a CDR3 comprising the amino acid sequence of SEQ ID NO: 1192 and wherein the light chain variable domain comprises: a CDR1 comprising the amino acid sequence of SEQ ID NO: 1222, a CDR2 comprising the amino acid sequence AAS, and a CDR3 comprising the amino acid sequence of SEQ ID NO: 1252;
(124) wherein the heavy chain variable domain comprises a CDR1 comprising the amino acid sequence of SEQ ID NO:1133, a CDR2 comprising the amino acid sequence of SEQ ID NO: 1163, and a CDR3 comprising the amino acid sequence of SEQ ID NO: 1193 and wherein the light chain variable domain comprises: a CDR1 comprising the amino acid sequence of SEQ ID NO: 1223, a CDR2 comprising the amino acid sequence KVS, and a CDR3 comprising the amino acid sequence of SEQ ID NO: 1253;
(125) wherein the heavy chain variable domain comprises a CDR1 comprising the amino acid sequence of SEQ ID NO:1134, a CDR2 comprising the amino acid sequence of SEQ ID NO: 1164, and a CDR3 comprising the amino acid sequence of SEQ ID NO: 1194 and wherein the light chain variable domain comprises: a CDR1 comprising the amino acid sequence of SEQ ID NO: 1224, a CDR2 comprising the amino acid sequence AAS, and a CDR3 comprising the amino acid sequence of SEQ ID NO: 1254;
(126) wherein the heavy chain variable domain comprises a CDR1 comprising the amino acid sequence of SEQ ID NO:1135, a CDR2 comprising the amino acid sequence of SEQ ID NO: 1165, and a CDR3 comprising the amino acid sequence of SEQ ID NO: 1195 and wherein the light chain variable domain comprises: a CDR1 comprising the amino acid sequence of SEQ ID NO: 1225, a CDR2 comprising the amino acid sequence QDS, and a CDR3 comprising the amino acid sequence of SEQ ID NO: 1255;
(127) wherein the heavy chain variable domain comprises a CDR1 comprising the amino acid sequence of SEQ ID NO:1136, a CDR2 comprising the amino acid sequence of SEQ ID NO: 1166, and a CDR3 comprising the amino acid sequence of SEQ ID NO: 1196 and wherein the light chain variable domain comprises: a CDR1 comprising the amino acid sequence of SEQ ID NO: 1226, a CDR2 comprising the amino acid sequence AAS, and a CDR3 comprising the amino acid sequence of SEQ ID NO: 1256;
(128) wherein the heavy chain variable domain comprises a CDR1 comprising the amino acid sequence of SEQ ID NO:1137, a CDR2 comprising the amino acid sequence of SEQ ID NO: 1167, and a CDR3 comprising the amino acid sequence of SEQ ID NO: 1197 and wherein the light chain variable domain comprises: a CDR1 comprising the amino acid sequence of SEQ ID NO: 1227, a CDR2 comprising the amino acid sequence TTS, and a CDR3 comprising the amino acid sequence of SEQ ID NO: 1257;
(129) wherein the heavy chain variable domain comprises a CDR1 comprising the amino acid sequence of SEQ ID NO:1138, a CDR2 comprising the amino acid sequence of SEQ ID NO: 1168, and a CDR3 comprising the amino acid sequence of SEQ ID NO: 1198 and wherein the light chain variable domain comprises: a CDR1 comprising the amino acid sequence of SEQ ID NO: 1228, a CDR2 comprising the amino acid sequence EDN, and a CDR3 comprising the amino acid sequence of SEQ ID NO: 1258.

2. The isolated recombinant monoclonal antibody, or antigen-binding fragment thereof, of claim 1, wherein said antibody comprises a heavy chain variable domain and a light chain variable domain, and:
(1) wherein the heavy chain variable domain comprises the amino acid sequence of SEQ ID NO:2 and the light chain variable domain comprises the amino acid sequence of SEQ ID NO:7;
(2) wherein the heavy chain variable domain comprises the amino acid sequence of SEQ ID NO:12 and the light chain variable domain comprises the amino acid sequence of SEQ ID NO:17;
(3) wherein the heavy chain variable domain comprises the amino acid sequence of SEQ ID NO:22 and the light chain variable domain comprises the amino acid sequence of SEQ ID NO:27;
(4) wherein the heavy chain variable domain comprises the amino acid sequence of SEQ ID NO:32 and the light chain variable domain comprises the amino acid sequence of SEQ ID NO:37;
(5) wherein the heavy chain variable domain comprises the amino acid sequence of SEQ ID NO:42 and the light chain variable domain comprises the amino acid sequence of SEQ ID NO:47;
(6) wherein the heavy chain variable domain comprises the amino acid sequence of SEQ ID NO:52 and the light chain variable domain comprises the amino acid sequence of SEQ ID NO:57;
(7) wherein the heavy chain variable domain comprises the amino acid sequence of SEQ ID NO:62 and the light chain variable domain comprises the amino acid sequence of SEQ ID NO:67;
(8) wherein the heavy chain variable domain comprises the amino acid sequence of SEQ ID NO:72 and the light chain variable domain comprises the amino acid sequence of SEQ ID NO:77;
(9) wherein the heavy chain variable domain comprises the amino acid sequence of SEQ ID NO:82 and the light chain variable domain comprises the amino acid sequence of SEQ ID NO:87;
(10) wherein the heavy chain variable domain comprises the amino acid sequence of SEQ ID NO:92 and the light chain variable domain comprises the amino acid sequence of SEQ ID NO:97;
(11) wherein the heavy chain variable domain comprises the amino acid sequence of SEQ ID NO:102 and the light chain variable domain comprises the amino acid sequence of SEQ ID NO:107;
(12) wherein the heavy chain variable domain comprises the amino acid sequence of SEQ ID NO:112 and the light chain variable domain comprises the amino acid sequence of SEQ ID NO:117;
(13) wherein the heavy chain variable domain comprises the amino acid sequence of SEQ ID NO:122 and the light chain variable domain comprises the amino acid sequence of SEQ ID NO:127;
(14) wherein the heavy chain variable domain comprises the amino acid sequence of SEQ ID NO:132 and the light chain variable domain comprises the amino acid sequence of SEQ ID NO:137;
(15) wherein the heavy chain variable domain comprises the amino acid sequence of SEQ ID NO:142 and the light chain variable domain comprises the amino acid sequence of SEQ ID NO:147;
(16) wherein the heavy chain variable domain comprises the amino acid sequence of SEQ ID NO:152 and the light chain variable domain comprises the amino acid sequence of SEQ ID NO:157;
(17) wherein the heavy chain variable domain comprises the amino acid sequence of SEQ ID NO:162 and the light chain variable domain comprises the amino acid sequence of SEQ ID NO:167;
(18) wherein the heavy chain variable domain comprises the amino acid sequence of SEQ ID NO:172 and the light chain variable domain comprises the amino acid sequence of SEQ ID NO:177;
(19) wherein the heavy chain variable domain comprises the amino acid sequence of SEQ ID NO:182 and the light chain variable domain comprises the amino acid sequence of SEQ ID NO:187;
(20) wherein the heavy chain variable domain comprises the amino acid sequence of SEQ ID NO:192 and the light chain variable domain comprises the amino acid sequence of SEQ ID NO:197;
(21) wherein the heavy chain variable domain comprises the amino acid sequence of SEQ ID NO:202 and the light chain variable domain comprises the amino acid sequence of SEQ ID NO:207;
(22) wherein the heavy chain variable domain comprises the amino acid sequence of SEQ ID NO:212 and the light chain variable domain comprises the amino acid sequence of SEQ ID NO:217;
(23) wherein the heavy chain variable domain comprises the amino acid sequence of SEQ ID NO:222 and the light chain variable domain comprises the amino acid sequence of SEQ ID NO:227;
(24) wherein the heavy chain variable domain comprises the amino acid sequence of SEQ ID NO:232 and the light chain variable domain comprises the amino acid sequence of SEQ ID NO:237;
(25) wherein the heavy chain variable domain comprises the amino acid sequence of SEQ ID NO:242 and the light chain variable domain comprises the amino acid sequence of SEQ ID NO:247;
(26) wherein the heavy chain variable domain comprises the amino acid sequence of SEQ ID NO:252 and the light chain variable domain comprises the amino acid sequence of SEQ ID NO:257;
(27) wherein the heavy chain variable domain comprises the amino acid sequence of SEQ ID NO:262 and the light chain variable domain comprises the amino acid sequence of SEQ ID NO:267;
(28) wherein the heavy chain variable domain comprises the amino acid sequence of SEQ ID NO:272 and the light chain variable domain comprises the amino acid sequence of SEQ ID NO:277;
(29) wherein the heavy chain variable domain comprises the amino acid sequence of SEQ ID NO:282 and the light chain variable domain comprises the amino acid sequence of SEQ ID NO:287;
(30) wherein the heavy chain variable domain comprises the amino acid sequence of SEQ ID NO:292 and the light chain variable domain comprises the amino acid sequence of SEQ ID NO:297;
(31) wherein the heavy chain variable domain comprises the amino acid sequence of SEQ ID NO:302 and the light chain variable domain comprises the amino acid sequence of SEQ ID NO:307;
(32) wherein the heavy chain variable domain comprises the amino acid sequence of SEQ ID NO:312 and the light chain variable domain comprises the amino acid sequence of SEQ ID NO:317;
(33) wherein the heavy chain variable domain comprises the amino acid sequence of SEQ ID NO:322 and the light chain variable domain comprises the amino acid sequence of SEQ ID NO:327;
(34) wherein the heavy chain variable domain comprises the amino acid sequence of SEQ ID NO:332 and the light chain variable domain comprises the amino acid sequence of SEQ ID NO:337;
(35) wherein the heavy chain variable domain comprises the amino acid sequence of SEQ ID NO:342 and the light chain variable domain comprises the amino acid sequence of SEQ ID NO:347;
(36) wherein the heavy chain variable domain comprises the amino acid sequence of SEQ ID NO:352 and the light chain variable domain comprises the amino acid sequence of SEQ ID NO:357;
(37) wherein the heavy chain variable domain comprises the amino acid sequence of SEQ ID NO:362 and the light chain variable domain comprises the amino acid sequence of SEQ ID NO:367;
(38) wherein the heavy chain variable domain comprises the amino acid sequence of SEQ ID NO:372 and the light chain variable domain comprises the amino acid sequence of SEQ ID NO:377;
(39) wherein the heavy chain variable domain comprises the amino acid sequence of SEQ ID NO:382 and the light chain variable domain comprises the amino acid sequence of SEQ ID NO:387;
(40) wherein the heavy chain variable domain comprises the amino acid sequence of SEQ ID NO:392 and the light chain variable domain comprises the amino acid sequence of SEQ ID NO:397;
(41) wherein the heavy chain variable domain comprises the amino acid sequence of SEQ ID NO:402 and the light chain variable domain comprises the amino acid sequence of SEQ ID NO:497;
(42) wherein the heavy chain variable domain comprises the amino acid sequence of SEQ ID NO:412 and the light chain variable domain comprises the amino acid sequence of SEQ ID NO:417; or
(43) wherein the heavy chain variable domain comprises the amino acid sequence of SEQ ID NO:422 and the light chain variable domain comprises the amino acid sequence of SEQ ID NO:427
(44) wherein the heavy chain variable domain comprises the amino acid sequence of SEQ ID NO:432 and the light chain variable domain comprises the amino acid sequence of SEQ ID NO:437;
(45) wherein the heavy chain variable domain comprises the amino acid sequence of SEQ ID NO:442 and the light chain variable domain comprises the amino acid sequence of SEQ ID NO:447;
(46) wherein the heavy chain variable domain comprises the amino acid sequence of SEQ ID NO:452 and the light chain variable domain comprises the amino acid sequence of SEQ ID NO:457;
(47) wherein the heavy chain variable domain comprises the amino acid sequence of SEQ ID NO:462 and the light chain variable domain comprises the amino acid sequence of SEQ ID NO:467;
(48) wherein the heavy chain variable domain comprises the amino acid sequence of SEQ ID NO:472 and the light chain variable domain comprises the amino acid sequence of SEQ ID NO:477;
(49) wherein the heavy chain variable domain comprises the amino acid sequence of SEQ ID NO:482 and the light chain variable domain comprises the amino acid sequence of SEQ ID NO:487;
(50) wherein the heavy chain variable domain comprises the amino acid sequence of SEQ ID NO:492 and the light chain variable domain comprises the amino acid sequence of SEQ ID NO:497;
(51) wherein the heavy chain variable domain comprises the amino acid sequence of SEQ ID NO:502 and the light chain variable domain comprises the amino acid sequence of SEQ ID NO:507;
(52) wherein the heavy chain variable domain comprises the amino acid sequence of SEQ ID NO:512 and the light chain variable domain comprises the amino acid sequence of SEQ ID NO:517;
(53) wherein the heavy chain variable domain comprises the amino acid sequence of SEQ ID NO:522 and the light chain variable domain comprises the amino acid sequence of SEQ ID NO:527;
(54) wherein the heavy chain variable domain comprises the amino acid sequence of SEQ ID NO:532 and the light chain variable domain comprises the amino acid sequence of SEQ ID NO:537;
(55) wherein the heavy chain variable domain comprises the amino acid sequence of SEQ ID NO:542 and the light chain variable domain comprises the amino acid sequence of SEQ ID NO:547;
(56) wherein the heavy chain variable domain comprises the amino acid sequence of SEQ ID NO:552 and the light chain variable domain comprises the amino acid sequence of SEQ ID NO:557;
(57) wherein the heavy chain variable domain comprises the amino acid sequence of SEQ ID NO:562 and the light chain variable domain comprises the amino acid sequence of SEQ ID NO:567;
(58) wherein the heavy chain variable domain comprises the amino acid sequence of SEQ ID NO:572 and the light chain variable domain comprises the amino acid sequence of SEQ ID NO:577;
(59) wherein the heavy chain variable domain comprises the amino acid sequence of SEQ ID NO:582 and the light chain variable domain comprises the amino acid sequence of SEQ ID NO:587;
(60) wherein the heavy chain variable domain comprises the amino acid sequence of SEQ ID NO:592 and the light chain variable domain comprises the amino acid sequence of SEQ ID NO:597;
(61) wherein the heavy chain variable domain comprises the amino acid sequence of SEQ ID NO:602 and the light chain variable domain comprises the amino acid sequence of SEQ ID NO:607;
(62) wherein the heavy chain variable domain comprises the amino acid sequence of SEQ ID NO:612 and the light chain variable domain comprises the amino acid sequence of SEQ ID NO:617;
(63) wherein the heavy chain variable domain comprises the amino acid sequence of SEQ ID NO:622 and the light chain variable domain comprises the amino acid sequence of SEQ ID NO:627;
(64) wherein the heavy chain variable domain comprises the amino acid sequence of SEQ ID NO:632 and the light chain variable domain comprises the amino acid sequence of SEQ ID NO:637;
(65) wherein the heavy chain variable domain comprises the amino acid sequence of SEQ ID NO:642 and the light chain variable domain comprises the amino acid sequence of SEQ ID NO:647;
(66) wherein the heavy chain variable domain comprises the amino acid sequence of SEQ ID NO:652 and the light chain variable domain comprises the amino acid sequence of SEQ ID NO:657;
(67) wherein the heavy chain variable domain comprises the amino acid sequence of SEQ ID NO:662 and the light chain variable domain comprises the amino acid sequence of SEQ ID NO:667;
(68) wherein the heavy chain variable domain comprises the amino acid sequence of SEQ ID NO:672 and the light chain variable domain comprises the amino acid sequence of SEQ ID NO:677;
(69) wherein the heavy chain variable domain comprises the amino acid sequence of SEQ ID NO:682 and the light chain variable domain comprises the amino acid sequence of SEQ ID NO:687;
(70) wherein the heavy chain variable domain comprises the amino acid sequence of SEQ ID NO:692 and the light chain variable domain comprises the amino acid sequence of SEQ ID NO:697;
(71) wherein the heavy chain variable domain comprises the amino acid sequence of SEQ ID NO:702 and the light chain variable domain comprises the amino acid sequence of SEQ ID NO:707;
(72) wherein the heavy chain variable domain comprises the amino acid sequence of SEQ ID NO:712 and the light chain variable domain comprises the amino acid sequence of SEQ ID NO:717;
(73) wherein the heavy chain variable domain comprises the amino acid sequence of SEQ ID NO:722 and the light chain variable domain comprises the amino acid sequence of SEQ ID NO:727;
(74) wherein the heavy chain variable domain comprises the amino acid sequence of SEQ ID NO:732 and the light chain variable domain comprises the amino acid sequence of SEQ ID NO:737;
(75) wherein the heavy chain variable domain comprises the amino acid sequence of SEQ ID NO:742 and the light chain variable domain comprises the amino acid sequence of SEQ ID NO:747;
(76) wherein the heavy chain variable domain comprises the amino acid sequence of SEQ ID NO:752 and the light chain variable domain comprises the amino acid sequence of SEQ ID NO:757;
(77) wherein the heavy chain variable domain comprises the amino acid sequence of SEQ ID NO:762 and the light chain variable domain comprises the amino acid sequence of SEQ ID NO:767;
(78) wherein the heavy chain variable domain comprises the amino acid sequence of SEQ ID NO:772 and the light chain variable domain comprises the amino acid sequence of SEQ ID NO:777;
(79) wherein the heavy chain variable domain comprises the amino acid sequence of SEQ ID NO:782 and the light chain variable domain comprises the amino acid sequence of SEQ ID NO:787;
(80) wherein the heavy chain variable domain comprises the amino acid sequence of SEQ ID NO:792 and the light chain variable domain comprises the amino acid sequence of SEQ ID NO:797;
(81) wherein the heavy chain variable domain comprises the amino acid sequence of SEQ ID NO:802 and the light chain variable domain comprises the amino acid sequence of SEQ ID NO:807;
(82) wherein the heavy chain variable domain comprises the amino acid sequence of SEQ ID NO:812 and the light chain variable domain comprises the amino acid sequence of SEQ ID NO:817;
(83) wherein the heavy chain variable domain comprises the amino acid sequence of SEQ ID NO:822 and the light chain variable domain comprises the amino acid sequence of SEQ ID NO:827;
(84) wherein the heavy chain variable domain comprises the amino acid sequence of SEQ ID NO:832 and the light chain variable domain comprises the amino acid sequence of SEQ ID NO:837;
(85) wherein the heavy chain variable domain comprises the amino acid sequence of SEQ ID NO:842 and the light chain variable domain comprises the amino acid sequence of SEQ ID NO:847;
(86) wherein the heavy chain variable domain comprises the amino acid sequence of SEQ ID NO:852 and the light chain variable domain comprises the amino acid sequence of SEQ ID NO:857;
(87) wherein the heavy chain variable domain comprises the amino acid sequence of SEQ ID NO:862 and the light chain variable domain comprises the amino acid sequence of SEQ ID NO:867;
(88) wherein the heavy chain variable domain comprises the amino acid sequence of SEQ ID NO:872 and the light chain variable domain comprises the amino acid sequence of SEQ ID NO:877;
(89) wherein the heavy chain variable domain comprises the amino acid sequence of SEQ ID NO:882 and the light chain variable domain comprises the amino acid sequence of SEQ ID NO:887;
(90) wherein the heavy chain variable domain comprises the amino acid sequence of SEQ ID NO:892 and the light chain variable domain comprises the amino acid sequence of SEQ ID NO:897;
(91) wherein the heavy chain variable domain comprises the amino acid sequence of SEQ ID NO:902 and the light chain variable domain comprises the amino acid sequence of SEQ ID NO:907;
(92) wherein the heavy chain variable domain comprises the amino acid sequence of SEQ ID NO:912 and the light chain variable domain comprises the amino acid sequence of SEQ ID NO:917;
(93) wherein the heavy chain variable domain comprises the amino acid sequence of SEQ ID NO:922 and the light chain variable domain comprises the amino acid sequence of SEQ ID NO:927;
(94) wherein the heavy chain variable domain comprises the amino acid sequence of SEQ ID NO:932 and the light chain variable domain comprises the amino acid sequence of SEQ ID NO:937;
(95) wherein the heavy chain variable domain comprises the amino acid sequence of SEQ ID NO:942 and the light chain variable domain comprises the amino acid sequence of SEQ ID NO:947;
(96) wherein the heavy chain variable domain comprises the amino acid sequence of SEQ ID NO:952 and the light chain variable domain comprises the amino acid sequence of SEQ ID NO:957;
(97) wherein the heavy chain variable domain comprises the amino acid sequence of SEQ ID NO:962 and the light chain variable domain comprises the amino acid sequence of SEQ ID NO:967;
(98) wherein the heavy chain variable domain comprises the amino acid sequence of SEQ ID NO:972 and the light chain variable domain comprises the amino acid sequence of SEQ ID NO:977; or
(99) wherein the heavy chain variable domain comprises the amino acid sequence of SEQ ID NO:982 and the light chain variable domain comprises the amino acid sequence of SEQ ID NO:987.

3. The isolated recombinant monoclonal antibody, or antigen-binding fragment thereof, of claim 1, wherein the coronavirus is Severe Acute Respiratory Syndrome (SARS)-CoV-2.

4. The isolated recombinant monoclonal antibody, or antigen-binding fragment thereof, of claim 1, wherein
(a) the coronavirus is SARS-CoV-2 and/or SARS-CoV-1 and the antibody is selected from one or more of the antibodies defined in (2)-(4), (10), (19), (21) (23) (24), (26), (27), (29), (30) (32)-(41), (44), (46)-(48), (50), (51), (53)-(60), (62), (63), (65), (68), (70)-(73), (76)-(79), (81)-(84), (86), (92), (95), (96), and (99);
(b) the coronavirus is SARS-CoV-2 and/or MERS-CoV and the antibody is selected from one or more of the antibodies defined in (1)-(4), (7), (9)-(41), (44)-(45), (46), (50)-(59), (70), (76), (82), (92), (96), and (99);
(c) the coronavirus is SARS-CoV-2 and/or hCoV-HKU1 and the antibody is selected from one or more of the antibodies defined in (10)-(4), (9), (10), (14), (19), (23), (27)-(29) (32)-(41), (44), (46), (48), (50)-(59), (70), (76), (79), (82), (92), (96), and (99);
(d) the coronavirus is SARS-CoV-2 and/or hCoV-OC43 and the antibody is selected from one or more of the antibodies defined in (2)-(4), (6), (19), (23) (32)-(41), (44), (46), (48), (50)-(60), (63), (70), (76), (79), (81), (82), (92), (96), (97), and (99);
(e) the coronavirus is SARS-CoV-2 and/or hCoV-NL63 and the antibody is selected from one or more of the antibodies defined in (2)-(4), (10), (19), (23), (27)-(29) (32)-(41), (44), (46), (48), (50)-(60), (63), (70), (76), (81), (82), (92), (96), (97), and (99); and/or
(f) the coronavirus is SARS-CoV-2 and/or hCoV-229E and the antibody is selected from one or more of the antibodies defined in (2)-(4), (10), (19), (23), (24), (27)-(29) (32)-(41), (44), (50)-(59), (70), (76), (82), (92), (96), and (99).

5. (canceled)

6. The isolated recombinant monoclonal antibody, or antigen-binding fragment thereof, of claim 1, wherein the antibody binds to at least one of an N terminal domain (NTD) and/or a receptor binding domain (RBD) of a coronavirus spike protein.

7. The isolated recombinant monoclonal antibody, or antigen-binding fragment thereof, of claim 1, wherein the antibody binds to an S1 or S2 subunit of a coronavirus spike protein.

8. The isolated recombinant monoclonal antibody, or antigen-binding fragment thereof, of claim 1, wherein the antibody binds to a coronavirus with a dissociation constant (K_{D}) equal to or less than 50 μM (10⁻¹¹M), as measured by BioLayer Interferometry.

9. (canceled)

10. The isolated recombinant monoclonal antibody, or antigen-binding fragment thereof, of claim 1, wherein the antibody is a human antibody.

11. The isolated recombinant monoclonal antibody, or antigen-binding fragment thereof, of claim 1, wherein the antibody is an IgG or IgM antibody.

12. The isolated recombinant monoclonal antibody, or antigen-binding fragment thereof, of claim 1, wherein the antibody is expressed from a transgenic animal or is expressed in vitro from an insect cell or a mammalian cell, such as a Chinese Hamster Ovary cell or a Human Embryonic Kidney 293T cell.

13. (canceled)

14. (canceled)

15. The isolated recombinant monoclonal antibody, or antigen-binding fragment thereof, of claim 1, further comprising a human constant region, wherein a source of the heavy chain variable domain and the light chain variable domain is different from a source of the human constant region.

16. (canceled)

17. A bispecific antibody comprising a first antigen-binding fragment of a first recombinant monoclonal antibody of claim 1, wherein:
(a) the first recombinant monoclonal antibody binds to a receptor-binding domain of the S1 subunit of SARS-CoV2 and the second recombinant monoclonal antibody binds to the N-terminal domain of the S1 subunit of SARS-CoV2;
(b) the first recombinant monoclonal antibody binds to a receptor-binding domain of the S1 subunit of SARS-CoV2 and the second recombinant monoclonal antibody binds to an S2 subunit of SARS-CoV2;
(c) the first recombinant monoclonal antibody binds to the N-terminal domain of the S1 subunit of SARS-CoV2 and the second recombinant monoclonal antibody binds to an S2 subunit of SARS-CoV2;
(d) the first recombinant monoclonal antibody binds to a receptor-binding domain of the S1 subunit of SARS-CoV2 and the second recombinant monoclonal antibody binds to the receptor-binding domain of the S1 subunit of SARS-CoV2, wherein the first and second recombinant monoclonal antibodies are not the same monoclonal antibody;
(e) the first recombinant monoclonal antibody binds to the N-terminal domain of the S1 subunit of SARS-CoV2 and the second recombinant monoclonal antibody binds to the N-terminal domain of the S1 subunit of SARS-CoV2, wherein the first and second recombinant monoclonal antibodies are not the same monoclonal antibody;
(f) the first recombinant monoclonal antibody binds to an S2 subunit of SARS-CoV2 and the second recombinant monoclonal antibody binds to an S2 subunit of SARS-CoV2, wherein the first and second recombinant monoclonal antibodies are not the same monoclonal antibody;
(g) the first recombinant monoclonal antibody binds to an binds to an S1 subunit of SARS-CoV2 and the second recombinant monoclonal antibody binds to an S1 subunit of SARS-CoV2, wherein the first and second recombinant monoclonal antibodies are not the same monoclonal antibody; or
(h) the first recombinant monoclonal antibody binds to an binds to an S1 subunit of SARS-CoV2 and the second recombinant monoclonal antibody binds to an S2 subunit of SARS-CoV2, wherein the first and second recombinant monoclonal antibodies are not the same monoclonal antibody.

18. A composition comprising one or more of the isolated recombinant monoclonal antibodies, or antigen-binding fragment thereof, of claim 1.

19. The composition of claim 18, comprising:
(a) at least a first recombinant monoclonal antibody, or antigen-binding fragment thereof, wherein the first recombinant monoclonal antibody, or antigen-binding fragment thereof, binds to a receptor-binding domain of the S1 subunit of SARS-CoV2 and the second recombinant monoclonal antibody, or antigen-binding fragment thereof, binds to the N-terminal domain of the S1 subunit of SARS-CoV2;
(b) at least a first recombinant monoclonal antibody, or antigen-binding fragment thereof, wherein the first recombinant monoclonal antibody, or antigen-binding fragment thereof, binds to a receptor-binding domain of the S1 subunit of SARS-CoV2 and the second recombinant monoclonal antibody, or antigen-binding fragment thereof, binds to an S2 subunit of SARS-CoV2;
(c) at least a first recombinant monoclonal antibody, or antigen-binding fragment thereof, wherein the first recombinant monoclonal antibody, or antigen-binding fragment thereof, binds to the N-terminal domain of the S1 subunit of SARS-CoV2 and the second recombinant monoclonal antibody, or antigen-binding fragment thereof, binds to an S2 subunit of SARS-CoV2;
(d) at least a first recombinant monoclonal antibody, or antigen-binding fragment thereof, wherein the first recombinant monoclonal antibody, or antigen-binding fragment thereof, binds to a receptor-binding domain of the S1 subunit of SARS-CoV2 and the second recombinant monoclonal antibody, or antigen-binding fragment thereof, binds to a receptor-binding domain of the S1 subunit of SARS-CoV2, wherein the first and second recombinant monoclonal antibodies are not the same monoclonal antibody;
(e) at least a first recombinant monoclonal antibody, or antigen-binding fragment thereof, wherein the first recombinant monoclonal antibody, or antigen-binding fragment thereof, binds to the N-terminal domain of the S1 subunit of SARS-CoV2 and the second recombinant monoclonal antibody, or antigen-binding fragment thereof, binds to the N-terminal domain of the S1 subunit of SARS-CoV2, wherein the first and second recombinant monoclonal antibodies are not the same monoclonal antibody; or
(f) at least a first recombinant monoclonal antibody, or antigen-binding fragment thereof, wherein the first recombinant monoclonal antibody, or antigen-binding fragment thereof, binds to an S2 subunit of SARS-CoV2 and the second recombinant monoclonal antibody, or antigen-binding fragment thereof, binds to an S2 subunit of SARS-CoV2, wherein the first and second recombinant monoclonal antibodies are not the same monoclonal antibody.

20. The bispecific antibody of claim 17, wherein the first recombinant monoclonal antibody, or antigen-binding fragment thereof, binds to a receptor-binding domain of the S1 subunit of SARS-CoV2 and is one of COV_2123, COV_2125, or COV_2173 and the second recombinant monoclonal antibody, or antigen-binding fragment thereof, binds to the N-terminal domain of the S1 subunit of SARS-CoV2 and is one of COV_2004, COV_2025, or COV_2039.

21. (canceled)

22. The composition of claim 18, wherein the composition comprises a therapeutically effective amount of the isolated recombinant monoclonal antibody, or antigen-binding fragment thereof or wherein the composition is formulated for subcutaneous, intravenous, intraarterial, or intramuscular injection.

23. (canceled)

24. An isolated nucleic acid that encodes the amino acid sequence of one or more of the complementarity determining regions (CDRs) of the heavy chain variable domain and/or light chain variable domain of the recombinant, monoclonal antibody, or antigen-binding fragment thereof, of claim 1.

25. An isolated nucleic acid that encodes the amino acid sequence of the heavy and/or light chain variable domain of the recombinant, monoclonal antibody, or antigen-binding fragment thereof, of claim 2, wherein
the isolated nucleic acid encodes the heavy chain variable domain of the antibody defined in (1) and the isolated nucleic acid comprises SEQ ID NO: 1 and/or the isolated nucleic acid encodes the light chain variable domain of the antibody defined in (1) and the isolated nucleic acid comprises SEQ ID NO: 6;
the isolated nucleic acid encodes the heavy chain variable domain of the antibody defined in (2) and the isolated nucleic acid comprises SEQ ID NO: 11 and/or the isolated nucleic acid encodes the light chain variable domain of the antibody defined in (2) and the isolated nucleic acid comprises SEQ ID NO: 16;
the isolated nucleic acid encodes the heavy chain variable domain of the antibody defined in (3) and the isolated nucleic acid comprises SEQ ID NO: 21 and/or the isolated nucleic acid encodes the light chain variable domain of the antibody defined in (3) and the isolated nucleic acid comprises SEQ ID NO: 26;
the isolated nucleic acid encodes the heavy chain variable domain of the antibody defined in (4) and the isolated nucleic acid comprises SEQ ID NO: 31 and/or the isolated nucleic acid encodes the light chain variable domain of the antibody defined in (4) and the isolated nucleic acid comprises SEQ ID NO: 36;
the isolated nucleic acid encodes the heavy chain variable domain of the antibody defined in (5) and the isolated nucleic acid comprises SEQ ID NO: 41 and/or the isolated nucleic acid encodes the light chain variable domain of the antibody defined in (5) and the isolated nucleic acid comprises SEQ ID NO: 46;
the isolated nucleic acid encodes the heavy chain variable domain of the antibody defined in (6) and the isolated nucleic acid comprises SEQ ID NO: 51 and/or the isolated nucleic acid encodes the light chain variable domain of the antibody defined in (6) and the isolated nucleic acid comprises SEQ ID NO: 56;
the isolated nucleic acid encodes the heavy chain variable domain of the antibody defined in (7) and the isolated nucleic acid comprises SEQ ID NO: 61 and/or the isolated nucleic acid encodes the light chain variable domain of the antibody defined in (7) and the isolated nucleic acid comprises SEQ ID NO: 66;
the isolated nucleic acid encodes the heavy chain variable domain of the antibody defined in (8) and the isolated nucleic acid comprises SEQ ID NO: 71 and/or the isolated nucleic acid encodes the light chain variable domain of the antibody defined in (8) and the isolated nucleic acid comprises SEQ ID NO: 76;
the isolated nucleic acid encodes the heavy chain variable domain of the antibody defined in (9) and the isolated nucleic acid comprises SEQ ID NO: 81 and/or the isolated nucleic acid encodes the light chain variable domain of the antibody defined in (9) and the isolated nucleic acid comprises SEQ ID NO: 86;
the isolated nucleic acid encodes the heavy chain variable domain of the antibody defined in (10) and the isolated nucleic acid comprises SEQ ID NO: 91 and/or the isolated nucleic acid encodes the light chain variable domain of the antibody defined in (10) and the isolated nucleic acid comprises SEQ ID NO: 96;
the isolated nucleic acid encodes the heavy chain variable domain of the antibody defined in (11) and the isolated nucleic acid comprises SEQ ID NO: 101 and/or the isolated nucleic acid encodes the light chain variable domain of the antibody defined in (11) and the isolated nucleic acid comprises SEQ ID NO: 106;
the isolated nucleic acid encodes the heavy chain variable domain of the antibody defined in (12) and the isolated nucleic acid comprises SEQ ID NO: 111 and/or the isolated nucleic acid encodes the light chain variable domain of the antibody defined in (12) and the isolated nucleic acid comprises SEQ ID NO: 116;
the isolated nucleic acid encodes the heavy chain variable domain of the antibody defined in (13) and the isolated nucleic acid comprises SEQ ID NO: 121 and/or the isolated nucleic acid encodes the light chain variable domain of the antibody defined in (13) and the isolated nucleic acid comprises SEQ ID NO: 126;
the isolated nucleic acid encodes the heavy chain variable domain of the antibody defined in (14) and the isolated nucleic acid comprises SEQ ID NO: 131 and/or the isolated nucleic acid encodes the light chain variable domain of the antibody defined in (14) and the isolated nucleic acid comprises SEQ ID NO: 136;
the isolated nucleic acid encodes the heavy chain variable domain of the antibody defined in (15) and the isolated nucleic acid comprises SEQ ID NO: 141 and/or the isolated nucleic acid encodes the light chain variable domain of the antibody defined in (15) and the isolated nucleic acid comprises SEQ ID NO: 146;
the isolated nucleic acid encodes the heavy chain variable domain of the antibody defined in (16) and the isolated nucleic acid comprises SEQ ID NO: 151 and/or the isolated nucleic acid encodes the light chain variable domain of the antibody defined in (16) and the isolated nucleic acid comprises SEQ ID NO: 156;
the isolated nucleic acid encodes the heavy chain variable domain of the antibody defined in (17) and the isolated nucleic acid comprises SEQ ID NO: 161 and/or the isolated nucleic acid encodes the light chain variable domain of the antibody defined in (17) and the isolated nucleic acid comprises SEQ ID NO: 166;
the isolated nucleic acid encodes the heavy chain variable domain of the antibody defined in (18) and the isolated nucleic acid comprises SEQ ID NO: 171 and/or the isolated nucleic acid encodes the light chain variable domain of the antibody defined in (18) and the isolated nucleic acid comprises SEQ ID NO: 176;
the isolated nucleic acid encodes the heavy chain variable domain of the antibody defined in (19) and the isolated nucleic acid comprises SEQ ID NO: 181 and/or the isolated nucleic acid encodes the light chain variable domain of the antibody defined in (19) and the isolated nucleic acid comprises SEQ ID NO: 186;
the isolated nucleic acid encodes the heavy chain variable domain of the antibody defined in (20) and the isolated nucleic acid comprises SEQ ID NO: 191 and/or the isolated nucleic acid encodes the light chain variable domain of the antibody defined in (20) and the isolated nucleic acid comprises SEQ ID NO: 196;
the isolated nucleic acid encodes the heavy chain variable domain of the antibody defined in (21) and the isolated nucleic acid comprises SEQ ID NO: 201 and/or the isolated nucleic acid encodes the light chain variable domain of the antibody defined in (21) and the isolated nucleic acid comprises SEQ ID NO: 206;
the isolated nucleic acid encodes the heavy chain variable domain of the antibody defined in (22) and the isolated nucleic acid comprises SEQ ID NO: 211 and/or the isolated nucleic acid encodes the light chain variable domain of the antibody defined in (22) and the isolated nucleic acid comprises SEQ ID NO: 216;
the isolated nucleic acid encodes the heavy chain variable domain of the antibody defined in (23) and the isolated nucleic acid comprises SEQ ID NO: 221 and/or the isolated nucleic acid encodes the light chain variable domain of the antibody defined in (23) and the isolated nucleic acid comprises SEQ ID NO: 226;
the isolated nucleic acid encodes the heavy chain variable domain of the antibody defined in (24) and the isolated nucleic acid comprises SEQ ID NO: 231 and/or the isolated nucleic acid encodes the light chain variable domain of the antibody defined in (24) and the isolated nucleic acid comprises SEQ ID NO: 236;
the isolated nucleic acid encodes the heavy chain variable domain of the antibody defined in (25) and the isolated nucleic acid comprises SEQ ID NO: 241 and/or the isolated nucleic acid encodes the light chain variable domain of the antibody defined in (25) and the isolated nucleic acid comprises SEQ ID NO: 246;
the isolated nucleic acid encodes the heavy chain variable domain of the antibody defined in (26) and the isolated nucleic acid comprises SEQ ID NO: 251 and/or the isolated nucleic acid encodes the light chain variable domain of the antibody defined in (26) and the isolated nucleic acid comprises SEQ ID NO: 256;
the isolated nucleic acid encodes the heavy chain variable domain of the antibody defined in (27) and the isolated nucleic acid comprises SEQ ID NO: 261 and/or the isolated nucleic acid encodes the light chain variable domain of the antibody defined in (27) and the isolated nucleic acid comprises SEQ ID NO: 266;
the isolated nucleic acid encodes the heavy chain variable domain of the antibody defined in (28) and the isolated nucleic acid comprises SEQ ID NO: 271 and/or the isolated nucleic acid encodes the light chain variable domain of the antibody defined in (28) and the isolated nucleic acid comprises SEQ ID NO: 276;
the isolated nucleic acid encodes the heavy chain variable domain of the antibody defined in (29) and the isolated nucleic acid comprises SEQ ID NO: 281 and/or the isolated nucleic acid encodes the light chain variable domain of the antibody defined in (29) and the isolated nucleic acid comprises SEQ ID NO: 286;
the isolated nucleic acid encodes the heavy chain variable domain of the antibody defined in (30) and the isolated nucleic acid comprises SEQ ID NO: 291 and/or the isolated nucleic acid encodes the light chain variable domain of the antibody defined in (30) and the isolated nucleic acid comprises SEQ ID NO: 296;
the isolated nucleic acid encodes the heavy chain variable domain of the antibody defined in (31) and the isolated nucleic acid comprises SEQ ID NO: 301 and/or the isolated nucleic acid encodes the light chain variable domain of the antibody defined in (31) and the isolated nucleic acid comprises SEQ ID NO: 306;
the isolated nucleic acid encodes the heavy chain variable domain of the antibody defined in (32) and the isolated nucleic acid comprises SEQ ID NO: 311 and/or the isolated nucleic acid encodes the light chain variable domain of the antibody defined in (32) and the isolated nucleic acid comprises SEQ ID NO: 316;
the isolated nucleic acid encodes the heavy chain variable domain of the antibody defined in (33) and the isolated nucleic acid comprises SEQ ID NO: 321 and/or the isolated nucleic acid encodes the light chain variable domain of the antibody defined in (33) and the isolated nucleic acid comprises SEQ ID NO: 326;
the isolated nucleic acid encodes the heavy chain variable domain of the antibody defined in (34) and the isolated nucleic acid comprises SEQ ID NO: 331 and/or the isolated nucleic acid encodes the light chain variable domain of the antibody defined in (34) and the isolated nucleic acid comprises SEQ ID NO: 336;
the isolated nucleic acid encodes the heavy chain variable domain of the antibody defined in (35) and the isolated nucleic acid comprises SEQ ID NO: 341 and/or the isolated nucleic acid encodes the light chain variable domain of the antibody defined in (35) and the isolated nucleic acid comprises SEQ ID NO: 346;
the isolated nucleic acid encodes the heavy chain variable domain of the antibody defined in (36) and the isolated nucleic acid comprises SEQ ID NO: 351 and/or the isolated nucleic acid encodes the light chain variable domain of the antibody defined in (36) and the isolated nucleic acid comprises SEQ ID NO: 356;
the isolated nucleic acid encodes the heavy chain variable domain of the antibody defined in (37) and the isolated nucleic acid comprises SEQ ID NO: 361 and/or the isolated nucleic acid encodes the light chain variable domain of the antibody defined in (37) and the isolated nucleic acid comprises SEQ ID NO: 366;
the isolated nucleic acid encodes the heavy chain variable domain of the antibody defined in (38) and the isolated nucleic acid comprises SEQ ID NO: 371 and/or the isolated nucleic acid encodes the light chain variable domain of the antibody defined in (38) and the isolated nucleic acid comprises SEQ ID NO: 376;
the isolated nucleic acid encodes the heavy chain variable domain of the antibody defined in (39) and the isolated nucleic acid comprises SEQ ID NO: 381 and/or the isolated nucleic acid encodes the light chain variable domain of the antibody defined in (39) and the isolated nucleic acid comprises SEQ ID NO: 386;
the isolated nucleic acid encodes the heavy chain variable domain of the antibody defined in (40) and the isolated nucleic acid comprises SEQ ID NO: 391 and/or the isolated nucleic acid encodes the light chain variable domain of the antibody defined in (40) and the isolated nucleic acid comprises SEQ ID NO: 396;
the isolated nucleic acid encodes the heavy chain variable domain of the antibody defined in (41) and the isolated nucleic acid comprises SEQ ID NO: 401 and/or the isolated nucleic acid encodes the light chain variable domain of the antibody defined in (41) and the isolated nucleic acid comprises SEQ ID NO: 406;
the isolated nucleic acid encodes the heavy chain variable domain of the antibody defined in (42) and the isolated nucleic acid comprises SEQ ID NO: 411 and/or the isolated nucleic acid encodes the light chain variable domain of the antibody defined in (42) and the isolated nucleic acid comprises SEQ ID NO: 416;
the isolated nucleic acid encodes the heavy chain variable domain of the antibody defined in (43) and the isolated nucleic acid comprises SEQ ID NO: 421 and/or the isolated nucleic acid encodes the light chain variable domain of the antibody defined in (43) and the isolated nucleic acid comprises SEQ ID NO: 426;
the isolated nucleic acid encodes the heavy chain variable domain of the antibody defined in (44) and the isolated nucleic acid comprises SEQ ID NO: 431 and/or the isolated nucleic acid encodes the light chain variable domain of the antibody defined in (44) and the isolated nucleic acid comprises SEQ ID NO: 436;
the isolated nucleic acid encodes the heavy chain variable domain of the antibody defined in (45) and the isolated nucleic acid comprises SEQ ID NO: 441 and/or the isolated nucleic acid encodes the light chain variable domain of the antibody defined in (45) and the isolated nucleic acid comprises SEQ ID NO: 446;
the isolated nucleic acid encodes the heavy chain variable domain of the antibody defined in (46) and the isolated nucleic acid comprises SEQ ID NO: 451 and/or the isolated nucleic acid encodes the light chain variable domain of the antibody defined in (46) and the isolated nucleic acid comprises SEQ ID NO: 456;
the isolated nucleic acid encodes the heavy chain variable domain of the antibody defined in (47) and the isolated nucleic acid comprises SEQ ID NO: 461 and/or the isolated nucleic acid encodes the light chain variable domain of the antibody defined in (47) and the isolated nucleic acid comprises SEQ ID NO: 466;
the isolated nucleic acid encodes the heavy chain variable domain of the antibody defined in (48) and the isolated nucleic acid comprises SEQ ID NO: 471 and/or the isolated nucleic acid encodes the light chain variable domain of the antibody defined in (48) and the isolated nucleic acid comprises SEQ ID NO: 476;
the isolated nucleic acid encodes the heavy chain variable domain of the antibody defined in (49) and the isolated nucleic acid comprises SEQ ID NO: 481 and/or the isolated nucleic acid encodes the light chain variable domain of the antibody defined in (49) and the isolated nucleic acid comprises SEQ ID NO: 486;
the isolated nucleic acid encodes the heavy chain variable domain of the antibody defined in (50) and the isolated nucleic acid comprises SEQ ID NO: 491 and/or the isolated nucleic acid encodes the light chain variable domain of the antibody defined in (50) and the isolated nucleic acid comprises SEQ ID NO: 496;
the isolated nucleic acid encodes the heavy chain variable domain of the antibody defined in (51) and the isolated nucleic acid comprises SEQ ID NO: 501 and/or the isolated nucleic acid encodes the light chain variable domain of the antibody defined in (51) and the isolated nucleic acid comprises SEQ ID NO: 506;
the isolated nucleic acid encodes the heavy chain variable domain of the antibody defined in (52) and the isolated nucleic acid comprises SEQ ID NO: 511 and/or the isolated nucleic acid encodes the light chain variable domain of the antibody defined in (52) and the isolated nucleic acid comprises SEQ ID NO: 516;
the isolated nucleic acid encodes the heavy chain variable domain of the antibody defined in (53) and the isolated nucleic acid comprises SEQ ID NO: 521 and/or the isolated nucleic acid encodes the light chain variable domain of the antibody defined in (53) and the isolated nucleic acid comprises SEQ ID NO: 526;
the isolated nucleic acid encodes the heavy chain variable domain of the antibody defined in (54) and the isolated nucleic acid comprises SEQ ID NO: 531 and/or the isolated nucleic acid encodes the light chain variable domain of the antibody defined in (54) and the isolated nucleic acid comprises SEQ ID NO: 536;
the isolated nucleic acid encodes the heavy chain variable domain of the antibody defined in (55) and the isolated nucleic acid comprises SEQ ID NO: 541 and/or the isolated nucleic acid encodes the light chain variable domain of the antibody defined in (55) and the isolated nucleic acid comprises SEQ ID NO: 546;
the isolated nucleic acid encodes the heavy chain variable domain of the antibody defined in (56) and the isolated nucleic acid comprises SEQ ID NO: 551 and/or the isolated nucleic acid encodes the light chain variable domain of the antibody defined in (56) and the isolated nucleic acid comprises SEQ ID NO: 556;
the isolated nucleic acid encodes the heavy chain variable domain of the antibody defined in (57) and the isolated nucleic acid comprises SEQ ID NO: 561 and/or the isolated nucleic acid encodes the light chain variable domain of the antibody defined in (57) and the isolated nucleic acid comprises SEQ ID NO: 566;
the isolated nucleic acid encodes the heavy chain variable domain of the antibody defined in (58) and the isolated nucleic acid comprises SEQ ID NO: 571 and/or the isolated nucleic acid encodes the light chain variable domain of the antibody defined in (58) and the isolated nucleic acid comprises SEQ ID NO: 576;
the isolated nucleic acid encodes the heavy chain variable domain of the antibody defined in (59) and the isolated nucleic acid comprises SEQ ID NO: 581 and/or the isolated nucleic acid encodes the light chain variable domain of the antibody defined in (59) and the isolated nucleic acid comprises SEQ ID NO: 586;
the isolated nucleic acid encodes the heavy chain variable domain of the antibody defined in (60) and the isolated nucleic acid comprises SEQ ID NO: 591 and/or the isolated nucleic acid encodes the light chain variable domain of the antibody defined in (60) and the isolated nucleic acid comprises SEQ ID NO: 596;
the isolated nucleic acid encodes the heavy chain variable domain of the antibody defined in (61) and the isolated nucleic acid comprises SEQ ID NO: 601 and/or the isolated nucleic acid encodes the light chain variable domain of the antibody defined in (61) and the isolated nucleic acid comprises SEQ ID NO: 606;
the isolated nucleic acid encodes the heavy chain variable domain of the antibody defined in (62) and the isolated nucleic acid comprises SEQ ID NO: 611 and/or the isolated nucleic acid encodes the light chain variable domain of the antibody defined in (62) and the isolated nucleic acid comprises SEQ ID NO: 616;
the isolated nucleic acid encodes the heavy chain variable domain of the antibody defined in (63) and the isolated nucleic acid comprises SEQ ID NO: 621 and/or the isolated nucleic acid encodes the light chain variable domain of the antibody defined in (63) and the isolated nucleic acid comprises SEQ ID NO: 626;
the isolated nucleic acid encodes the heavy chain variable domain of the antibody defined in (64) and the isolated nucleic acid comprises SEQ ID NO: 631 and/or the isolated nucleic acid encodes the light chain variable domain of the antibody defined in (64) and the isolated nucleic acid comprises SEQ ID NO: 636;
the isolated nucleic acid encodes the heavy chain variable domain of the antibody defined in (65) and the isolated nucleic acid comprises SEQ ID NO: 641 and/or the isolated nucleic acid encodes the light chain variable domain of the antibody defined in (65) and the isolated nucleic acid comprises SEQ ID NO: 646;
the isolated nucleic acid encodes the heavy chain variable domain of the antibody defined in (66) and the isolated nucleic acid comprises SEQ ID NO: 651 and/or the isolated nucleic acid encodes the light chain variable domain of the antibody defined in (66) and the isolated nucleic acid comprises SEQ ID NO: 656;
the isolated nucleic acid encodes the heavy chain variable domain of the antibody defined in (67) and the isolated nucleic acid comprises SEQ ID NO: 661 and/or the isolated nucleic acid encodes the light chain variable domain of the antibody defined in (67) and the isolated nucleic acid comprises SEQ ID NO: 666;
the isolated nucleic acid encodes the heavy chain variable domain of the antibody defined in (68) and the isolated nucleic acid comprises SEQ ID NO: 671 and/or the isolated nucleic acid encodes the light chain variable domain of the antibody defined in (68) and the isolated nucleic acid comprises SEQ ID NO: 676;
the isolated nucleic acid encodes the heavy chain variable domain of the antibody defined in (69) and the isolated nucleic acid comprises SEQ ID NO: 681 and/or the isolated nucleic acid encodes the light chain variable domain of the antibody defined in (69) and the isolated nucleic acid comprises SEQ ID NO: 686;
the isolated nucleic acid encodes the heavy chain variable domain of the antibody defined in (70) and the isolated nucleic acid comprises SEQ ID NO: 691 and/or the isolated nucleic acid encodes the light chain variable domain of the antibody defined in (70) and the isolated nucleic acid comprises SEQ ID NO: 696;
the isolated nucleic acid encodes the heavy chain variable domain of the antibody defined in (71) and the isolated nucleic acid comprises SEQ ID NO: 701 and/or the isolated nucleic acid encodes the light chain variable domain of the antibody defined in (71) and the isolated nucleic acid comprises SEQ ID NO: 706;
the isolated nucleic acid encodes the heavy chain variable domain of the antibody defined in (72) and the isolated nucleic acid comprises SEQ ID NO: 711 and/or the isolated nucleic acid encodes the light chain variable domain of the antibody defined in (72) and the isolated nucleic acid comprises SEQ ID NO: 716;
the isolated nucleic acid encodes the heavy chain variable domain of the antibody defined in (73) and the isolated nucleic acid comprises SEQ ID NO: 721 and/or the isolated nucleic acid encodes the light chain variable domain of the antibody defined in (73) and the isolated nucleic acid comprises SEQ ID NO: 726;
the isolated nucleic acid encodes the heavy chain variable domain of the antibody defined in (74) and the isolated nucleic acid comprises SEQ ID NO: 731 and/or the isolated nucleic acid encodes the light chain variable domain of the antibody defined in (74) and the isolated nucleic acid comprises SEQ ID NO: 736;
the isolated nucleic acid encodes the heavy chain variable domain of the antibody defined in (75) and the isolated nucleic acid comprises SEQ ID NO: 741 and/or the isolated nucleic acid encodes the light chain variable domain of the antibody defined in (75) and the isolated nucleic acid comprises SEQ ID NO: 746;
the isolated nucleic acid encodes the heavy chain variable domain of the antibody defined in (76) and the isolated nucleic acid comprises SEQ ID NO: 751 and/or the isolated nucleic acid encodes the light chain variable domain of the antibody defined in (76) and the isolated nucleic acid comprises SEQ ID NO: 756;
the isolated nucleic acid encodes the heavy chain variable domain of the antibody defined in (77) and the isolated nucleic acid comprises SEQ ID NO: 761 and/or the isolated nucleic acid encodes the light chain variable domain of the antibody defined in (77) and the isolated nucleic acid comprises SEQ ID NO: 766;
the isolated nucleic acid encodes the heavy chain variable domain of the antibody defined in (78) and the isolated nucleic acid comprises SEQ ID NO: 771 and/or the isolated nucleic acid encodes the light chain variable domain of the antibody defined in (78) and the isolated nucleic acid comprises SEQ ID NO: 776;
the isolated nucleic acid encodes the heavy chain variable domain of the antibody defined in (79) and the isolated nucleic acid comprises SEQ ID NO: 781 and/or the isolated nucleic acid encodes the light chain variable domain of the antibody defined in (79) and the isolated nucleic acid comprises SEQ ID NO: 786;
the isolated nucleic acid encodes the heavy chain variable domain of the antibody defined in (80) and the isolated nucleic acid comprises SEQ ID NO: 791 and/or the isolated nucleic acid encodes the light chain variable domain of the antibody defined in (80) and the isolated nucleic acid comprises SEQ ID NO: 796;
the isolated nucleic acid encodes the heavy chain variable domain of the antibody defined in (81) and the isolated nucleic acid comprises SEQ ID NO: 801 and/or the isolated nucleic acid encodes the light chain variable domain of the antibody defined in (81) and the isolated nucleic acid comprises SEQ ID NO: 806;
the isolated nucleic acid encodes the heavy chain variable domain of the antibody defined in (82) and the isolated nucleic acid comprises SEQ ID NO: 811 and/or the isolated nucleic acid encodes the light chain variable domain of the antibody defined in (82) and the isolated nucleic acid comprises SEQ ID NO: 816;
the isolated nucleic acid encodes the heavy chain variable domain of the antibody defined in (83) and the isolated nucleic acid comprises SEQ ID NO: 821 and/or the isolated nucleic acid encodes the light chain variable domain of the antibody defined in (83) and the isolated nucleic acid comprises SEQ ID NO: 826;
the isolated nucleic acid encodes the heavy chain variable domain of the antibody defined in (84) and the isolated nucleic acid comprises SEQ ID NO: 831 and/or the isolated nucleic acid encodes the light chain variable domain of the antibody defined in (84) and the isolated nucleic acid comprises SEQ ID NO: 836;
the isolated nucleic acid encodes the heavy chain variable domain of the antibody defined in (85) and the isolated nucleic acid comprises SEQ ID NO: 841 and/or the isolated nucleic acid encodes the light chain variable domain of the antibody defined in (85) and the isolated nucleic acid comprises SEQ ID NO: 846;
the isolated nucleic acid encodes the heavy chain variable domain of the antibody defined in (86) and the isolated nucleic acid comprises SEQ ID NO: 851 and/or the isolated nucleic acid encodes the light chain variable domain of the antibody defined in (86) and the isolated nucleic acid comprises SEQ ID NO: 856;
the isolated nucleic acid encodes the heavy chain variable domain of the antibody defined in (87) and the isolated nucleic acid comprises SEQ ID NO: 861 and/or the isolated nucleic acid encodes the light chain variable domain of the antibody defined in (87) and the isolated nucleic acid comprises SEQ ID NO: 866;
the isolated nucleic acid encodes the heavy chain variable domain of the antibody defined in (88) and the isolated nucleic acid comprises SEQ ID NO: 871 and/or the isolated nucleic acid encodes the light chain variable domain of the antibody defined in (88) and the isolated nucleic acid comprises SEQ ID NO: 876;
the isolated nucleic acid encodes the heavy chain variable domain of the antibody defined in (89) and the isolated nucleic acid comprises SEQ ID NO: 881 and/or the isolated nucleic acid encodes the light chain variable domain of the antibody defined in (89) and the isolated nucleic acid comprises SEQ ID NO: 886;
the isolated nucleic acid encodes the heavy chain variable domain of the antibody defined in (90) and the isolated nucleic acid comprises SEQ ID NO: 891 and/or the isolated nucleic acid encodes the light chain variable domain of the antibody defined in (90) and the isolated nucleic acid comprises SEQ ID NO: 896;
the isolated nucleic acid encodes the heavy chain variable domain of the antibody defined in (91) and the isolated nucleic acid comprises SEQ ID NO: 901 and/or the isolated nucleic acid encodes the light chain variable domain of the antibody defined in (91) and the isolated nucleic acid comprises SEQ ID NO: 906;
the isolated nucleic acid encodes the heavy chain variable domain of the antibody defined in (92) and the isolated nucleic acid comprises SEQ ID NO: 911 and/or the isolated nucleic acid encodes the light chain variable domain of the antibody defined in (92) and the isolated nucleic acid comprises SEQ ID NO: 916;
the isolated nucleic acid encodes the heavy chain variable domain of the antibody defined in (93) and the isolated nucleic acid comprises SEQ ID NO: 921 and/or the isolated nucleic acid encodes the light chain variable domain of the antibody defined in (93) and the isolated nucleic acid comprises SEQ ID NO: 926;
the isolated nucleic acid encodes the heavy chain variable domain of the antibody defined in (94) and the isolated nucleic acid comprises SEQ ID NO: 931 and/or the isolated nucleic acid encodes the light chain variable domain of the antibody defined in (94) and the isolated nucleic acid comprises SEQ ID NO: 936;
the isolated nucleic acid encodes the heavy chain variable domain of the antibody defined in (95) and the isolated nucleic acid comprises SEQ ID NO: 941 and/or the isolated nucleic acid encodes the light chain variable domain of the antibody defined in (95) and the isolated nucleic acid comprises SEQ ID NO: 946;
the isolated nucleic acid encodes the heavy chain variable domain of the antibody defined in (96) and the isolated nucleic acid comprises SEQ ID NO: 951 and/or the isolated nucleic acid encodes the light chain variable domain of the antibody defined in (96) and the isolated nucleic acid comprises SEQ ID NO: 956;
the isolated nucleic acid encodes the heavy chain variable domain of the antibody defined in (97) and the isolated nucleic acid comprises SEQ ID NO: 961 and/or the isolated nucleic acid encodes the light chain variable domain of the antibody defined in (97) and the isolated nucleic acid comprises SEQ ID NO: 966;
the isolated nucleic acid encodes the heavy chain variable domain of the antibody defined in (98) and the isolated nucleic acid comprises SEQ ID NO: 971 and/or the isolated nucleic acid encodes the light chain variable domain of the antibody defined in (98) and the isolated nucleic acid comprises SEQ ID NO: 976; and/or
the isolated nucleic acid encodes the heavy chain variable domain of the antibody defined in (99) and the isolated nucleic acid comprises SEQ ID NO: 981 and/or the isolated nucleic acid encodes the light chain variable domain of the antibody defined in (99) and the isolated nucleic acid comprises SEQ ID NO: 986.

26. A recombinant expression vector comprising the nucleic acid of claim 24.

27. An isolated host cell comprising the recombinant expression vector of claim 26.

28. A method of preventing or treating a coronavirus infection, comprising administering to a subject one or more of the antibodies, or antigen-binding fragments thereof, of claim 1 in an amount effective to treat, prevent, or diagnose the coronavirus infection.

29. The method of claim 28, wherein the subject is a human.

30. The method of claim 28, wherein at least two of the antibodies or antigen-binding fragments thereof are administered to the subject or wherein the at least two antibodies or antigen-binding fragments thereof comprise an anti-RBD antibody or antigen binding-fragment thereof and an anti-NTD antibody or antigen binding fragment thereof.

31. (canceled)

32. The method of claim 30, wherein the anti-RBD antibody or binding-fragment thereof comprises:
a heavy chain variable domain comprising a CDR1 comprising the amino acid sequence of SEQ ID NO:233, a CDR2 comprising the amino acid sequence of SEQ ID NO:234, and a CDR3 comprising the amino acid sequence of SEQ ID NO:235 and a light chain variable domain comprising a CDR1 comprising the amino acid sequence of SEQ ID NO:238, a CDR2 comprising the amino acid sequence of SEQ ID NO:239, and a CDR3 comprising the amino acid sequence of SEQ ID NO:240 or
wherein the anti-RBD antibody or binding-fragment thereof comprises a heavy chain variable domain comprising a CDR1 comprising the amino acid sequence of SEQ ID NO:243, a CDR2 comprising the amino acid sequence of SEQ ID NO:244, and a CDR3 comprising the amino acid sequence of SEQ ID NO:245 and a light chain variable domain comprising a CDR1 comprising the amino acid sequence of SEQ ID NO:248, a CDR2 comprising the amino acid sequence of SEQ ID NO:249, and a CDR3 comprising the amino acid sequence of SEQ ID NO:250 and
wherein the anti-NTD antibody or antigen-binding fragment thereof comprises a heavy chain variable domain comprising a CDR1 comprising the amino acid sequence of SEQ ID NO:152, a CDR2 comprising the amino acid sequence of SEQ ID NO:153, and a CDR3 comprising the amino acid sequence of SEQ ID NO:154 and a light chain variable domain comprising a CDR1 comprising the amino acid sequence of SEQ ID NO:158, a CDR2 comprising the amino acid sequence of SEQ ID NO:159, and a CDR3 comprising the amino acid sequence of SEQ ID NO:160.

33. The method of claim 30, wherein the at least two antibodies comprise COV_2173 and COV_2039.

34. (canceled)

35. A method of diagnosing a coronavirus infection comprising the steps of:
contacting a sample from a subject suspected of having a coronavirus infection with one or more antibodies, or antigen-binding fragment thereof, of claim 1; and
determining the presence of the coronavirus infection upon detection of antibody binding, optionally wherein the coronavirus infection is a SARS-CoV-2 virus infection and optionally wherein the one or more antibodies, or antigen-binding fragment thereof.
